(19) United States
(12) Patent Application Publication
(10) Pub. No.: US 2024/0368131 A1
(43) Pub. Date: Nov. 7, 2024

(54) OXOINDOLINYL AMIDE DERIVATIVES FOR INHIBITING NLRP3 AND USES THEREOF

(71) Applicant: VENTUS THERAPEUTICS U.S. INC., Waltham, MA (US)

(72) Inventors: Stéphane Dorich, Pointe-Claire (CA); Miguel ST-ONGE, Saint-Lazare (CA); Jason Burch, Pointe-Claire (CA); Ramsay Beveridge, Pointe-Claire (CA); Stéphane Ciblat, Montreal (CA); Alexandre CÔTÉ, Laval (CA); Patrick Cyr, Saint-Constant (CA)

(21) Appl. No.: 18/754,527

(22) Filed: Jun. 26, 2024

Related U.S. Application Data
(63) Continuation of application No. PCT/US2023/066934, filed on May 12, 2023.
(60) Provisional application No. 63/341,614, filed on May 13, 2022.

(51) Int. Cl. C07D 403/12; A61K 31/4015; A61K 31/404; A61K 31/41; A61K 31/4439; C07D 209/34; C07D 401/12; C07D 401/14; C07D 405/12

(52) U.S. Cl. CPC C07D 403/12; A61K 31/4015; A61K 31/404; A61K 31/41; A61K 31/4439; C07D 209/34; C07D 401/12; C07D 401/14; C07D 405/12; C07B 2200/09

(57) ABSTRACT

The present disclosure relates to compounds of Formula (I):

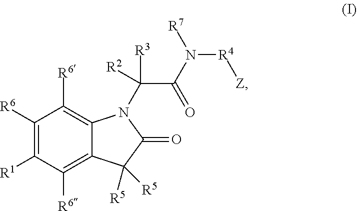

or a pharmaceutically acceptable salt, solvate, clathrate, hydrate, stereoisomer, tautomer, isotopic derivative, prodrug or polymorph thereof. Further provided are pharmaceutical compositions comprising the same, methods of preparation, and methods of use and treatment, e.g., as inhibitors of NLRP3 useful in the treatment of diseases and disorders inhibited by said protein.

### RELATED APPLICATIONS

This application is a continuation of International Application No. PCT/US2023/066934, filed May 12, 2023, which claims the benefit of U.S. Provisional Patent Application No. 63/341,614, filed on May 13, 2022, the entire contents of which is incorporated herein by reference in its entirety.

### BACKGROUND

Innate immune responses are mediated by different types of receptors termed pattern-recognition receptors (PRRs). PRRs recognize the presence of pathogen-associated molecular patterns (PAMPs) and damage-associated molecular patterns (DAMPs). Once engaged these receptors trigger the activation of downstream inflammatory pathways that will help resolve injury. However, in many instances this activation can be uncontrolled and leads to disease.

The inflammasomes represent a class of PRRs that are crucial components of the innate immune response. Activation of the inflammasomes trigger a cascade of events that releases IL-1β, IL-18, and promotes an inflammatory form of cell death called pyroptosis induced by the activation of Gasdermin. Pyroptosis is a unique form of inflammatory cell death that leads to the release of not only cytokines but also other intracellular components that promote a broader immune response both of the innate and acquired immune system. Thus, inflammasome activation is a major regulatory of the inflammatory cascade.

NLRP3 is the most characterized inflammasome and has been shown to be critical in innate immunity and inflammatory responses. While several other NLR complexes, such as NLRC4, are activated under very specific circumstances, NLRP3 can be activated by numerous stimuli and should be seen as a sensor of intracellular homeostatic imbalance. Therefore, its precise functioning is essential. In addition to playing a role in host immune defense, dysregulation of NLRP3 has been linked to the pathogenesis of many inflammatory disorders. These include genetic diseases such as cryopyrin-associated periodic syndromes (CAPS) which is caused by gain-of-function mutations in the NLRP3 gene, as well as many prevalent neurologic and systemic diseases. Importantly, NLRP3 hyperactivation has been demonstrated pre-clinically to play a critical role in a plethora of inflammatory and degenerative diseases including, NASH, atherosclerosis and other cardiovascular diseases, Alzheimer's disease, Parkinson's disease, diabetes, gout, and numerous other autoinflammatory diseases. Thus, there is an unmet need in the field to develop small molecules for modulating NLRP3 activity to treat various diseases and disorders.

### SUMMARY

In one aspect, the present disclosure provides oxoindolinyl amide compounds of Formula (I):

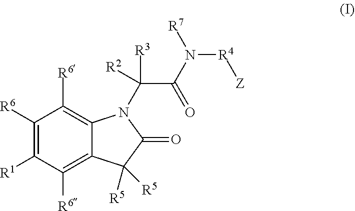

or pharmaceutically acceptable salts, solvates, clathrates, hydrates, stereoisomers, tautomers, isotopic derivatives, or prodrugs thereof, wherein:
R¹ is halo, —CN, C₁-C₆ alkyl, C₂-C₆ alkenyl, C₂-C₆ alkynyl, C₁-C₆ haloalkyl, C₁-C₆ alkoxy, or C₃-C₁₂ cycloalkyl;
each R² and R³ independently is H, C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or R² and R³ cyclize, together with the atom to which they are attached, to form a C₃-C₁₂ cycloalkyl or 3- to 12-membered heterocyclyl, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁴ is —C₁-C₆ alkyl-, -(L₁)ₚ-(C₃-C₁₂ cycloalkyl)-, -(L₁)ₚ-(3- to 12-membered heterocyclyl)-, -(L₁)ₚ—(C₆-C₁₀ aryl)-, or -(L₁)ₚ-(5- to 10-membered heteroaryl)-, wherein each instance of L₁ is independently —(C(R^{L1})₂)—, further wherein each instance of R^{L1} is independently H, halo, C₁-C₃ alkyl, or two R^{L1} groups, together with the atom to which they are attached, form a C₃₋₄ cycloalkyl; and wherein the alkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
Z is a -(L₂)ₙ-(carboxylic acid) or -(L₂)ₙ-tetrazole, wherein each instance of L₂ is independently —(C(R^{L2})₂)—, further wherein each instance of R^{L2} is independently H, halo, C₁-C₃ alkyl, or two R^{L2} groups, together with the atom to which they are attached, to form a C₃₋₄ cycloalkyl; and wherein the alkyl or cycloalkyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
each R⁵ is independently halo or C₁-C₆ alkyl, wherein the alkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or both R⁵ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁶ is H, halo, —OH, C₁-C₆ alkyl, C₂-C₆ alkenyl, C₂-C₆ alkynyl, C₁-C₆ haloalkyl, or C₁-C₆ alkoxy;
R^{6′} is H, halo, —OH, C₁-C₆ alkyl, C₂-C₆ alkenyl, C₂-C₆ alkynyl, C₁-C₆ haloalkyl, or C₁-C₆ alkoxy;
R^{6″} is H, halo, —OH, C₁-C₆ alkyl, C₂-C₆ alkenyl, C₂-C₆ alkynyl, C₁-C₆ haloalkyl, or C₁-C₆ alkoxy;
R⁷ is independently H or C₁-C₄ alkyl;
n is an integer from 0, 1, 2, or 3; and
p is an integer from 0, 1, or 2.

In some aspects, provided are pharmaceutical compositions comprising a compound of Formula (I), or a pharmaceutically acceptable salt, solvate, clathrate, hydrate, stereoisomer, tautomer, isotopic derivative, prodrug or polymorph thereof, and one or more pharmaceutically acceptable excipients.

In some aspects, provided are methods of preparing a compound of Formula (I). In other aspects, provided are intermediates suitable for use in a method for preparing a compound of Formula (I).

In yet other aspects, provided are methods of treating a disease or disorder in a subject in need thereof, comprising administering to the subject an effective amount of a compound of Formula (I), or a pharmaceutically acceptable salt, solvate, clathrate, hydrate, stereoisomer, tautomer, isotopic derivative, prodrug or polymorph thereof.

In yet other aspects, provided are methods of inhibiting NLRP3 activity in a cell, comprising contacting the cell with an effective amount of a compound of Formula (I), or a pharmaceutically acceptable salt, solvate, clathrate, hydrate, stereoisomer, tautomer, isotopic derivative, prodrug or polymorph thereof.

### Definitions

Unless otherwise stated, the following terms used in the specification and claims have the following meanings set out below.

“Aliphatic hydrocarbon” refers to an acyclic hydrocarbon groups comprising carbon atoms in its backbone and which may further contain one or more double or triple bonds within the hydrocarbon backbone. Such groups include alkyl, alkenyl, and alkynyl groups, as defined herein.

As used herein, “alkyl,” “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups, each containing no double or triple bonds in its backbone. For example, C₁-C₆ alkyl intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain) in its backbone, and in another embodiment, a straight chain or branched alkyl has four or fewer carbon atoms in its backbone.

As used herein, the term “optionally substituted alkyl” refers to unsubstituted alkyl or substituted alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, “alkenyl,” “C₂, C₃, C₄, C₅ or C₆ alkenyl” or “C₂-C₆ alkenyl” is intended to include unsaturated aliphatic hydrocarbon groups containing two to six carbon atoms and at least one double bond in its backbone, and includes C₂, C₃, C₄, C₅ or C₆ straight chain (linear) alkenyl groups and C₃, C₄, C₅ or C₆ branched alkenyl groups. For example, the term “alkenyl” includes straight chain alkenyl groups (e.g., ethenyl, propenyl, butenyl, pentenyl, hexenyl) and branched alkenyl groups. In some embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms in its backbone, and the term “C₃-C₆” includes alkenyl groups containing three to six carbon atoms in its backbone.

As used herein, the term “optionally substituted alkenyl” refers to unsubstituted alkenyl or substituted alkenyl, as described above, having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, “alkynyl,” “C₂, C₃, C₄, C₅ or C₆ alkynyl” or “C₂-C₆ alkynyl” is intended to include unsaturated aliphatic hydrocarbon groups containing two to six carbon atoms and at least one triple bond in its backbone, and includes C₂, C₃, C₄, C₅ or C₆ straight chain (linear) unsaturated alkynyl groups and C₃, C₄, C₅ or C₆ branched unsaturated alkynyl groups. For example, “alkynyl” includes straight chain alkynyl groups (e.g., ethynyl, propynyl, butynyl, pentynyl, hexynyl), and branched alkynyl groups. In some embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms in its backbone, and the term “C₃-C₆” includes alkynyl groups containing three to six carbon atoms in its backbone.

As used herein, the term “optionally substituted alkynyl” refers to unsubstituted alkynyl or substituted alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic hydrocarbon moiety or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a non-aromatic, saturated or partially unsaturated cyclic hydrocarbon, which is a monocyclic or polycyclic (e.g., fused, bridged, or spiro rings) system having 3 to 12 ring carbon atoms (e.g., C₃-C₁₂, C₃-C₁₀, or C₃-C₅). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “optionally substituted cycloalkyl” refers to unsubstituted cycloalkyl or substituted cycloalkyl having designated substituents replacing one or more hydrogen atoms on one or more carbon or heteroatom. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic hydrocarbon moiety or heteroaromatic moiety.

As used herein, the term “heterocyclyl” or “heterocycloalkyl” refers to a non-aromatic, saturated or partially unsaturated 3-8 membered monocyclic or bicyclic, 7-12 membered bicyclic (fused, bridged, or spiro rings), or 11-14 membered tricyclic ring system (fused, bridged, or spiro rings) having one or more ring heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms in the ring backbone, independently selected from the group consisting of nitrogen, oxygen and sulfur, unless specified otherwise. Examples of heterocyclyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocyclyl, only one of the rings in the heterocyclyl needs to be non-aromatic (e.g., 4,5,6,7-tetrahydrobenzo[c]isoxazolyl).

As used herein, the term “optionally substituted heterocyclyl” refers to unsubstituted heterocyclyl or substituted heterocyclyl having designated substituents replacing one or more hydrogen atoms on one or more carbon or heteroatom. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic hydrocarbon moiety or heteroaromatic moiety.

Unless otherwise specifically defined, the term “aryl” or an “aromatic hydrocarbon” moiety or group refers to a cyclic, C₆-C₁₄ aromatic hydrocarbon group (comprising carbon in the ring backbone) that has 1 to 3 aromatic hydrocarbon rings, including monocyclic or bicyclic groups such as phenyl (C₆), biphenyl (C₆aryl substituted by C₆aryl), or naphthyl (C₁₀). Where containing two aromatic hydrocarbon rings (bicyclic, etc.), the aromatic hydrocarbon rings of the aryl group may be joined at a single point (e.g., biphenyl) or fused (e.g., naphthyl). The aryl group may be optionally substituted by one or more substituents, e.g., 1 to 5 substituents, at any position on the aryl ring. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C₁-C₆) alkyl), N((C₁-C₆) alkyl)₂, —S(O)₂—(C₁-C₆) alkyl, —S(O)NH(C₁-C₆) alkyl, and —S(O)N((C₁-C₆) alkyl)₂. The substituents can themselves be optionally substituted. Furthermore, when containing two or more fused aromatic hydrocarbon rings, the aryl groups herein defined may have a saturated or partially unsaturated cycloalkyl or heterocyclyl rings fused with a fully unsaturated aromatic hydrocarbon ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, 10,11-dihydro-5H-dibenzo[a,d][7]annulenyl, and the like.

Unless otherwise specifically defined, “heteroaryl” or a “heteroaromatic” moiety or group refers to a monocyclic or polycyclic aromatic radical of 5 to 14 ring atoms, containing one or more ring heteroatoms selected from N, O, S, P, Se, or B within the aromatic ring system, the remaining ring atoms being C. Heteroaryl as herein defined also means a bicyclic heteroaromatic group wherein the ring heteroatom is selected from N, O, S, P, Se, or B. Heteroaryl as herein defined also means a tricyclic heteroaromatic group containing one or more ring heteroatoms selected from N, O, S, P, Se, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolinyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1λ²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4] thiazinyl, benzoxazolyl, benzisoxazolyl, furo[2,3-b]pyridinyl, benzothiophenyl, 1,5-naphthyridinyl, furo[3,2-b]pyridine, [1,2,4]triazolo[1,5-a]pyridinyl, benzo [1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo [1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated cycloalkyl or heterocyclyl rings fused with a fully unsaturated heteroaromatic ring, e.g., a 5-membered heteroaromatic ring containing 1 to 3 heteroatoms selected from N, O, S, P, Se, or B, or a 6-membered heteroaromatic ring containing 1 to 3 nitrogens, wherein the saturated or partially unsaturated cycloalkyl or heterocyclyl ring includes 0 to 4 heteroatoms selected from N, O, S, P, Se, or B, and is optionally substituted with one or more oxo groups (i.e., a C═O group). In heteroaryl ring systems containing more than two fused rings, a saturated or partially unsaturated cycloalkyl or heterocyclyl ring may further be fused with a saturated or partially unsaturated cycloalkyl or heterocyclyl ring described herein. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, orbenzo[c][1,2]oxaborol-1(3H)-olyl.

“Arylalkyl” or “aralkyl” refers to an optionally substituted C₆-C₁₀ aryl group attached to a C₁-C₆ alkyl group or optionally substituted 5- to 6-membered heteroaryl group attached to a C₁-C₆ alkyl group, wherein the point of attachment to the parent molecule is on the alkyl group. In some embodiments, arylalkyl is aryl-C₁₋₆ alkyl.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with cycloalkyl or heterocyclic rings, which are not aromatic so as to form a multicyclic system (e.g., tetralin, methylenedioxyphenyl such as benzo[d][1,3]dioxole-5-yl), provided the point of attachment to the parent molecule is on the aromatic aryl or heteroaryl ring.

As used herein, the term “divalent radical” refers to a group, such as an alkyl, alkenyl, alkynyl, cycloalkyl, heterocyclyl, aryl, or heteroaryl group, as defined herein, with two points of attachment.

As used herein, the term “nitrile” and “cyano” are used interchangeably herein, and each refer to —CN.

As used herein, the term “carboxylic acid” refers to the group —CO₂H.

As used herein, the term “tetrazole” refers to the each of the following tautomeric structures:

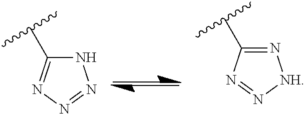 [SMILES: *c1nnn[nH]1 ; *c1nn[nH]n1]

As used herein, the term “hydroxy” or “hydroxyl” refers to an —OH group.

As used herein, the term “amino” refers to a primary, secondary, or tertiary amine. In some embodiments, amino is —NH₂, C₁-C₆ alkylamino-, C₁-C₆ dialkylamino-, C₆-C₁₀ arylamino-, C₆-C₁₀ diarylamino-, or (C₁-C₆ alkyl)(C₆-C₁₀ aryl)amino-.

As used herein, the terms “halo” or “halogen” are used interchangeably herein, and refer to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo group (i.e., a C═O group), then 2 hydrogen atoms on the atom are replaced. Oxo substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R¹, R², R³, R⁴, R⁵, R⁶, R^{6′}, R^{6″}, R⁷, Z, L₁, L₂) occurs more than one time in any constituent or formula for a compound, its definition at each occurrence is independent of its definition at every other occurrence. Thus, for example, if a group is shown to be substituted with 0-3 R moieties, then the group may optionally be substituted with up to two R moieties and R at each occurrence is selected independently from the definition of R. Also, combinations of substituents and/or variables are permissible, but only if such combinations result in stable compounds.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” Compounds with more than one chiral (asymmetric) center may exist either as an individual diastereomer or as a mixture of diastereomers, termed “diastereomeric mixture.” Likewise, compounds with only one chiral (asymmetric) center may exist either as an individual enantiomer or as a mixture of enantiomers. A mixture containing a mixture of diastereomers or enantiomers (of approximately equal proportions) is called a “racemic mixture.” Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116). The methods for the determination of stereochemistry and the separation of stereoisomers are well-known in the art (see discussion in Chapter 4 of “Advanced Organic Chemistry,” 4th edition J. March, John Wiley and Sons, New York, 2001), for example by synthesis from optically active starting materials or by resolution of a racemic form.

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules. Some of the compounds of the disclosure may have geometric isomeric centers (E- and Z-isomers). It is to be understood that the present disclosure encompasses all geometric isomers and mixtures thereof.

“Salts” include any and all salts, including pharmaceutically acceptable salts.

As used herein, the term “pharmaceutically acceptable salts” refer to compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, formic, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, trifluoroacetic and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, methylamine, dimethylamine, diethylamine, trimethylamine, piperidine, morpholine or tris-(2-hydroxyethyl)amine and the like.

A salt can be formed between an anion and a positively charged group (e.g., amino) on a substituted compound disclosed herein. As used herein, the term “pharmaceutically acceptable anion” refers to an anion suitable for forming a pharmaceutically acceptable salt. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms. In the salt form, it is understood that the ratio of the compound to the cation or anion of the salt can be 1:1, or any ratio other than 1:1, e.g., 3:1, 2:1, 1:2, or 1:3.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

It is to be understood that all references to pharmaceutically acceptable salts include solvent addition forms (solvates) or crystal forms (polymorphs) as defined herein, of the same salt.

Compounds are also provided herein as “neutral” compounds. “Neutral” and “free base” are used interchangeably herein, and refer to compounds which are not salts. It is understood that all references to neutral (free base) forms include solvent addition forms (solvates) or crystal forms (polymorphs) as defined herein, of the same neutral (free base) compound. For exemplary purpose, neutral compounds may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt). In some embodiments, solvate or crystal form have different properties in comparison to the neutral compound.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a “hydrate”; and if the solvent is alcohol, the solvate formed is an alcoholate. Hydrates are formed by the combination of one or more molecules of water with one molecule of the substance in which the water retains its molecular state as H₂O, for example, a hydrate such as hemi-hydrate, a mono-hydrate, a di-hydrate or a tri-hydrate. It is to be understood that the compounds of the present disclosure can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

A “clathrate” is a host molecule in which the guest molecule (i.e., the compound of Formula (I)) is in a cage formed by the host molecule or by a lattice of host molecules.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

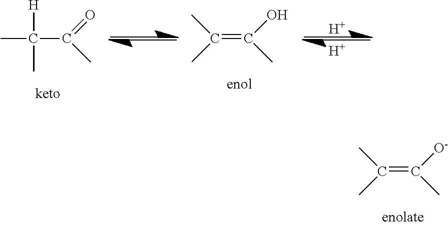

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

It is also to be understood that certain compounds of any one of the Formulae disclosed herein may exhibit polymorphism (“polymorphs”), and that the disclosure encompasses all such forms, or mixtures thereof. It is generally known that crystalline materials may be analyzed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis. In some embodiments, the polymorph may have different properties in comparison to the neutral compound.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. Examples of prodrugs include derivatives containing in vivo cleavable alkyl or acyl substituents at the ester or amide group in any one of the Formulae disclosed herein. Suitable prodrugs of a compound of any one of the Formulae disclosed herein is one that is based on reasonable medical judgment as being suitable for administration to the human or animal body without undesirable pharmacological activities and without undue toxicity. Various forms of prodrugs have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs,” by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems,” A.C.S. Symposium Series, Volume 14; and h) E. Roche (editor), “Bioreversible Carriers in Drug Design,” Pergamon Press, 1987.

A suitable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂-amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

The term “isotopic derivative,” as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I).

As used herein, the term “pharmaceutical composition” or “pharmaceutical formulation” are used interchangeably herein, and refer to a formulation containing a compound of Formula (I) in a form suitable for administration to a subject and a pharmaceutically acceptable carrier, diluent, adjuvant, or excipient, or a combination thereof. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient in a unit dose of composition may be varied according to the particular treatment involved. A variety of routes for administration are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” or “pharmaceutically acceptable carrier” are used interchangeably herein, and means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that, unless otherwise stated, any description of a method of treatment includes use of the compounds (e.g., a compound of Formula (I)) to provide such treatment as is described herein. It is to be further understood, unless otherwise stated, any description of a method of treatment includes use of the compounds to prepare a medicament to treat such condition. The treatment includes treatment of human or non-human animals including rodents and other disease models. As used herein, the term “subject” is interchangeable with the term “subject in need thereof,” both of which refer to a subject having a disease or disorder, or having an increased risk of developing the disease or disorder.

As use herein, a “subject” is a mammal. The mammal can be e.g., a human or appropriate non-human mammal, such as primate, mouse, rat, dog, cat, cow, horse, goat, camel, sheep or a pig. In one embodiment, the mammal is a human. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy. “Subject” and “patient” are used interchangeably herein.

As used herein, the term “treating” or “treat” describes the management and care of a subject for the purpose of combating a disease, condition, or disorder and includes the administration of a compound of the present disclosure, or a pharmaceutically acceptable salt, solvate, clathrate, hydrate, stereoisomer, tautomer, isotopic derivative, prodrug or polymorph thereof, to alleviate the symptoms or complications of a disease, condition or disorder, or to eliminate the disease, condition or disorder from which a subject suffers from (“therapeutic treatment”; “therapeutically treating”) or can or may also be used to prevent a disease, condition or disorder (“prophylactic treatment”; “prophylactically treating”) from which a subject has been diagnosed with or has a predisposition for but has not yet exhibited symptoms. The term “treat” can also include treatment of a cell in vitro or an animal model. “Treating” or “treatment” of a state, disorder or condition therefore includes: (1) preventing or delaying the appearance of clinical symptoms of the state, disorder or condition developing in a human that may be afflicted with or predisposed to the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

In some embodiments, treating a disease or disorder is not preventing a disease or disorder.

As used herein, the term “therapeutically effective amount” refers to an amount of a compound to treat or ameliorate an identified disease or condition from which a subject suffers, or to exhibit a detectable therapeutic or inhibitory effect, and “prophylactically effective amount” refers to an amount of a compound to prevent an identified disease or condition from which a subject has been diagnosed with or has a predisposition for but has not yet exhibited symptoms. “Therapeutically effective” and “prophylactically effective” are collectively referred to as an “effective amount,” which is an amount sufficient to treat or prevent an inflammasome related condition referred to herein, slow its progression and/or reduce the symptoms associated with the condition. The size of the dose for therapeutic or prophylactic purposes of a compound of Formula (I) will naturally vary according to the nature and severity of the conditions, the age and sex of the animal or patient and the route of administration, according to well-known principles of medicine. The effect can be detected by any assay method known in the art. The precise effective amount for a subject will depend upon the subject's body weight, size, and health; the nature and extent of the condition; and the therapeutic or combination of therapeutics selected for administration. Effective amounts, whether therapeutically or prophylactically effective, for a given situation can be determined by routine experimentation that is within the skill and judgment of the clinician.

“Inhibition,” “inhibiting,” “inhibit,” and “inhibitor,” and the like, refer to the ability of a compound to reduce, slow, halt or prevent activity of a particular biological process (e.g., NLRP3 activity) in a cell relative to vehicle.

The phrase “at least one” refers to one instance or more than one instance.

As used herein, the term “about” refers to a recited amount, value, or duration ±10% or less of said amount, value, or duration. In some embodiments, “about” refers to a recited amount, value, or duration ±10%, ±8%, ±6%, ±5%, ±4%, ±2%, ±100 or ±0.5%. In some embodiments, “about” refers to a recited amount, value, or duration ±10%, ±8%, ±6%, ±5%, ±4%, or ±2%. In some embodiments, “about” refers to a recited amount, value, or duration ±5%. In some embodiments, “about” refers to a listed amount, value, or duration ±2% or ±1%. For example, in some embodiments, when the term “about” is used when reciting a temperature or temperature range, these terms refer to the recited temperature or temperature range ±5° C., ±2° C., or ±1° C. In some embodiments, the term “about” refers to the recited temperature or temperature range ±2° C.

The articles “a” and “an” are used in this disclosure to refer to one or more than one (i.e., to at least one) of the grammatical object of the article.

The term “and/or” is used in this disclosure to mean either “and” or “or” unless indicated otherwise.

All percentages and ratios used herein, unless otherwise indicated, are by weight.

### DETAILED DESCRIPTION

The present disclosure relates to oxoindolinyl amide derivatives, pharmaceutically acceptable salts, solvates, clathrates, hydrates, single stereoisomers, mixtures of stereoisomers, or racemic mixtures of stereoisomers thereof, tautomers, isotopic derivatives, prodrugs and polymorphs thereof, which inhibit NLRP3 activity and are accordingly useful in methods of treatment of the human or animal body. The present disclosure also relates to processes for the preparation of these compounds, to pharmaceutical compositions comprising them and to their use in the treatment of disorders in which NLRP3 is implicated, such as inflammation, an auto-immune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or an NLRP3-related disease in a subject that has been determined to carry a germline or somatic non-silent mutation in NLRP3.

### (1) Compounds

In one aspect, the present disclosure provides oxoindolinyl amide compounds of Formula (I):

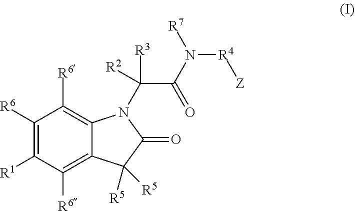

or pharmaceutically acceptable salts, solvates, clathrates, hydrates, stereoisomers, tautomers, isotopic derivatives, prodrugs and/or polymorphs thereof, wherein:
R¹ is halo, —CN, C₁-C₆ alkyl, C₂-C₆ alkenyl, C₂-C₆ alkynyl, C₁-C₆ haloalkyl, C₁-C₆ alkoxy, or C₃-C₁₂ cycloalkyl;
each R² and R³ independently is H, C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or R² and R³ cyclize, together with the atom to which they are attached, to form a C₃-C₁₂ cycloalkyl or 3- to 12-membered heterocyclyl, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁴ is —C₁-C₆ alkyl-, -(L₁)ₚ-(C₃-C₁₂ cycloalkyl)-, -(L₁)ₚ-(3- to 12-membered heterocyclyl)-, -(L₁)ₚ-(C₆-C₁₀ aryl)-, or -(L₁)ₚ-(5- to 10-membered heteroaryl)-, wherein each instance of L₁ is independently —(C(R^{L})₂)—, further wherein each instance of R^{L1} is independently H, halo or C₁-C₃ alkyl, or two R^{L1} groups, together with the atom to which they are attached, form a C₃₋₄ cycloalkyl; and wherein the alkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
Z is a -(L₂)ₙ-(carboxylic acid) or -(L₂)ₙ-tetrazole, wherein each instance of L₂ is independently —(C(R^{L2})₂)—, further wherein each instance of R^{L2} is independently H, halo or C₁-C₃ alkyl, or two R^{L2} groups, together with the atom to which they are attached, to form a C₃₋₄ cycloalkyl; and wherein the alkyl or cycloalkyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
each R⁵ is independently halo or C₁-C₆ alkyl, wherein the alkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or both R⁵ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁶ is H, halo, —OH, C₁-C₆ alkyl, C₂-C₆ alkenyl, C₂-C₆ alkynyl, C₁-C₆ haloalkyl, or C₁-C₆ alkoxy;
R^{6′} is H, halo, —OH, C₁-C₆ alkyl, C₂-C₆ alkenyl, C₂-C₆ alkynyl, C₁-C₆ haloalkyl, or C₁-C₆ alkoxy;
R^{6″} is H, halo, —OH, C₁-C₆ alkyl, C₂-C₆ alkenyl, C₂-C₆ alkynyl, C₁-C₆ haloalkyl, or C₁-C₆ alkoxy;
R⁷ is independently H or C₁-C₄ alkyl;
n is an integer from 0, 1, 2, or 3; and
p is an integer from 0, 1, or 2.

In some embodiments of Formula (I):
R¹ is bromo, chloro, —CN, C₁-C₆ alkyl, C₁-C₆ haloalkyl, or C₃-C₁₂ cycloalkyl;
each R² and R³ independently is H, C₁-C₆ alkyl, C₁-C₆ haloalkyl, C₃-C₁₂ cycloalkyl, 3- to 12-membered heterocyclyl, C₆-C₁₀ aryl, or 5- to 10-membered heteroaryl, where the alkyl, haloalkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or R² and R³ cyclize together to form a C₃-C₁₂ cycloalkyl or 3- to 12-membered heterocyclyl, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁴ is -(n-propyl)-, -(L₁)ₚ-(C₃-C₁₂ cycloalkyl)-, -(L₁)ₚ-(3- to 12-membered heterocyclyl)-, -(L₁)ₚ-(C₆-C₁₀ aryl)-, or -(L₁)ₚ-(5- to 10-membered heteroaryl)-, wherein each instance of R^{L1} is independently H, halo or C₁-C₃ alkyl, or two R^{L1} groups, together with the atom to which they are attached, form a C₃ cycloalkyl; and wherein n-propyl, alkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
Z is a -(L₂)ₙ-(carboxylic acid) or -(L₂)ₙ-tetrazole, wherein each instance of L₂ is independently —(C(R^{L2})₂)—, further wherein each instance of R^{L2} is independently H, halo or C₁-C₃ alkyl, or two R^{L2} groups, together with the atom to which they are attached, to form a C₃ cycloalkyl; and wherein the alkyl or cycloalkyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
each R⁵ is independently halo or C₁-C₆ alkyl, wherein the alkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or both R⁵ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁶ is H, halo, —OH, C₁-C₆ alkyl, or C₁-C₆ alkoxy;
R^{6′} is H, halo, —OH, C₁-C₆ alkyl, or C₁-C₆ alkoxy;
R^{6″} is H, halo, —OH, C₁-C₆ alkyl, or C₁-C₆ alkoxy;
R⁷ is independently H or C₁-C₄ alkyl;
n is an integer from 0, 1, 2, or 3; and
p is an integer from 0, 1, or 2.

In some embodiments of Formula (I):
R¹ is bromo, chloro, —CN, C₁-C₆ alkyl, C₁-C₆ haloalkyl, or C₃-C₁₂ cycloalkyl;
each R² and R³ independently is H, C₁-C₆ alkyl, C₁-C₆ haloalkyl, C₃-C₁₂ cycloalkyl, 3- to 12-membered heterocyclyl, C₆-C₁₀ aryl, or 5- to 10-membered heteroaryl, where the alkyl, haloalkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or R² and R³ cyclize together to form a C₃-C₁₂ cycloalkyl or 3- to 12-membered heterocyclyl, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁴ is -(n-propyl)-, -(L₁)ₚ-(C₃-C₁₂ cycloalkyl)-, -(L₁)ₚ-(3- to 12-membered heterocyclyl)-, -(L₁)ₚ-(C₆-C₁₀ aryl)-, or -(L₁)ₚ-(5- to 10-membered heteroaryl)-, wherein each instance of R^{L1} is independently H, halo or C₁-C₃ alkyl, or two R^{L1} groups, together with the atom to which they are attached, form a C₃ cycloalkyl; and wherein n-propyl, alkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
Z is a -(L₂)ₙ-(carboxylic acid) or -(L₂)ₙ-tetrazole, wherein each instance of L₂ is independently —(C(R^{L2})₂)-, further wherein each instance of R^{L2} is independently H, halo or C₁-C₃ alkyl, or two R^{L2} groups, together with the atom to which they are attached, to form a C₃ cycloalkyl; and wherein the alkyl or cycloalkyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
each R⁵ is independently fluoro or C₁-C₆ alkyl, wherein the alkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or both R⁵ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁶ is H, halo, —OH, C₁-C₆ alkyl, or C₁-C₆ alkoxy;
R^{6′} is H, halo, —OH, C₁-C₆ alkyl, or C₁-C₆ alkoxy;
R^{6″} is H, halo, —OH, C₁-C₆ alkyl, or C₁-C₆ alkoxy;
R⁷ is independently H or C₁-C₄ alkyl;
n is an integer from 0, 1, 2, or 3; and
p is an integer from 0, 1, or 2.

In some embodiments, each R⁵ is independently fluoro or C₁-C₆ alkyl, wherein the alkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or both R⁵ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, the compound of Formula (I) is a compound of Formula (I-A):

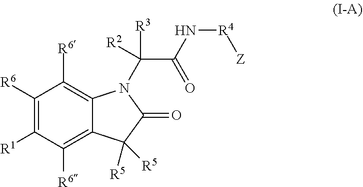

or a pharmaceutically acceptable salt, solvate, clathrate, hydrate, stereoisomer, tautomer, isotopic derivative, prodrug or polymorph thereof, wherein:
R¹ is halo, —CN, C₁-C₆ alkyl, C₂-C₆ alkenyl, C₂-C₆ alkynyl, C₁-C₆ haloalkyl, C₁-C₆ alkoxy, or C₃-C₁₂ cycloalkyl;
each R² and R³ independently is H, C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or R² and R³ cyclize, together with the atom to which they are attached, to form a C₃-C₁₂ cycloalkyl or 3- to 12-membered heterocyclyl, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁴ is —C₁-C₆ alkyl-, -(L₁)ₚ-(C₃-C₁₂ cycloalkyl)-, -(L₁)ₚ-(3- to 12-membered heterocyclyl)-, -(L₁)ₚ-(C₆-C₁₀ aryl)-, or -(L₁)ₚ-(5- to 10-membered heteroaryl)-, each instance of R^{L1} is independently H, halo or C₁-C₃ alkyl, or two R^{L1} groups, together with the atom to which they are attached, form a C₃ cycloalkyl; and wherein the alkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
Z is a -(L₂)ₙ-(carboxylic acid) or -(L₂)ₙ-tetrazole, wherein each instance of L₂ is independently —(C(R^{L2})₂)—, further wherein each instance of R^{L2} is independently H, halo or C₁-C₃ alkyl, or two R^{L2} groups, together with the atom to which they are attached, to form a C₃ cycloalkyl; and wherein the alkyl or cycloalkyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
each R⁵ is independently fluoro or C₁-C₆ alkyl, wherein the alkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or both R⁵ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁶ is H, halo, —OH, C₁-C₆ alkyl, C₂-C₆ alkenyl, C₂-C₆ alkynyl, C₁-C₆ haloalkyl, or C₁-C₆ alkoxy;
R^{6′} is H, halo, —OH, C₁-C₆ alkyl, C₂-C₆ alkenyl, C₂-C₆ alkynyl, C₁-C₆ haloalkyl, or C₁-C₆ alkoxy;
R^{6″} is H, halo, —OH, C₁-C₆ alkyl, C₂-C₆ alkenyl, C₂-C₆ alkynyl, C₁-C₆ haloalkyl, or C₁-C₆ alkoxy;
n is an integer from 0, 1, 2, or 3; and
p is an integer from 0, 1, or 2.

In some embodiments of Formula (I-A):
R¹ is bromo, chloro, —CN, C₁-C₆ alkyl, C₁-C₆ haloalkyl, or C₃-C₁₂ cycloalkyl;
each R² and R³ independently is H, C₁-C₆ alkyl, C₁-C₆ haloalkyl, C₃-C₁₂ cycloalkyl, 3- to 12-membered heterocyclyl, C₆-C₁₀ aryl, or 5- to 10-membered heteroaryl, where the alkyl, haloalkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or R² and R³ cyclize together to form a C₃-C₁₂ cycloalkyl or 3- to 12-membered heterocyclyl, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁴ is an -(n-propyl)-, -(L₁)ₚ-(C₃-C₁₂ cycloalkyl), -(L₁)ₚ-(3- to 12-membered heterocyclyl), -(L₁)ₚ-(C₆-C₁₀ aryl)-, or -(L₁)ₚ-(5- to 10-membered heteroaryl)-, wherein each instance of R^{L1} is independently H, halo or C₁-C₃ alkyl, or two R^{L1} groups, together with the atom to which they are attached, form a C₃ cycloalkyl; and wherein the n-propyl, alkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
Z is a -(L₂)ₙ-(carboxylic acid) or -(L₂)ₙ-tetrazole, wherein each instance of L₂ is independently —(C(R^{L2})₂)—, further wherein each instance of R^{L2} is independently H, halo or C₁-C₃ alkyl, or two R^{L2} groups, together with the atom to which they are attached, to form a C₃ cycloalkyl; and wherein the alkyl or cycloalkyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
each R⁵ is independently fluoro or C₁-C₆ alkyl, wherein the alkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or both R⁵ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁶ is H, halo, —OH, C₁-C₆ alkyl, or C₁-C₆ alkoxy;
R^{6′} is H, halo, —OH, C₁-C₆ alkyl, or C₁-C₆ alkoxy;
R^{6″} is H, halo, —OH, C₁-C₆ alkyl, or C₁-C₆ alkoxy;
n is an integer from 0, 1, 2, or 3; and
p is an integer from 0, 1, or 2.

Additional embodiments are further contemplated herein.

(a) Groups R² and R³

As generally defined herein, each R² and R³ independently is H, C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or R² and R³ cyclize, together with the atom to which they are attached, to form a C₃-C₁₂ cycloalkyl or 3- to 12-membered heterocyclyl, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, each R² and R³ independently is H, C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, each R² and R³ independently is H.

In some embodiments, each R² and R³ independently is C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is H, C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is H.

In some embodiments, R² is C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl.

In some embodiments, R² is C₁-C₆ alkyl, C₂-C₆ alkenyl, C₂-C₆ alkynyl, C₁-C₆ haloalkyl, or C₁-C₆ alkoxy, where the alkyl, alkenyl, alkynyl, haloalkyl, or alkoxy is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₁-C₆ alkyl, C₂-C₆ alkenyl, C₂-C₆ alkynyl, C₁-C₆ haloalkyl, or C₁-C₆ alkoxy.

In some embodiments, R² is C₁-C₆ alkyl, C₂-C₆ alkenyl, or C₂-C₆ alkynyl.

In some embodiments, R² is C₁-C₆ alkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₁-C₆ alkyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₁-C₆ alkyl.

In some embodiments, R² is methyl. In some embodiments, R² is ethyl. In some embodiments, R² is propyl. In some embodiments, R² is butyl. In some embodiments, R² is pentyl. In some embodiments, R² is hexyl. In some embodiments, R² is isopropyl. In some embodiments, R² is isobutyl. In some embodiments, R² is isopentyl. In some embodiments, R² is isohexyl. In some embodiments, R² is secbutyl. In some embodiments, R² is secpentyl. In some embodiments, R² is sechexyl. In some embodiments, R² is tertbutyl.

In some embodiments, R² is C₂-C₆ alkenyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₂-C₆ alkenyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₂-C₆ alkenyl.

In some embodiments, R² is C₂ alkenyl. In some embodiments, R² is C₃ alkenyl. In some embodiments, R² is C₄ alkenyl. In some embodiments, R² is C₅ alkenyl. In some embodiments, R² is C₆ alkenyl.

In some embodiments, R² is C₂-C₆ alkynyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₂-C₆ alkynyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₂-C₆ alkynyl.

In some embodiments, R² is C₂ alkynyl. In some embodiments, R² is C₃ alkynyl. In some embodiments, R² is C₄ alkynyl. In some embodiments, R² is C₅ alkynyl. In some embodiments, R² is C₆ alkynyl.

In some embodiments, R² is C₁-C₆ haloalkyl or C₁-C₆ alkoxy.

In some embodiments, R² is C₁-C₆ haloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₁-C₆ haloalkyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₁-C₆ haloalkyl.

In some embodiments, R² is halomethyl. In some embodiments, R² is haloethyl. In some embodiments, R² is halopropyl. In some embodiments, R² is halobutyl. In some embodiments, R² is halopentyl. In some embodiments, R² is halohexyl.

In some embodiments, R² is C₁-C₆ alkoxy optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₁-C₆ alkoxy substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₁-C₆ alkoxy.

In some embodiments, R² is methoxy. In some embodiments, R² is ethoxy. In some embodiments, R² is propoxy. In some embodiments, R² is butoxy. In some embodiments, R² is pentoxy. In some embodiments, R² is hexyloxy.

In some embodiments, R² is C₃-C₁₂ cycloalkyl, 3- to 12-membered heterocyclyl, C₆-C₁₀ aryl, or 5- to 10-membered heteroaryl, where the cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₃-C₁₂ cycloalkyl, 3- to 12-membered heterocyclyl, C₆-C₁₀ aryl, or 5- to 10-membered heteroaryl.

In some embodiments, R² is C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₃-C₁₂ cycloalkyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₃-C₁₂ cycloalkyl.

In some embodiments, R² is C₃-C₆ cycloalkyl.

In some embodiments, R² is C₃ cycloalkyl. In some embodiments, R² is C₄ cycloalkyl. In some embodiments, R² is C₅ cycloalkyl. In some embodiments, R² is C₆ cycloalkyl.

In some embodiments, R² is C₇ cycloalkyl. In some embodiments, R² is C₈ cycloalkyl. In some embodiments, R² is C₉ cycloalkyl. In some embodiments, R² is C₁₀ cycloalkyl. In some embodiments, R² is C₁₁ cycloalkyl. In some embodiments, R² is C₁₂ cycloalkyl.

In some embodiments, R² is 3- to 12-membered heterocyclyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is 3- to 12-membered heterocyclyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is 3- to 12-membered heterocyclyl.

In some embodiments, R² is 3-membered heterocyclyl. In some embodiments, R² is 4-membered heterocyclyl. In some embodiments, R² is 5-membered heterocyclyl. In some embodiments, R² is 6-membered heterocyclyl. In some embodiments, R² is 7-membered heterocyclyl. In some embodiments, R² is 8-membered heterocyclyl. In some embodiments, R² is 9-membered heterocyclyl. In some embodiments, R² is 10-membered heterocyclyl. In some embodiments, R² is 11-membered heterocyclyl.

In some embodiments, R² is 12-membered heterocyclyl.

In some embodiments, R² is C₆-C₁₀ aryl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₆-C₁₀ aryl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is C₆-C₁₀ aryl.

In some embodiments, R² is C₆ aryl. In some embodiments, R² is C₈ aryl. In some embodiments, R² is C₁₀ aryl.

In some embodiments, R² is 5- to 10-membered heteroaryl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is 5- to 10-membered heteroaryl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² is 5- to 10-membered heteroaryl.

In some embodiments, R² is 5-membered heteroaryl. In some embodiments, R² is 6-membered heteroaryl. In some embodiments, R² is 7-membered heteroaryl. In some embodiments, R² is 8-membered heteroaryl. In some embodiments, R² is 9-membered heteroaryl. In some embodiments, R² is 10-membered heteroaryl.

In some embodiments, R³ is H, C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is H.

In some embodiments, R³ is C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl.

In some embodiments, R³ is C₁-C₆ alkyl, C₂-C₆ alkenyl, C₂-C₆ alkynyl, C₁-C₆ haloalkyl, or C₁-C₆ alkoxy, where the alkyl, alkenyl, alkynyl, haloalkyl, or alkoxy is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₁-C₆ alkyl, C₂-C₆ alkenyl, C₂-C₆ alkynyl, C₁-C₆ haloalkyl, or C₁-C₆ alkoxy.

In some embodiments, R³ is C₁-C₆ alkyl, C₂-C₆ alkenyl, or C₂-C₆ alkynyl.

In some embodiments, R³ is C₁-C₆ alkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₁-C₆ alkyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₁-C₆ alkyl.

In some embodiments, R³ is methyl. In some embodiments, R³ is ethyl. In some embodiments, R³ is propyl. In some embodiments, R³ is butyl. In some embodiments, R³ is pentyl. In some embodiments, R³ is hexyl. In some embodiments, R³ is isopropyl. In some embodiments, R³ is isobutyl. In some embodiments, R³ is isopentyl. In some embodiments, R³ is isohexyl. In some embodiments, R³ is secbutyl. In some embodiments, R³ is secpentyl. In some embodiments, R³ is sechexyl. In some embodiments, R³ is tertbutyl.

In some embodiments, R³ is C₂-C₆ alkenyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₂-C₆ alkenyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₂-C₆ alkenyl.

In some embodiments, R³ is C₂ alkenyl. In some embodiments, R³ is C₃ alkenyl. In some embodiments, R³ is C₄ alkenyl. In some embodiments, R³ is C₅ alkenyl. In some embodiments, R³ is C₆ alkenyl.

In some embodiments, R³ is C₂-C₆ alkynyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₂-C₆ alkynyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₂-C₆ alkynyl.

In some embodiments, R³ is C₂ alkynyl. In some embodiments, R³ is C₃ alkynyl. In some embodiments, R³ is C₄ alkynyl. In some embodiments, R³ is C₅ alkynyl. In some embodiments, R³ is C₆ alkynyl.

In some embodiments, R³ is C₁-C₆ haloalkyl or C₁-C₆ alkoxy

In some embodiments, R³ is C₁-C₆ haloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₁-C₆ haloalkyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₁-C₆ haloalkyl.

In some embodiments, R³ is halomethyl. In some embodiments, R³ is haloethyl. In some embodiments, R³ is halopropyl. In some embodiments, R³ is halobutyl. In some embodiments, R³ is halopentyl. In some embodiments, R³ is halohexyl.

In some embodiments, R³ is C₁-C₆ alkoxy optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₁-C₆ alkoxy substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₁-C₆ alkoxy.

In some embodiments, R³ is methoxy. In some embodiments, R³ is ethoxy. In some embodiments, R³ is propoxy. In some embodiments, R³ is butoxy. In some embodiments, R³ is pentoxy. In some embodiments, R³ is hexyloxy.

In some embodiments, R³ is C₃-C₁₂ cycloalkyl, 3- to 12-membered heterocyclyl, C₆-C₁₀ aryl, or 5- to 10-membered heteroaryl, where the cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₃-C₁₂ cycloalkyl, 3- to 12-membered heterocyclyl, C₆-C₁₀ aryl, or 5- to 10-membered heteroaryl.

In some embodiments, R³ is C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₃-C₁₂ cycloalkyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₃-C₁₂ cycloalkyl.

In some embodiments, R³ is C₃-C₆ cycloalkyl.

In some embodiments, R³ is C₃ cycloalkyl. In some embodiments, R³ is C₄ cycloalkyl. In some embodiments, R³ is C₅ cycloalkyl. In some embodiments, R³ is C₆ cycloalkyl.

In some embodiments, R³ is C₇ cycloalkyl. In some embodiments, R³ is C₈ cycloalkyl. In some embodiments, R³ is C₉ cycloalkyl. In some embodiments, R³ is C₁₀ cycloalkyl. In some embodiments, R³ is C₁₁ cycloalkyl. In some embodiments, R³ is C₁₂ cycloalkyl.

In some embodiments, R³ is 3- to 12-membered heterocyclyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is 3- to 12-membered heterocyclyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is 3- to 12-membered heterocyclyl.

In some embodiments, R³ is 3-membered heterocyclyl. In some embodiments, R³ is 4-membered heterocyclyl. In some embodiments, R³ is 5-membered heterocyclyl. In some embodiments, R³ is 6-membered heterocyclyl. In some embodiments, R³ is 7-membered heterocyclyl. In some embodiments, R³ is 8-membered heterocyclyl. In some embodiments, R³ is 9-membered heterocyclyl. In some embodiments, R³ is 10-membered heterocyclyl. In some embodiments, R³ is 11-membered heterocyclyl.

In some embodiments, R³ is 12-membered heterocyclyl.

In some embodiments, R³ is C₆-C₁₀ aryl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₆-C₁₀ aryl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is C₆-C₁₀ aryl.

In some embodiments, R³ is C₆ aryl. In some embodiments, R³ is C₈ aryl. In some embodiments, R³ is C₁₀ aryl.

In some embodiments, R³ is 5- to 10-membered heteroaryl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is 5- to 10-membered heteroaryl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R³ is 5- to 10-membered heteroaryl.

In some embodiments, R³ is 5-membered heteroaryl. In some embodiments, R³ is 6-membered heteroaryl. In some embodiments, R³ is 7-membered heteroaryl. In some embodiments, R³ is 8-membered heteroaryl. In some embodiments, R³ is 9-membered heteroaryl. In some embodiments, R³ is 10-membered heteroaryl.

In some embodiments, R² and R³ cyclize, together with the atom to which they are attached, to form a C₃-C₁₂ cycloalkyl or 3- to 12-membered heterocyclyl, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² and R³ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² and R³ cyclize, together with the atom to which they are attached, to form a C₃-C₁₂ cycloalkyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² and R³ cyclize, together with the atom to which they are attached, to form a C₃-C₁₂ cycloalkyl.

In some embodiments, R² and R³ cyclize, together with the atom to which they are attached, to form a C₃ cycloalkyl. In some embodiments, R² and R³ cyclize, together with the atom to which they are attached, to form a C₄ cycloalkyl. In some embodiments, R² and R³ cyclize, together with the atom to which they are attached, to form a C₅ cycloalkyl. In some embodiments, R² and R³ cyclize, together with the atom to which they are attached, to form a C₆ cycloalkyl. In some embodiments, R² and R³ cyclize, together with the atom to which they are attached, to form a C₇ cycloalkyl. In some embodiments, R² and R³ cyclize, together with the atom to which they are attached, to form a C₈ cycloalkyl. In some embodiments, R² and R³ cyclize, together with the atom to which they are attached, to form a C₉ cycloalkyl. In some embodiments, R² and R³ cyclize, together with the atom to which they are attached, to form a C₁₀ cycloalkyl. In some embodiments, R² and R³ cyclize, together with the atom to which they are attached, to form a C₁₁ cycloalkyl. In some embodiments, R² and R³ cyclize, together with the atom to which they are attached, to form a C₁₂ cycloalkyl.

In some embodiments, R² and R³ cyclize, together with the atom to which they are attached, to form a C₃ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² and R³ cyclize, together with the atom to which they are attached, to form a C₃ cycloalkyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² and R³ cyclize, together with the atom to which they are attached, to form a 3- to 12-membered heterocyclyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² and R³ cyclize, together with the atom to which they are attached, to form a 3- to 12-membered heterocyclyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R² and R³ cyclize, together with the atom to which they are attached, to form a 3- to 12-membered heterocyclyl.

In some embodiments, R² and R³ cyclize, together with the atom to which they are attached, to form a 3-membered heterocyclyl. In some embodiments, R² and R³ cyclize, together with the atom to which they are attached, to form a 4-membered heterocyclyl. In some embodiments, R² and R³ cyclize, together with the atom to which they are attached, to form a 5-membered heterocyclyl. In some embodiments, R² and R³ cyclize, together with the atom to which they are attached, to form a 6-membered heterocyclyl. In some embodiments, R² and R³ cyclize, together with the atom to which they are attached, to form a 7-membered heterocyclyl. In some embodiments, R² and R³ cyclize, together with the atom to which they are attached, to form a 8-membered heterocyclyl. In some embodiments, R² and R³ cyclize, together with the atom to which they are attached, to form a 9-membered heterocyclyl. In some embodiments, R² and R³ cyclize, together with the atom to which they are attached, to form a 10-membered heterocyclyl. In some embodiments, R² and R³ cyclize, together with the atom to which they are attached, to form a 11-membered heterocyclyl. In some embodiments, R² and R³ cyclize, together with the atom to which they are attached, to form a 12-membered heterocyclyl.

In some embodiments, each instance of R² and R³ is independently H, C₁-C₃ alkyl, C₁-C₃ haloalkyl, or R² and R³ cyclize, together with the atom to which they are attached, to form a C₃ cycloalkyl. In some embodiments, each instance of R² and R³ is H. In some embodiments, R² and R³ cyclize, together with the atom to which they are attached, to form a C₃ cycloalkyl.

(b) Groups L₁, L₂, R⁴, R⁷, Z, p, and n

As generally defined herein, R⁴ is —C₁-C₆ alkyl-, -(L₁)ₚ-(C₃-C₁₂ cycloalkyl)-, -(L₁)ₚ-(3- to 12-membered heterocyclyl)-, -(L₁)ₚ-(C₆-C₁₀ aryl)-, or -(L₁)ₚ-(5- to 10-membered heteroaryl)-, wherein each instance of L₁ is independently —(C(R^{L1})₂)—, further wherein each instance of R^{L1} is independently H, halo or C₁-C₃ alkyl, or two R^{L1} groups, together with the atom to which they are attached, to form a C₃₋₄ cycloalkyl; wherein the alkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; and p is 0, 1, or 2. In some embodiment, p is 0. In some embodiments, p is 1. In some embodiments, p is 2.

As further generally defined herein, Z is a -(L₂)ₙ-(carboxylic acid) or -(L₂)ₙ-tetrazole, each instance of L₂ is independently —(C(R^{L2})₂)-, further wherein each instance of R^{L2} is independently H, halo or C₁-C₃ alkyl, or two R^{L2} groups, together with the atom to which they are attached, form a C₃ cycloalkyl; wherein the alkyl or cycloalkyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; and n is 0, 1, 2, or 3. In some embodiments, n is 0. In some embodiments, n is 1. In some embodiments, n is 2.

In some embodiments, n is 3.

As further generally defined herein, R⁷ is independently H or C₁-C₄ alkyl. In some embodiments, R⁷ is H. In some embodiments, R⁷ is C₁-C₄ alkyl. In some embodiments, R⁷ is methyl. In some embodiments, R⁷ is ethyl. In some embodiments, R⁷ is propyl. In some embodiments, R⁷ is isopropyl. In some embodiments, R⁷ is n-butyl. In some embodiments, R⁷ is sec-butyl. In some embodiments, R⁷ is tert-butyl.

In some embodiments, R⁷ is H or methyl.

It is understood that R⁴ is a divalent moiety, with two points of attachment, one point of attachment to the NR⁷ amide nitrogen, and the other point of attachment to the Z moiety. Groups recited herein are read left to right, with the left recitation being bound to the NR⁷ amide nitrogen and the right recitation being bound to Z. For example, for groups -(L₁)ₚ-(C₃-C₁₂ cycloalkyl)-, -(L₁)ₚ-(3- to 12-membered heterocyclyl)-, and -(L₁)ₚ-(5- to 10-membered heteroaryl)-, the left -(L₁)ₚ- group is bound to the NR⁷ amide nitrogen. It is further understood that a portion of the Z group, that is -(L₂)ₙ- wherein n is 0, 1, 2, or 3, also comprises part of the linker that joins the NR⁷ amide nitrogen to the terminal carboxylic acid or tetrazole group.

In some embodiments of -(L₁)ₚ-, wherein each instance of L₁ is independently —(C(R^{L})₂)-, at least one instance of R^{L1} is —C₁-C₃ alkyl optionally substituted with one or more halo. In some embodiments, at least one instance of R^{L1} is H. In some embodiments, each instance of R^{L1} is H, i.e., wherein each instance of L₁ is —(CH₂)—.

In some embodiments of -(L₂)ₙ-, wherein each instance of L₂ is independently —(C(R^{L2})₂)—, at least one instance of R^{L2} is —C₁-C₃ alkyl optionally substituted with one or more halo. In some embodiments, at least one instance of R^{L2} is H. In some embodiments, each instance of R^{L2} is H, i.e., wherein each instance of L₂ is —(CH₂)—.

In some embodiments, each instance of L₁ is —(CH₂)— and each instance of L₂ is —(CH₂)—. In some embodiments, p is 1, L₁ is —(CH(CH₃))— and each instance of L₂ is —(CH₂)—.

In some embodiments, R⁴ is a —C₁-C₆ alkyl-, —(CH₂)ₚ—(C₃-C₁₂ cycloalkyl)-, -(3- to 12-membered heterocyclyl)-, —C₆-C₁₀ aryl-, or —(CH₂)ₚ-(5- to 10-membered heteroaryl)-, wherein the alkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy. In some embodiment, p is 0. In some embodiments, p is 1. In some embodiments, p is 2.

In some embodiments, R⁴ is a —C₁-C₆ alkyl- optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R⁴ is a —C₁-C₆ alkyl- substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R⁴ is —(CH₂)ₚ—(C₃-C₁₂ cycloalkyl)-, -(3- to 12-membered heterocyclyl)-, —C₆-C₁₀ aryl-, or —(CH₂)ₚ-(5- to 10-membered heteroaryl)-, wherein the cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy. In some embodiment, p is 0. In some embodiments, p is 1. In some embodiments, p is 2.

In some embodiments, R⁴ is —(CH₂)ₚ—(C₃-C₁₂ cycloalkyl)-. In some embodiment, p is 0. In some embodiments, p is 1. In some embodiments, p is 2.

In some embodiments, R⁴ is —(CH₂)ₚ—(C₃-C₁₂ cycloalkyl)-, wherein the cycloalkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy. In some embodiment, p is 0. In some embodiments, p is 1. In some embodiments, p is 2.

In some embodiments, R⁴ is —(CH₂)ₚ-(3- to 12-membered heterocyclyl)-. In some embodiment, p is 0. In some embodiments, p is 1. In some embodiments, p is 2.

In some embodiments, R⁴ is —(CH₂)ₚ-(3- to 12-membered heterocyclyl)-, wherein the heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy. In some embodiment, p is 0. In some embodiments, p is 1. In some embodiments, p is 2.

In some embodiments, R⁴ is -(3- to 12-membered heterocyclyl)-.

In some embodiments, R⁴ is -(3- to 12-membered heterocyclyl)-, wherein the heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R⁴ is —C₆-C₁₀ aryl-.

In some embodiments, R⁴ is —C₆-C₁₀ aryl-, wherein the aryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R⁴ is —C₆ aryl-.

In some embodiments, R⁴ is —C₆ aryl-, wherein the aryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R⁴ is —(CH₂)ₚ-(5- to 10-membered heteroaryl)-. In some embodiment, p is 0. In some embodiments, p is 1. In some embodiments, p is 2.

In some embodiments, R⁴ is —(CH₂)ₚ-(5- to 10-membered heteroaryl)-, wherein the heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy. In some embodiment, p is 0. In some embodiments, p is 1. In some embodiments, p is 2.

In some embodiments, R⁴ is —(CH₂)ₚ-(6-membered heteroaryl)-. In some embodiment, p is 0. In some embodiments, p is 1. In some embodiments, p is 2.

In some embodiments, R⁴ is —(CH₂)ₚ-(6-membered heteroaryl)-, wherein the heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy. In some embodiment, p is 0. In some embodiments, p is 1. In some embodiments, p is 2.

In some embodiments, R⁴ is -(n-propyl)-, —(CH₂)ₚ—(C₃-C₁₂ cycloalkyl)-, -(3- to 12-membered heterocyclyl)-, —C₆-C₁₀ aryl-, or —(CH₂)ₚ-(5- to 10-membered heteroaryl)-, wherein the n-propyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy. In some embodiment, p is 0. In some embodiments, p is 1. In some embodiments, p is 2.

In some embodiments, R⁴ is -(n-propyl)- optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R⁴ is -(n-propyl)- substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, R⁴ is -(n-propyl)-.

In some embodiments, Z is a —(CH₂)ₙ-(carboxylic acid) or a tetrazole. In some embodiments, n is 0. In some embodiments, n is 1. In some embodiments, n is 2. In some embodiments, n is 3.

In some embodiments, Z is a —(CH₂)ₙ-(carboxylic acid). In some embodiments, n is 0. In some embodiments, n is 1. In some embodiments, n is 2. In some embodiments, n is 3.

In some embodiments, n is 0 and Z is a carboxylic acid.

In some embodiments, Z is —(CH₂)ₙ-tetrazole. In some embodiments, n is 0. In some embodiments, n is 1. In some embodiments, n is 2. In some embodiments, n is 3.

In some embodiments, n is 0 and Z is a tetrazole.

As noted herein, the combination of R⁴ is a divalent moiety and the -(L)ₙ- of group Z form a linker group joining the NR⁷ amide nitrogen to the terminal carboxylic acid or tetrazole group. In some embodiments, the least number of consecutively covalently bonded atoms between the NR⁷ amide nitrogen to the terminal carboxylic acid or tetrazole group is 3 consecutively covalently bonded atoms.

In some embodiments, R⁴ is -(L₁)ₚ-(C₃-C₁₂ cycloalkyl)- or -(L₁)ₚ-(3- to 12-membered heterocyclyl)-, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; p is 0; n is 2; and Z is a -(L₂)ₙ-(carboxylic acid) or -(L₂)ₙ-tetrazole; such that the least number of consecutively covalently bonded atoms between the NR⁷ amide nitrogen to the terminal carboxylic acid or tetrazole group is 3 consecutively covalently bonded atoms. For example, in some embodiments, —R⁴—Z is a group of formula (i):

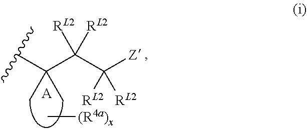

wherein Z′ is tetrazole or carboxylic acid; Ring A is C₃-C₅ cycloalkyl or 4- to 5-membered heterocyclyl; each R^{4a} is independently halo, C₁-C₃ alkyl, C₁-C₃ haloalkyl, or C₃ cycloalkyl; and x is 0, 1, 2, or 3. In some embodiments, at least one instance of R^{L2} is —C₁-C₃ alkyl optionally substituted with one or more halo. In some embodiments, each instance of R^{L2} is H.

In some embodiments, the group of formula (i) is:

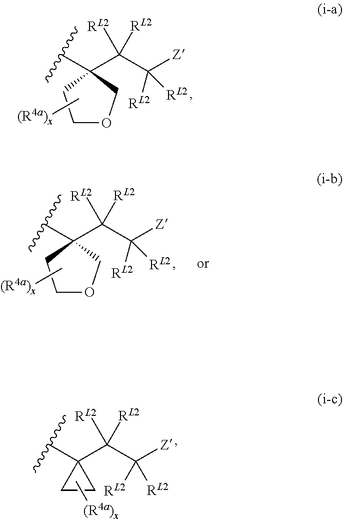

wherein Z′ is tetrazole or carboxylic acid.

In some embodiments, the group of formula (i) is:

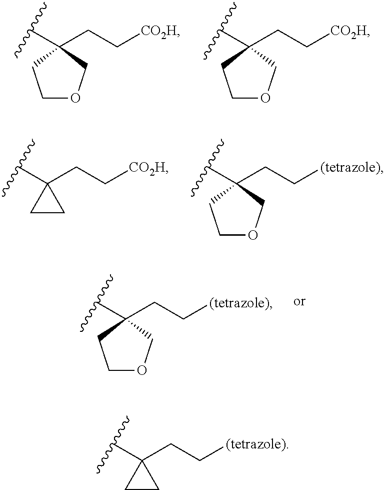

In some embodiments, R⁴ is -(L₁)ₚ-(C₃-C₁₂ cycloalkyl)- or -(L₁)ₚ-(3- to 12-membered heterocyclyl)-, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; p is 1; n is 1; and Z is a -(L₂)ₙ-(carboxylic acid) or -(L₂)ₙ-tetrazole; such that the least number of consecutively covalently bonded atoms between the NR⁷ amide nitrogen to the terminal carboxylic acid or tetrazole group is 3 consecutively covalently bonded atoms. For example, in some embodiments, —R⁴—Z is a group of formula (ii):

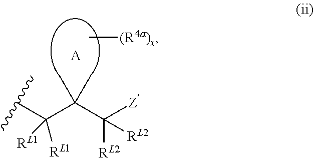

wherein Z′ is tetrazole or carboxylic acid; Ring A is C₃-C₅ cycloalkyl or 4- to 5-membered heterocyclyl; each R^{4a} is independently halo, C₁-C₃ alkyl, C₁-C₃ haloalkyl, or C₃ cycloalkyl; and x is 0, 1, 2, or 3. In some embodiments, at least one instance of R^{L1} is —C₁-C₃ alkyl optionally substituted with one or more halo. In some embodiments, at least one instance of R^{L2} is —C₁-C₃ alkyl optionally substituted with one or more halo. In some embodiments, each instance of R^{L1} is H. In some embodiments, each instance of R^{L2} is H. In some embodiments, each instance of R^{L1} and R^{L2} is H.

In some embodiments, the group of formula (ii) is:

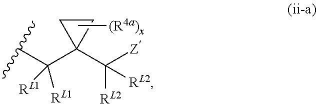

wherein Z′ is tetrazole or carboxylic acid.

In some embodiments, the group of formula (ii-a) is:

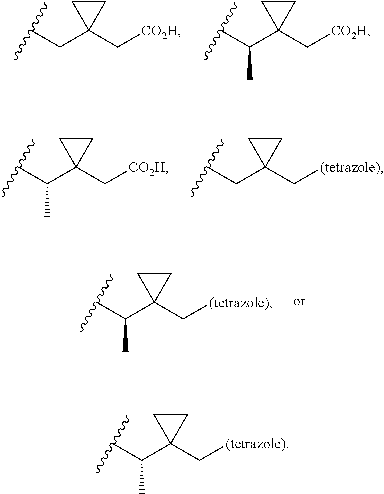

In some embodiments, R⁴ is -(L₁)ₚ-(C₃-C₁₂ cycloalkyl)-, -(L₁)ₚ-(3- to 12-membered heterocyclyl)-, -(L₂)ₚ-(C₆-C₁₀ aryl)-, or -(L₂)ₚ-(5- to 10-membered heteroaryl)-, wherein the cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; p is 0; n is 0; and Z is a -(L₂)ₙ-(carboxylic acid) or -(L₂)ₙ-tetrazole; such that the least number of consecutively covalently bonded atoms between the NR⁷ amide nitrogen to the terminal carboxylic acid or tetrazole group is 3 consecutively covalently bonded atoms. For example, in some embodiments, —R⁴—Z is a group of formula (iii):

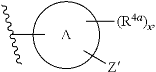

wherein Z′ is tetrazole or carboxylic acid; Ring A is C₃-C₅ cycloalkyl or 4- to 5-membered heterocyclyl; each R^{4a} is independently halo, C₁-C₃ alkyl, C₁-C₃ haloalkyl, or C₃ cycloalkyl; and x is 0, 1, 2, or 3.

In some embodiments, the group of formula (iii) is:

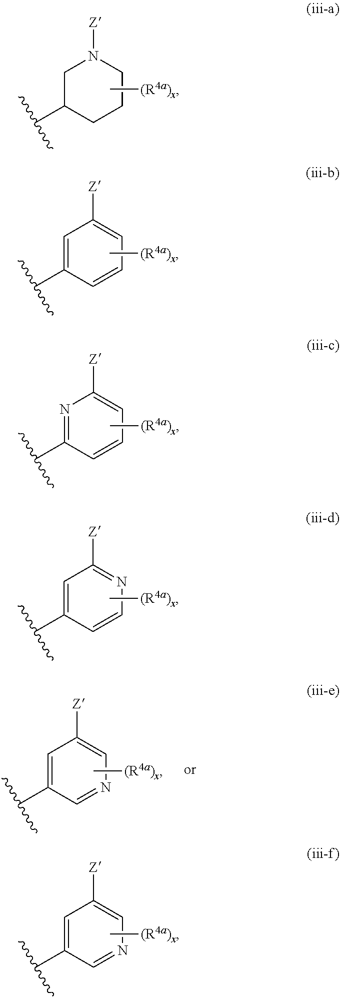

wherein Z′ is tetrazole or carboxylic acid.

In some embodiments, the group of formula (iii-a) is:

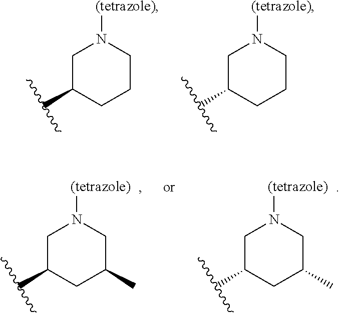

In some embodiments, R⁴ is -(n-propyl)- optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; n is 0; and Z is a -(L₂)ₙ-(carboxylic acid) or -(L₂)ₙ-tetrazole; such that the number of consecutively covalently bonded atoms between the NR⁷ amide nitrogen to the terminal carboxylic acid or tetrazole group is 3 consecutively covalently bonded atoms. In some embodiments, R⁴ is -(n-propyl)-independently substituted with 0, 1, 2, or 3 halo, C₁-C₃ alkyl, C₁-C₃ haloalkyl, or C₃ cycloalkyl; n is 0; and Z is a -(L₂)ₙ-(carboxylic acid) or -(L₂)ₙ-tetrazole. In some embodiments, R⁴ is -(n-propyl)-independently substituted with 0, 1, 2, or 3 fluoro, —CH₃, —CF₃, or C₃ cycloalkyl; n is 0; and Z is a -(L₂)ₙ (carboxylic acid) or -(L₂)ₙ-tetrazole. In some embodiments, —R⁴—Z is a group of formula (iv):

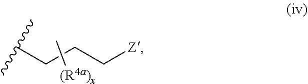

wherein Z′ is tetrazole or carboxylic acid; each R^{4a} is independently halo, C₁-C₃ alkyl, C₁-C₃ haloalkyl, or C₃ cycloalkyl; and x is 0, 1, 2, or 3. In some embodiments, each R^{4a} is independently —CH₃, —CF₃, C₃ cycloalkyl, or fluoro. In some embodiments, each R^{4a} is independently bonded to any one of the carbon atoms of formula (iv).

In some embodiments, the group of formula (iv) is of formula:

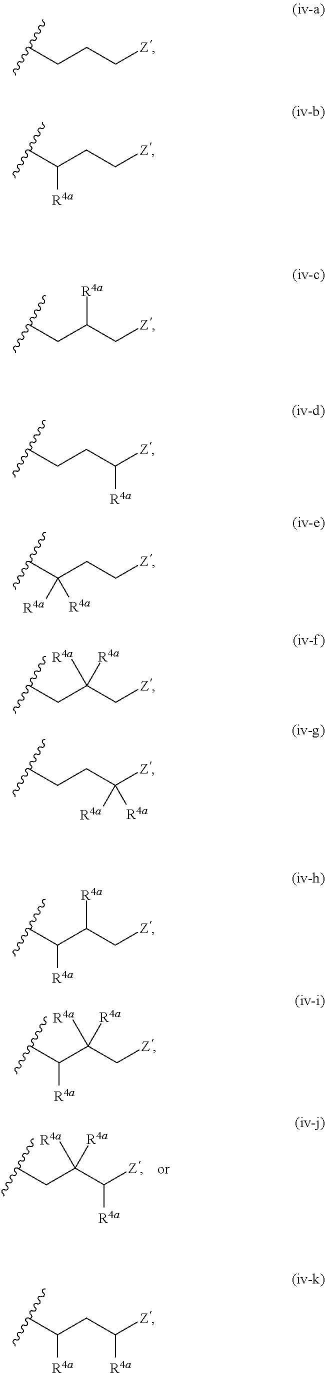

wherein Z′ is tetrazole or carboxylic acid; and each R^{4a} is independently halo, C₁-C₃ alkyl, C₁-C₃ haloalkyl, or C₃ cycloalkyl. In some embodiments, each R^{4a} is independently —CH₃, —CF₃, C₃ cycloalkyl, or fluoro.

In some embodiments, the group of formula (iv) is of formula:

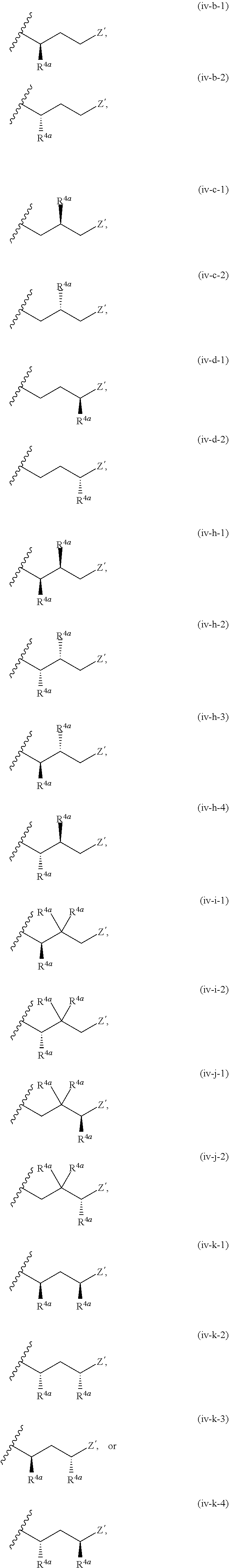

wherein Z′ is tetrazole or carboxylic acid; and each R^{4a} is independently halo, C₁-C₃ alkyl, C₁-C₃ haloalkyl, or C₃ cycloalkyl. In some embodiments, each R^{4a} is independently —CH₃, —CF₃, C₃ cycloalkyl, or fluoro.

In some embodiments, the group of formula (iv) is of formula:

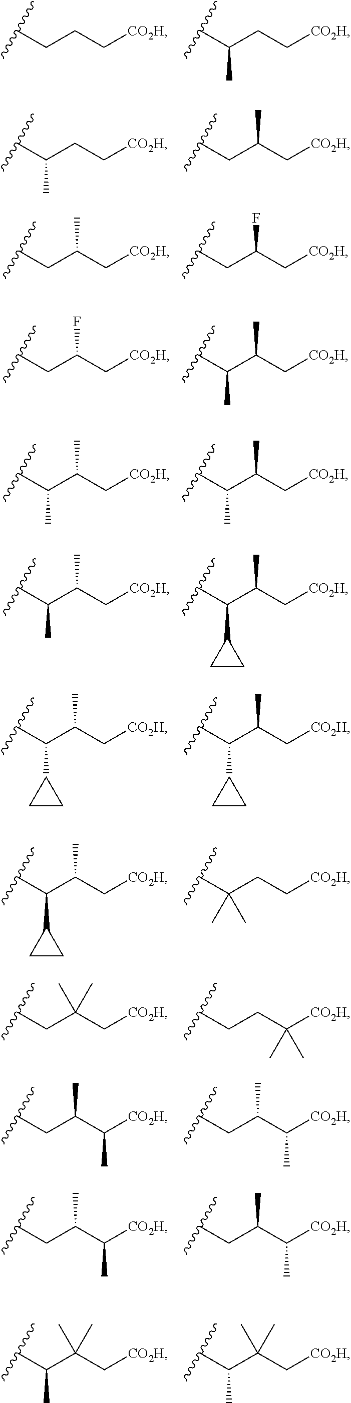

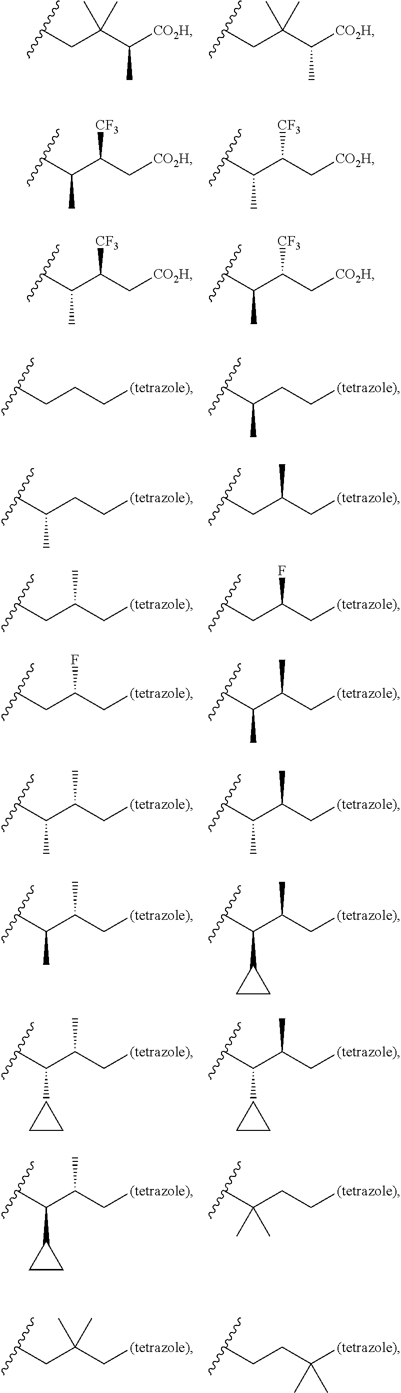

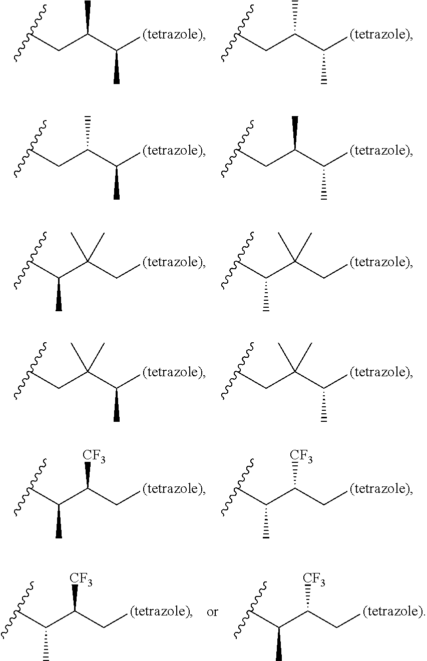

In some embodiments, p is 1; n is 0; Z is a -(L₂)ₙ-(carboxylic acid) or -(L₂)ₙ-tetrazole; and R⁴ is -(L₁)ₚ-(C₆-C₁₀ aryl)- or -(L₁)ₚ-(5- to 10-membered heteroaryl), wherein the aryl or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; such that the least number of consecutively covalently bonded atoms between the NR⁷ amide nitrogen to the terminal carboxylic acid or tetrazole group is 3 consecutively covalently bonded atoms. In some embodiments, provided is an —R⁴—Z group of formula (v):

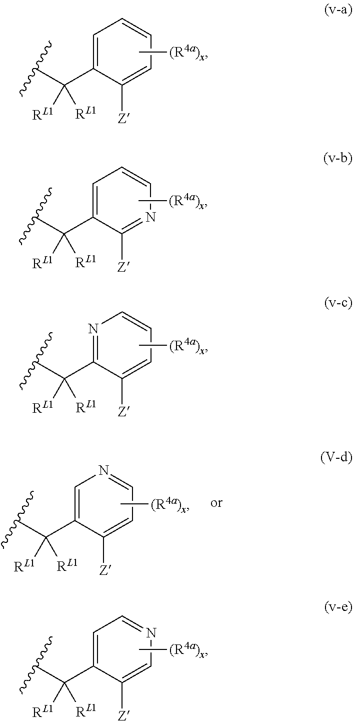

wherein Z′ is tetrazole or carboxylic acid; each R^{4a} is independently halo, C₁-C₃ alkyl, C₁-C₃ haloalkyl; and x is 0, 1, 2, or 3. In some embodiments, at least one instance of R^{L2} is C₁-C₃ alkyl optionally substituted with one or more halo. In some embodiments, each instance of R^{L2} is H.

In some embodiments, the R⁴—Z group of formula (v-a) is:

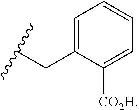 [SMILES: *Cc1ccccc1C(=O)O]

In some embodiments, the least number of consecutively covalently bonded atoms between the NR⁷ amide nitrogen to the terminal carboxylic acid or tetrazole group is 2 consecutively covalently bonded atoms. For example, in some embodiments, p is 0; n is 0; Z is a -(L₂)ₙ-(carboxylic acid) or -(L₂)ₙ-tetrazole; and R⁴ is -(L₁)ₚ-(C₃-C₁₂ cycloalkyl)-, -(L₁)ₚ-(3- to 12-membered heterocyclyl)-, -(L₁)ₚ-(C₆-C₁₀ aryl)-, or -(L₁)ₚ-(5- to 10-membered heteroaryl), wherein the cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; such that the least number of consecutively covalently bonded atoms between the NR⁷ amide nitrogen to the terminal carboxylic acid or tetrazole group is 2 consecutively covalently bonded atoms. In some embodiments, provided is an —R⁴—Z group of formula (vi):

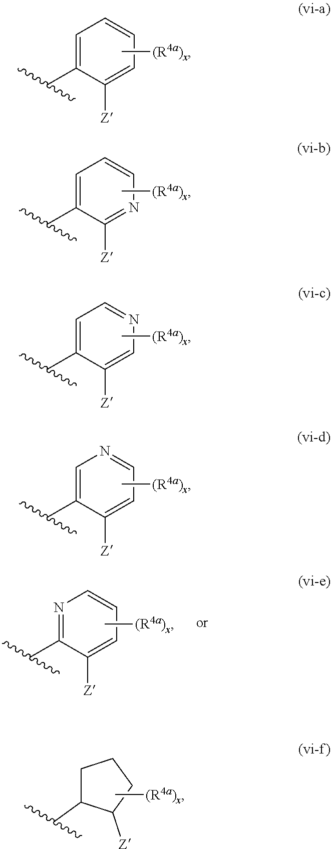

wherein Z′ is tetrazole or carboxylic acid; each R^{4a} is independently halo, C₁-C₃ alkyl, C₁-C₃ haloalkyl; and x is 0, 1, 2, or 3.

In some embodiments, the R⁴—Z group of formula (vi) is:

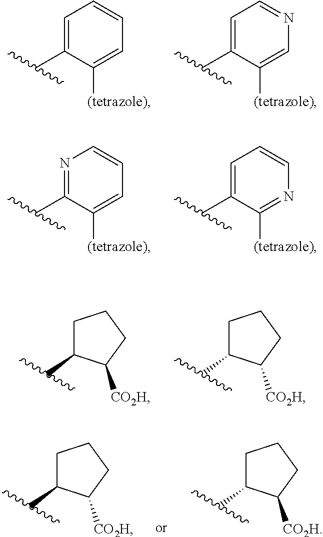

In some embodiments, the least number of consecutively covalently bonded atoms between the NR⁷ amide nitrogen to the terminal carboxylic acid or tetrazole group is 4 consecutively covalently bonded atoms (e.g., carbon atoms). For example, in some embodiments, p is 0; n is 0; Z is a -(L₂)ₙ-(carboxylic acid) or -(L₂)ₙ-tetrazole; and R⁴ is -(L₁)ₚ-(C₆-C₁₀ aryl)-, or -(L₁)ₚ-(5- to 10-membered heteroaryl)- wherein the aryl or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; such that the least number of consecutively covalently bonded atoms between the NR⁷ amide nitrogen to the terminal carboxylic acid or tetrazole group is 4 consecutively covalently bonded atoms. In some embodiments, provided is an —R⁴—Z group of formula (vii):

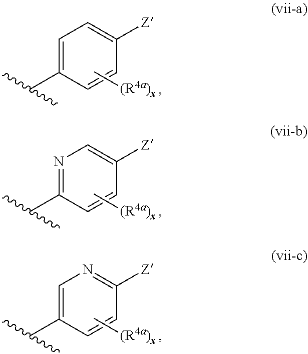

wherein Z′ is tetrazole or carboxylic acid; and each R^{4a} is independently halo, C₁-C₃ alkyl, C₁-C₃ haloalkyl, and x is 0, 1, 2, or 3.

In some embodiments, the R⁴—Z group of formula (vii) is:

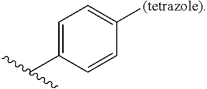

(c) Groups R¹, R⁵, R⁶, R^{6′}, R⁶″

As generally defined herein, each R⁵ is independently fluoro or C₁-C₆ alkyl, wherein the alkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or both R⁵ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, each instance of R⁵ is the same group. In some embodiments, each instance of R⁵ is halo. For example, in some embodiments, each instance of R⁵ is fluoro. In some embodiments, each instance of R⁵ is the same optionally substituted C₁-C₆ alkyl group, e.g., —CH₃. In some embodiments, each instance of R⁵ is different.

In some embodiments, each instance of R⁵ is independently C₁-C₆ alkyl, wherein the alkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, each instance of R⁵ is independently C₁-C₆ alkyl, wherein the alkyl is substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, each instance of R⁵ is independently C₁-C₆ alkyl.

In some embodiments, at least one instance of R⁵ is methyl. In some embodiments, at least one instance of R⁵ is ethyl. In some embodiments, at least one instance of R⁵ is propyl. In some embodiments, at least one instance of R⁵ is butyl. In some embodiments, at least one instance of R⁵ is pentyl. In some embodiments, at least one instance of R⁵ is hexyl. In some embodiments, at least one instance of R⁵ is isopropyl. In some embodiments, at least one instance of R⁵ is isobutyl. In some embodiments, at least one instance of R⁵ is isopentyl. In some embodiments, at least one instance of R⁵ is isohexyl. In some embodiments, at least one instance of R⁵ is secbutyl. In some embodiments, at least one instance of R⁵ is secpentyl. In some embodiments, at least one instance of R⁵ is sechexyl. In some embodiments, at least one instance of R⁵ is tertbutyl.

In some embodiments, both R⁵ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, both R⁵ cyclize, together with the atom to which they are attached, to form a C₃-C₁₂ cycloalkyl substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy.

In some embodiments, both R⁵ cyclize, together with the atom to which they are attached, to form a C₃-C₁₂ cycloalkyl.

In some embodiments, both R⁵ cyclize, together with the atom to which they are attached, to form a C₃ cycloalkyl. In some embodiments, both R⁵ cyclize, together with the atom to which they are attached, to form a C₄ cycloalkyl. In some embodiments, both R⁵ cyclize, together with the atom to which they are attached, to form a C₅ cycloalkyl. In some embodiments, both R⁵ cyclize, together with the atom to which they are attached, to form a C₆ cycloalkyl. In some embodiments, both R⁵ cyclize, together with the atom to which they are attached, to form a C₇ cycloalkyl. In some embodiments, both R⁵ cyclize, together with the atom to which they are attached, to form a C₈ cycloalkyl. In some embodiments, both R⁵ cyclize, together with the atom to which they are attached, to form a C₉ cycloalkyl. In some embodiments, both R⁵ cyclize, together with the atom to which they are attached, to form a C₁₀ cycloalkyl. In some embodiments, both R⁵ cyclize, together with the atom to which they are attached, to form a C₁₁ cycloalkyl. In some embodiments, both R⁵ cyclize, together with the atom to which they are attached, to form a C₁₂ cycloalkyl.

In some embodiments, each instance of R⁵ is selected from the group consisting of fluoro and C₁-C₆ alkyl, or both R⁵ cyclize, together with the atom to which they are attached, to form a C₃ cycloalkyl.

In some embodiments, each instance of R⁵ is selected from the group consisting of fluoro and C₁-C₆ alkyl, or both R⁵ cyclize, together with the atom to which they are attached, to form a C₃ or C₅ cycloalkyl.

In some embodiments, each instance of R⁵ is the same selected from the group consisting of fluoro and C₁-C₆ alkyl, or both R⁵ cyclize, together with the atom to which they are attached, to form a C₃ or C₅ cycloalkyl. In some embodiments, each instance of R⁵ is the same selected from the group consisting of fluoro and —CH₃, or both R⁵ cyclize, together with the atom to which they are attached, to form a C₃ or C₅ cycloalkyl. In some embodiments, each instance of R⁵ is fluoro. In some embodiments, each instance of R⁵ is —CH₃. In some embodiments, both R⁵ cyclize, together with the atom to which they are attached, to form a C₃ or C₅ cycloalkyl.

In some embodiments, each instance of R⁵ is the same selected from the group consisting of fluoro and C₁-C₆ alkyl, or both R⁵ cyclize, together with the atom to which they are attached, to form a C₃ cycloalkyl. In some embodiments, each instance of R⁵ is the same selected from the group consisting of fluoro and —CH₃, or both R⁵ cyclize, together with the atom to which they are attached, to form a C₃ cycloalkyl. In some embodiments, each instance of R⁵ is fluoro. In some embodiments, each instance of R⁵ is —CH₃. In some embodiments, both R⁵ cyclize, together with the atom to which they are attached, to form a C₃ cycloalkyl.

As generally defined herein, R¹ is halo, —CN, C₁-C₆ alkyl, C₂-C₆ alkenyl, C₂-C₆ alkynyl, C₁-C₆ haloalkyl, C₁-C₆ alkoxy, or C₃-C₁₂ cycloalkyl.

In some embodiments, R¹ is halo or —CN.

In some embodiments, R¹ is halo. In some embodiments, R¹ is F, Br, Cl, or I. In some embodiments, R¹ is F, Br, or C₁. In some embodiments, R¹ is F. In some embodiments, R¹ is Br. In some embodiments, R¹ is Cl. In some embodiments, R¹ is I.

In some embodiments, R¹ is —CN.

In some embodiments, R¹ is C₁-C₆ alkyl, C₂-C₆ alkenyl, C₂-C₆ alkynyl, C₁-C₆ haloalkyl, C₁-C₆ alkoxy, or C₃-C₁₂ cycloalkyl.

In some embodiments, R¹ is C₁-C₆ alkyl, C₂-C₆ alkenyl, or C₂-C₆ alkynyl.

In some embodiments, R¹ is C₁-C₆ alkyl.

In some embodiments, R¹ is methyl. In some embodiments, R¹ is ethyl. In some embodiments, R¹ is propyl. In some embodiments, R¹ is butyl. In some embodiments, R¹ is pentyl. In some embodiments, R¹ is hexyl. In some embodiments, R¹ is isopropyl. In some embodiments, R¹ is isobutyl. In some embodiments, R¹ is isopentyl. In some embodiments, R¹ is isohexyl. In some embodiments, R¹ is secbutyl. In some embodiments, R¹ is secpentyl. In some embodiments, R¹ is sechexyl. In some embodiments, R¹ is tertbutyl.

In some embodiments, R¹ is C₂-C₆ alkenyl.

In some embodiments, R¹ is C₂ alkenyl. In some embodiments, R¹ is C₃ alkenyl. In some embodiments, R¹ is C₄ alkenyl. In some embodiments, R¹ is C₅ alkenyl. In some embodiments, R¹ is C₆ alkenyl.

In some embodiments, R¹ is C₂-C₆ alkynyl.

In some embodiments, R¹ is C₂ alkynyl. In some embodiments, R¹ is C₃ alkynyl. In some embodiments, R¹ is C₄ alkynyl. In some embodiments, R¹ is C₅ alkynyl. In some embodiments, R¹ is C₆ alkynyl.

In some embodiments, R¹ is C₁-C₆ haloalkyl or C₁-C₆ alkoxy.

In some embodiments, R¹ is C₁-C₆ haloalkyl.

In some embodiments, R¹ is halomethyl. In some embodiments, R¹ is haloethyl. In some embodiments, R¹ is halopropyl. In some embodiments, R¹ is halobutyl. In some embodiments, R¹ is halopentyl. In some embodiments, R¹ is halohexyl.

In some embodiments, R¹ is C₁-C₆ alkoxy.

In some embodiments, R¹ is methoxy. In some embodiments, R¹ is ethoxy. In some embodiments, R¹ is propoxy. In some embodiments, R¹ is butoxy. In some embodiments, R¹ is pentoxy. In some embodiments, R¹ is hexyloxy.

In some embodiments, R¹ is C₃-C₁₂ cycloalkyl.

In some embodiments, R¹ is C₃-C₆ cycloalkyl.

In some embodiments, R¹ is C₃ cycloalkyl. In some embodiments, R¹ is C₄ cycloalkyl. In some embodiments, R¹ is C₅ cycloalkyl. In some embodiments, R¹ is C₆ cycloalkyl.

In some embodiments, R¹ is C₇ cycloalkyl. In some embodiments, R¹ is C₈ cycloalkyl. In some embodiments, R¹ is C₉ cycloalkyl. In some embodiments, R¹ is C₁₀ cycloalkyl. In some embodiments, R¹ is C₁₁ cycloalkyl. In some embodiments, R¹ is C₁₂ cycloalkyl.

In some embodiments, R¹ is Cl, Br, I, CF₃, cyclopropyl, methyl, isopropyl, or cyclopentyl.

As generally defined herein, R⁶ is H, halo, —OH, C₁-C₆ alkyl, C₂-C₆ alkenyl, C₂-C₆ alkynyl, C₁-C₆ haloalkyl, or C₁-C₆ alkoxy.

In some embodiments, R⁶ is H.

In some embodiments, R⁶ is halo, —OH, C₁-C₆ alkyl, C₂-C₆ alkenyl, C₂-C₆ alkynyl, C₁-C₆ haloalkyl, or C₁-C₆ alkoxy.

In some embodiments, R⁶ is halo or —OH.

In some embodiments, R⁶ is halo.

In some embodiments, R⁶ is F, Cl, Br, or I. In some embodiments, R⁶ is F, Cl, or Br.

In some embodiments, R⁶ is F. In some embodiments, R⁶ is Cl. In some embodiments, R⁶ is Br. In some embodiments, R⁶ is I.

In some embodiments, R⁶ is —OH.

In some embodiments, R⁶ is C₁-C₆ alkyl, C₂-C₆ alkenyl, C₂-C₆ alkynyl, C₁-C₆ haloalkyl, or C₁-C₆ alkoxy.

In some embodiments, R⁶ is C₁-C₆ alkyl, C₂-C₆ alkenyl, or C₂-C₆ alkynyl.

In some embodiments, R⁶ is C₁-C₆ alkyl.

In some embodiments, R⁶ is methyl. In some embodiments, R⁶ is ethyl. In some embodiments, R⁶ is propyl. In some embodiments, R⁶ is butyl. In some embodiments, R⁶ is pentyl. In some embodiments, R⁶ is hexyl. In some embodiments, R⁶ is isopropyl. In some embodiments, R⁶ is isobutyl. In some embodiments, R⁶ is isopentyl. In some embodiments, R⁶ is isohexyl. In some embodiments, R⁶ is secbutyl. In some embodiments, R⁶ is secpentyl. In some embodiments, R⁶ is sechexyl. In some embodiments, R⁶ is tertbutyl.

In some embodiments, R⁶ is C₂-C₆ alkenyl.

In some embodiments, R⁶ is C₂ alkenyl. In some embodiments, R⁶ is C₃ alkenyl. In some embodiments, R⁶ is C₄ alkenyl. In some embodiments, R⁶ is C₅ alkenyl. In some embodiments, R⁶ is C₆ alkenyl.

In some embodiments, R⁶ is C₂-C₆ alkynyl.

In some embodiments, R⁶ is C₂ alkynyl. In some embodiments, R⁶ is C₃ alkynyl. In some embodiments, R⁶ is C₄ alkynyl. In some embodiments, R⁶ is C₅ alkynyl. In some embodiments, R⁶ is C₆ alkynyl.

In some embodiments, R⁶ is C₁-C₆ haloalkyl or C₁-C₆ alkoxy.

In some embodiments, R⁶ is C₁-C₆ haloalkyl.

In some embodiments, R⁶ is halomethyl. In some embodiments, R⁶ is haloethyl. In some embodiments, R⁶ is halopropyl. In some embodiments, R⁶ is halobutyl. In some embodiments, R⁶ is halopentyl. In some embodiments, R⁶ is halohexyl.

In some embodiments, R⁶ is C₁-C₆ alkoxy.

In some embodiments, R⁶ is methoxy. In some embodiments, R⁶ is ethoxy. In some embodiments, R⁶ is propoxy. In some embodiments, R⁶ is butoxy. In some embodiments, R⁶ is pentoxy. In some embodiments, R⁶ is hexyloxy.

In some embodiments, R⁶ is H, F, or —OH.

As generally defined herein, R^{6′} is H, halo, —OH, C₁-C₆ alkyl, C₂-C₆ alkenyl, C₂-C₆ alkynyl, C₁-C₆ haloalkyl, or C₁-C₆ alkoxy.

In some embodiments, R^{6′} is H.

In some embodiments, R^{6′} is halo, —OH, C₁-C₆ alkyl, C₂-C₆ alkenyl, C₂-C₆ alkynyl, C₁-C₆ haloalkyl, or C₁-C₆ alkoxy.

In some embodiments, R^{6′} is halo or —OH.

In some embodiments, R^{6′} is halo.

In some embodiments, R^{6′} is F, Cl, Br, or I. In some embodiments, R^{6′} is F, C₁, or Br.

In some embodiments, R^{6′} is F. In some embodiments, R^{6′} is C₁. In some embodiments, R^{6′} is Br.

In some embodiments, R^{6′} is I.

In some embodiments, R^{6′} is —OH.

In some embodiments, R^{6′} is C₁-C₆ alkyl, C₂-C₆ alkenyl, C₂-C₆ alkynyl, C₁-C₆ haloalkyl, or C₁-C₆ alkoxy.

In some embodiments, R^{6′} is C₁-C₆ alkyl, C₂-C₆ alkenyl, or C₂-C₆ alkynyl.

In some embodiments, R^{6′} is C₁-C₆ alkyl.

In some embodiments, R^{6′} is methyl. In some embodiments, R^{6′} is ethyl. In some embodiments, R^{6′} is propyl. In some embodiments, R^{6′} is butyl. In some embodiments, R^{6′} is pentyl. In some embodiments, R^{6′} is hexyl. In some embodiments, R^{6′} is isopropyl. In some embodiments, R^{6′} is isobutyl.

In some embodiments, R^{6′} is isopentyl. In some embodiments, R^{6′} is isohexyl. In some embodiments, R^{6′} is secbutyl. In some embodiments, R^{6′} is secpentyl. In some embodiments, R^{6′} is sechexyl. In some embodiments, R^{6′} is tertbutyl.

In some embodiments, R^{6′} is C₂-C₆ alkenyl.

In some embodiments, R^{6′} is C₂ alkenyl. In some embodiments, R^{6′} is C₃ alkenyl. In some embodiments, R^{6′} is C₄ alkenyl. In some embodiments, R^{6′} is C₅ alkenyl. In some embodiments, R^{6′} is C₆ alkenyl.

In some embodiments, R^{6′} is C₂-C₆ alkynyl.

In some embodiments, R^{6′} is C₂ alkynyl. In some embodiments, R^{6′} is C₃ alkynyl. In some embodiments, R^{6′} is C₄ alkynyl. In some embodiments, R^{6′} is C₅ alkynyl. In some embodiments, R^{6′} is C₆ alkynyl.

In some embodiments, R^{6′} is C₁-C₆ haloalkyl or C₁-C₆ alkoxy.

In some embodiments, R^{6′} is C₁-C₆ haloalkyl.

In some embodiments, R^{6′} is halomethyl. In some embodiments, R^{6′} is haloethyl. In some embodiments, R^{6′} is halopropyl. In some embodiments, R^{6′} is halobutyl. In some embodiments, R^{6′} is halopentyl. In some embodiments, R^{6′} is halohexyl.

In some embodiments, R^{6′} is C₁-C₆ alkoxy.

In some embodiments, R^{6′} is methoxy. In some embodiments, R^{6′} is ethoxy. In some embodiments, R^{6′} is propoxy. In some embodiments, R^{6′} is butoxy. In some embodiments, R^{6′} is pentoxy.

In some embodiments, R^{6′} is hexyloxy.

In some embodiments, R^{6′} is H, F, CF₃, methoxy, or —OH.

As generally defined herein, R^{6″} is H, halo, —OH, C₁-C₆ alkyl, C₂-C₆ alkenyl, C₂-C₆ alkynyl, C₁-C₆ haloalkyl, or C₁-C₆ alkoxy.

In some embodiments, R^{6″} is H.

In some embodiments, R^{6″} is halo, —OH, C₁-C₆ alkyl, C₂-C₆ alkenyl, C₂-C₆ alkynyl, C₁-C₆ haloalkyl, or C₁-C₆ alkoxy.

In some embodiments, R^{6″} is halo or —OH.

In some embodiments, R^{6″} is halo.

In some embodiments, R^{6″} is F, Cl, Br, or I. In some embodiments, R^{6″} is F, C₁, or Br.

In some embodiments, R^{6″} is F. In some embodiments, R^{6″} is C₁. In some embodiments, R^{6″} is Br.

In some embodiments, R^{6″} is I.

In some embodiments, R^{6″} is —OH.

In some embodiments, R^{6″} is C₁-C₆ alkyl, C₂-C₆ alkenyl, C₂-C₆ alkynyl, C₁-C₆ haloalkyl, or C₁-C₆ alkoxy.

In some embodiments, R^{6″} is C₁-C₆ alkyl, C₂-C₆ alkenyl, or C₂-C₆ alkynyl.

In some embodiments, R^{6″} is C₁-C₆ alkyl.

In some embodiments, R^{6″} is methyl. In some embodiments, R^{6″} is ethyl. In some embodiments, R^{6″} is propyl. In some embodiments, R^{6″} is butyl. In some embodiments, R^{6″} is pentyl. In some embodiments, R^{6″} is hexyl. In some embodiments, R^{6″} is isopropyl. In some embodiments, R^{6″} is isobutyl.

In some embodiments, R^{6″} is isopentyl. In some embodiments, R^{6″} is isohexyl. In some embodiments, R^{6″} is secbutyl. In some embodiments, R^{6″} is secpentyl. In some embodiments, R^{6″} is sechexyl. In some embodiments, R^{6″} is tertbutyl.

In some embodiments, R^{6″} is C₂-C₆ alkenyl.

In some embodiments, R^{6″} is C₂ alkenyl. In some embodiments, R^{6″} is C₃ alkenyl. In some embodiments, R^{6″} is C₄ alkenyl. In some embodiments, R^{6″} is C₅ alkenyl. In some embodiments, R^{6″} is C₆ alkenyl.

In some embodiments, R^{6″} is C₂-C₆ alkynyl.

In some embodiments, R^{6″} is C₂ alkynyl. In some embodiments, R^{6″} is C₃ alkynyl. In some embodiments, R^{6″} is C₄ alkynyl. In some embodiments, R^{6″} is C₅ alkynyl. In some embodiments, R^{6″} is C₆ alkynyl.

In some embodiments, R^{6″} is C₁-C₆ haloalkyl or C₁-C₆ alkoxy.

In some embodiments, R^{6″} is C₁-C₆ haloalkyl.

In some embodiments, R^{6″} is halomethyl. In some embodiments, R^{6″} is haloethyl. In some embodiments, R^{6″} is halopropyl. In some embodiments, R^{6″} is halobutyl. In some embodiments, R^{6″} is halopentyl. In some embodiments, R^{6″} is halohexyl.

In some embodiments, R^{6″} is C₁-C₆ alkoxy.

In some embodiments, R^{6″} is methoxy. In some embodiments, R^{6″} is ethoxy. In some embodiments, R^{6″} is propoxy. In some embodiments, R^{6″} is butoxy. In some embodiments, R^{6″} is pentoxy.

In some embodiments, R^{6″} is hexyloxy.

In some embodiments, R^{6″} is H or F.

In some embodiments, R¹ is halo, C₁-C₃ alkyl, C₁-C₃ haloalkyl, or C₃-C₅ cycloalkyl; R⁶ is H, halo, or —OH; R^{6′} is H, halo, —OH, C₁-C₃ alkyl, C₁-C₃ haloalkyl, or C₁-C₃ alkoxy; and R^{6″} is H or halo.

In some embodiments, the 6,5-bicyclic core of formula (viii):

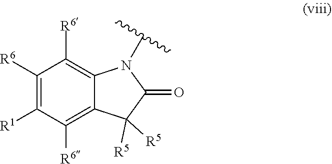

is of formula:

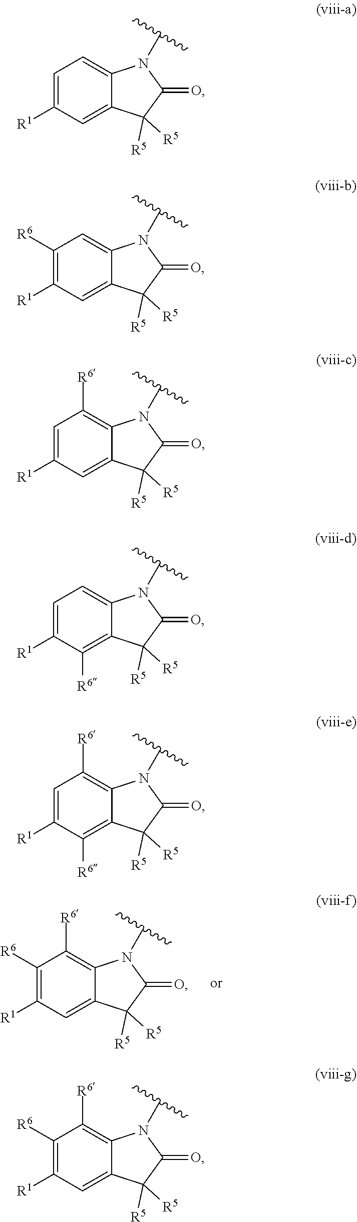

wherein at least one of R⁶, R^{6′} and R^{6″} is not H. In some embodiments of formula (viii), R¹ is halo, C₁-C₃ alkyl, C₁-C₃ haloalkyl, or C₃-C₅ cycloalkyl; R⁶ is halo, or —OH; R^{6′} is halo, —OH, C₁-C₃ alkyl, C₁-C₃ haloalkyl, or C₁-C₃ alkoxy; R^{6″} halo. In some embodiments, each instance of R⁵ is independently the same or different selected from fluoro or C₁₋₃ alkyl, or both R⁵ cyclize, together with the atom to which they are attached, to form a C₃-C₅ cycloalkyl.

In some embodiments of formula (viii-a), the 6,5-bicyclic core is of formula:

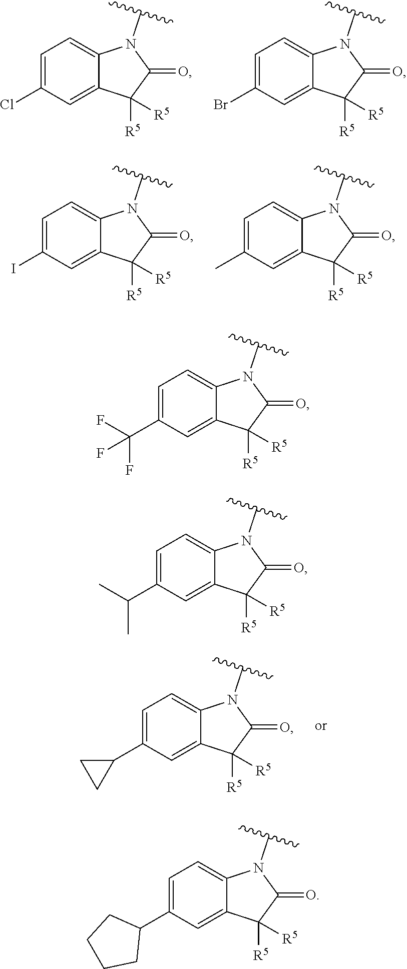

In some embodiments of formula (viii-c), the 6,5-bicyclic core is of formula:

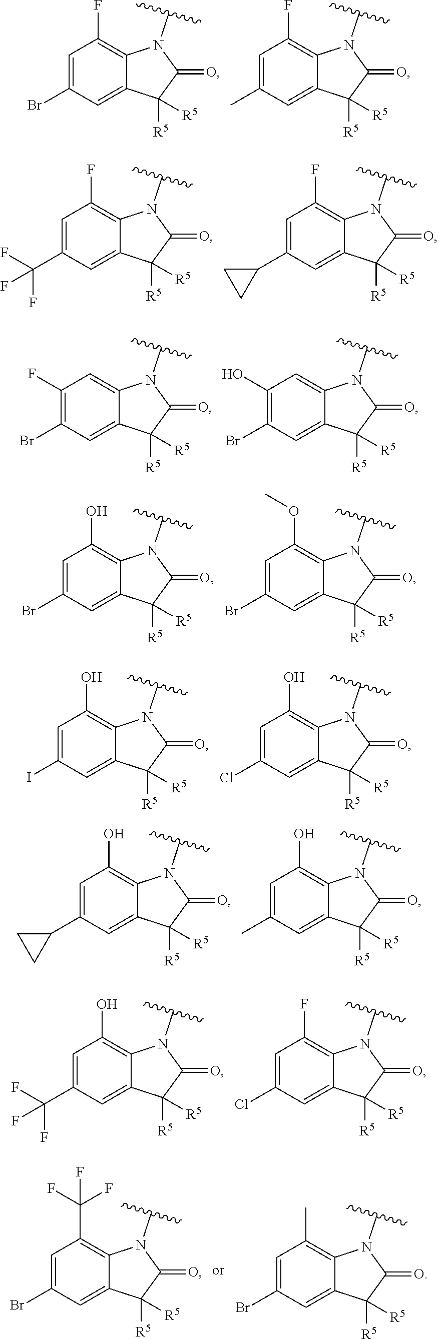

In some embodiments of formula (viii-d), the 6,5-bicyclic core is of formula:

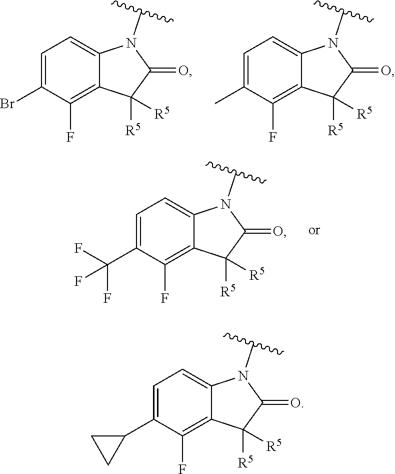

In some embodiments of formula (viii-e), the 6,5-bicyclic core is of formula:

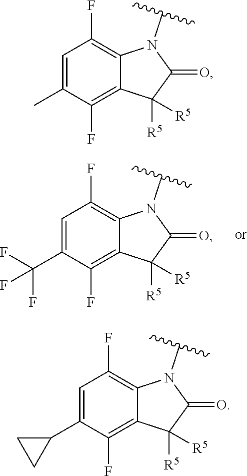

### (d) Additional Embodiments

It is understood that, for a compound of the present disclosure, variables R¹, R², R³, R⁴, R⁵, R⁶, R^{6′}, R^{6″}, R⁷, Z, L₁, L₂, n, and p can each be, where applicable, selected from the groups described herein, and any group described herein for any of variables R¹, R², R³, R⁴, R⁵, R⁶, R^{6′}, R^{6″}, R⁷, Z, L₁, L₂, n, and p can be combined, where applicable, with any group described herein for one or more of the remainder of variables R¹, R², R³, R⁴, R⁵, R⁶, R^{6′}, R^{6″}, R⁷, Z, L₁, L₂, n, and p.

For example, in some embodiments when —R⁴—Z is a group of formula (i), provided is a compound of Formula (I-B):

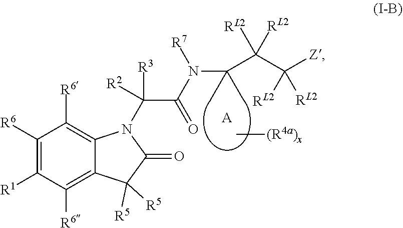

or a pharmaceutically acceptable salt, solvate, clathrate, hydrate, stereoisomer, tautomer, isotopic derivative, prodrug or polymorph thereof, wherein Z′ is tetrazole or carboxylic acid; Ring A is C₃-C₅ cycloalkyl or 4- to 5-membered heterocyclyl; each R^{4a} is independently halo, C₁-C₃ alkyl, C₁-C₃ haloalkyl, or C₃ cycloalkyl; and x is 0, 1, 2, or 3. In some embodiments, each R^{4a} is independently —CH₃, —CF₃, C₃ cycloalkyl, or fluoro. In some embodiments, n is 0 and R^{4a} is absent. In some embodiments, n is 1, 2, or 3, and each instance of R^{4a} is independently —CH₃, —CF₃, C₃ cycloalkyl, or fluoro. In some embodiments, each instance of R² and R³ is H. In some embodiments, Z′ is carboxylic acid. In some embodiments, at least one instance of R^{L2} is —C₁-C₃ alkyl optionally substituted with one or more halo. In some embodiments, each instance of R^{L2} is H. In some embodiments, the —R⁴—Z group of formula (i) is of formula (i-c). In some embodiments, R¹ is halo, C₁-C₃ alkyl, C₁-C₃ haloalkyl, or C₃-C₅ cycloalkyl. In some embodiments, R⁶ is H, halo, or —OH. In some embodiments, R^{6′} is H, halo, —OH, C₁-C₃ alkyl, C₁-C₃ haloalkyl, or C₁-C₃ alkoxy. In some embodiments, R^{6″} is H or halo. In some embodiments, the 6,5-bicyclic core of formula (viii) is of formula (viii-c), (viii-d), or (viii-e). In some embodiments, each instance of R⁵ is independently the same or different selected from fluoro or C₁₋₃ alkyl. In some embodiments, both R⁵ cyclize, together with the atom to which they are attached, to form a C₃ cycloalkyl.

In some embodiments when —R⁴—Z is a group of formula (ii), provided is a compound of Formula (I-C):

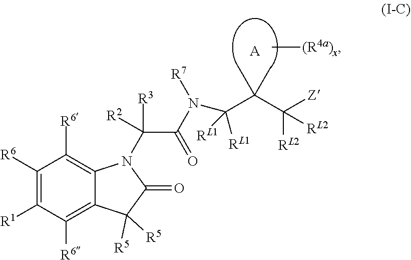

or a pharmaceutically acceptable salt, solvate, clathrate, hydrate, stereoisomer, tautomer, isotopic derivative, prodrug or polymorph thereof, wherein Z′ is tetrazole or carboxylic acid; Ring A is C₃-C₅ cycloalkyl or 4- to 5-membered heterocyclyl; each R^{4a} is independently halo, C₁-C₃ alkyl, C₁-C₃ haloalkyl, or C₃ cycloalkyl; and x is 0, 1, 2, or 3. In some embodiments, each R^{4a} is independently —CH₃, —CF₃, C₃ cycloalkyl, or fluoro. In some embodiments, n is 0 and R^{4a} is absent. In some embodiments, n is 1, 2, or 3, and each instance of R^{4a} is independently —CH₃, —CF₃, C₃ cycloalkyl, or fluoro. In some embodiments, each instance of R² and R³ is H. In some embodiments, Z′ is carboxylic acid. In some embodiments, at least one instance of R^{L2} is —C₁-C₃ alkyl optionally substituted with one or more halo. In some embodiments, each instance of R^{L2} is H. In some embodiments, the —R⁴—Z group of formula (ii) is of formula (ii-a). In some embodiments, R¹ is halo, C₁-C₃ alkyl, C₁-C₃ haloalkyl, or C₃-C₅ cycloalkyl. In some embodiments, R⁶ is H, halo, or —OH. In some embodiments, R^{6′} is H, halo, —OH, C₁-C₃ alkyl, C₁-C₃ haloalkyl, or C₁-C₃ alkoxy. In some embodiments, R^{6″} is H or halo. In some embodiments, the 6,5-bicyclic core of formula (viii) is of formula (viii-c), (viii-d), or (viii-e). In some embodiments, each instance of R⁵ is independently the same or different selected from fluoro or C₁₋₃ alkyl. In some embodiments, both R⁵ cyclize, together with the atom to which they are attached, to form a C₃ cycloalkyl.

In some embodiments when —R⁴—Z is a group of formula (iv), provided is a compound of Formula (I-D):

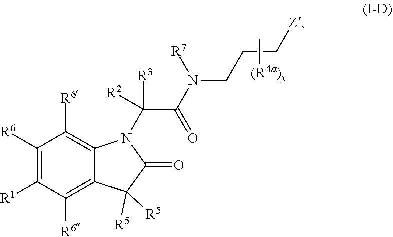

or a pharmaceutically acceptable salt, solvate, clathrate, hydrate, stereoisomer, tautomer, isotopic derivative, prodrug or polymorph thereof, wherein Z′ is tetrazole or carboxylic acid; each R^{4a} is independently halo, C₁-C₃ alkyl, C₁-C₃ haloalkyl, or C₃ cycloalkyl; and x is 0, 1, 2, or 3. In some embodiments, each R^{4a} is independently —CH₃, —CF₃, C₃ cycloalkyl, or fluoro. In some embodiments, n is 0 and R^{4a} is absent. In some embodiments, n is 1, 2, or 3, and each instance of R^{4a} is independently —CH₃, —CF₃, C₃ cycloalkyl, or fluoro. In some embodiments, each instance of R² and R³ is H. In some embodiments, Z′ is carboxylic acid. In some embodiments, the —R⁴—Z group of formula (iv) is of formula (iv-a), (iv-b), (iv-c), or (iv-h). In some embodiments, the —R⁴—Z group of formula (iv-b) is of formula (iv-b-2). In some embodiments, the —R⁴—Z group of formula (iv-c) is of formula (iv-c-1). In some embodiments, the —R⁴—Z group of formula (iv-c) is of formula (iv-c-2). In some embodiments, the —R⁴—Z group of formula (iv-h) is of formula (iv-h-1). In some embodiments, the —R⁴—Z group of formula (iv-h) is of formula (iv-h-2). In some embodiments, the —R⁴—Z group of formula (iv-h) is of formula (iv-h-3). In some embodiments, the —R⁴—Z group of formula (iv-h) is of formula (iv-h-4). In some embodiments, R¹ is halo, C₁-C₃ alkyl, C₁-C₃ haloalkyl, or C₃-C₅ cycloalkyl. In some embodiments, R⁶ is H, halo, or —OH. In some embodiments, R^{6′} is H, halo, —OH, C₁-C₃ alkyl, C₁-C₃ haloalkyl, or C₁-C₃ alkoxy. In some embodiments, R^{6″} is H or halo. In some embodiments, the 6,5-bicyclic core of formula (viii) is of formula (viii-c), (viii-d), or (viii-e). In some embodiments, each instance of R⁵ is independently the same or different selected from fluoro or C₁₋₃ alkyl. In some embodiments, both R⁵ cyclize, together with the atom to which they are attached, to form a C₃ cycloalkyl.

In some embodiments when the 6,5-bicyclic core of formula (viii) is of formula (viii-c), provided is a compound of Formula (I-E):

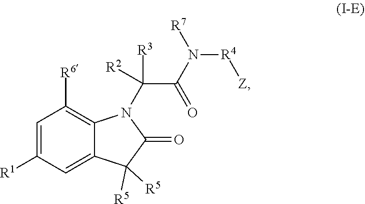

or a pharmaceutically acceptable salt, solvate, clathrate, hydrate, stereoisomer, tautomer, isotopic derivative, prodrug or polymorph thereof. In some embodiments, R^{6′} is H or halo, and R¹ is halo, C₁-C₃ alkyl, C₁-C₃ haloalkyl, or C₃-C₅ cycloalkyl. In some embodiments, R^{6′} is halo and R¹ is C₃-C₅ cycloalkyl. In some embodiments, R^{6′} is fluoro and R¹ is C₃-C₅ cycloalkyl. In some embodiments, R^{6′} is halo and R¹ is C₃ cycloalkyl. In some embodiments, each instance of R² and R³ is H. In some embodiments, Z is -(L₂)ₙ-carboxylic acid. In some embodiments, the —R⁴—Z group is of formula (iv). In some embodiments, the —R⁴—Z group is of formula (iv-a), (iv-b), (iv-c), or (iv-h). In some embodiments, the —R⁴—Z group of formula (iv-b) is of formula (iv-b-2). In some embodiments, the —R⁴—Z group of formula (iv-c) is of formula (iv-c-1). In some embodiments, the —R⁴—Z group of formula (iv-c) is of formula (iv-c-2). In some embodiments, the —R⁴—Z group of formula (iv-h) is of formula (iv-h-1). In some embodiments, the —R⁴—Z group of formula (iv-h) is of formula (iv-h-2). In some embodiments, the —R⁴—Z group of formula (iv-h) is of formula (iv-h-3). In some embodiments, the —R⁴—Z group of formula (iv-h) is of formula (iv-h-4). In some embodiments, each instance of R⁵ is independently the same or different selected from fluoro or C₁₋₃ alkyl. In some embodiments, both R⁵ cyclize, together with the atom to which they are attached, to form a C₃ cycloalkyl.

In some embodiments when the 6,5-bicyclic core of formula (viii) is of formula (viii-d), provided is a compound of Formula (I-F):

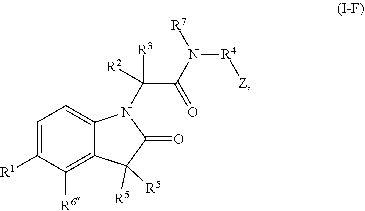

or a pharmaceutically acceptable salt, solvate, clathrate, hydrate, stereoisomer, tautomer, isotopic derivative, prodrug or polymorph thereof. In some embodiments, R^{6″} is H or halo, and R¹ is halo, C₁-C₃ alkyl, C₁-C₃ haloalkyl, or C₃-C₅ cycloalkyl. In some embodiments, R^{6′} is halo and R¹ is C₃-C₅ cycloalkyl. In some embodiments, R^{6′} is fluoro and R¹ is C₃-C₅ cycloalkyl. In some embodiments, R^{6′} is halo and R¹ is C₃ cycloalkyl. In some embodiments, each instance of R² and R³ is H. In some embodiments, Z is -(L₂)ₙ-carboxylic acid. In some embodiments, the —R⁴—Z group is of formula (iv). In some embodiments, the —R⁴—Z group is of formula (iv-a), (iv-b), (iv-c), or (iv-h). In some embodiments, the —R⁴—Z group of formula (iv-b) is of formula (iv-b-2). In some embodiments, the —R⁴—Z group of formula (iv-c) is of formula (iv-c-1). In some embodiments, the —R⁴—Z group of formula (iv-c) is of formula (iv-c-2). In some embodiments, the —R⁴—Z group of formula (iv-h) is of formula (iv-h-1). In some embodiments, the —R⁴—Z group of formula (iv-h) is of formula (iv-h-2). In some embodiments, the —R⁴—Z group of formula (iv-h) is of formula (iv-h-3). In some embodiments, the —R⁴—Z group of formula (iv-h) is of formula (iv-h-4). In some embodiments, each instance of R⁵ is independently the same or different selected from fluoro or C₁₋₃ alkyl. In some embodiments, both R⁵ cyclize, together with the atom to which they are attached, to form a C₃ cycloalkyl.

In some embodiments when the 6,5-bicyclic core of formula (viii) is of formula (viii-e), provided is a compound of Formula (I-G):

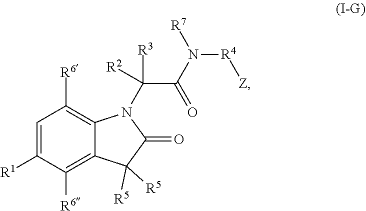

or a pharmaceutically acceptable salt, solvate, clathrate, hydrate, stereoisomer, tautomer, isotopic derivative, prodrug or polymorph thereof. In some embodiments, R¹ is halo, C₁-C₃ alkyl, C₁-C₃haloalkyl, or C₃-C₅ cycloalkyl, R^{6′} is halo, —OH, C₁-C₃ alkyl, C₁-C₃ haloalkyl, or C₁-C₃ alkoxy, and R^{6″} is halo. In some embodiments, R¹ is halo, C₁-C₃ alkyl, C₁-C₃ haloalkyl, or C₃-C₅ cycloalkyl, R^{6′} is halo, and R^{6″} is halo. In some embodiments, R¹ is C₃ cycloalkyl, R^{6′} is halo, and R^{6″} is halo. In some embodiments, R¹ is C₃ cycloalkyl, R^{6′} is fluoro, and R^{6″} is fluoro. In some embodiments, each instance of R² and R³ is H. In some embodiments, Z is -(L₂)ₙ-carboxylic acid. In some embodiments, the —R⁴—Z group is of formula (iv). In some embodiments, the —R⁴—Z group is of formula (iv-a), (iv-b), (iv-c), or (iv-h). In some embodiments, the —R⁴—Z group of formula (iv-b) is of formula (iv-b-2). In some embodiments, the —R⁴—Z group of formula (iv-c) is of formula (iv-c-1). In some embodiments, the —R⁴—Z group of formula (iv-c) is of formula (iv-c-2). In some embodiments, the —R⁴—Z group of formula (iv-h) is of formula (iv-h-1). In some embodiments, the —R⁴—Z group of formula (iv-h) is of formula (iv-h-2). In some embodiments, the —R⁴—Z group of formula (iv-h) is of formula (iv-h-3). In some embodiments, the —R⁴—Z group of formula (iv-h) is of formula (iv-h-4). In some embodiments, each instance of R⁵ is independently the same or different selected from fluoro or C₁₋₃ alkyl. In some embodiments, both R⁵ cyclize, together with the atom to which they are attached, to form a C₃ cycloalkyl.

In some embodiments, the compound is selected from a compound in Table 1 or 2 or a pharmaceutically acceptable salt, solvate, clathrate, hydrate, stereoisomer, tautomer, isotopic derivative, prodrug or polymorph thereof. Compounds comprising a tetrazolyl (Z) group may comprise a mixture of tetrazolyl tautomers. In some embodiments, the compound is a compound in Tables 1 or 2 or a pharmaceutically acceptable salt thereof. In some embodiments, the compound is a neutral (free base) compound in Table 1 or 2. In some embodiments, the compound is a pharmaceutically acceptable salt of a compound in Table 1 or 2. Tables 1 and 2 provide the location of the compound in the Examples (Ex) by Example Number or as provided in Table A (TA) of the Examples. The Asterix (*) next to the Compound Number (#) signifies that arbitrary stereochemistry has been assigned.

TABLE 1
Compounds of Formula (I)

| # | Ex | Compound Name/Structure |
|---|---|---|
| 1 | 1 | N-(3-(1H-tetrazol-5-yl)propyl)- |
| | | 2-(5-bromo-3,3-dimethyl-2- |
| | | oxoindolin-1-yl)acetamide |
| | | 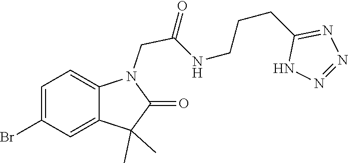 [SMILES: CC1(C)C(=O)N(CC(=O)NCCCc2nnn[nH]2)c2ccc(Br)cc21] |
| | | (tautomer 1) |
| | | N-(3-(2H-tetrazol-5-yl)propyl)- |
| | | 2-(5-bromo-3,3-dimethyl-2- |
| | | oxoindolin-1-yl)acetamide |
| | | 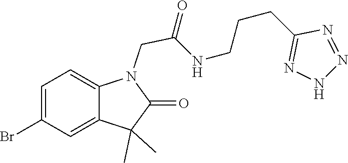 [SMILES: CC1(C)C(=O)N(CC(=O)NCCCc2nn[nH]n2)c2ccc(Br)cc21] |
| | | (tautomer 2) |
| 2 | 2 | 4-(2-(5-bromo-3,3-dimethyl-2- |
| | | oxoindolin-1- |
| | | yl)acetamido)butanoic acid |
| | | 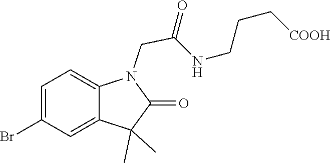 [SMILES: CC1(C)C(=O)N(CC(=O)NCCCC(=O)O)c2ccc(Br)cc21] |
| 3 | TA | 3-(3-(2-(5-bromo-3,3- |
| | | dimethyl-2-oxoindolin-1- |
| | | yl)acetamido)tetrahydrofuran-3- |
| | | yl)propanoic acid |
| | | 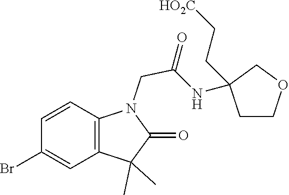 [SMILES: CC1(C)C(=O)N(CC(=O)NC2(CCC(=O)O)CCOC2)c2ccc(Br)cc21] |
| 3A* | TA | (S)-3-(3-(2-(5-bromo-3,3- |
| | | dimethyl-2-oxoindolin-1- |
| | | yl)acetamido)tetrahydrofuran-3- |
| | | yl)propanoic acid |
| | | 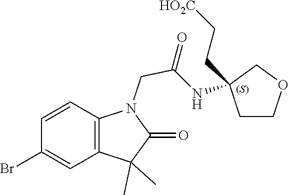 |
| 3B* | TA | (R)-3-(3-(2-(5-bromo-3,3- |
| | | dimethyl-2-oxoindolin-1- |
| | | yl)acetamido)tetrahydrofuran-3- |
| | | yl)propanoic acid |
| | | 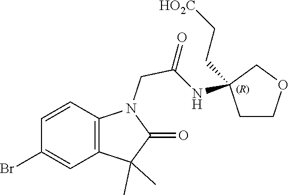 |
| 4 | TA | 4-(2-(5-chloro-3,3-dimethyl-2- |
| | | oxoindolin-1- |
| | | yl)acetamido)butanoic acid |
| | | 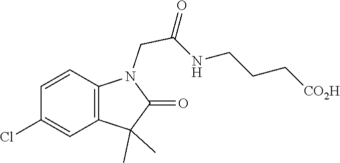 [SMILES: CC1(C)C(=O)N(CC(=O)NCCCC(=O)O)c2ccc(Cl)cc21] |
| 5 | 3 | 4-(2-(5′-bromo-2′- |
| | | oxospiro[cyclopentane-1,3′- |
| | | indolin]-1′- |
| | | yl)acetamido)butanoic acid |
| | | 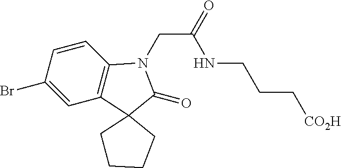 [SMILES: O=C(O)CCCNC(=O)CN1C(=O)C2(CCCC2)c2cc(Br)ccc21] |
| 6 | TA | 4-(2-(5′-bromo-2′- |
| | | oxospiro[cyclopropane-1,3′- |
| | | indolin]-1′- |
| | | yl)acetamido)butanoic acid |
| | | 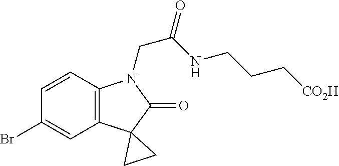 [SMILES: O=C(O)CCCNC(=O)CN1C(=O)C2(CC2)c2cc(Br)ccc21] |
| 7 | TA | 4-(2-(3,3,5-trimethyl-2- |
| | | oxoindolin-1- |
| | | yl)acetamido)butanoic acid |
| | | 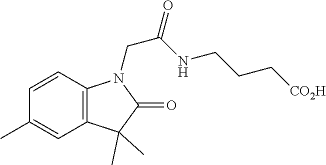 [SMILES: Cc1ccc2c(c1)C(C)(C)C(=O)N2CC(=O)NCCCC(=O)O] |
| 8 | 4 | 4-(2-(3,3-dimethyl-2-oxo-5- |
| | | (trifluoromethyl)indolin-1- |
| | | yl)acetamido)-4-methylpentanoic |
| | | acid |
| | | 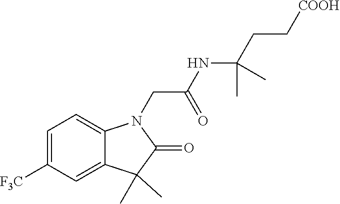 [SMILES: CC(C)(CCC(=O)O)NC(=O)CN1C(=O)C(C)(C)c2cc(C(F)(F)F)ccc21] |
| 9 | 5 | 4-(2-(5-cyclopropyl-3,3- |
| | | dimethyl-2-oxoindolin-1- |
| | | yl)acetamido)-4-methylpentanoic |
| | | acid |
| | | 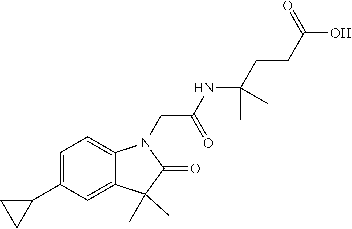 [SMILES: CC(C)(CCC(=O)O)NC(=O)CN1C(=O)C(C)(C)c2cc(C3CC3)ccc21] |
| 10 | TA | 4-(2-(5-isopropyl-3,3-dimethyl- |
| | | 2-oxoindolin-1-yl)acetamido)- |
| | | 4-methylpentanoic acid |
| | | 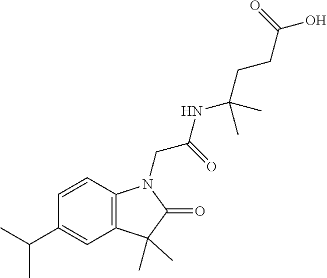 [SMILES: CC(C)c1ccc2c(c1)C(C)(C)C(=O)N2CC(=O)NC(C)(C)CCC(=O)O] |
| 11A | TA | (R)-N-(1-(1H-tetrazol-5- |
| (11) | | yl)piperidin-3-yl)-2-(5-bromo- |
| | | 3,3-dimethyl-2-oxoindolin-1- |
| | | yl)acetamide |
| | | 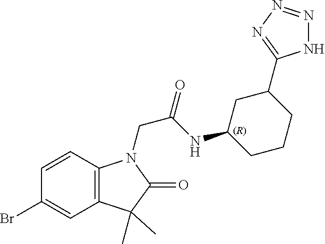 |
| | | (tautomer 1) |
| | | (R)-N-(1-(2H-tetrazol-5- |
| | | yl)piperidin-3-yl)-2-(5-bromo- |
| | | 3,3-dimethyl-2-oxoindolin-1- |
| | | yl)acetamide |
| | | 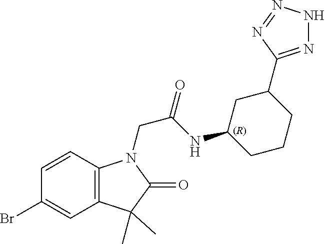 |
| | | (tautomer 2) |
| 11B | TA | (S)-N-(1-(1H-tetrazol-5- |
| | | yl)piperidin-3-yl)-2-(5-bromo- |
| | | 3,3-dimethyl-2-oxoindolin-1- |
| | | yl)acetamide |
| | | 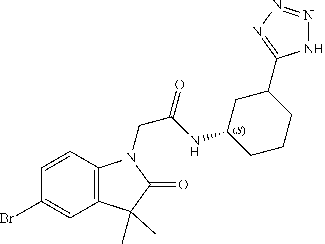 |
| | | (tautomer 1) |
| | | (S)-N-(1-(2H-tetrazol-5- |
| | | yl)piperidin-3-yl)-2-(5-bromo- |
| | | 3,3-dimethyl-2-oxoindolin-1- |
| | | yl)acetamide |
| | | 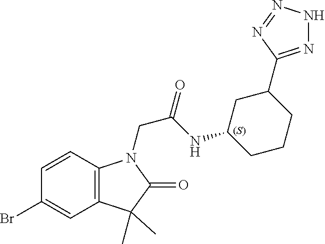 |
| | | (tautomer 2) |
| 12 | TA | N-(3-(1H-tetrazol-5-yl)propyl)- |
| | | 2-(5-chloro-3,3-dimethyl-2- |
| | | oxoindolin-1-yl)acetamide |
| | | 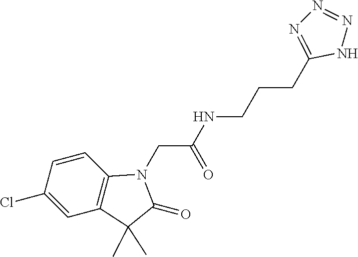 [SMILES: CC1(C)C(=O)N(CC(=O)NCCCc2nnn[nH]2)c2ccc(Cl)cc21] |
| | | (tautomer 1) |
| | | N-(3-(2H-tetrazol-5-yl)propyl)- |
| | | 2-(5-chloro-3,3-dimethyl-2- |
| | | oxoindolin-1-yl)acetamide |
| | | 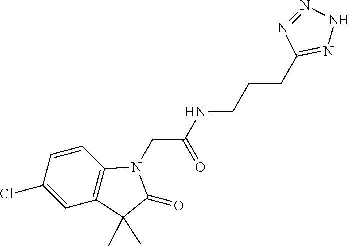 [SMILES: CC1(C)C(=O)N(CC(=O)NCCCc2nn[nH]n2)c2ccc(Cl)cc21] |
| | | (tautomer 2) |
| 13 | TA | 4-(2-(5-bromo-3,3-dimethyl-2- |
| | | oxoindolin-1-yl)acetamido)-3,3- |
| | | dimethylbutanoic acid |
| | | 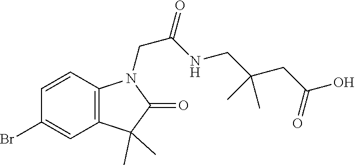 [SMILES: CC(C)(CNC(=O)CN1C(=O)C(C)(C)c2cc(Br)ccc21)CC(=O)O] |
| 14 | TA | 4-(2-(5-bromo-3,3-dimethyl-2- |
| | | oxoindolin-1-yl)acetamido)-2,2- |
| | | dimethylbutanoic acid |
| | | 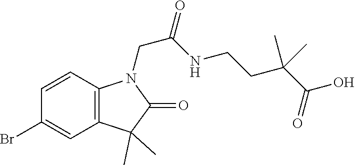 [SMILES: CC(C)(CCNC(=O)CN1C(=O)C(C)(C)c2cc(Br)ccc21)C(=O)O] |
| 15 | 6 | 4-(1-(5-bromo-3,3-dimethyl-2- |
| | | oxoindolin-1-yl)cyclopropane- |
| | | 1-carboxamido)butanoic acid |
| | | 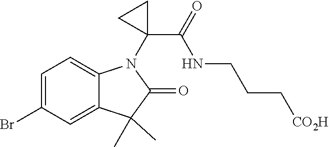 [SMILES: CC1(C)C(=O)N(C2(C(=O)NCCCC(=O)O)CC2)c2ccc(Br)cc21] |
| 16 | 7 | 4-(2-(5-bromo-7-hydroxy-3,3- |
| | | dimethyl-2-oxoindolin-1- |
| | | yl)acetamido)butanoic acid |
| | | 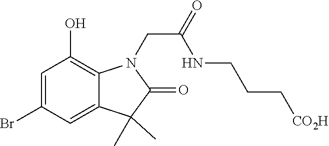 [SMILES: CC1(C)C(=O)N(CC(=O)NCCCC(=O)O)c2c(O)cc(Br)cc21] |
| 17 | TA | N-(2-(1H-tetrazol-5- |
| | | yl)phenyl)-2-(5-bromo-3,3- |
| | | dimethyl-2-oxoindolin-1- |
| | | yl)acetamide |
| | | 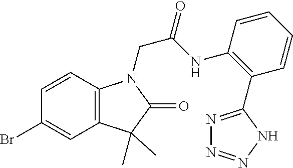 [SMILES: CC1(C)C(=O)N(CC(=O)Nc2ccccc2-c2nnn[nH]2)c2ccc(Br)cc21] |
| | | (tautomer 1) |
| | | N-(2-(2H-tetrazol-5- |
| | | yl)phenyl)-2-(5-bromo-3,3- |
| | | dimethyl-2-oxoindolin-1- |
| | | yl)acetamide |
| | | 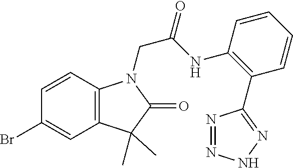 [SMILES: CC1(C)C(=O)N(CC(=O)Nc2ccccc2-c2nn[nH]n2)c2ccc(Br)cc21] |
| | | (tautomer 2) |
| 18 | TA | N-(3-(1H-tetrazol-5- |
| | | yl)phenyl)-2-(5-bromo-3,3- |
| | | dimethyl-2-oxoindolin-1- |
| | | yl)acetamide |
| | | 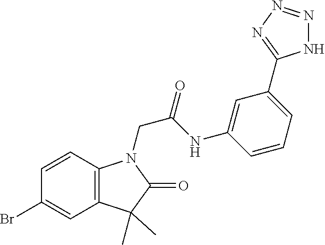 [SMILES: CC1(C)C(=O)N(CC(=O)Nc2cccc(-c3nnn[nH]3)c2)c2ccc(Br)cc21] |
| | | (tautomer 1) |
| | | N-(3-(2H-tetrazol-5- |
| | | yl)phenyl)-2-(5-bromo-3,3- |
| | | dimethyl-2-oxoindolin-1- |
| | | yl)acetamide |
| | | 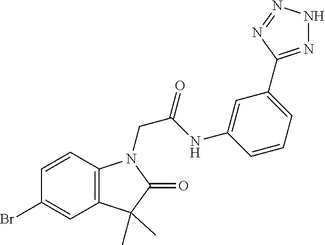 [SMILES: CC1(C)C(=O)N(CC(=O)Nc2cccc(-c3nn[nH]n3)c2)c2ccc(Br)cc21] |
| | | (tautomer 2) |
| 19 | TA | N-(4-(1H-tetrazol-5- |
| | | yl)phenyl)-2-(5-bromo-3,3- |
| | | dimethyl-2-oxoindolin-1- |
| | | yl)acetamide |
| | | 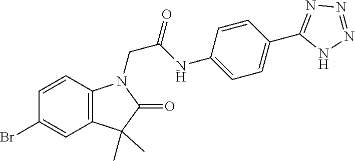 [SMILES: CC1(C)C(=O)N(CC(=O)Nc2ccc(-c3nnn[nH]3)cc2)c2ccc(Br)cc21] |
| | | (tautomer 1) |
| | | N-(4-(2H-tetrazol-5- |
| | | yl)phenyl)-2-(5-bromo-3,3- |
| | | dimethyl-2-oxoindolin-1- |
| | | yl)acetamide |
| | | 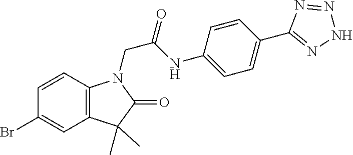 [SMILES: CC1(C)C(=O)N(CC(=O)Nc2ccc(-c3nn[nH]n3)cc2)c2ccc(Br)cc21] |
| | | (tautomer 2) |
| 20 | TA | 4-(2-(5-bromo-6-fluoro-3,3- |
| | | dimethyl-2-oxoindolin-1- |
| | | yl)acetamido)butanoic acid |
| | | 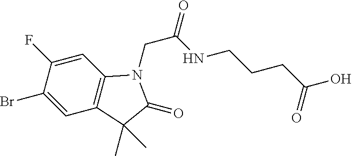 [SMILES: CC1(C)C(=O)N(CC(=O)NCCCC(=O)O)c2cc(F)c(Br)cc21] |
| 21 | TA | 4-(2-(5-bromo-4-fluoro-3,3- |
| | | dimethyl-2-oxoindolin-1- |
| | | yl)acetamido)butanoic acid |
| | | 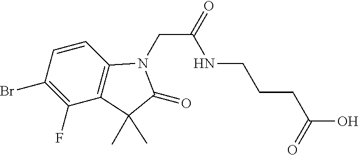 [SMILES: CC1(C)C(=O)N(CC(=O)NCCCC(=O)O)c2ccc(Br)c(F)c21] |
| 22 | TA | 4-(2-(5-iodo-3,3-dimethyl-2- |
| | | oxoindolin-1- |
| | | yl)acetamido)butanoic acid |
| | | 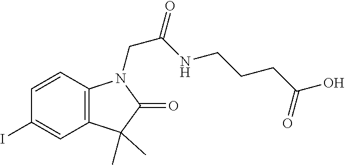 [SMILES: CC1(C)C(=O)N(CC(=O)NCCCC(=O)O)c2ccc(I)cc21] |
| 23 | TA | N-(3-(1H-tetrazol-5-yl)propyl)- |
| | | 2-(5-bromo-3,3-dimethyl-2- |
| | | oxoindolin-1-yl)-N- |
| | | methylacetamide |
| | | 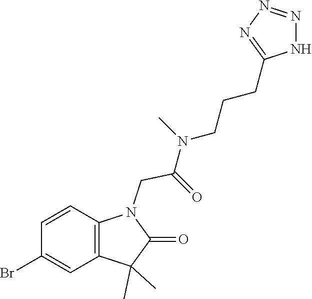 [SMILES: CN(CCCc1nnn[nH]1)C(=O)CN1C(=O)C(C)(C)c2cc(Br)ccc21] |
| | | (tautomer 1) |
| | | N-(3-(2H-tetrazol-5-yl)propyl)- |
| | | 2-(5-bromo-3,3-dimethyl-2- |
| | | oxoindolin-1-yl)-N- |
| | | methylacetamide |
| | | 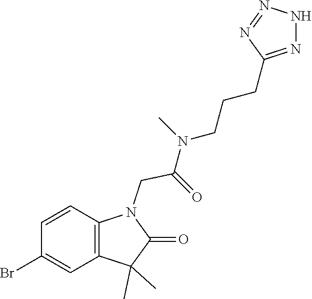 [SMILES: CN(CCCc1nn[nH]n1)C(=O)CN1C(=O)C(C)(C)c2cc(Br)ccc21] |
| | | (tautomer 2) |
| 24 | 8 | 4-(2-(5-bromo-7-fluoro-3,3- |
| | | dimethyl-2-oxoindolin-1- |
| | | yl)acetamido)butanoic acid |
| | | 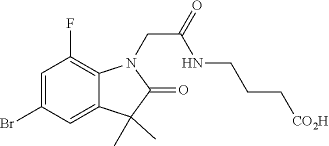 [SMILES: CC1(C)C(=O)N(CC(=O)NCCCC(=O)O)c2c(F)cc(Br)cc21] |
| 25 | TA | 4-(2-(5-cyclopentyl-3,3- |
| | | dimethyl-2-oxoindolin-1- |
| | | yl)acetamido)butanoic acid |
| | | 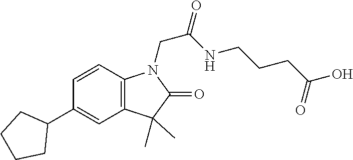 [SMILES: CC1(C)C(=O)N(CC(=O)NCCCC(=O)O)c2ccc(C3CCCC3)cc21] |
| 26 | TA | 2-(5-bromo-7-hydroxy-3,3- |
| | | dimethyl-2-oxoindolin-1-yl)-N- |
| | | ((3R,5S)-5-methyl-1-(1H- |
| | | tetrazol-5-yl)piperidin-3- |
| | | yl)acetamide |
| | | 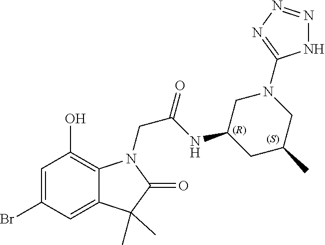 |
| | | (tautomer 1) |
| | | 2-(5-bromo-7-hydroxy-3,3- |
| | | dimethyl-2-oxoindolin-1-yl)-N- |
| | | ((3R,5S)-5-methyl-1-(2H- |
| | | tetrazol-5-yl)piperidin-3- |
| | | yl)acetamide |
| | | 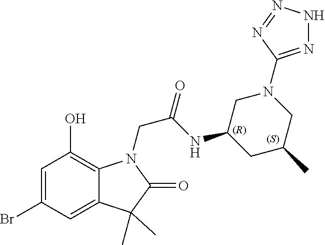 |
| | | (tautomer 2) |
| 27 | 9 | 4-(2-(5-bromo-7-methoxy-3,3- |
| | | dimethyl-2-oxoindolin-1- |
| | | yl)acetamido)butanoic acid |
| | | 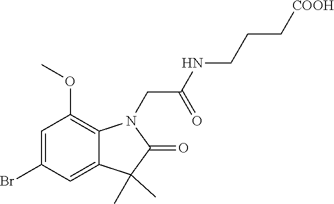 [SMILES: COc1cc(Br)cc2c1N(CC(=O)NCCCC(=O)O)C(=O)C2(C)C] |
| 28 | 10 | 4-(2-(7-hydroxy-5-iodo-3,3- |
| | | dimethyl-2-oxoindolin-1- |
| | | yl)acetamido)butanoic acid |
| | | 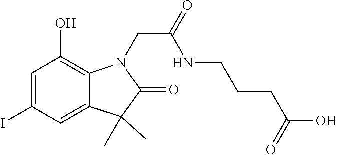 [SMILES: CC1(C)C(=O)N(CC(=O)NCCCC(=O)O)c2c(O)cc(I)cc21] |
| 29 | 11 | N-(3-(1H-tetrazol-5-yl)propyl)- |
| | | 2-(5-cyclopropyl-7-hydroxy-3,3- |
| | | dimethyl-2-oxoindolin-1- |
| | | yl)acetamide |
| | | 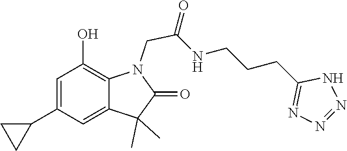 [SMILES: CC1(C)C(=O)N(CC(=O)NCCCc2nnn[nH]2)c2c(O)cc(C3CC3)cc21] |
| | | (tautomer 1) |
| | | N-(3-(2H-tetrazol-5-yl)propyl)- |
| | | 2-(5-cyclopropyl-7-hydroxy-3,3- |
| | | dimethyl-2-oxoindolin-1- |
| | | yl)acetamide |
| | | 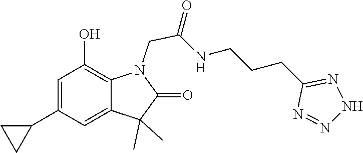 [SMILES: CC1(C)C(=O)N(CC(=O)NCCCc2nn[nH]n2)c2c(O)cc(C3CC3)cc21] |
| | | (tautomer 2) |
| 30 | TA | 4-(2-(5-bromo-7-hydroxy-3,3- |
| | | dimethyl-2-oxoindolin-1-yl)-N- |
| | | methylacetamido)butanoic acid |
| | | 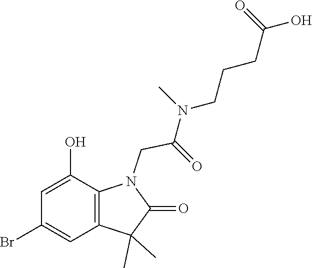 [SMILES: CN(CCCC(=O)O)C(=O)CN1C(=O)C(C)(C)c2cc(Br)cc(O)c21] |
| 31 | TA | 4-(2-(5-bromo-7-hydroxy-3,3- |
| | | dimethyl-2-oxoindolin-1- |
| | | yl)acetamido)-4-methylpentanoic |
| | | acid |
| | | 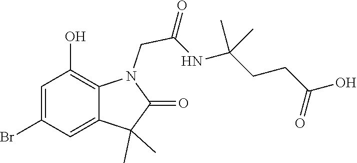 [SMILES: CC(C)(CCC(=O)O)NC(=O)CN1C(=O)C(C)(C)c2cc(Br)cc(O)c21] |
| 32 | TA | 2-(5-bromo-7-hydroxy-3,3- |
| | | dimethyl-2-oxoindolin-1-yl)-N- |
| | | (2-methyl-4-(1H-tetrazol-5- |
| | | yl)butan-2-yl)acetamide |
| | | 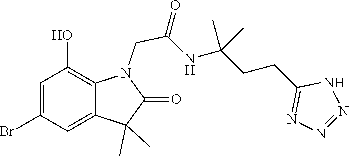 [SMILES: CC(C)(CCc1nnn[nH]1)NC(=O)CN1C(=O)C(C)(C)c2cc(Br)cc(O)c21] |
| | | (tautomer 1) |
| | | 2-(5-bromo-7-hydroxy-3,3- |
| | | dimethyl-2-oxoindolin-1-yl)-N- |
| | | (2-methyl-4-(2H-tetrazol-5- |
| | | yl)butan-2-yl)acetamide |
| | | 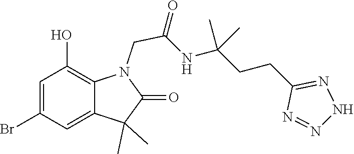 [SMILES: CC(C)(CCc1nn[nH]n1)NC(=O)CN1C(=O)C(C)(C)c2cc(Br)cc(O)c21] |
| | | (tautomer 2) |
| 33 | TA | N-(3-(1H-tetrazol-5-yl)propyl)- |
| | | 2-(5-bromo-7-hydroxy-3,3- |
| | | dimethyl-2-oxoindolin-1- |
| | | yl)acetamide |
| | | 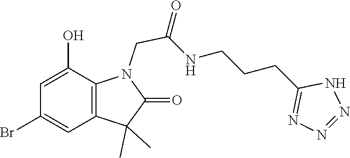 [SMILES: CC1(C)C(=O)N(CC(=O)NCCCc2nnn[nH]2)c2c(O)cc(Br)cc21] |
| | | (tautomer 1) |
| | | N-(3-(2H-tetrazol-5-yl)propyl)- |
| | | 2-(5-bromo-7-hydroxy-3,3- |
| | | dimethyl-2-oxoindolin-1- |
| | | yl)acetamide |
| | | 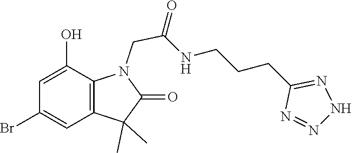 [SMILES: CC1(C)C(=O)N(CC(=O)NCCCc2nn[nH]n2)c2c(O)cc(Br)cc21] |
| | | (tautomer 2) |
| 35 | TA | 2-(1-((2-(5-bromo-7-hydroxy- |
| | | 3,3-dimethyl-2-oxoindolin-1- |
| | | yl)acetamido)methyl)cyclopropyl) |
| | | acetic acid |
| | | 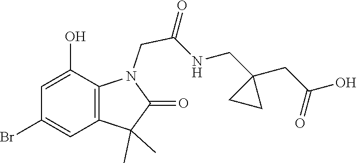 [SMILES: CC1(C)C(=O)N(CC(=O)NCC2(CC(=O)O)CC2)c2c(O)cc(Br)cc21] |
| 36 | 12 | 4-(2-(5-bromo-6-hydroxy-3,3- |
| | | dimethyl-2-oxoindolin-1- |
| | | yl)acetamido)butanoic acid |
| | | 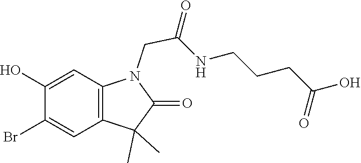 [SMILES: CC1(C)C(=O)N(CC(=O)NCCCC(=O)O)c2cc(O)c(Br)cc21] |
| 37 | TA | N-(2-(1H-tetrazol-5-yl)pyridin- |
| | | 3-yl)-2-(5-bromo-7-hydroxy- |
| | | 3,3-dimethyl-2-oxoindolin-1- |
| | | yl)acetamide |
| | | 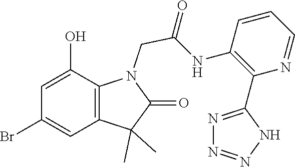 [SMILES: CC1(C)C(=O)N(CC(=O)Nc2cccnc2-c2nnn[nH]2)c2c(O)cc(Br)cc21] |
| | | (tautomer 1) |
| | | N-(2-(2H-tetrazol-5-yl)pyridin- |
| | | 3-yl)-2-(5-bromo-7-hydroxy- |
| | | 3,3-dimethyl-2-oxoindolin-1- |
| | | yl)acetamide |
| | | 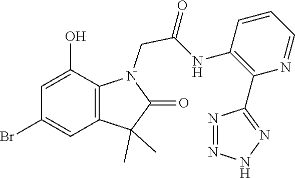 [SMILES: CC1(C)C(=O)N(CC(=O)Nc2cccnc2-c2nn[nH]n2)c2c(O)cc(Br)cc21] |
| | | (tautomer 2) |
| 38 | TA | N-(3-(1H-tetrazol-5-yl)pyridin- |
| | | 2-yl)-2-(5-bromo-7-hydroxy- |
| | | 3,3-dimethyl-2-oxoindolin-1- |
| | | yl)acetamide |
| | | 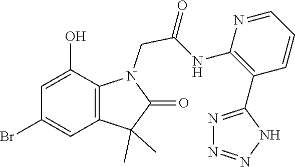 [SMILES: CC1(C)C(=O)N(CC(=O)Nc2ncccc2-c2nnn[nH]2)c2c(O)cc(Br)cc21] |
| | | (tautomer 1) |
| | | N-(3-(2H-tetrazol-5-yl)pyridin- |
| | | 2-yl)-2-(5-bromo-7-hydroxy- |
| | | 3,3-dimethyl-2-oxoindolin-1- |
| | | yl)acetamide |
| | | 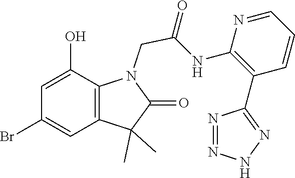 [SMILES: CC1(C)C(=O)N(CC(=O)Nc2ncccc2-c2nn[nH]n2)c2c(O)cc(Br)cc21] |
| | | (tautomer 2) |
| 39 | TA | 4-(2-(7-hydroxy-3,3,5-trimethyl- |
| | | 2-oxoindolin-1- |
| | | yl)acetamido)butanoic acid |
| | | 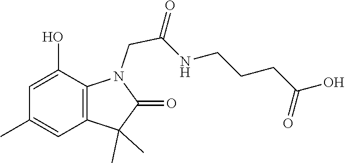 [SMILES: Cc1cc(O)c2c(c1)C(C)(C)C(=O)N2CC(=O)NCCCC(=O)O] |
| 40A* | TA | (R)-3-(3-(2-(5-bromo-7- |
| (40) | | hydroxy-3,3-dimethyl-2- |
| | | oxoindolin-1- |
| | | yl)acetamido)tetrahydrofuran-3- |
| | | yl)propanoic acid |
| | | 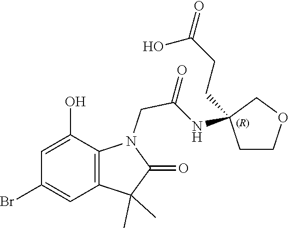 |
| 40B* | TA | (S)-3-(3-(2-(5-bromo-7- |
| (46) | | hydroxy-3,3-dimethyl-2- |
| | | oxoindolin-1- |
| | | yl)acetamido)tetrahydrofuran-3- |
| | | yl)propanoic acid |
| | | 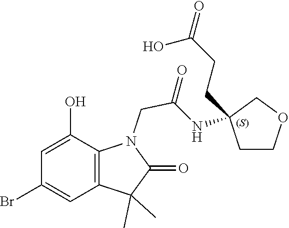 |
| 41A | TA | (R)-4-(2-(5-bromo-7-hydroxy- |
| (41) | | 3,3-dimethyl-2-oxoindolin-1- |
| | | yl)acetamido)pentanoic acid |
| | | 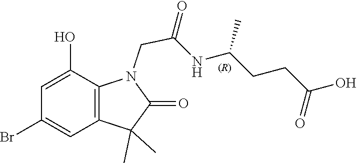 |
| 41B | TA | (S)-4-(2-(5-bromo-7-hydroxy- |
| (34) | | 3,3-dimethyl-2-oxoindolin-1- |
| | | yl)acetamido)pentanoic acid |
| | | 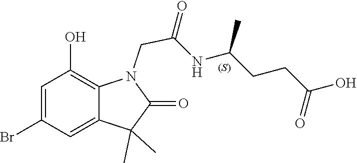 |
| 42A* | TA | (R)-4-(2-(5-bromo-7-hydroxy- |
| (42) | | 3,3-dimethyl-2-oxoindolin-1- |
| | | yl)acetamido)-3-methylbutanoic |
| | | acid |
| | | 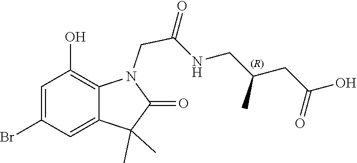 |
| 42B* | TA | (S)-4-(2-(5-bromo-7-hydroxy- |
| (43) | | 3,3-dimethyl-2-oxoindolin-1- |
| | | yl)acetamido)-3-methylbutanoic |
| | | acid |
| | | 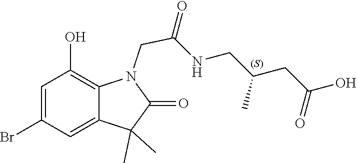 |
| 44 | TA | 3-(1-(2-(5-bromo-7-hydroxy- |
| | | 3,3-dimethyl-2-oxoindolin-1- |
| | | yl)acetamido)cyclopropyl)propanoic |
| | | acid |
| | | 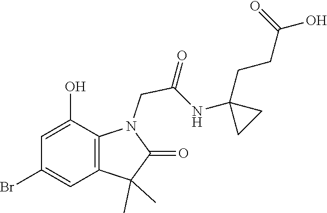 [SMILES: CC1(C)C(=O)N(CC(=O)NC2(CCC(=O)O)CC2)c2c(O)cc(Br)cc21] |
| 45 | TA | N-(3-(1H-tetrazol-5-yl)pyridin- |
| | | 4-yl)-2-(5-bromo-7-hydroxy- |
| | | 3,3-dimethyl-2-oxoindolin-1- |
| | | yl)acetamide |
| | | 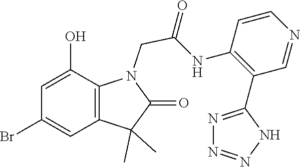 [SMILES: CC1(C)C(=O)N(CC(=O)Nc2ccncc2-c2nnn[nH]2)c2c(O)cc(Br)cc21] |
| | | (tautomer 1) |
| | | N-(3-(2H-tetrazol-5-yl)pyridin- |
| | | 4-yl)-2-(5-bromo-7-hydroxy- |
| | | 3,3-dimethyl-2-oxoindolin-1- |
| | | yl)acetamide |
| | | 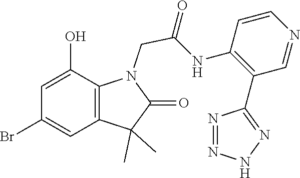 [SMILES: CC1(C)C(=O)N(CC(=O)Nc2ccncc2-c2nn[nH]n2)c2c(O)cc(Br)cc21] |
| | | (tautomer 2) |
| 47 | TA | 4-(2-(5-chloro-7-hydroxy-3,3- |
| | | dimethyl-2-oxoindolin-1- |
| | | yl)acetamido)butanoic acid |
| | | 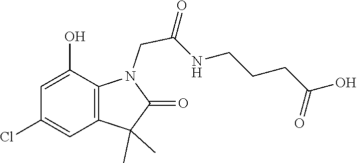 [SMILES: CC1(C)C(=O)N(CC(=O)NCCCC(=O)O)c2c(O)cc(Cl)cc21] |
| 48 | TA | 4-(2-(5-chloro-7-fluoro-3,3- |
| | | dimethyl-2-oxoindolin-1- |
| | | yl)acetamido)butanoic acid |
| | | 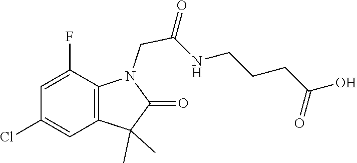 [SMILES: CC1(C)C(=O)N(CC(=O)NCCCC(=O)O)c2c(F)cc(Cl)cc21] |
| 49 | TA | 2-((2-(5-bromo-3,3-dimethyl-2- |
| | | oxoindolin-1- |
| | | yl)acetamido)methyl)benzoic acid |
| | | 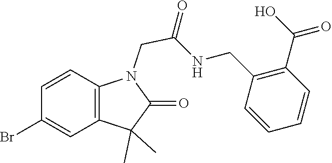 [SMILES: CC1(C)C(=O)N(CC(=O)NCc2ccccc2C(=O)O)c2ccc(Br)cc21] |
| 50 | TA | 4-(2-(5-bromo-3,3-dimethyl-2- |
| | | oxo-7-(trifluoromethyl)indolin-1- |
| | | yl)acetamido)butanoic acid |
| | | 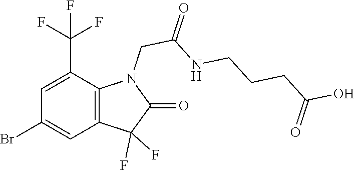 [SMILES: O=C(O)CCCNC(=O)CN1C(=O)C(F)(F)c2cc(Br)cc(C(F)(F)F)c21] |
| 51 | TA | 4-(2-(5-bromo-3,3-difluoro-2- |
| | | oxoindolin-1- |
| | | yl)acetamido)butanoic acid |
| | | 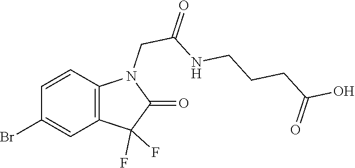 [SMILES: O=C(O)CCCNC(=O)CN1C(=O)C(F)(F)c2cc(Br)ccc21] |
| 52A* | TA | (1S,2R)-2-(2-(5-bromo-3,3- |
| (52) | | dimethyl-2-oxoindolin-1- |
| | | yl)acetamido)cyclopentane-1- |
| | | carboxylic acid |
| | | 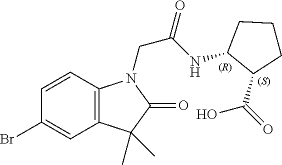 |
| 52B* | TA | (1R,2S)-2-(2-(5-bromo-3,3- |
| (53) | | dimethyl-2-oxoindolin-1- |
| | | yl)acetamido)cyclopentane-1- |
| | | carboxylic acid |
| | | 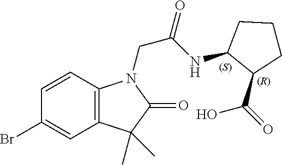 |
| 52C* | TA | (1S,2S)-2-(2-(5-bromo-3,3- |
| (54) | | dimethyl-2-oxoindolin-1- |
| | | yl)acetamido)cyclopentane-1- |
| | | carboxylic acid |
| | | 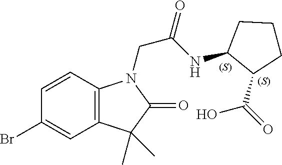 |
| 52D* | TA | (1R,2R)-2-(2-(5-bromo-3,3- |
| (55) | | dimethyl-2-oxoindolin-1- |
| | | yl)acetamido)cyclopentane-1- |
| | | carboxylic acid |
| | | 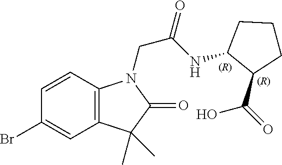 |

TABLE 2
Compounds of Formula (I)

| # | Ex | Compound Name/Structure |
|---|---|---|
| 56 | 13 | 4-(2-(5-cyclopropyl-4-fluoro- |
| | | 3,3-dimethyl-2-oxoindolin-1- |
| | | yl)acetamido)butanoic acid |
| | | 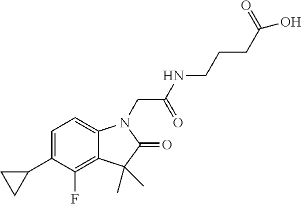 [SMILES: CC1(C)C(=O)N(CC(=O)NCCCC(=O)O)c2ccc(C3CC3)c(F)c21] |
| 57 | 14 | 4-(2-(5-cyclopropyl-4,7- |
| | | difluoro-3,3-dimethyl-2- |
| | | oxoindolin-1- |
| | | yl)acetamido)butanoic acid |
| | | 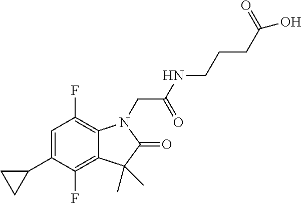 [SMILES: CC1(C)C(=O)N(CC(=O)NCCCC(=O)O)c2c(F)cc(C3CC3)c(F)c21] |
| 58 | 15 | 4-(2-(5-cyclopropyl-4-fluoro- |
| | | 3,3-dimethyl-2-oxoindolin-1- |
| | | yl)acetamido)-3-methylpentanoic |
| | | 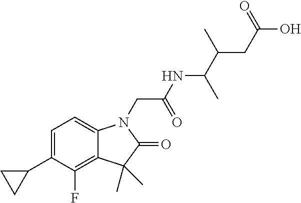 [SMILES: CC(CC(=O)O)C(C)NC(=O)CN1C(=O)C(C)(C)c2c1ccc(C1CC1)c2F] |
| 58A* | 15 | (3S,4R)-4-(2-(5-cyclopropyl-4- |
| | | fluoro-3,3-dimethyl-2- |
| | | oxoindolin-1-yl)acetamido)-3- |
| | | methylpentanoic acid |
| | | 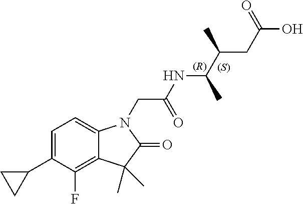 |
| 58B* | 15 | (3R,4S)-4-(2-(5-cyclopropyl-4- |
| | | fluoro-3,3-dimethyl-2- |
| | | oxoindolin-lyl)acetamido)-3- |
| | | methylpentanoic acid |
| | | 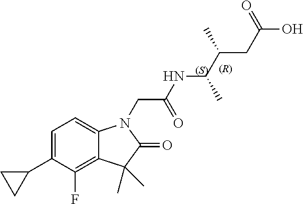 |
| 58C* | | (3S,4S)-4-(2-(5-cyclopropyl-4- |
| | | fluoro-3,3-dimethyl-2- |
| | | oxoindolin-1-yl)acetamido)-3- |
| | | methylpentanoic acid |
| | | 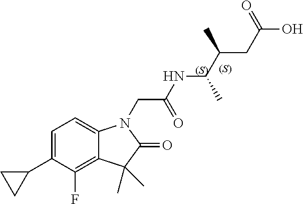 |
| 58D* | 15 | (3R,4R)-4-(2-(5-cyclopropyl-4- |
| | | fluoro-3,3-dimethyl-2- |
| | | oxoindolin-1-yl)acetamido)-3- |
| | | methylpentanoic acid |
| | | 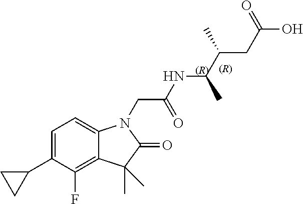 |
| 59A | 16 | (S)-4-(2-(5-cyclopropyl-4,7- |
| | | difluoro-3,3-dimethyl-2- |
| | | oxoindolin-1- |
| | | yl)acetamido)pentanoic acid |
| | | 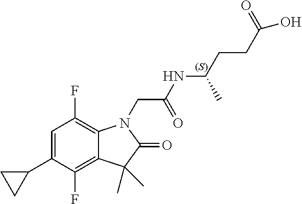 |
| 59B | 16 | (R)-4-(2-(5-cyclopropyl-4,7- |
| | | difluoro-3,3-dimethyl-2- |
| | | oxoindolin-1- |
| | | yl)acetamido)pentanoic acid |
| | | 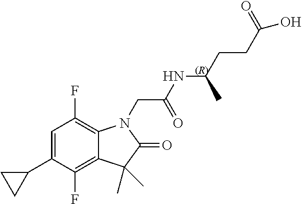 |
| 60A* | 17 | (3S,4S)-4-cyclopropyl-4-(2-(5- |
| | | cyclopropyl-4-fluoro-3,3- |
| | | dimethyl-2-oxoindolin-1- |
| | | yl)acetamido)-3-methylbutanoic |
| | | acid |
| | | 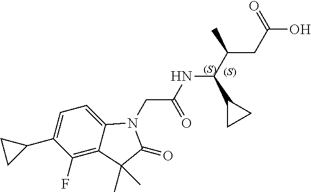 |
| 60B* | 17 | (3R,4R)-4-cyclopropyl-4-(2-(5- |
| | | cyclopropyl-4-fluoro-3,3- |
| | | dimethyl-2-oxoindolin-1- |
| | | yl)acetamido)-3-methylbutanoic |
| | | acid |
| | | 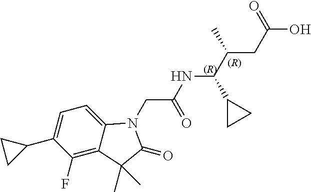 |
| 60C* | 17 | (3S,4R)-4-cyclopropyl-4-(2-(5- |
| | | cyclopropyl-4-fluoro-3,3- |
| | | dimethyl-2-oxoindolin-1- |
| | | yl)acetamido)-3-methylbutanoic |
| | | acid |
| | | 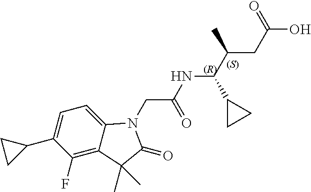 |
| 60D* | 17 | (3R,4S)-4-cyclopropyl-4-(2-(5- |
| | | cyclopropyl-4-fluoro-3,3- |
| | | dimethyl-2-oxoindolin-1- |
| | | yl)acetamido)-3-methylbutanoic |
| | | acid |
| | | 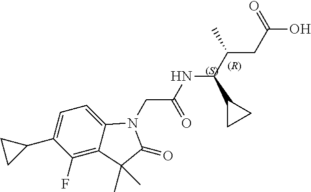 |
| 61A | 18 | (R)-4-(2-(5-cyclopropyl-4,7- |
| | | difluoro-3,3-dimethyl-2- |
| | | oxoindolin-1-yl)acetamido)-3- |
| | | methylbutanoic acid |
| | | 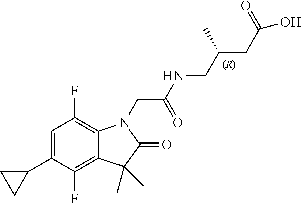 |
| 61B | 18 | (S)-4-(2-(5-cyclopropyl-4,7- |
| | | difluoro-3,3-dimethyl-2- |
| | | oxoindolin-1-yl)acetamido)-3- |
| | | methylbutanoic acid |
| | | 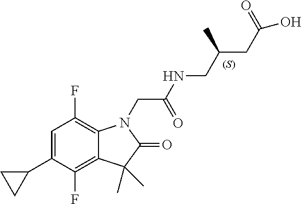 |
| 62A | 19 | (R)-4-(2-(5-cyclopropyl-4,7- |
| | | difluoro-3,3-dimethyl-2- |
| | | oxoindolin-1-yl)acetamido)-3- |
| | | fluorobutanoic acid |
| | | 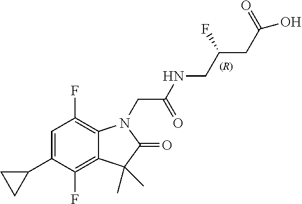 |
| 62B | 19 | (S)-4-(2-(5-cyclopropyl-4,7- |
| | | difluoro-3,3-dimethyl-2- |
| | | oxoindolin-1-yl)acetamido)-3- |
| | | fluorobutanoic acid |
| | | 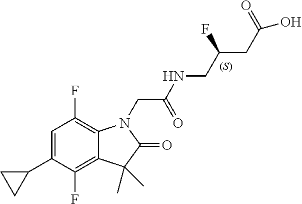 |
| 63A | 20 | (R)-4-(2-(5-cyclopropyl-4- |
| | | fluoro-3,3-dimethyl-2- |
| | | oxoindolin-1-yl)acetamido)-3- |
| | | fluorobutanoic acid |
| | | 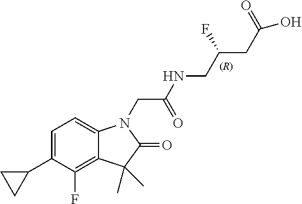 |
| 63B | 20 | (S)-4-(2-(5-cyclopropyl-4- |
| | | fluoro-3,3-dimethyl-2- |
| | | oxoindolin-1-yl)acetamido)-3- |
| | | fluorobutanoic acid |
| | | 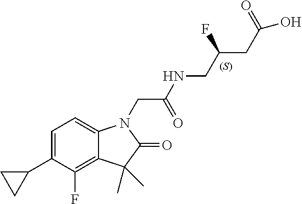 |
| 64A | 21 | (R)-4-(2-(4,7-difluoro-3,3- |
| | | dimethyl-2-oxo-5- |
| | | (trifluoromethyl)indolin-1- |
| | | yl)acetamido)-3-fluorobutanoic |
| | | acid |
| | | 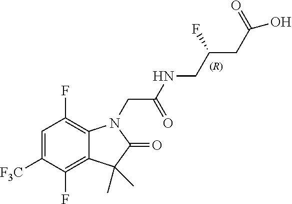 |
| 64B | 21 | (S)-4-(2-(4,7-difluoro-3,3- |
| | | dimethyl-2-oxo-5- |
| | | (trifluoromethyl)indolin-1- |
| | | yl)acetamido)-3-fluorobutanoic |
| | | acid |
| | | 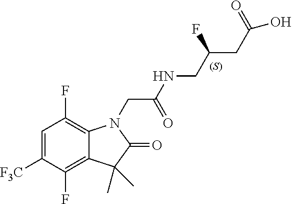 |
| 65 | TA | N-(3-(1H-tetrazol-5-yl)propyl)- |
| | | 2-(5-cyclopropyl-4,7-difluoro- |
| | | 3,3-dimethyl-2-oxoindolin-1- |
| | | yl)acetamide |
| | | 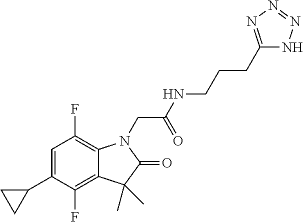 [SMILES: CC1(C)C(=O)N(CC(=O)NCCCc2nnn[nH]2)c2c(F)cc(C3CC3)c(F)c21] |
| | | (tautomer 1) |
| | | N-(3-(2H-tetrazol-5-yl)propyl)- |
| | | 2-(5-cyclopropyl-4,7-difluoro- |
| | | 3,3-dimethyl-2-oxoindolin-1- |
| | | yl)acetamide |
| | | 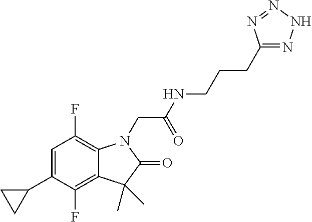 [SMILES: CC1(C)C(=O)N(CC(=O)NCCCc2nn[nH]n2)c2c(F)cc(C3CC3)c(F)c21] |
| | | (tautomer 2) |
| 66 | 22 | 4-(2-(5-cyclopropyl-7-fluoro- |
| | | 3,3-dimethyl-2-oxoindolin-1- |
| | | yl)acetamido)butanoic acid |
| | | 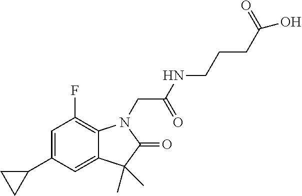 [SMILES: CC1(C)C(=O)N(CC(=O)NCCCC(=O)O)c2c(F)cc(C3CC3)cc21] |
| 67A* | 23 | (3S,4R)-4-(2-(4,7-difluoro-3,3- |
| | | dimethyl-2-oxo-5- |
| | | (trifluoromethyl)indolin-1- |
| | | yl)acetamido)-3-methylpentanoic |
| | | acid |
| | | 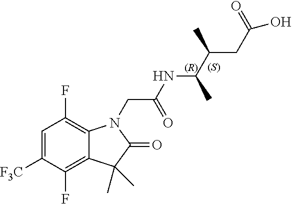 |
| 67B* | 23 | (3R,4S)-4-(2-(4,7-difluoro-3,3- |
| | | dimethyl-2-oxo-5- |
| | | (trifluoromethyl)indolin-1- |
| | | yl)acetamido)-3-methylpentanoic |
| | | acid |
| | | 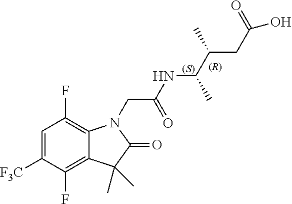 |
| 67C* | 23 | (3S,4S)-4-(2-(4,7-difluoro-3,3- |
| | | dimethyl-2-oxo-5- |
| | | (trifluoromethyl)indolin-1- |
| | | yl)acetamido)-3-methylpentanoic |
| | | acid |
| | | 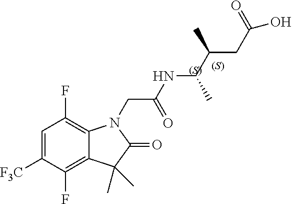 |
| 67D* | 23 | (3R,4R)-4-(2-(4,7-difluoro-3,3- |
| | | dimethyl-2-oxo-5- |
| | | (trifluoromethyl)indolin-1- |
| | | yl)acetamido)-3-methylpentanoic |
| | | acid |
| | | 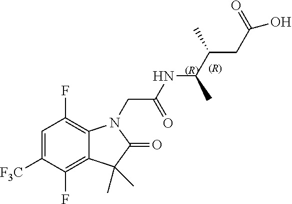 |
| 68A* | TA | (3S,4S)-4-cyclopropyl-4-(2-(5- |
| | | cyclopropyl-4,7-difluoro-3,3- |
| | | dimethyl-2-oxoindolin-1- |
| | | yl)acetamido)-3-methylbutanoic |
| | | 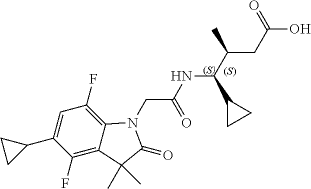 |
| 68B* | TA | (3R,4R)-4-cyclopropyl-4-(2-(5- |
| | | cyclopropyl-4,7-difluoro-3,3- |
| | | dimethyl-2-oxoindolin-1- |
| | | yl)acetamido)-3-methylbutanoic |
| | | 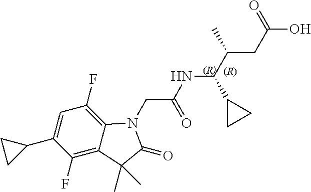 |
| 68C* | TA | (3S,4R)-4-cyclopropyl-4-(2-(5- |
| | | cyclopropyl-4,7-difluoro-3,3- |
| | | dimethyl-2-oxoindolin-1- |
| | | yl)acetamido)-3-methylbutanoic |
| | | acid |
| | | 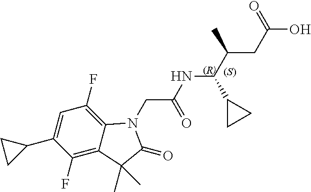 |
| 68D* | TA | (3R,4S)-4-cyclopropyl-4-(2-(5- |
| | | cyclopropyl-4,7-difluoro-3,3- |
| | | dimethyl-2-oxoindolin-1- |
| | | yl)acetamido)-3-methylbutanoic |
| | | acid |
| | | 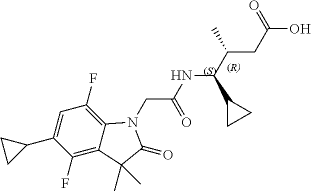 |
| 69A* | 24 | (R)-2-(1-(1-(2-(5-cyclopropyl- |
| | | 4,7-difluoro-3,3-dimethyl-2- |
| | | oxoindolin-1- |
| | | yl)acetamido)ethyl)cyclopropyl)acetic |
| | | acid |
| | | 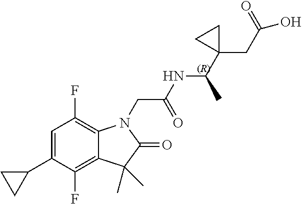 |
| 69B* | 24 | (S)-2-(1-(1-(2-(5-cyclopropyl- |
| | | 4,7-difluoro-3,3-dimethyl-2- |
| | | oxoindolin-1- |
| | | yl)acetamido)ethyl)cyclopropyl)acetic |
| | | acid |
| | | 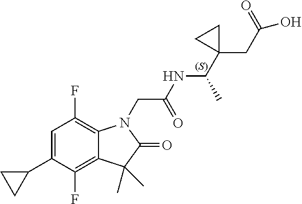 |
| 70A* | 25 | (2S,3S)-4-(2-(5-cyclopropyl-4,7- |
| | | difluoro-3,3-dimethyl-2- |
| | | oxoindolin-1-yl)acetamido)-2,3- |
| | | dimethylbutanoic acid |
| | | 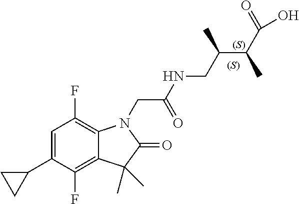 |
| 70B* | 25 | (2R,3R)-4-(2-(5-cyclopropyl-4,7- |
| | | difluoro-3,3-dimethyl-2- |
| | | oxoindolin-1-yl)acetamido)-2,3- |
| | | dimethylbutanoic acid |
| | | 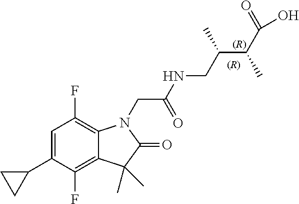 |
| 70C* | 25 | (2S,3R)-4-(2-(5-cyclopropyl-4,7- |
| | | difluoro-3,3-dimethyl-2- |
| | | oxoindolin-1-yl)acetamido)-2,3- |
| | | dimethylbutanoic acid |
| | | 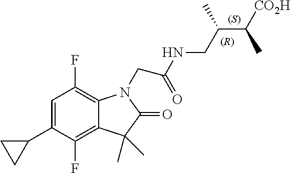 |
| 70D* | 25 | (2R,3S)-4-(2-(5-cyclopropyl-4,7- |
| | | difluoro-3,3-dimethyl-2- |
| | | oxoindolin-1-yl)acetamido)-2,3- |
| | | dimethylbutanoic acid |
| | | 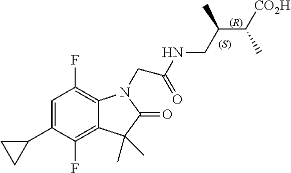 |
| 71A* | 26 | (R)-4-(2-(5-cyclopropyl-4,7- |
| | | difluoro-3,3-dimethyl-2- |
| | | oxoindolin-1-yl)acetamido)-3,3- |
| | | dimethylpentanoic acid |
| | | 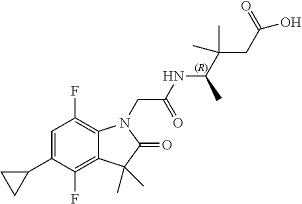 |
| 71B* | 26 | (S)-4-(2-(5-cyclopropyl-4,7- |
| | | difluoro-3,3-dimethyl-2- |
| | | oxoindolin-1-yl)acetamido)-3,3- |
| | | dimethylpentanoic acid |
| | | 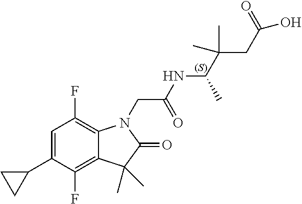 |
| 72A* | 27 | (3S,4R)-4-(2-(5-cyclopropyl-4,7- |
| | | difluoro-3,3-dimethyl-2- |
| | | oxoindolin-1-yl)acetamido)-3- |
| | | (trifluoromethyl)pentanoic acid |
| | | 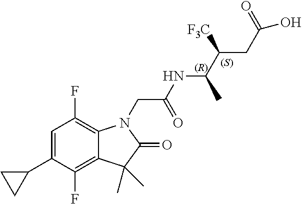 |
| 72B* | 27 | (3R,4S)-4-(2-(5-cyclopropyl-4,7- |
| | | difluoro-3,3-dimethyl-2- |
| | | oxoindolin-1-yl)acetamido)-3- |
| | | (trifluoromethyl)pentanoic acid |
| | | 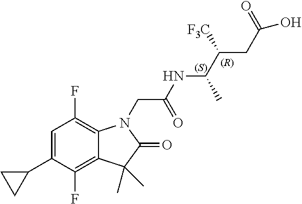 |
| 72C* | 27 | (3S,4S)-4-(2-(5-cyclopropyl-4,7- |
| | | difluoro-3,3-dimethyl-2- |
| | | oxoindolin-1-yl)acetamido)-3- |
| | | (trifluoromethyl)pentanoic acid |
| | | 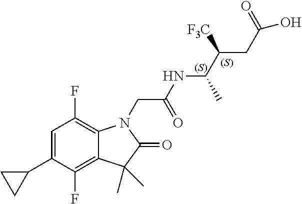 |
| 72D* | 27 | (3R,4R)-4-(2-(5-cyclopropyl-4,7- |
| | | difluoro-3,3-dimethyl-2- |
| | | oxoindolin-1-yl)acetamido)-3- |
| | | (trifluoromethyl)pentanoic acid |
| | | 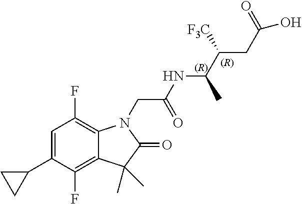 |

In some embodiments, the compound is Compound 58 (e.g., Compound 58A*), Compound 59A, Compound 60A*, Compound 61A, Compound 611B, Compound 62A, Compound 63A, Compound 64A, Compound 66, Compound 67C* or Compound 67D*, or a pharmaceutically acceptable salt, solvate, clathrate, hydrate, stereoisomer, tautomer, isotopic derivative, prodrug or polymorph thereof.

In some embodiments, the compound is Compound 58 (e.g., Compound 58A*), Compound 59A, Compound 60A*, Compound 61A, Compound 611B, Compound 62A, Compound 63A, Compound 64A, Compound 66, Compound 67C* or Compound 67D*, or a pharmaceutically acceptable salt thereof.

In some embodiments, the compound is a free base of Compound 58 (e.g., Compound 58A*), Compound 59A, Compound 60A*, Compound 61A, Compound 611B, Compound 62A, Compound 63A, Compound 64A, Compound 66, Compound 67C* or Compound 67D*.

In some embodiments, the compound is an isotopic derivative of a compound of Formula (I), or any of the compounds provided in Tables 1 or 2. In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S.

In some embodiments, the isotopic derivative is a deuterium labeled compound (i.e., being enriched with ²H with regard to one or more atoms thereof). In some embodiments, the compound is a ¹⁸F labeled compound. In some embodiments, the compound is a ¹²³I labeled compound, a ¹²⁴I labeled compound, a ¹²⁵ I labeled compound, a ¹²⁹I labeled compound, a ¹³¹I labeled compound, a ¹³⁵I labeled compound, or any combination thereof. In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³S, ³²S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³²S, ³⁴S, ³⁵S, and/or ³⁶S labeled reagent for a non-isotope labeled reagent. A compound containing one or more of the aforementioned ¹⁸F, ¹²³I, ¹²⁴I, ¹²⁵I, ¹²⁹I, ¹³¹I, ¹³⁵I, ³²S, ³⁴S, ³⁵S, and ³⁶S atom(s) is within the scope of the present disclosure. Further, substitution with an isotope (e.g., ¹⁸F, ¹²³I, ¹²⁴I, ¹²⁵I, ¹²⁹I, ¹³¹I, ¹³⁵I, ³²S, ³⁴S, ³⁵S, and/or ³⁶S) may afford certain therapeutic advantages resulting from greater metabolic stability, e.g., increased in vivo half-life or reduced dosage requirements.

In some embodiments, the isotopic derivative is a deuterium labeled compound of any one of the compounds of the Formulae disclosed herein.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

### (2) Methods of Preparation

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; Greene, T. W., Wuts, P. G. M., Protective Groups in Organic Synthesis, 3rd edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art.

One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3rd edition, John Wiley & Sons: New York, 1999. By way of example, carboxylic acid protecting groups (PG) may include C₁₋₆alkyl, C₆aryl, or an arylC₁₋₆alkyl group, wherein the alkyl and aryl are optionally substituted with halo, alkyl, or alkoxy groups. In some embodiments, the carboxylic acid protecting group (PG) is methyl (—CH₃, Me), ethyl (—CH₂CH₃, Et), or t-Butyl (—C(CH₃)₃, tBu).

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

Suitable General Preparative Methods of compounds of Formula (I), and intermediates useful in the synthesis of said compounds, are provided below in General Schemes 1-12. The Examples also describe non-limiting procedures in the preparation of such compounds.

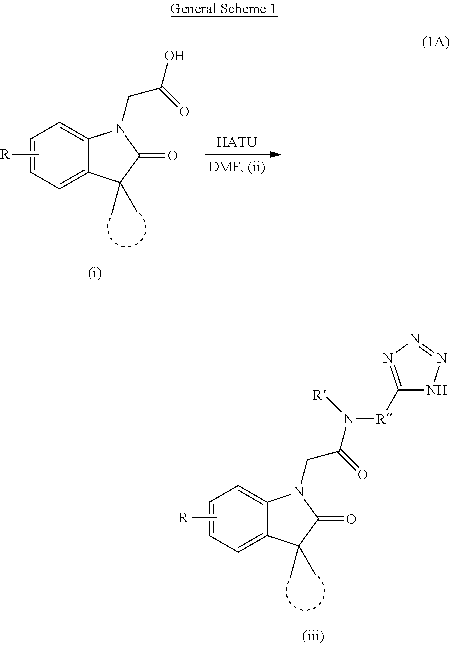

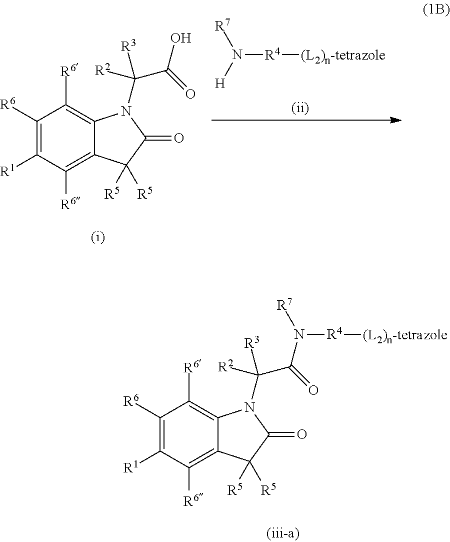

Compounds of Formula (I), wherein Z is -(L₂)ₙ-tetrazole, may be prepared as shown in General Scheme 1, through the amide coupling of a substituted indolinone(acetic acid) (i), or salt thereof, with an aminotetrazole (ii), or salt thereof, to provide the target tetrazole analogs (iii) or (iii-a), or salt thereof, wherein indolinone(acetic acid) (i) and aminotetrazoles (ii) are either commercially available or known in the chemical literature, unless otherwise indicated, and the dashed lines correspond to an optional spirofused cycloalkyl.

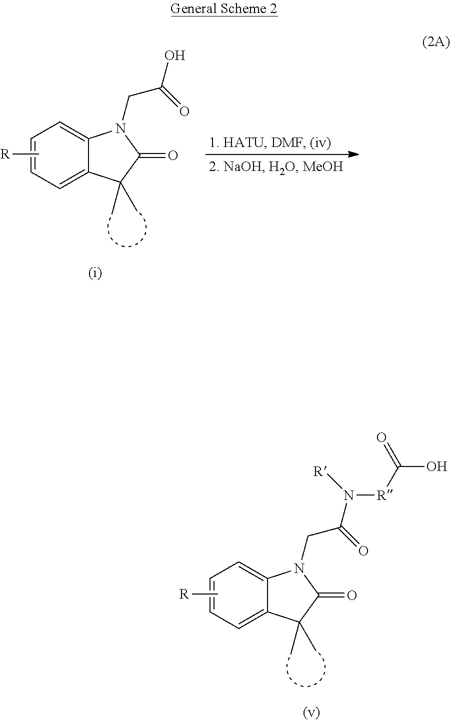

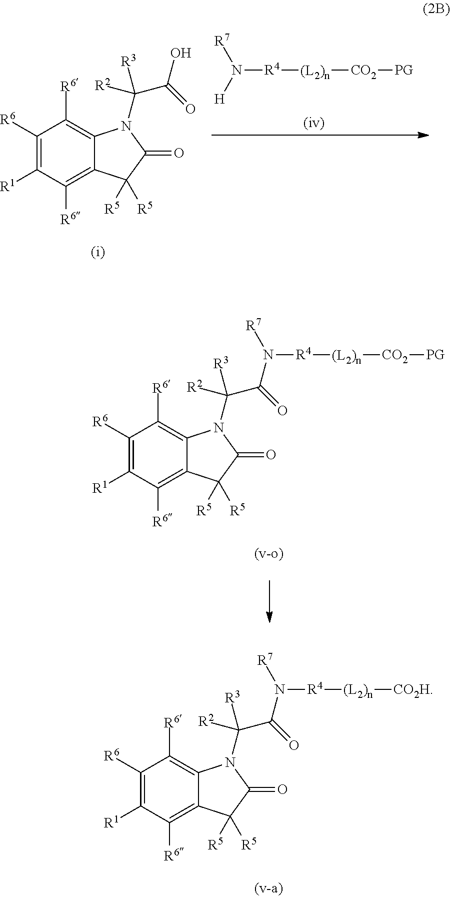

Compounds of Formula (I), wherein Z is -(L₂)ₙ-carboxylic acid, may be prepared in a two-step process as shown in General Scheme 2, through the amide coupling of a substituted indolinone(acetic acid) (i), or salt thereof, with an aminoester (iv), or salt thereof, wherein PG is a protecting group, such as an C₁₋₆ alkyl, C₆ aryl, or an aryl-C₁₋₆ alkyl group, wherein the alkyl and aryl are optionally substituted with one or more halo, C₁₋₆ alkyl, or C₁₋₆ alkoxy groups, followed by hydrolysis to provide the target carboxylic acid analog (v) or (v-a), or salt thereof, wherein indolinone(acetic acid) (i) and aminoesters (iv) are either commercially available or known in the chemical literature, unless otherwise indicated, and the dashed lines correspond to an optional spirofused cycloalkyl.

In some embodiments, the compound of the present disclosure is:

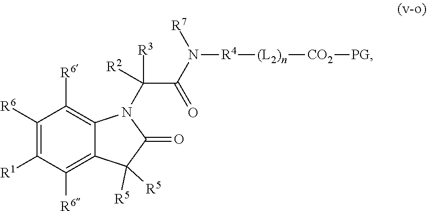

or a salt thereof.

In some embodiments, PG is C₁₋₆ alkyl, C₆ aryl, or aryl-C₁₋₆ alkyl, wherein the alkyl and aryl are optionally substituted with one or more halo, C₁₋₆ alkyl, or C₁₋₆ alkoxy.

In some embodiments, PG is C₁₋₆ alkyl, C₆ aryl, or aryl-C₁₋₆ alkyl.

In some embodiments, PG is C₁₋₆ alkyl optionally substituted with one or more halo, C₁₋₆ alkyl, or C₁₋₆ alkoxy.

In some embodiments, PG is C₁₋₆ alkyl.

In some embodiments, PG is C₆ aryl optionally substituted with one or more halo, C₁₋₆ alkyl, or C₁₋₆ alkoxy.

In some embodiments, PG is C₆ aryl.

In some embodiments, PG is aryl-C₁₋₆ alkyl, wherein the alkyl and aryl are optionally substituted with one or more halo, C₁₋₆ alkyl, or C₁₋₆alkoxy.

In some embodiments, PG is aryl-C₁₋₆ alkyl.

Exemplary compounds of Formula (v-o) include any compounds provided in Table 3, or salt thereof, or a pharmaceutically acceptable salt, solvate, clathrate, hydrate, stereoisomer, tautomer, isotopic derivative, or polymorph thereof. In some embodiments, the compound of Formula (v-o) is a prodrug.

TABLE 3

| # | Ex | Name/Structure |
|---|---|---|
| 2- OMe | 2 | 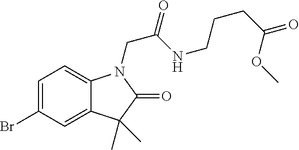 [SMILES: COC(=O)CCCNC(=O)CN1C(=O)C(C)(C)c2cc(Br)ccc21] |
| | | methyl 4-(2-(5-bromo-3,3- |
| | | dimethyl-2-oxoindolin-1- |
| | | yl)acetamido)butanoate |
| 5- OMe | 3 | 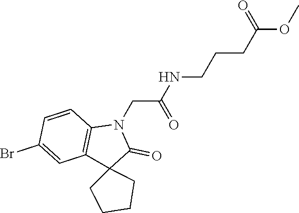 [SMILES: COC(=O)CCCNC(=O)CN1C(=O)C2(CCCC2)c2cc(Br)ccc21] |
| | | methyl 4-(2-(5′-bromo-2′- |
| | | oxospiro[cyclopentane-1,3′- |
| | | indolin]-1′- |
| | | yl)acetamido)butanoate |
| 8- OMe | 4 | 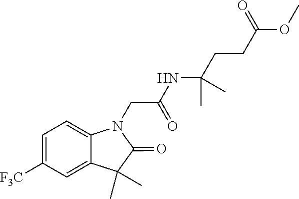 [SMILES: COC(=O)CCC(C)(C)NC(=O)CN1C(=O)C(C)(C)c2cc(C(F)(F)F)ccc21] |
| | | methyl 4-(2-(3,3-dimethyl-2-oxo- |
| | | 5-(trifluoromethyl)indolin-1- |
| | | yl)acetamido)-4- |
| | | methylpentanoate |
| 9- OMe | 5 | 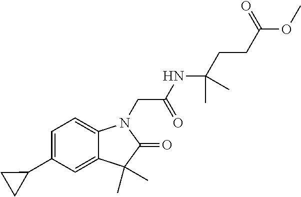 [SMILES: COC(=O)CCC(C)(C)NC(=O)CN1C(=O)C(C)(C)c2cc(C3CC3)ccc21] |
| | | methyl 4-(2-(5-cyclopropyl-3,3- |
| | | dimethyl-2-oxoindolin-1- |
| | | yl)acetamido)-4- |
| | | methylpentanoate |
| 15- OMe | 6 | 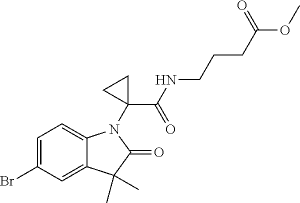 [SMILES: COC(=O)CCCNC(=O)C1(N2C(=O)C(C)(C)c3cc(Br)ccc32)CC1] |
| | | methyl 4-(1-(5-bromo-3,3- |
| | | dimethyl-2-oxoindolin-1- |
| | | yl)cyclopropane-1- |
| | | carboxamido)butanoate |
| 16- OMe | 7 | 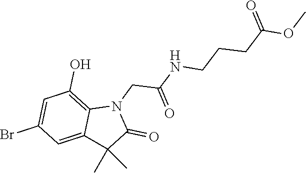 [SMILES: COC(=O)CCCNC(=O)CN1C(=O)C(C)(C)c2cc(Br)cc(O)c21] |
| | | 4-(2-(5-bromo-7-hydroxy-3,3- |
| | | dimethyl-2-oxoindolin-1- |
| | | yl)acetamido)butanoic acid |
| 24- OMe | 8 | 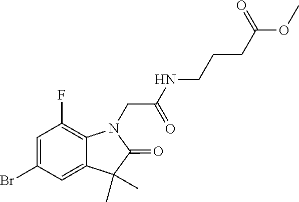 [SMILES: COC(=O)CCCNC(=O)CN1C(=O)C(C)(C)c2cc(Br)cc(F)c21] |
| | | methyl 4-(2-(5-bromo-7-fluoro- |
| | | 3,3-dimethyl-2-oxoindolin-1- |
| | | yl)acetamido)butanoate |
| 27- OMe | 9 | 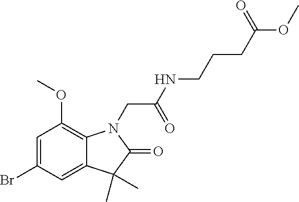 [SMILES: COC(=O)CCCNC(=O)CN1C(=O)C(C)(C)c2cc(Br)cc(OC)c21] |
| | | methyl 4-(2-(5-bromo-7- |
| | | methoxy-3,3-dimethyl-2- |
| | | oxoindolin-1- |
| | | yl)acetamido)butanoate |
| 28- OMe | 10 | 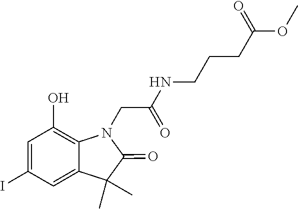 [SMILES: COC(=O)CCCNC(=O)CN1C(=O)C(C)(C)c2cc(I)cc(O)c21] |
| | | methyl 4-(2-(7-hydroxy-5-iodo- |
| | | 3,3-dimethyl-2-oxoindolin-1- |
| | | yl)acetamido)butanoate |
| 36- OMe | 12 | 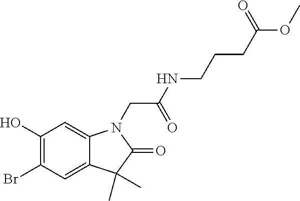 [SMILES: COC(=O)CCCNC(=O)CN1C(=O)C(C)(C)c2cc(Br)c(O)cc21] |
| | | methyl 4-(2-(5-bromo-6- |
| | | hydroxy-3,3-dimethyl-2- |
| | | oxoindolin-1- |
| | | yl)acetamido)butanoate |
| 56- OMe | 13 | 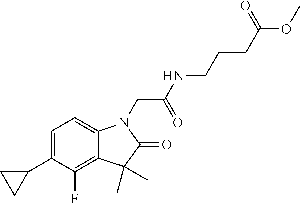 [SMILES: COC(=O)CCCNC(=O)CN1C(=O)C(C)(C)c2c1ccc(C1CC1)c2F] |
| | | methyl 4-(2-(5-cyclopropyl-4- |
| | | fluoro-3,3-dimethyl-2- |
| | | oxoindolin-1- |
| | | yl)acetamido)butanoate |
| 57- OtBu | 14 | 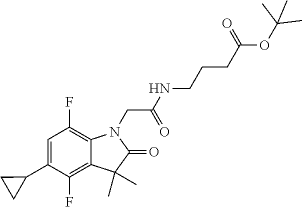 [SMILES: CC(C)(C)OC(=O)CCCNC(=O)CN1C(=O)C(C)(C)c2c(F)c(C3CC3)cc(F)c21] |
| | | tert-butyl 4-(2-(5-cyclopropyl- |
| | | 4,7-difluoro-3,3-dimethyl-2- |
| | | oxoindolin-1- |
| | | yl)acetamido)butanoate |
| 58A*- OtBu | 15 | 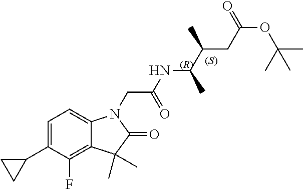 |
| | | tert-butyl (3S,4R)-4-(2-(5- |
| | | cyclopropyl-4-fluoro-3,3- |
| | | dimethyl-2-oxoindolin-1- |
| | | yl)acetamido)-3- |
| | | methylpentanoate |
| 58B*- OtBu | 15 | 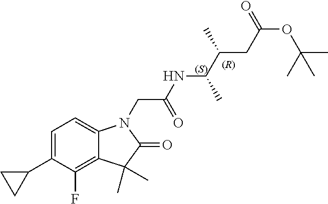 |
| | | tert-butyl (3R,4S)-4-(2-(5- |
| | | cyclopropyl-4-fluoro-3,3- |
| | | dimethyl-2-oxoindolin-1- |
| | | yl)acetamido)-3- |
| | | methylpentanoate |
| 58C*- OtBu | 15 | 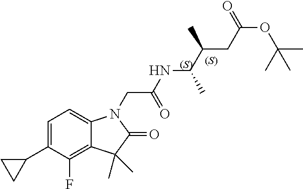 |
| | | tert-butyl (3S,4S)-4-(2-(5- |
| | | cyclopropyl-4-fluoro-3,3- |
| | | dimethyl-2-oxoindolin-1- |
| | | yl)acetamido)-3- |
| | | methylpentanoate |
| 58D*- OtBu | 15 | 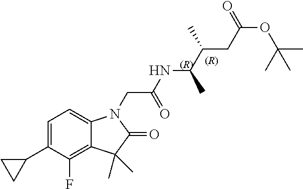 |
| | | tert-butyl (3R,4R)-4-(2-(5- |
| | | cyclopropyl-4-fluoro-3,3- |
| | | dimethyl-2-oxoindolin-1- |
| | | yl)acetamido)-3- |
| | | methylpentanoate |
| 59A- OMe | 16 | 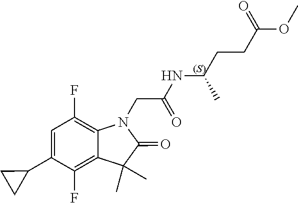 |
| | | methyl (S)-4-(2-(5-cyclopropyl- |
| | | 4,7-difluoro-3,3-dimethyl-2- |
| | | oxoindolin-1- |
| | | yl)acetamido)pentanoate |
| 59B- OMe | 16 | 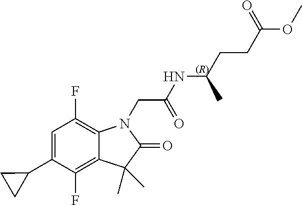 |
| | | methyl (R)-4-(2-(5-cyclopropyl- |
| | | 4,7-difluoro-3,3-dimethyl-2- |
| | | oxoindolin-1- |
| | | yl)acetamido)pentanoate |
| 60A* OtBu | 17 | 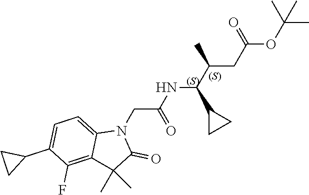 |
| | | tert-butyl (3S,4S)-4-cyclopropyl- |
| | | 4-(2-(5-cyclopropyl-4-fluoro-3,3- |
| | | dimethyl-2-oxoindolin-1- |
| | | yl)acetamido)-3-methylbutanoate |
| 60B* OtBu | 17 | 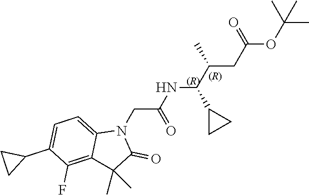 |
| | | tert-butyl (3R,4R)-4-cyclopropyl- |
| | | 4-(2-(5-cyclopropyl-4-fluoro-3,3- |
| | | dimethyl-2-oxoindolin-1- |
| | | yl)acetamido)-3-methylbutanoate |
| 60C*- OtBu | 17 | 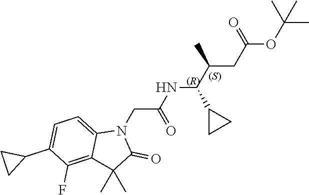 |
| | | tert-butyl (3S,4R)-4-cyclopropyl- |
| | | 4-(2-(5-cyclopropyl-4-fluoro-3,3- |
| | | dimethyl-2-oxoindolin-1- |
| | | yl)acetamido)-3-methylbutanoate |
| 60D*- OtBu | 17 | 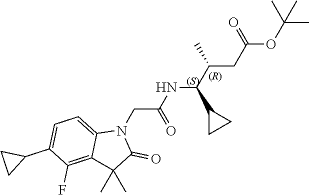 |
| | | tert-butyl (3R,4S)-4-cyclopropyl- |
| | | 4-(2-(5-cyclopropyl-4-fluoro-3,3- |
| | | dimethyl-2-oxoindolin-1- |
| | | yl)acetamido)-3-methylbutanoate |
| 61A- OMe | 18 | 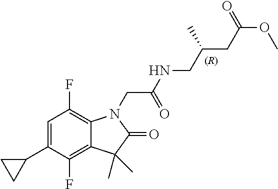 |
| | | methyl (R)-4-(2-(5-cyclopropyl- |
| | | 4,7-difluoro-3,3-dimethyl-2- |
| | | oxoindolin-1-yl)acetamido)-3- |
| | | methylbutanoate |
| 61B- OEt | 18 | 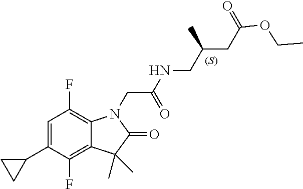 |
| | | ethyl (S)-4-(2-(5-cyclopropyl- |
| | | 4,7-difluoro-3,3-dimethyl-2- |
| | | oxoindolin-1-yl)acetamido)-3- |
| | | methylbutanoate |
| 62A- OMe | 19 | 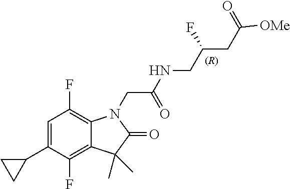 |
| | | methyl (R)-4-(2-(5-cyclopropyl-4,7- |
| | | difluoro-3,3-dimethyl-2-oxoindolin- |
| | | 1-yl)acetamido)-3-fluorobutanoate |
| 62B- OMe | 19 | 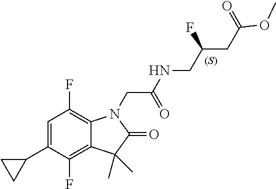 |
| | | methyl (S)-4-(2-(5-cyclopropyl-4,7- |
| | | difluoro-3,3-dimethyl-2-oxoindolin- |
| | | 1-yl)acetamido)-3-fluorobutanoate |
| 63A- OMe | 20 | 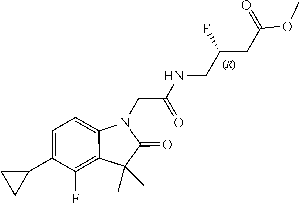 |
| | | methyl (R)-4-(2-(5-cyclopropyl-4- |
| | | fluoro-3,3-dimethyl-2-oxoindolin-1- |
| | | yl)acetamido)-3-fluorobutanoate |
| 63B- OMe | 20 | 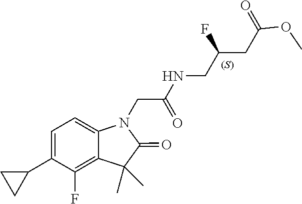 |
| | | methyl (S)-4-(2-(5-cyclopropyl-4- |
| | | fluoro-3,3-dimethyl-2-oxoindolin-1- |
| | | yl)acetamido)-3-fluorobutanoate |
| 64A- OMe | 21 | 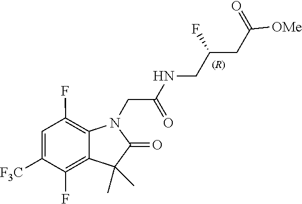 |
| | | methyl (R)-4-(2-(4,7-difluoro-3,3- |
| | | dimethyl-2-oxo-5- |
| | | (trifluoromethyl)indolin-1- |
| | | yl)acetamido)-3-fluorobutanoate |
| 64B- OMe | 21 | 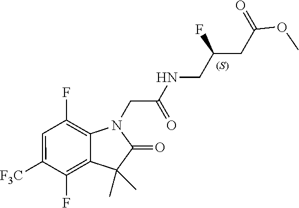 |
| | | methyl (S)-4-(2-(4,7-difluoro-3,3- |
| | | dimethyl-2-oxo-5- |
| | | (trifluoromethyl)indolin-1- |
| | | yl)acetamido)-3-fluorobutanoate |
| 66- OtBu | 22 | 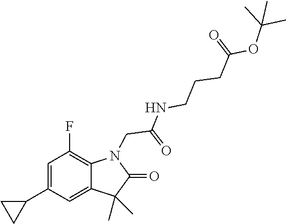 [SMILES: CC(C)(C)OC(=O)CCCNC(=O)CN1C(=O)C(C)(C)c2cc(C3CC3)cc(F)c21] |
| | | tert-butyl 4-(2-(5-cyclopropyl-7- |
| | | fluoro-3,3-dimethyl-2-oxoindolin-1- |
| | | yl)acetamido)butanoate |
| 67A*- OtBu | 23 | 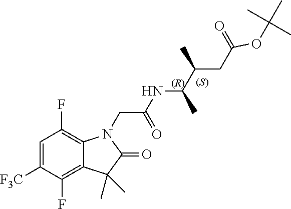 |
| | | tert-butyl (3S,4R)-4-(2-(4,7-difluoro- |
| | | 3,3-dimethyl-2-oxo-5- |
| | | (trifluoromethyl)indolin-1- |
| | | yl)acetamido)-3-methylpentanoate |
| 67B*- OtBu | 23 | 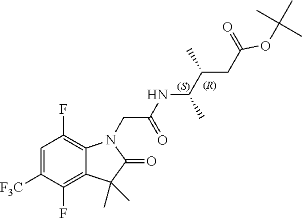 |
| | | tert-butyl (3R,4S)-4-(2-(4,7-difluoro- |
| | | 3,3-dimethyl-2-oxo-5- |
| | | (trifluoromethyl)indolin-1- |
| | | yl)acetamido)-3-methylpentanoate |
| 67C*- OtBu | 23 | 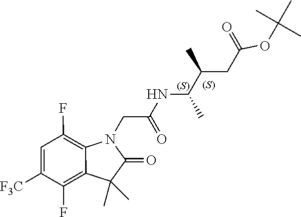 |
| | | tert-butyl (3S,4S)-4-(2-(4,7-difluoro- |
| | | 3,3-dimethyl-2-oxo-5- |
| | | (trifluoromethyl)indolin-1- |
| | | yl)acetamido)-3-methylpentanoate |
| 67D*- OtBu | 23 | 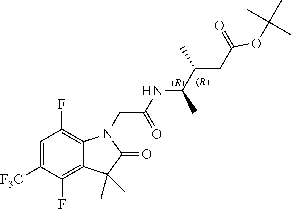 |
| | | tert-butyl (3R,4R)-4-(2-(4,7- |
| | | difluoro-3,3-dimethyl-2-oxo-5- |
| | | (trifluoromethyl)indolin-1- |
| | | yl)acetamido)-3-methylpentanoate |
| 69A*- OMe | 24 | 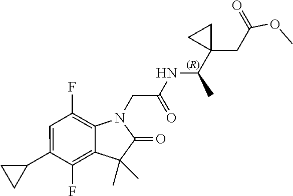 |
| | | methyl (R)-2-(1-(1-(2-(5- |
| | | cyclopropyl-4,7-difluoro-3,3- |
| | | dimethyl-2-oxoindolin-1- |
| | | yl)acetamido)ethyl)cyclopropyl)acetate |
| 69B*- OMe | 24 | 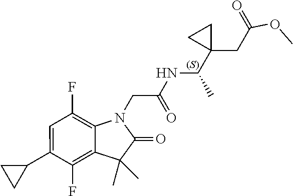 |
| | | methyl (S)-2-(1-(1-(2-(5- |
| | | cyclopropyl-4,7-difluoro-3,3- |
| | | dimethyl-2-oxoindolin-1- |
| | | yl)acetamido)ethyl)cyclopropyl)acetate |
| 70A*- OMe | 25 | 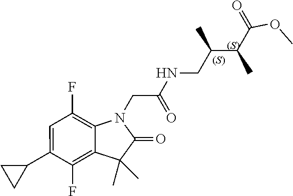 |
| | | methyl (2S,3S)-4-(2-(5-cyclopropyl- |
| | | 4,7-difluoro-3,3-dimethyl-2- |
| | | oxoindolin-1-yl)acetamido)-2,3- |
| | | dimethylbutanoate |
| 70B*- OMe | 25 | 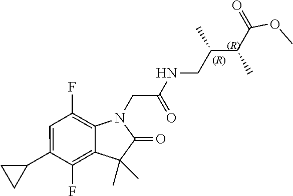 |
| | | methyl (2R,3R)-4-(2-(5-cyclopropyl- |
| | | 4,7-difluoro-3,3-dimethyl-2- |
| | | oxoindolin-1-yl)acetamido)-2,3- |
| | | dimethylbutanoate |
| 70C*- OMe | 25 | 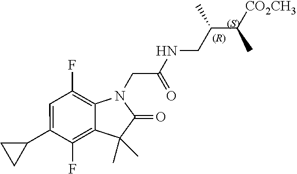 |
| | | methyl (2S,3R)-4-(2-(5-cyclopropyl- |
| | | 4,7-difluoro-3,3-dimethyl-2- |
| | | oxoindolin-1-yl)acetamido)-2,3- |
| | | dimethylbutanoate |
| 70D*- OMe | 25 | 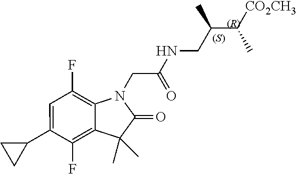 |
| | | methyl (2R,3S)-4-(2-(5-cyclopropyl- |
| | | 4,7-difluoro-3,3-dimethyl-2- |
| | | oxoindolin-1-yl)acetamido)-2,3- |
| | | dimethylbutanoate |
| 71A*- OEt | 26 | 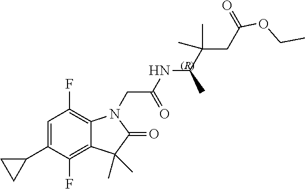 |
| | | ethyl (R)-4-(2-(5-cyclopropyl-4,7- |
| | | difluoro-3,3-dimethyl-2-oxoindolin- |
| | | 1-yl)acetamido)-3,3- |
| | | dimethylpentanoate |
| 71B*- OEt | 26 | 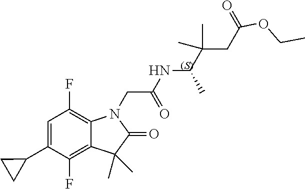 |
| | | ethyl (S)-4-(2-(5-cyclopropyl-4,7- |
| | | difluoro-3,3-dimethyl-2-oxoindolin- |
| | | 1-yl)acetamido)-3,3- |
| | | dimethylpentanoate |
| 72A*- OEt | 27 | 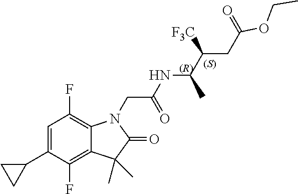 |
| | | ethyl (3S,4R)-4-(2-(5-cyclopropyl- |
| | | 4,7-difluoro-3,3-dimethyl-2- |
| | | oxoindolin-1-yl)acetamido)-3- |
| | | (trifluoromethyl)pentanoate |
| 72B*- OEt | 27 | 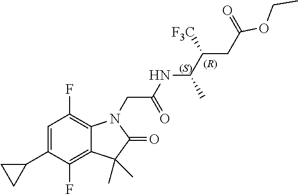 |
| | | ethyl (3R,4S)-4-(2-(5-cyclopropyl- |
| | | 4,7-difluoro-3,3-dimethyl-2- |
| | | oxoindolin-1-yl)acetamido)-3- |
| | | (trifluoromethyl)pentanoate |
| 72C*- OEt | 27 | 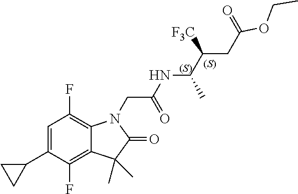 |
| | | ethyl (3S,4S)-4-(2-(5-cyclopropyl- |
| | | 4,7-difluoro-3,3-dimethyl-2- |
| | | oxoindolin-1-yl)acetamido)-3- |
| | | (trifluoromethyl)pentanoate |
| 72D*- OEt | 27 | 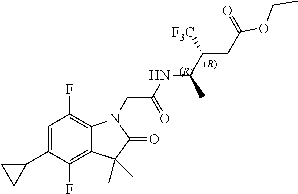 |
| | | ethyl (3R,4R)-4-(2-(5-cyclopropyl- |
| | | 4,7-difluoro-3,3-dimethyl-2- |
| | | oxoindolin-1-yl)acetamido)-3- |
| | | (trifluoromethyl)pentanoate |

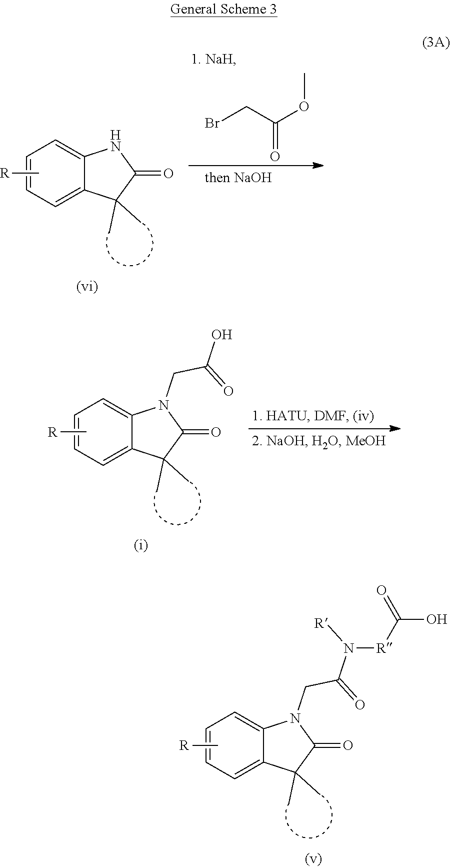

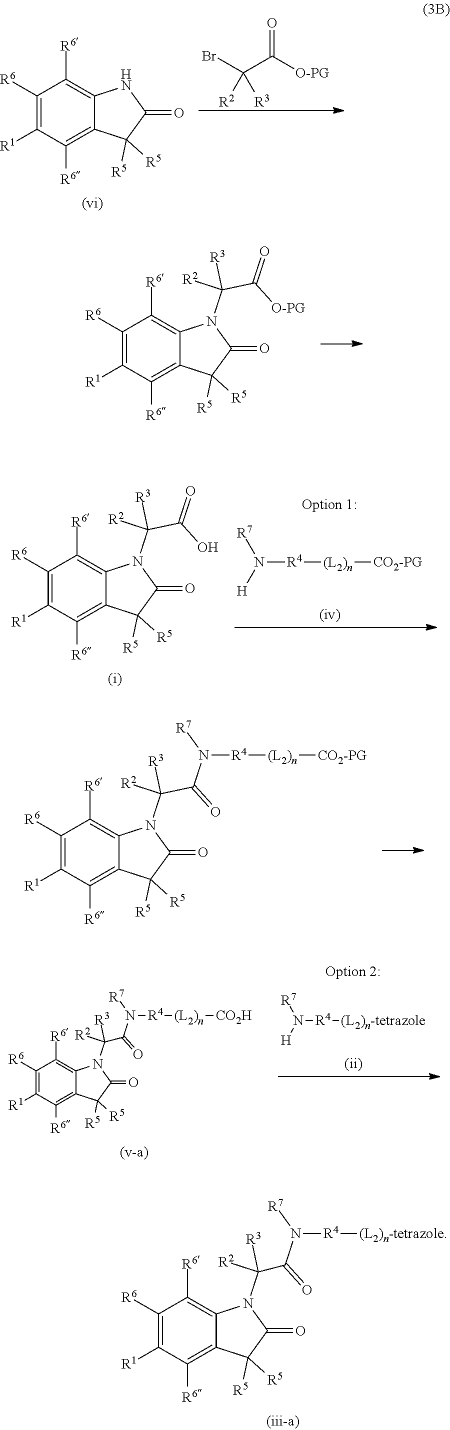

Compounds of Formula (I) may be prepared as shown in General Scheme 3, through the alkylation of indolinone (vi), or salt thereof, followed by hydrolysis, to provide indolinone(acetic acid) (i), or salt thereof. Then, amide coupling (and subsequent hydrolysis when the target is a carboxylic acid and not a tetrazole) provides the target carboxylic analog (v) or (v-a), or salt thereof, or target tetrazole analog (iii-a), or salt thereof, wherein the starting material indolinone (vi), aminoester (iv) and aminotetrazole (ii) are either commercially available or known in the chemical literature, unless otherwise indicated, and the dashed lines correspond to an optional spirofused cycloalkyl.

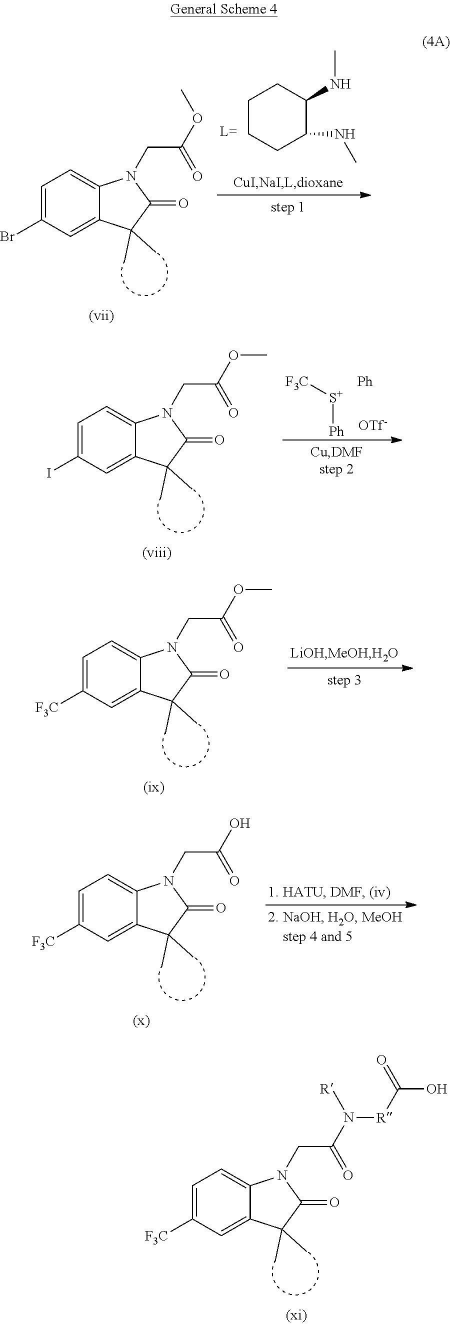

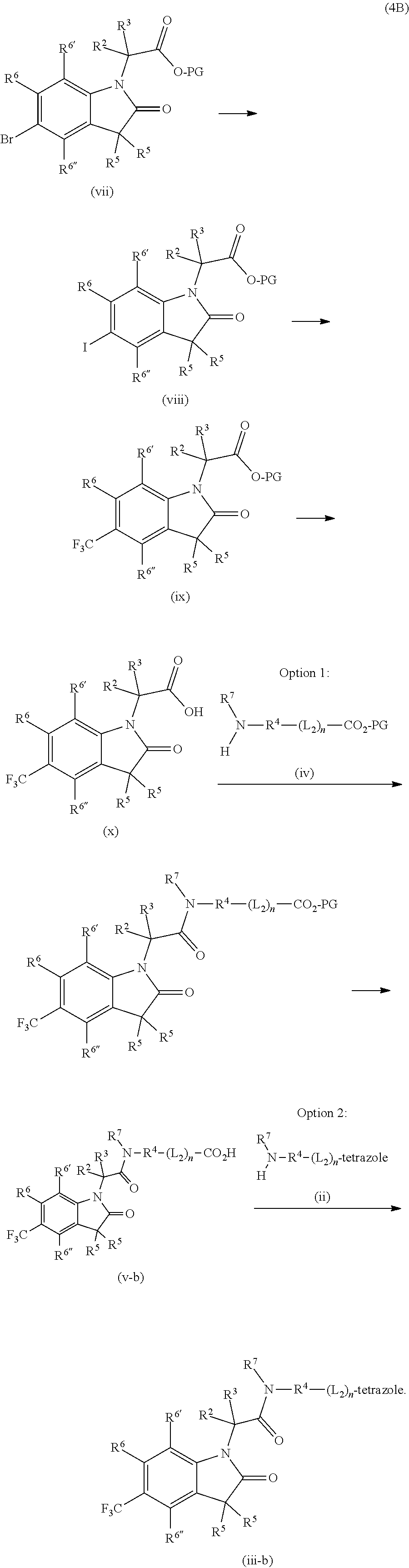

Compounds of Formula (I) may be prepared as shown in General Scheme 4, through the transhalogenation of bromo indolinone (vii), or salt thereof, wherein PG is a protecting group, such as an C₁₋₆ alkyl, C₆ aryl, or an aryl-C₁₋₆ alkyl group, wherein the alkyl and aryl are optionally substituted with one or more halo, C₁₋₆ alkyl, or C₁₋₆ alkoxy groups, to form an iodo indolinone (viii), or salt thereof, then copper coupling to afford the —CF₃ analog (ix), or salt thereof, then ester hydrolysis to provide carboxylic acid intermediate (x), or salt thereof, followed by amide coupling with (iv) or (ii), or salt thereof (and subsequent hydrolysis when the target is a carboxylic acid and not a tetrazole) to provide the carboxylic target analogs (xi) or (v-b), or salt thereof, or tetrazole target analog (iii-b), or salt thereof, wherein the starting material indolinone (vii) aminoester (iv) and aminotetrazole (ii) are either commercially available, known in the chemical literature, or prepared through previous schemes herein, unless otherwise indicated, and the dashed lines correspond to an optional spirofused cycloalkyl.

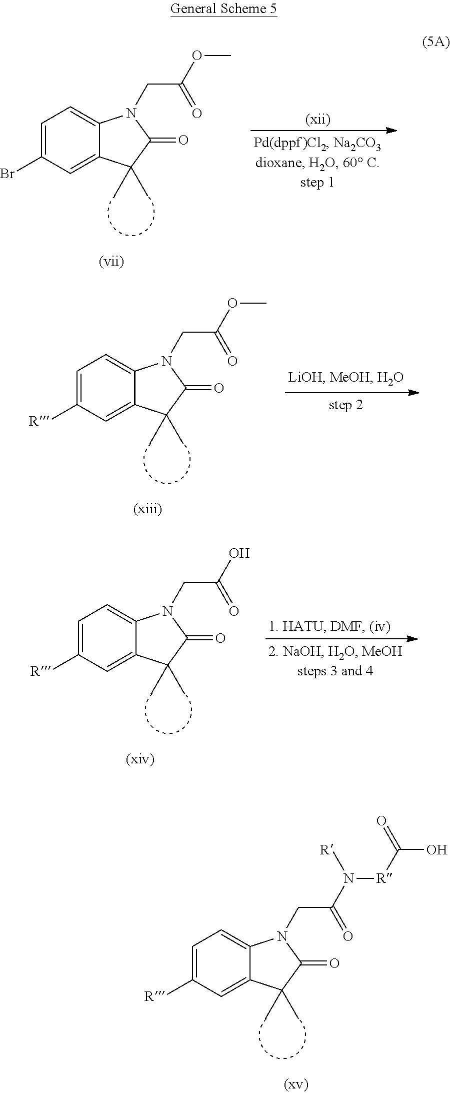

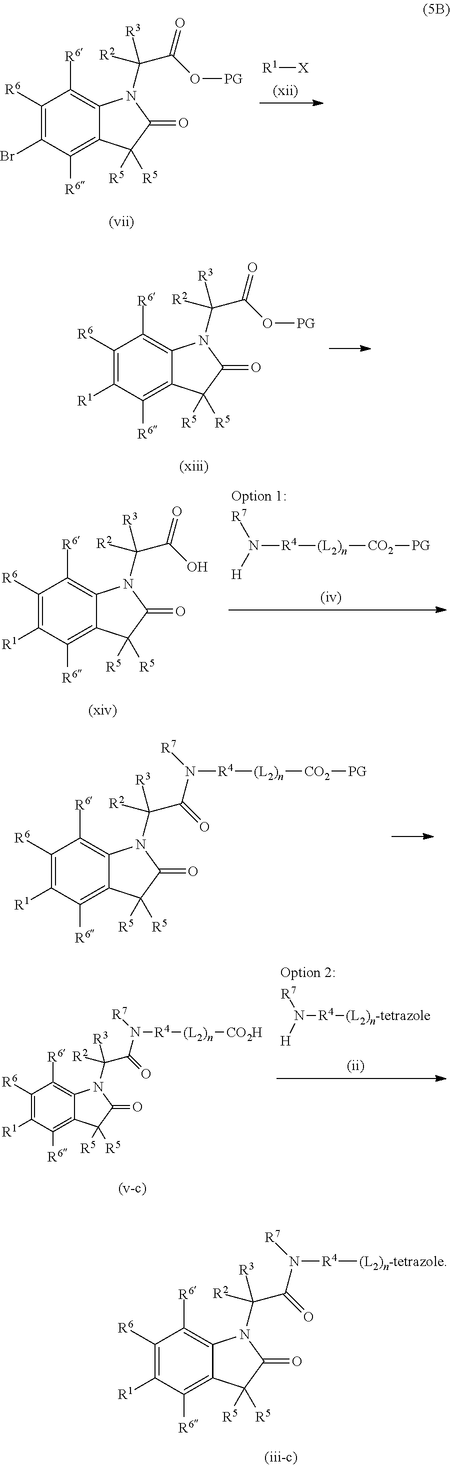

Compounds of Formula (I) may be prepared in a process as shown in General Scheme 5, through the palladium cross-coupling of indolinone (vii), or salt thereof, with a coupling partner (xii), or salt thereof, such as R′″—X or R¹—X, wherein X a leaving group, and R′″ or R¹ is C₁-C₆ alkyl, C₂-C₆ alkenyl, C₂-C₆ alkynyl, C₁-C₆ haloalkyl, C₁-C₆ alkoxy, or C₃-C₁₂ cycloalkyl, to furnish the alkylated ester product (xiii), or salt thereof, which is then hydrolyzed to the carboxylic acid intermediate (xiv), or salt thereof. As used herein, a “leaving group” is an art-understood term referring to a molecular fragment that departs with a pair of electrons in heterolytic bond cleavage, wherein the molecular fragment is an anion or neutral molecule. In some embodiments, X is a boronic acid or boronic ester. See, for example, Smith, March Advanced Organic Chemistry 6th ed. (501-502). Exemplary leaving groups include, but are not limited to, halo (e.g., chloro, bromo, iodo) and sulfonyl substituted hydroxyl groups (e.g., tosyl, mesyl, besyl). Amide coupling (and subsequent hydrolysis when the target is a carboxylic acid and not a tetrazole) with aminoester (iv), or salt thereof, or aminotetrazole (ii), or salt thereof, then provides the target analogs, carboxylic acid target analogs (xv) or (v-c), or salt thereof, and tetrazole target analog (iii-c), or salt thereof, wherein starting material bromoindolinone (vii), coupling partner (xii), aminoester (iv), and aminotetrazole (ii) are either commercially available, known in the chemical literature, or prepared through previous schemes herein, unless otherwise indicated, and the dashed lines correspond to an optional spirofused cycloalkyl.

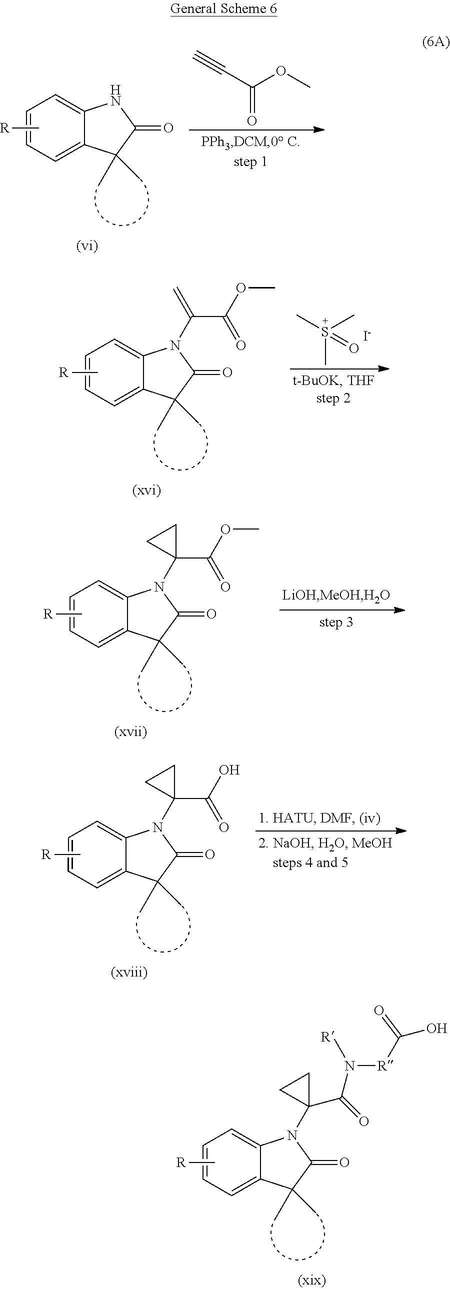

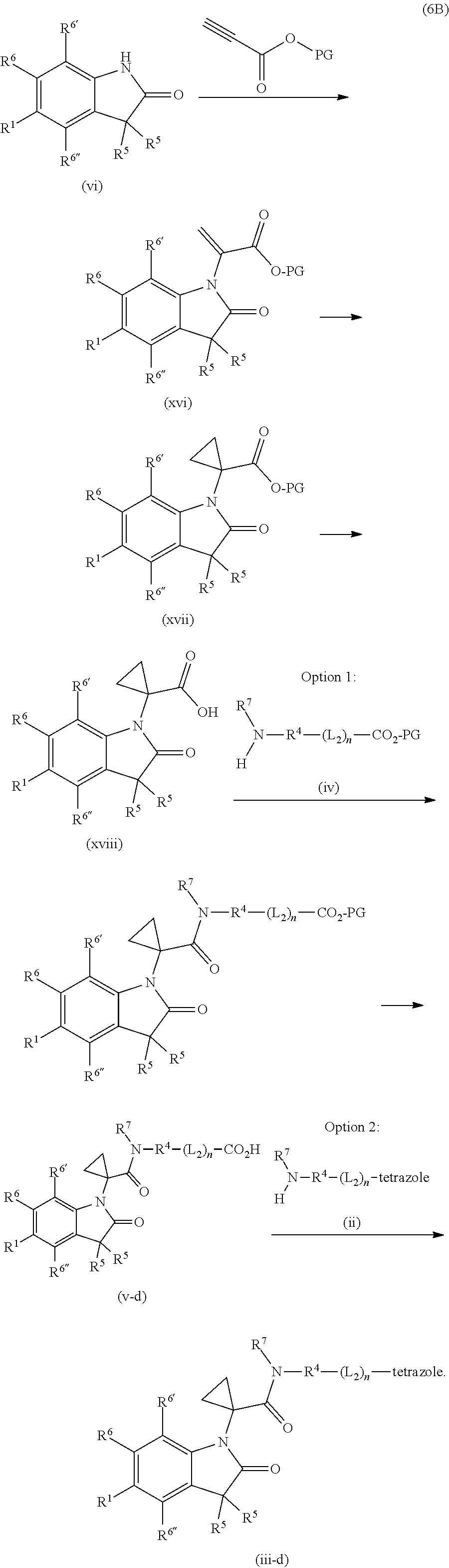

Compounds of Formula (I), wherein R² and R³ are joined to form a C₃ cycloalkyl, may be prepared in a process as shown in General Scheme 6, through the substitution of indolinone (vi), or salt thereof, with an alkyl (e.g., methyl) propiolate, wherein PG is an C₁₋₆ alkyl, C₆ aryl, or an aryl-C₁₋₆ alkyl group, wherein the alkyl and aryl are optionally substituted with one or more halo, C₁₋₆ alkyl, or C₁₋₆ alkoxy groups, to provide the alpha,beta-unsaturated ester (xvi), or salt thereof, followed by cyclopropanation of the alkenyl moiety (such as by Corey-Chaykovsky cyclopropanation) to provide cyclopropanated intermediate (xvii), or salt thereof. Hydrolysis to the carboxylic acid intermediate (xviii), or salt thereof, followed by amide coupling (and subsequent hydrolysis when the target is a carboxylic acid and not a tetrazole) with aminoester (iv), or salt thereof, or aminotetrazole (ii), or salt thereof, then provides the target analogs, wherein indolinones (vi), amino esters (iv) and aminotetrazoles (ii) are either commercially available, known in the chemical literature, or prepared through previous schemes herein, unless otherwise indicated, and the dashed lines correspond to an optional spirofused cycloalkyl.

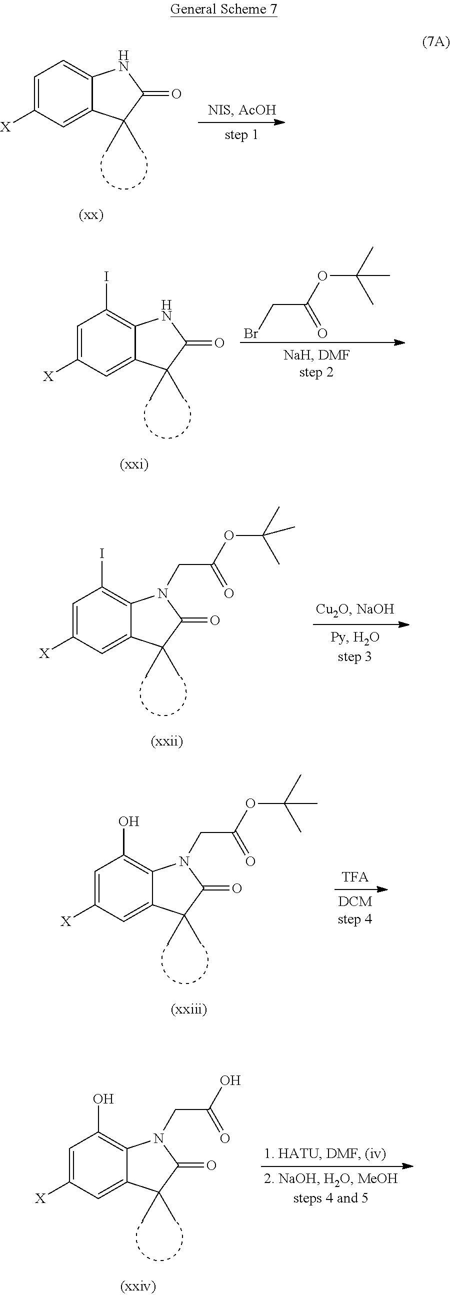

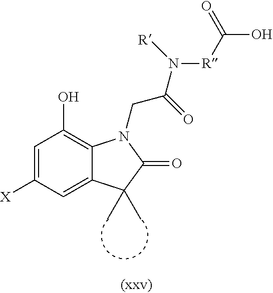

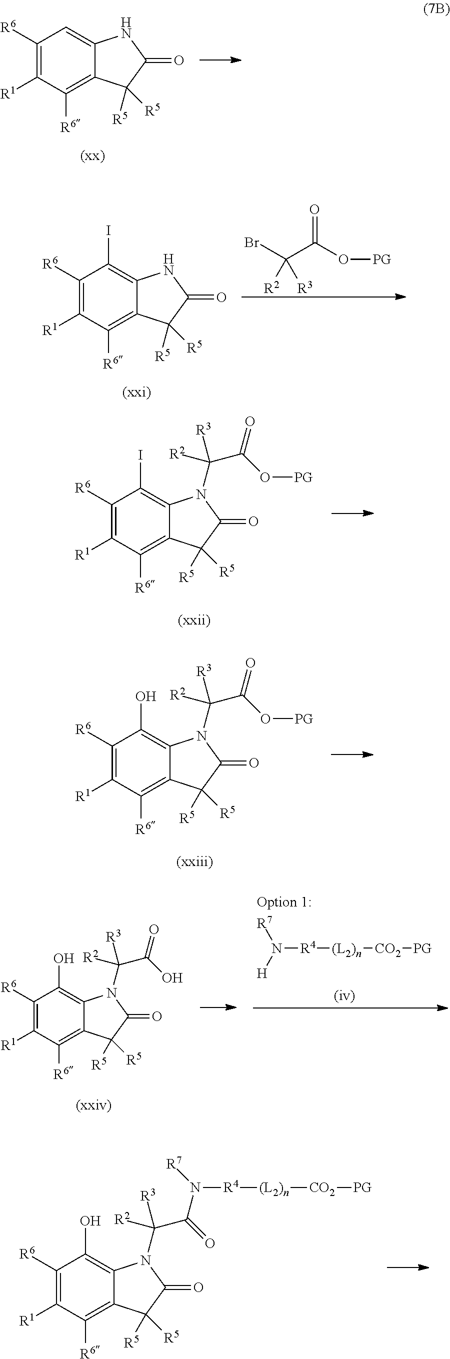

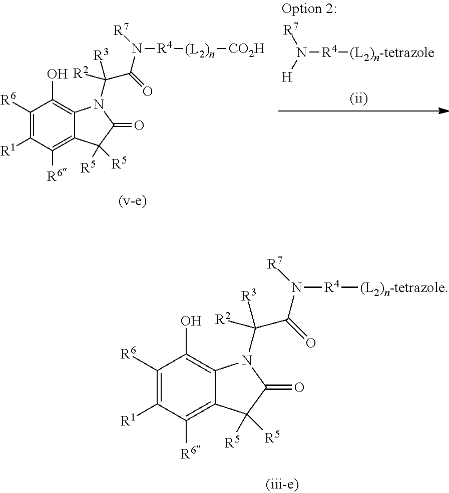

Compounds of Formula (I), wherein R^{6′} is —OH and R¹ is —Br or —Cl, may be prepared in a process as shown in General Scheme 7, through the iodination of indolinone (xx), or salt thereof, then alkylation to provide (xxii), or salt thereof. Oxidation to the phenol (xxiii), or salt thereof, then hydrolysis to the carboxylic acid intermediate (xxiv), or salt thereof, and amide coupling (and subsequent hydrolysis when the target is a carboxylic acid and not a tetrazole) then provides the carboxylic acid target analogs (xxv) or (v-e), or salt thereof, and tetrazole target analog (iii-e), or salt thereof, wherein indolinones (xx), amino esters (iv) and aminotetrazoles (ii) are either commercially available, known in the chemical literature, or prepared through previous schemes herein, and X and R¹ is chloro or bromo, unless otherwise indicated, and the dashed lines correspond to an optional spirofused cycloalkyl.

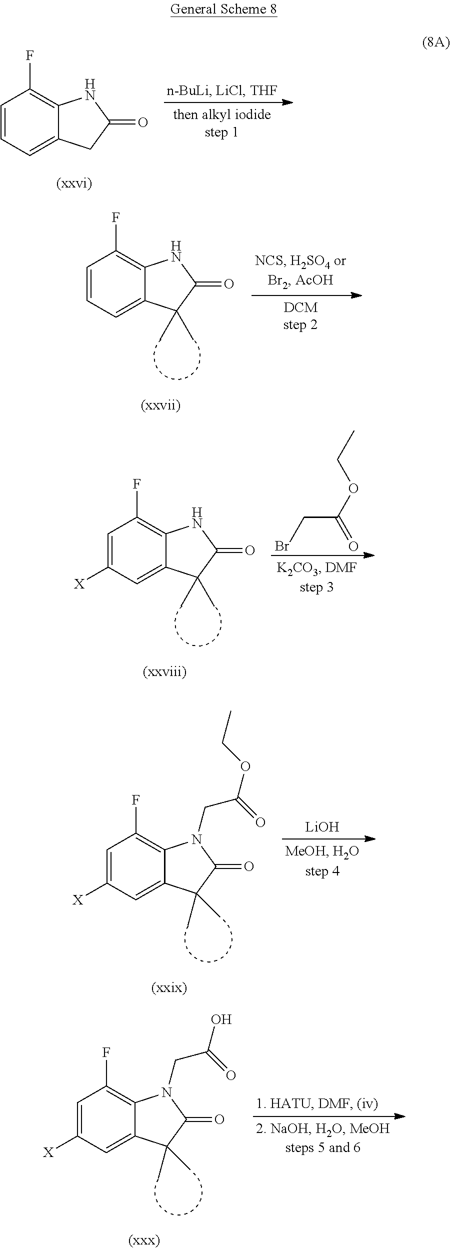

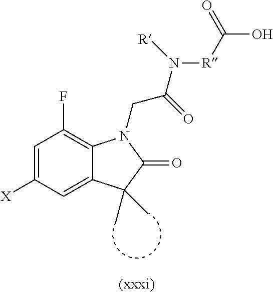

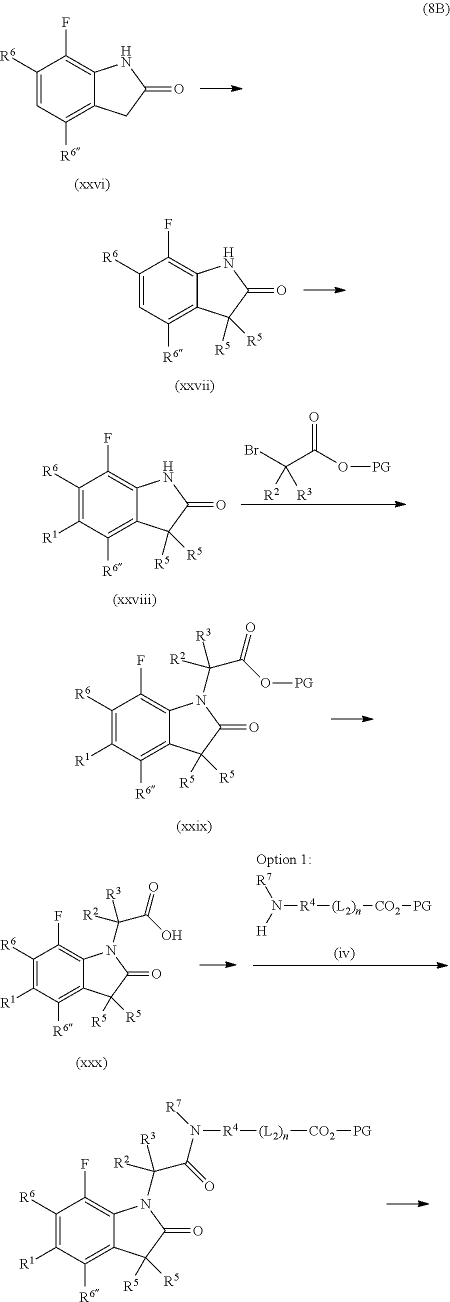

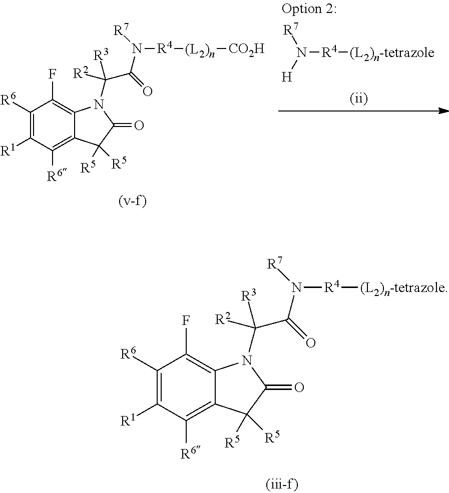

Compounds of Formula (I), wherein R^{6′} is —F and R¹ is —Br or —C₁, may be prepared in a process as shown in General Scheme 8, through the alkylation of indolinone (xxvi), or salt thereof, to provide (xxvii), or salt thereof, followed by halogenation (e.g., bromination or chlorination, wherein X or R¹ is —Br or —Cl) provide halogenated intermediate (xxviii), or salt thereof. N-alkylation to provide the ester (xxix), or salt thereof, followed by hydrolysis to provide the carboxylic acid intermediate (xxx), or salt thereof, then amide coupling (and subsequent hydrolysis when the target is a carboxylic acid and not a tetrazole) provides the carboxylic target analog (xxxi) or (v-f), or salt thereof, and tetrazole target analog (iii-f), or salt thereof, wherein the indolinones (xxvi), aminoesters (iv), and aminotetrazoles (ii) are either commercially available, known in the chemical literature, or prepared through previous schemes herein, unless otherwise indicated, and the dashed lines correspond to an optional spirofused cycloalkyl.

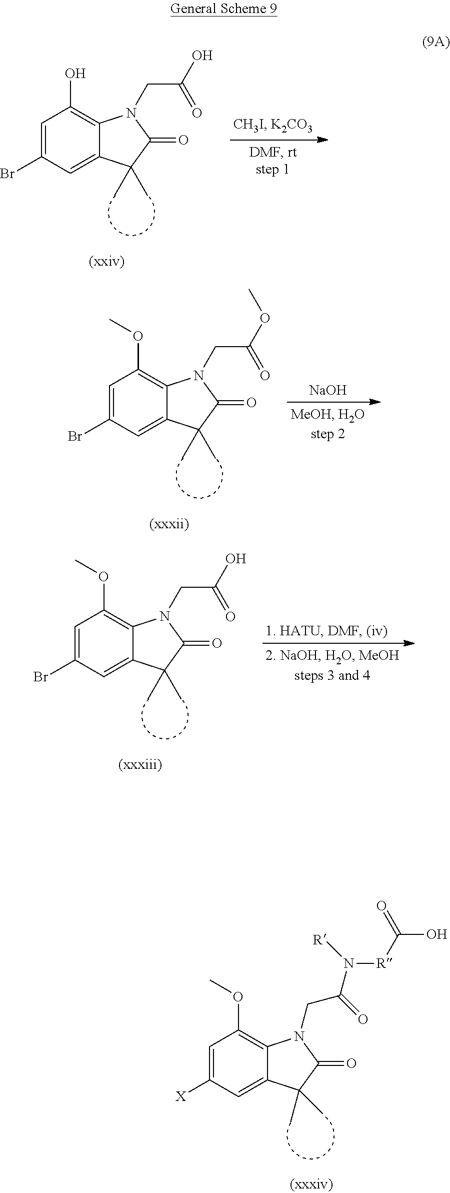

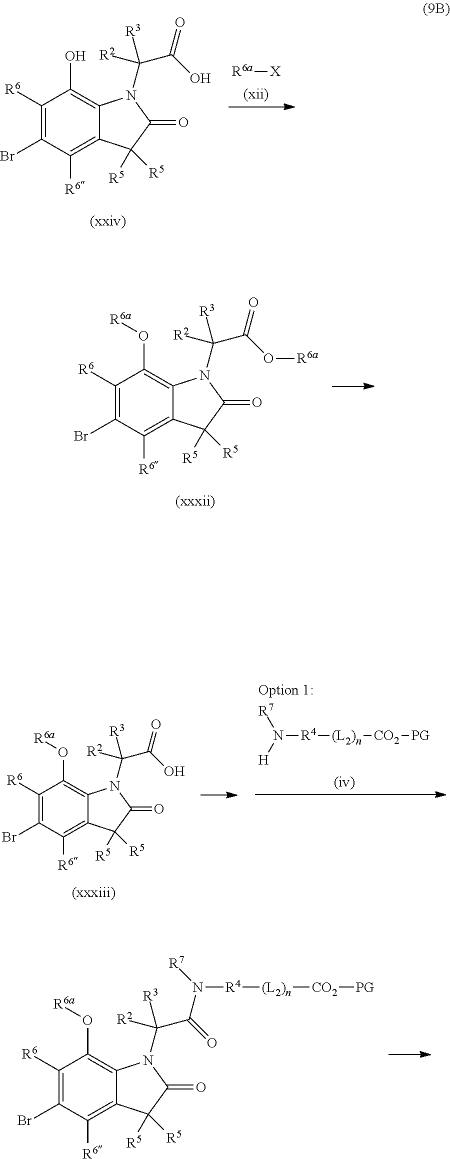

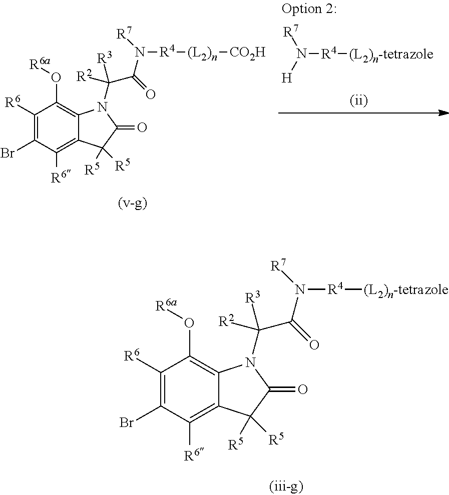

Compounds of Formula (I), wherein R¹ is —Br and R^{6′} is C₁-C₆ alkoxy (e.g., —OR^{6a}, wherein R^{6a} is C₁-C₆ alkyl), may be prepared in a process as shown in General Scheme 9, through the alkylation of hydroxyindolinone carboxylic acid (xxiv), or salt thereof, then ester hydrolysis to provide carboxylic acid intermediate (xxxiii), or salt thereof. Amide coupling (and subsequent hydrolysis when the target is a carboxylic acid and not a tetrazole) then provides the carboxylic acid target analog (xxxiv) and (v-g), or salt thereof, and the tetrazole target analog (iii-g), or salt thereof, wherein indolinones (xxiv), aminoesters (iv), and aminotetrazoles (ii) are either commercially available, known in the chemical literature, or prepared through previous schemes herein, unless otherwise indicated, and the dashed lines correspond to an optional spirofused cycloalkyl.

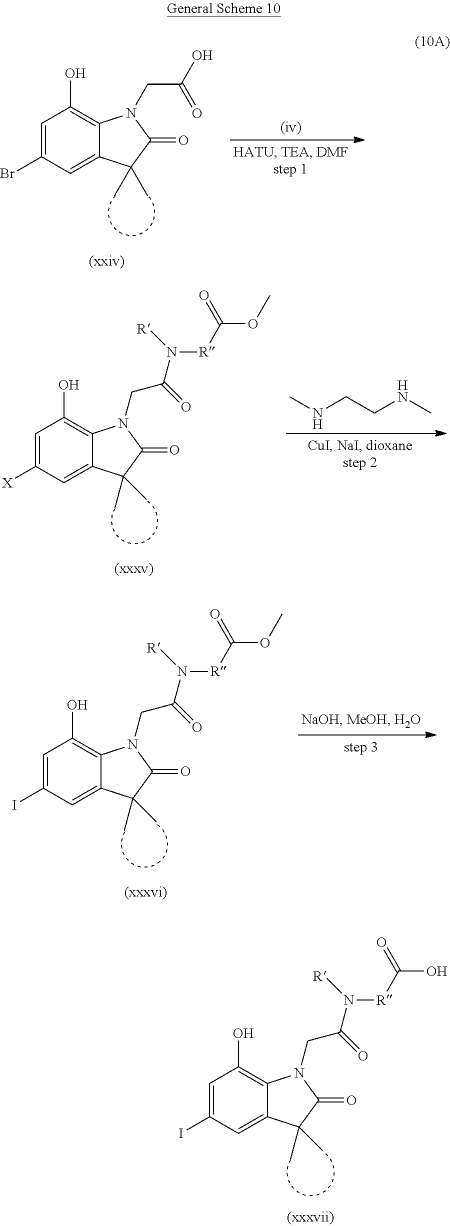

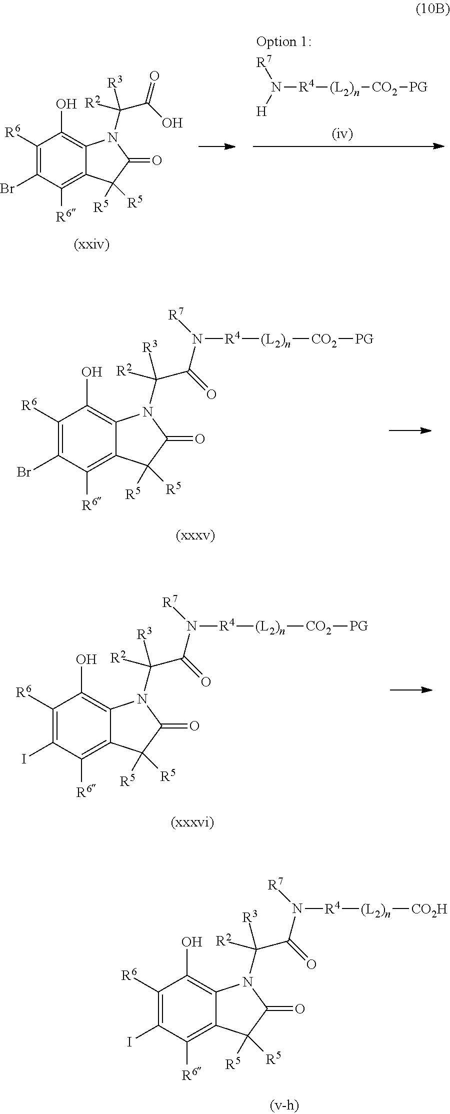

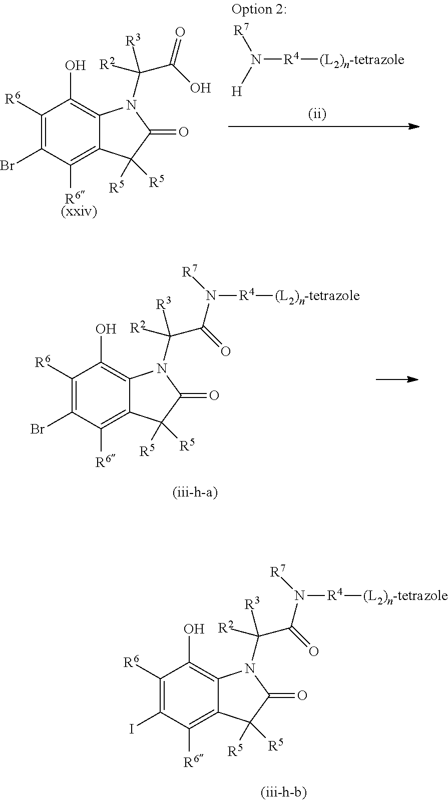

Compounds of Formula (I), wherein R¹ is —I or —Br and R^{6′} is —OH, may be prepared in a process as shown in General Scheme 10, through the amide coupling of (xxiv), or salt thereof, to provide the ester intermediate (xxxv), or salt thereof, wherein PG is a protecting group, such as an C₁₋₆ alkyl, C₆ aryl, or an aryl-C₁₋₆ alkyl group, wherein the alkyl and aryl are optionally substituted with one or more halo, C₁₋₆ alkyl, or C₁₋₆ alkoxy groups, then copper-mediated transhalogenation (and subsequent hydrolysis when the target is a carboxylic acid and not a tetrazole) to provide the carboxylic acid target analog (xxxvii) or (v-h), or salt thereof, and the tetrazole target analog (iii-h-a) and (iii-h-b), or salt thereof, wherein indolinones (xxiv), aminoesters (iv), and aminotetrazoles (ii) are either commercially available, known in the chemical literature, or prepared through previous schemes herein, unless otherwise indicated, and the dashed lines correspond to an optional spirofused cycloalkyl.

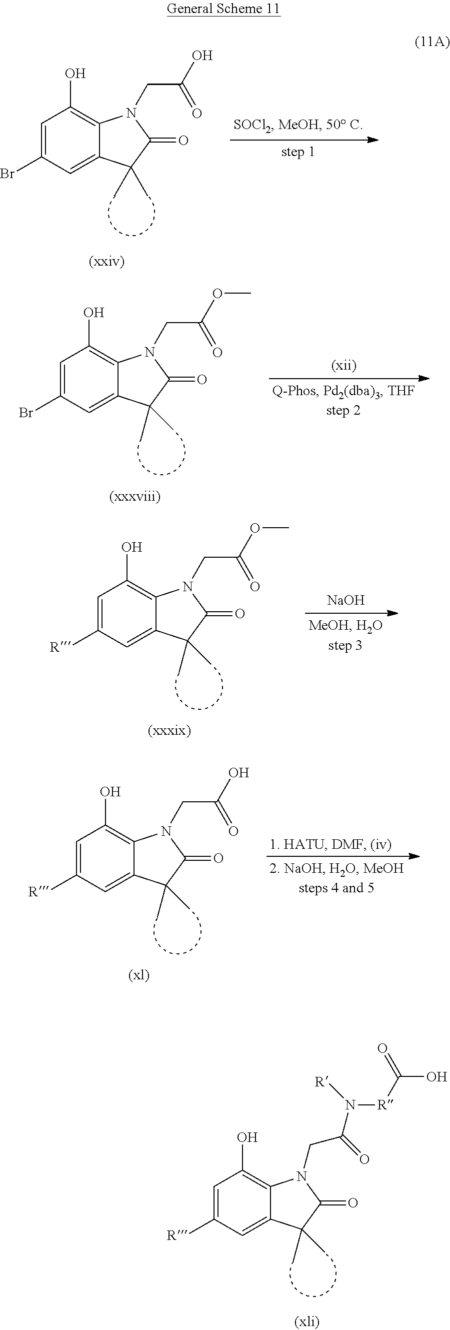

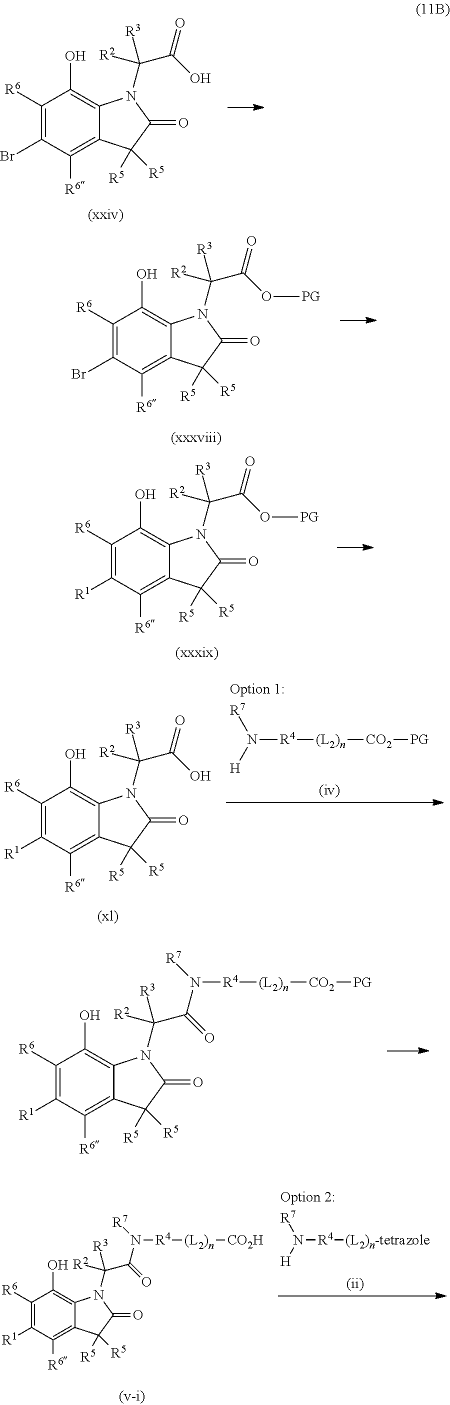

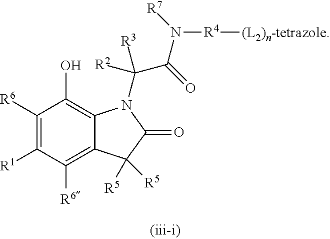

Compounds of Formula (I), wherein R^{6′} is —OH, may be prepared in a process as shown in General Scheme 11, through the esterification of the carboxylic acid (xxiv), or salt thereof, to provide the ester intermediate (xxxviii), or salt thereof, then palladium coupling with to provide with a coupling partner (xii), or salt thereof, such as R′″—X or R¹—X, wherein X a leaving group and R′″ or R¹ is C₁-C₆ alkyl, C₂-C₆ alkenyl, C₂-C₆ alkynyl, C₁-C₆ haloalkyl, C₁-C₆ alkoxy, or C₃-C₁₂ cycloalkyl, which may be optionally substituted as defined herein, to furnish the alkylated ester product (xxxix), or salt thereof. Hydrolysis and amide coupling (and subsequent hydrolysis when the target is a carboxylic acid and not a tetrazole) then provides the carboxylic acid target analog (xli) or (v-i), or salt thereof, and the tetrazole target analog (iii-i), or salt thereof, wherein the indolinones (xxiv), aminoesters (iv), and aminotetrazoles (ii) are either commercially available, known in the chemical literature, or prepared through previous schemes herein, unless otherwise indicated, and the dashed lines correspond to an optional spirofused cycloalkyl.

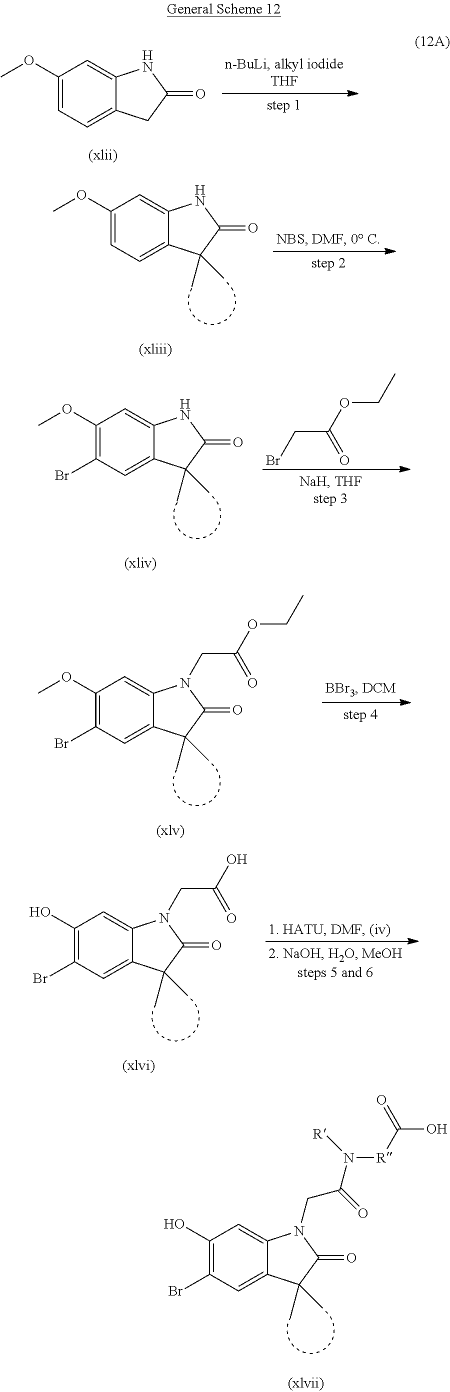

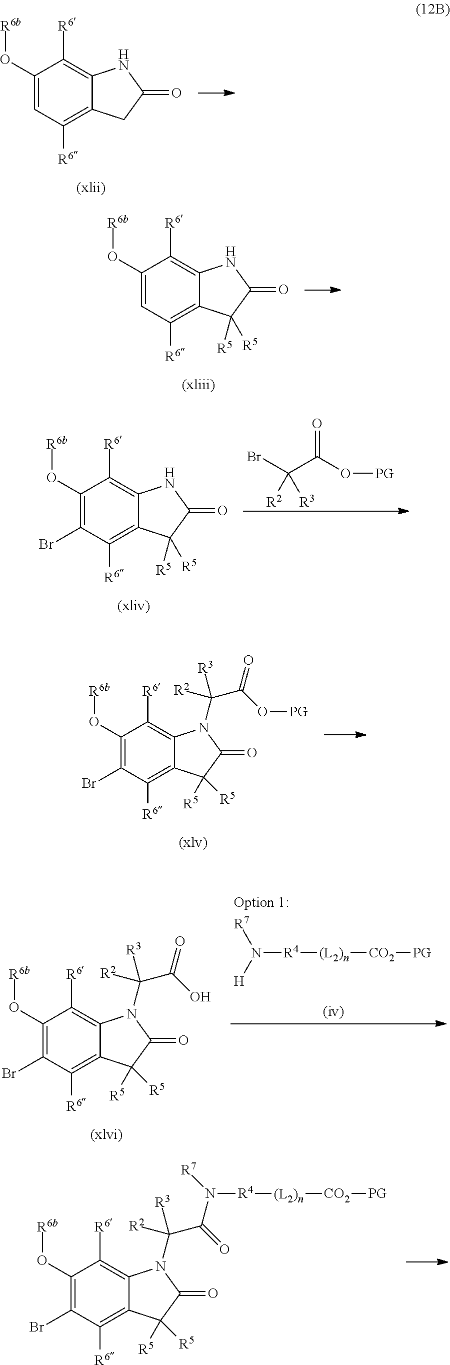

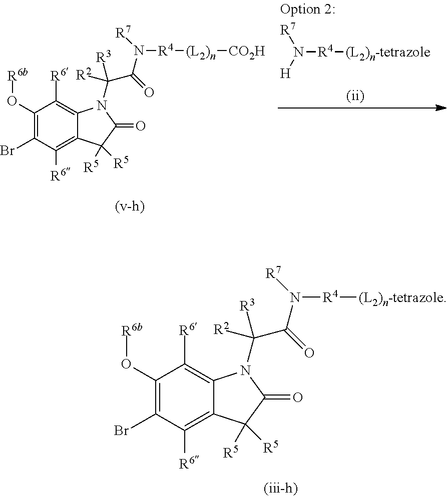

Compounds of Formula (I), wherein R⁶ is C₁-C₆ alkoxy (e.g., —OR^{6b}, wherein R^{6b} is C₁-C₆ alkyl) and R¹ is —Br, may be prepared in a process as shown in General Scheme 12, through the alkylation of (xlii), or salt thereof, to provide the dialkylated product (xliii), or salt thereof, followed by bromination to provide the brominated intermediate (xliv), or salt thereof, and N-alkylation to provide the ester intermediate (xlv), or salt thereof. Hydrolysis and amide coupling (and subsequent hydrolysis when the target is a carboxylic acid and not a tetrazole) then provides the carboxylic acid target analog (xlvii) or (v-h), or salt thereof, and the tetrazole target analog (iii-i), or salt thereof, wherein the indolinones (xlii), aminoesters (iv), and aminotetrazoles (ii) are either commercially available, known in the chemical literature, or prepared through previous schemes herein, unless otherwise indicated, and the dashed lines correspond to an optional spirofused cycloalkyl.

### (3) Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity. Effectiveness of compounds of the disclosure can be determined by industry-accepted assays/disease models according to standard practices of elucidating the same as described in the art and are found in the current general knowledge.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays are may be suitable for detecting the effect of the compounds of the present disclosure. These in vitro or in vivo biological assays can include, but are not limited to, enzymatic activity assays, electrophoretic mobility shift assays, reporter gene assays, binding assays, cellular assays (cell lines, primary cells and whole blood), in vitro cell viability assays, as well as assays for determining NLRP3 potency, unbound clearance, solubility, and permeability.

In some embodiments, the compounds of the instant disclosure may be tested for their human-NLRP3 inhibitory activity using known procedures, such as the methodology reported in Coll et al. Nat Med. (2015) 21(3):248-255. See also the Examples, Biological Assay Methods section.

In some embodiments, the compounds of the instant disclosure may be tested for unbound clearance (Clu) following known procedures, such as described in Miller et al., J. Med. Chem. (2020) 63:12156-12170. For example, unbound clearance (Clu) may be calculated by dividing total clearance (‘CL’ in mL/min/kg) as measured in blood or plasma by the unbound fraction in plasma (fu).

In some embodiments, the solubility of compounds of the instant disclosure may be determined following known procedures, such as described in Alsenz and Kansy, Advanced Drug Delivery Reviews (2007) 59:546-567, and Wang et al. J Mass Spectrom. (2000) 35:71-76. For example, the kinetic solubility in physiologically relevant media may be measured using serial dilution and two-hour incubation period, followed by filtration, and reported in mM by LC-MS/MS. Thermodynamic solubility in physiologically relevant media may be measured by LC-MS/MS, after a twenty-four-hour incubation, followed by filtration, and reported in mg/mL.

### (4) Pharmaceutical Compositions

In some aspects, the present disclosure provides a pharmaceutical composition comprising a compound of Formula (I), or a pharmaceutically acceptable salt, solvate, clathrate, hydrate, stereoisomer, tautomer, isotopic derivative, prodrug or polymorph thereof, as an active ingredient, and one or more of a pharmaceutically acceptable diluent, carrier, or excipient. In some embodiments, the present disclosure provides a pharmaceutical composition comprising a compound of Formula (I) selected from Tables 1 or 2, or a pharmaceutically acceptable salt, solvate, clathrate, hydrate, stereoisomer, tautomer, isotopic derivative, prodrug or polymorph thereof, and one or more of a pharmaceutically acceptable diluent, carrier, or excipient. In some embodiments, the present disclosure provides a pharmaceutical composition comprising a compound of Formula (I) selected from Table 1, or a pharmaceutically acceptable salt, solvate, clathrate, hydrate, stereoisomer, tautomer, isotopic derivative, prodrug or polymorph thereof, and one or more of a pharmaceutically acceptable diluent, carrier, or excipient. In some embodiments, the present disclosure provides a pharmaceutical composition comprising a compound of Formula (I) selected from Table 2, or a pharmaceutically acceptable salt, solvate, clathrate, hydrate, stereoisomer, tautomer, isotopic derivative, prodrug or polymorph thereof, and one or more of a pharmaceutically acceptable diluent, carrier, or excipient.

The pharmaceutical composition may be administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the pharmaceutical composition is administered orally.

The compound of Formula (I) can be formulated for oral use, e.g., for example as tablets, pills, hard or soft capsules (each of which includes sustained release or timed release formulations), lozenges, suspensions (e.g., aqueous or oily suspensions), emulsions, powders (e.g., dispersible powders), granules, syrups, elixirs, and tinctures; for topical use (for example as creams, ointments, gels, or aqueous or oily solutions or suspensions); for administration by inhalation (for example as a finely divided powder or a liquid aerosol); for administration by insufflation (for example as a finely divided powder) or for parenteral administration (for example as a sterile aqueous or oily solution for intravenous (bolus or in-fusion), subcutaneous, intramuscular, intraperitoneal or intramuscular dosing or as a suppository for rectal dosing); or transdermal (e.g., patch) administration, all using forms well known to those of ordinary skill in the pharmaceutical arts.

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

The formulation may be in the form of an aqueous solution comprising an aqueous vehicle. The aqueous vehicle component may comprise water and at least one pharmaceutically acceptable excipient. Suitable acceptable excipients include a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Compositions intended for oral use may further contain, for example, one or more coloring, sweetening, flavoring and/or preservative agents.

Compounds of Formula (I), or a pharmaceutically acceptable salt, solvate, clathrate, hydrate, stereoisomer, tautomer, isotopic derivative, prodrug or polymorph thereof, or a pharmaceutical composition comprising same, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced). Alternatively, by way of example only, the benefit experienced by a subject may be increased by administering the compound of Formula (I) with another therapeutic agent (which also includes a therapeutic regimen) that also has therapeutic benefit.

In the instances where the compound of Formula (I) is administered in combination with other therapeutic agents, the compound need not be administered via the same route as other therapeutic agents, and may, because of different physical and chemical characteristics, be administered by a different route. For example, the compound of Formula (I) may be administered orally to generate and maintain good blood levels thereof, while the other therapeutic agent may be administered intravenously.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease or disorder comprising a compound of Formula (I) and another therapeutic agent.

According to a further aspect of the disclosure there is provided a pharmaceutical composition comprising a compound of Formula (I), or a pharmaceutically acceptable salt, solvate, clathrate, hydrate, stereoisomer, tautomer, isotopic derivative, prodrug or polymorph thereof, in combination with another therapeutic agent, and a pharmaceutically excipient.

### (5) Methods of Use and Treatment

Compounds of Formula (I) have been found useful as inhibitors of NLRP3 activity.

In some embodiments, a compound of Formula (I) modulates NLRP3. In some embodiments, modulation is inhibition.

In some aspects, provided is a method of treating a disease or disorder in a subject in need thereof, comprising administering to the subject an effective amount of a compound of Formula (I) or a pharmaceutically acceptable salt, solvate, clathrate, hydrate, stereoisomer, tautomer, isotopic derivative, prodrug or polymorph thereof, or a pharmaceutical composition comprising same. In some embodiments, the disease or disorder is associated with aberrant NLRP3 activity, and the method comprises inhibiting the aberrant NLRP3 activity such that the subject is treated.

In some aspects, provided is a method of treating a disease or disorder in a subject in need thereof, comprising administering to the subject a compound of Formula (I) or a pharmaceutically acceptable salt, solvate, clathrate, hydrate, stereoisomer, tautomer, isotopic derivative, prodrug or polymorph thereof, or a pharmaceutical composition comprising same.

In some aspects, provided is a method of therapeutically treating a disease or disorder disclosed herein in a subject in need thereof, comprising administering to the subject a therapeutically effective amount of a compound of Formula (I) or a pharmaceutically acceptable salt, solvate, clathrate, hydrate, stereoisomer, tautomer, isotopic derivative, prodrug or polymorph thereof, or a pharmaceutical composition comprising same.

In some aspects, provided is a method of treating a disease or disorder disclosed herein in a subject in need thereof, comprising administering to the subject a compound of Formula (I) or a pharmaceutically acceptable salt, solvate, clathrate, hydrate, stereoisomer, tautomer, isotopic derivative, prodrug or polymorph thereof, or a pharmaceutical composition comprising same.

In some aspects, provided is a method of prophylactically treating a disease or disorder disclosed herein in a subject in need thereof, comprising administering to the subject a prophylactically effective amount of a compound of Formula (I) or a pharmaceutically acceptable salt, solvate, clathrate, hydrate, stereoisomer, tautomer, isotopic derivative, prodrug or polymorph thereof, or a pharmaceutical composition comprising same.

In some aspects, provided is a method of prophylactically treating a disease or disorder disclosed herein in a subject in need thereof, comprising administering to the subject a compound of Formula (I) or a pharmaceutically acceptable salt, solvate, clathrate, hydrate, stereoisomer, tautomer, isotopic derivative, prodrug or polymorph thereof, or a pharmaceutical composition comprising same.

In some aspects, the present disclosure provides a compound of the present disclosure or a pharmaceutically acceptable salt thereof for use in modulating NLRP3.

In some aspects, the present disclosure provides a compound of the present disclosure or a pharmaceutically acceptable salt thereof for use in treating a disease or disorder disclosed herein.

In some aspects, the present disclosure provides use of a compound of the present disclosure or a pharmaceutically acceptable salt thereof in the manufacture of a medicament for inhibiting NLRP3 activity.

In some aspects, the present disclosure provides use of a compound of the present disclosure or a pharmaceutically acceptable salt thereof in the manufacture of a medicament for treating a disease or disorder disclosed herein.

In some embodiments, the disease or disorder is a disease or disorder in which NLRP3 activity is implicated.

In some embodiments, the disease or disorder is a disease or disorder of the central nervous system (CNS), a disease or disorder of the peripheral nervous system (PNS), a primary neurological disease of the muscles, an inflammatory disorder, an autoimmune disorder, cancer, an infection, a metabolic disease, a cardiovascular disease, a respiratory disease, a kidney disease, a liver disease, an ocular disease, a skin disease, a lymphatic disease, a rheumatic disease, a psychological disease, graft versus host disease, pain (including disorders related to pain management, such as allodynia), or an NLRP3-related disease in a subject that has been determined to carry a germline or somatic non-silent mutation in NLRP3.

In some embodiments, the disease or disorder is a disease or disorder of central nervous system and/or peripheral nervous system (“PNS”), such as dementia, Alzheimer's disease (“AD”) epilepsy, traumatic brain injury (“TBI”), multiple sclerosis (“MS”), a developmental disturbance, acute disseminated encephalopathy, transverse myelitis, Parkinson's disease (“PD”), amyotrophic lateral sclerosis (“ALS”), Huntington's disease (“HD”), or spinal cord injury.

In some embodiments, the disease or disorder is a primary neurological disease of the muscle, such as a dystrophy or spinal muscular atrophy.

In some embodiments, the disease or disorder is an inflammatory disorder, such as gout or anemia of inflammation.

In some embodiments, the disease or disorder is an autoimmune disease, such as ulcerative colitis.

In some embodiments, the disease or disorder is cancer, such as skin cancer or colon cancer.

In some embodiments, the disease or disorder is an infection, such as a neuro-infection.

In some embodiments, the disease or disorder is a metabolic disease, such as diabetes, e.g., type 2 diabetes.

In some embodiments, the disease or disorder is a cardiovascular disease, such as stroke.

In some embodiments, the disease or disorder is a respiratory disease, such as asthma (e.g., steroid-resistant asthma, severe steroid-resistant asthma) or chronic obstructive pulmonary disease (“COPD”).

In some embodiments, the disease or disorder is a kidney disease, such as acute kidney disease, a chronic kidney disease, or a rare kidney disease.

In some embodiments, the disease or disorder is a liver disease, such as nonalcoholic fatty liver disease (NAFLD) or non-alcoholic steatohepatitis (NASH).

In some embodiments, the disease or disorder is an ocular disease, such as optic neuritis or macular degeneration.

In some embodiments, the disease or disorder is a skin disease, such as psoriasis, hidradenitis suppurativa (HS), or atopic dermatitis.

In some embodiments, the disease or disorder is a lymphatic disease.

In some embodiments, the disease or disorder is a rheumatic disease, such as osteoarthritis, dermatomyositis, Still's disease, or juvenile idiopathic arthritis.

In some embodiments, the disease or disorder is a psychological disease, such as a neuropsychiatric condition, including depression, major depressive disorder, or refractory depression.

In some embodiments, the disease or disorder is a graft versus host disease,

In some embodiments, the disease or disorder is pain (including disorders related to pain management), such as pain management addiction, osteoarthritis pain, or allodynia.

In some embodiments, the NLRP3-related disease in a subject that has been determined to carry a germline or somatic non-silent mutation in NLRP3 is cryopyrin-associated autoinflammatory syndrome. In some embodiments, the cryopyrin-associated autoinflammatory syndrome is familial cold autoinflammatory syndrome, Muckle-Wells syndrome, or neonatal onset multisystem inflammatory disease (NOMID).

In some embodiments, the disease or disorder is dementia, Alzheimer's disease (“AD”), epilepsy, traumatic brain injury (“TBI”), multiple sclerosis (“MS”), developmental disturbances, acute disseminated encephalopathy, transverse myelitis, Parkinson's disease (“PD”), amyotrophic lateral sclerosis (“ALS”), spinal muscular atrophy, Huntington's disease (“HD”), a spinal cord injury, a dystrophy, a neuro-infection, a pain management addiction, a neuropsychiatric condition (e.g. depression, major depressive disorder, refractory depression), neonatal onset multisystem inflammatory disease (“NOMID”), asthma, osteoarthritis, ulcerative colitis, gout, anemia of inflammation, Still's disease, chronic obstructive pulmonary disease (“COPD”), osteoarthritis pain, or hidradenitis suppurativa.

In other aspects, provided is a method of inhibiting NLRP3 activity (e.g., in vitro or in vivo) in a cell, comprising contacting the cell with an effective amount of a compound of Formula (I), or a pharmaceutically acceptable salt, solvate, clathrate, hydrate, stereoisomer, tautomer, isotopic derivative, prodrug or polymorph thereof.

### (6) Additional Embodiments

Embodiments 1-36 are further contemplated herein.

Embodiment 1: A compound of Formula (I):

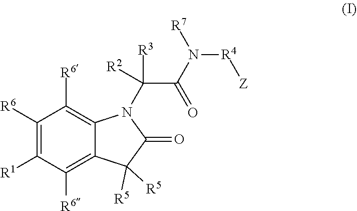

or a pharmaceutically acceptable salt, solvate, clathrate, hydrate, stereoisomer, or tautomer thereof, wherein:
R¹ is halo, nitrile, C₁-C₆ alkyl, C₂-C₆ alkenyl, C₂-C₆ alkynyl, C₁-C₆ haloalkyl, C₁-C₆ alkoxy, or C₃-C₁₂ cycloalkyl;
each R² and R³ independently is H, C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy, or
R² and R³ cyclize, together with the atom to which they are attached, to form a C₃-C₁₂ cycloalkyl or 3- to 12-membered heterocyclyl, wherein the cycloalkyl or heterocyclyl is optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy;
R⁴ is C₁-C₆ alkyl, —(CH₂)ₚ—(C₃-C₁₂ cycloalkyl), —(CH₂)ₚ-(3- to 12-membered heterocyclyl), C₆-C₁₀ aryl, or —(CH₂)ₚ-(5- to 10-membered heteroaryl), wherein the alkyl, cycloalkyl, heterocyclyl, aryl, or heteroaryl is optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy;
Z is a —(CH₂)ₙ-(carboxylic acid) or —(CH₂)ₙ-tetrazole;
each R⁵ is independently C₁-C₆ alkyl, wherein the alkyl is optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy, or
both R⁵ cyclize, together with the atom to which they are attached, to form a C₃-C₁₂ cycloalkyl optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy;
R⁶ is H, halogen, —OH, C₁-C₆ alkyl, C₂-C₆ alkenyl, C₂-C₆ alkynyl, C₁-C₆ haloalkyl, or C₁-C₆ alkoxy;
R^{6′} is H, halogen, —OH, C₁-C₆ alkyl, C₂-C₆ alkenyl, C₂-C₆ alkynyl, C₁-C₆ haloalkyl, or C₁-C₆ alkoxy;
R^{6″} is H, halogen, —OH, C₁-C₆ alkyl, C₂-C₆ alkenyl, C₂-C₆ alkynyl, C₁-C₆ haloalkyl, or C₁-C₆ alkoxy;
R⁷ is independently H or C₁-C₄ alkyl;
n is an integer from 0-3; and
p is an integer from 0-2.

Embodiment 2: The compound of Embodiment 1, wherein:
R¹ is bromo, chloro, nitrile, C₁-C₆ alkyl, C₁-C₆ haloalkyl, or C₃-C₁₂ cycloalkyl; each R² and R³ independently is H, C₁-C₆ alkyl, C₁-C₆ haloalkyl, C₃-C₁₂ cycloalkyl, 3- to 12-membered heterocyclyl, C₆-C₁₀ aryl, or 5- to 10-membered heteroaryl, where the alkyl, haloalkyl, cycloalkyl, heterocyclyl, aryl, or heteroaryl is optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy, or
R² and R³ cyclize together to form a C₃-C₁₂ cycloalkyl or 3- to 12-membered heterocyclyl, wherein the cycloalkyl or heterocyclyl is optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy;
R⁴ is an n-propyl, —(CH₂)ₚ—(C₃-C₁₂ cycloalkyl), —(CH₂)ₚ-(3- to 12-membered heterocyclyl), C₆-C₁₀ aryl, or —(CH₂)ₚ-(5- to 10-membered heteroaryl), wherein the n-propyl, cycloalkyl, heterocyclyl, aryl, or heteroaryl is optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy;
Z is a —(CH₂)ₙ-(carboxylic acid) or —(CH₂)ₙ-tetrazole;
each R⁵ is C₁-C₆ alkyl optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy, or
both R⁵ cyclize, together with the atom to which they are attached, to form a C₃-C₁₂ cycloalkyl optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy;
R⁶ is H, halogen, —OH, C₁-C₆ alkyl, or C₁-C₆ alkoxy;
R^{6′} is H, halogen, —OH, C₁-C₆ alkyl, or C₁-C₆ alkoxy;
R^{6″} is H, halogen, —OH, C₁-C₆ alkyl, or C₁-C₆ alkoxy;
R⁷ is independently H or C₁-C₄ alkyl;
n is an integer from 0-3; and
p is integer from 0-2.

Embodiment 3: The compound of any one of the preceding Embodiments 1-2, wherein R¹ is bromo, chloro, or nitrile.

Embodiment 4: The compound of any one of the preceding Embodiments 1-3, wherein R¹ is C₁-C₆ alkyl or C₁-C₆ haloalkyl.

Embodiment 5: The compound of any one of the preceding Embodiments 1-4, wherein R¹ is C₃-C₁₂ cycloalkyl.

Embodiment 6: The compound of any one of the preceding Embodiments 1-5, wherein each R² and R³ independently is H, C₁-C₆ alkyl, C₁-C₆ haloalkyl, C₃-C₁₂ cycloalkyl, 3- to 12-membered heterocyclyl, C₆-C₁₀ aryl, or 5- to 10-membered heteroaryl, where the alkyl, haloalkyl, cycloalkyl, heterocyclyl, aryl, or heteroaryl is optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy.

Embodiment 7: The compound of any one of the preceding Embodiments 1-6, wherein R² and R³ cyclize together to form a C₃-C₁₂ cycloalkyl or 3- to 12-membered heterocyclyl, wherein the cycloalkyl or heterocyclyl is optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy.

Embodiment 8: The compound of any one of the preceding Embodiments 1-7, wherein R⁴ is an n-propyl.

Embodiment 9: The compound of any one of the preceding Embodiments 1-8, wherein Z is carboxylic acid.

Embodiment 10: The compound of any one of the preceding Embodiments 1-9, wherein Z is tetrazole.

Embodiment 11: The compound of any one of the preceding Embodiments 1-10, wherein R⁵ is C₁-C₆ alkyl optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy.

Embodiment 12: The compound of any one of the preceding Embodiments 1-11, wherein both R⁵ cyclize, together with the atom to which they are attached, to form a C₃-C₁₂ cycloalkyl optionally substituted with one or more halo, cyano, hydroxy, amino, C₁-C₆ alkyl, C₂-C₆ alkenyl, C₂-C₆ alkynyl, C₁-C₆ haloalkyl, or C₁-C₆ alkoxy.

Embodiment 13: The compound of any one of the preceding Embodiments 1-12, wherein R⁶ is H.

Embodiment 14: The compound of any one of the preceding Embodiments 1-13, wherein R⁶ is halogen, —OH, C₁-C₆ alkyl, or C₁-C₆ alkoxy.

Embodiment 15: The compound of any one of the preceding Embodiments 1-14, wherein R^{6′} is H.

Embodiment 16: The compound of any one of the preceding Embodiments 1-15, wherein R^{6′} is halogen, —OH, C₁-C₆ alkyl, or C₁-C₆ alkoxy.

Embodiment 17: The compound of any one of the preceding Embodiments 1-16, wherein R^{6″} is H.

Embodiment 18: The compound of any one of the preceding Embodiments 1-17, wherein R^{6″} is halogen, —OH, C₁-C₆ alkyl, or C₁-C₆ alkoxy.

Embodiment 19: The compound of any one of the preceding Embodiments 1-18, wherein R⁷ is H.

Embodiment 20: The compound of any one of the preceding Embodiments 1-19, wherein the compound is selected from the compounds described in Table 1.

Embodiment 21: The compound of Embodiment 20, or a pharmaceutically acceptable salt or stereoisomer thereof.

Embodiment 22: The compound of Embodiment 20, or a pharmaceutically acceptable salt thereof.

Embodiment 23: An isotopic derivative of the compound of any one of the preceding Embodiments 1-22.

Embodiment 24: A pharmaceutical composition comprising the compound of any one of the preceding Embodiments 1-23 and one or more pharmaceutically acceptable carriers.

Embodiment 25: A method of treating or preventing an NLRP3-related disease or disorder, the method comprising administering to the subject a compound of any one of the preceding Embodiments 1-23.

Embodiment 26: A method of inhibiting NLRP3, the method comprising administering to the subject a compound of any one of the preceding Embodiments 1-23.

Embodiment 27: The method of Embodiment 25 or Embodiment 26, wherein the subject is a human.

Embodiment 28: The method of any one of Embodiments 25-27, wherein the NLRP3-related disease or disorder is inflammation, an auto-immune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or an NLRP3-related disease in a subject that has been determined to carry a germline or somatic non-silent mutation in NLRP3.

Embodiment 29: The method of any one of Embodiments 25-27, wherein the disease or disorder of the central nervous system is Parkinson's disease, Alzheimer's disease, traumatic brain injury, spinal cord injury, amyotrophic lateral sclerosis, or multiple sclerosis.

Embodiment 30: The method of any one of Embodiments 25-27, wherein the kidney disease is an acute kidney disease, a chronic kidney disease, or a rare kidney disease.

Embodiment 31: The method of any one of Embodiments 25-27, wherein the skin disease is psoriasis, hidradenitis suppurativa (HS), or atopic dermatitis.

Embodiment 32: The method of any one of Embodiments 25-27, wherein the rheumatic disease is dermatomyositis, Still's disease, or juvenile idiopathic arthritis.

Embodiment 33: The method of any one of Embodiments 25-27, wherein the NLRP3-related disease in a subject that has been determined to carry a germline or somatic non-silent mutation in NLRP3 is cryopyrin-associated autoinflammatory syndrome.

Embodiment 34: The method of any one of Embodiments 25-27, wherein the cryopyrin-associated autoinflammatory syndrome is familial cold autoinflammatory syndrome, Muckle-Wells syndrome, or neonatal onset multisystem inflammatory disease.

Embodiment 35: The compound of any one of the preceding Embodiments 1-34 for use in treating or preventing an NLRP3-related disease or disorder.

Embodiment 36: Use of the compound of any one of the preceding Embodiments 1-34, in the manufacture of a medicament, for the treatment or prevention of an NLRP3-related disease or disorder.

Embodiments A1-A76 are further contemplated herein.

Embodiment A1: A compound of Formula (I):

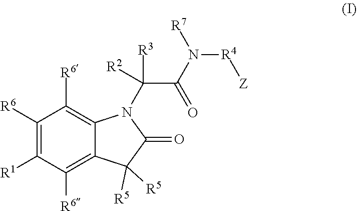

or a pharmaceutically acceptable salt thereof, wherein:
R¹ is halo, —CN, C₁-C₆ alkyl, C₂-C₆ alkenyl, C₂-C₆ alkynyl, C₁-C₆ haloalkyl, C₁-C₆ alkoxy, or C₃-C₁₂ cycloalkyl;
each R² and R³ independently is H, C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or R² and R³ cyclize, together with the atom to which they are attached, to form a C₃-C₁₂ cycloalkyl or 3- to 12-membered heterocyclyl, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁴ is —C₁-C₆ alkyl-, -(L₁)ₚ-(C₃-C₁₂ cycloalkyl)-, -(L₁)ₚ-(3- to 12-membered heterocyclyl)-, -(L₁)ₚ-(C₆-C₁₀ aryl)-, or -(L₁)ₚ-(5- to 10-membered heteroaryl)-, wherein each instance of L₁ is independently —(C(R^{L})₂)—, further wherein each instance of R^{L1} is independently H, halo or C₁-C₃ alkyl, or two R^{L1} groups, together with the atom to which they are attached, form a C₃₋₄ cycloalkyl; and wherein the alkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
Z is a -(L₂)ₙ-(carboxylic acid) or -(L₂)ₙ-tetrazole, wherein each instance of L₂ is independently —(C(R^{L2})₂)—, further wherein each instance of R^{L2} is independently H, halo or C₁-C₃ alkyl, or two R^{L2} groups, together with the atom to which they are attached, to form a C₃₋₄ cycloalkyl; and wherein the alkyl or cycloalkyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
each R⁵ is independently halo or C₁-C₆ alkyl, wherein the alkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or both R⁵ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁶ is H, halo, —OH, C₁-C₆ alkyl, C₂-C₆ alkenyl, C₂-C₆ alkynyl, C₁-C₆ haloalkyl, or C₁-C₆ alkoxy;
R^{6′} is H, halo, —OH, C₁-C₆ alkyl, C₂-C₆ alkenyl, C₂-C₆ alkynyl, C₁-C₆ haloalkyl, or C₁-C₆ alkoxy;
R^{6″} is H, halo, —OH, C₁-C₆ alkyl, C₂-C₆ alkenyl, C₂-C₆ alkynyl, C₁-C₆ haloalkyl, or C₁-C₆ alkoxy;
R⁷ is independently H or C₁-C₄ alkyl;
n is an integer from 0, 1, 2, or 3; and
p is an integer from 0, 1, or 2.

Embodiment A2: The compound of Embodiment A1, or a pharmaceutically acceptable salt thereof, wherein each instance of R² and R³ is independently H, C₁-C₃ alkyl, C₁-C₃ haloalkyl, or R² and R³ cyclize, together with the atom to which they are attached, to form a C₃ cycloalkyl.

Embodiment A3: The compound of Embodiment A2, or a pharmaceutically acceptable salt thereof, wherein each instance of R² and R³ is H.

Embodiment A4: The compound of Embodiment A2, or a pharmaceutically acceptable salt thereof, wherein R² and R³ cyclize, together with the atom to which they are attached, to form a C₃ cycloalkyl.

Embodiment A5: The compound of any one of Embodiments A1-A4, or a pharmaceutically acceptable salt thereof, wherein R⁴ is -(L₁)ₚ-(C₃-C₁₂ cycloalkyl)- or -(L₁)ₚ-(3- to 12-membered heterocyclyl)-, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; p is 0; n is 2; and Z is a -(L₂)ₙ-(carboxylic acid) or -(L₂)ₙ-tetrazole; such that the least number of consecutively covalently bonded atoms between the NR⁷ amide nitrogen to the terminal carboxylic acid or tetrazole group is 3 consecutively covalently bonded atoms.

Embodiment A6: The compound of Embodiment A5, or a pharmaceutically acceptable salt thereof, wherein —R⁴—Z is a group of formula (i):

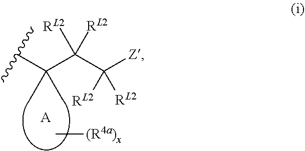

wherein Z′ is tetrazole or carboxylic acid; Ring A is C₃-C₅ cycloalkyl or 4- to 5-membered heterocyclyl; each R^{4a} is independently halo, C₁-C₃ alkyl, C₁-C₃ haloalkyl, or C₃ cycloalkyl; and x is 0, 1, 2, or 3.

Embodiment A7: The compound of Embodiment A6, or a pharmaceutically acceptable salt thereof, wherein the group of formula (i) is:

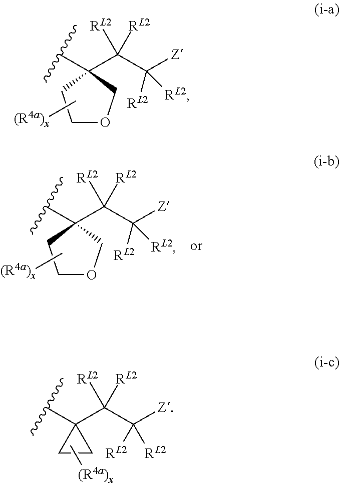

Embodiment A8: The compound of Embodiment A7, or a pharmaceutically acceptable salt thereof, wherein the group of formula (i) is:

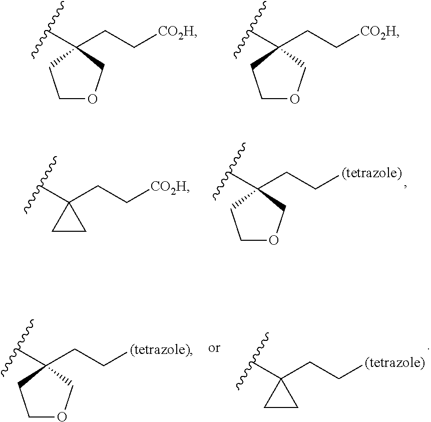

Embodiment A9: The compound of any one of Embodiments A1-A4, or a pharmaceutically acceptable salt thereof, wherein R⁴ is -(L₁)ₚ-(C₃-C₁₂ cycloalkyl)- or -(L₁)ₚ-(3- to 12-membered heterocyclyl)-, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; p is 1; n is 1; and Z is a -(L₂)ₙ-(carboxylic acid) or -(L₂)ₙ-tetrazole; such that the least number of consecutively covalently bonded atoms between the NR⁷ amide nitrogen to the terminal carboxylic acid or tetrazole group is 3 consecutively covalently bonded atoms.

Embodiment A10: The compound of Embodiment A9, or a pharmaceutically acceptable salt thereof, wherein —R⁴—Z is a group of formula (ii):

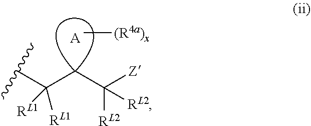

wherein Z′ is tetrazole or carboxylic acid; Ring A is C₃-C₅ cycloalkyl or 4- to 5-membered heterocyclyl; each R^{4a} is independently halo, C₁-C₃ alkyl, C₁-C₃ haloalkyl, or C₃ cycloalkyl; and x is 0, 1, 2, or 3.

Embodiment A11: The compound of Embodiment A10, or a pharmaceutically acceptable salt thereof, wherein at least one instance of R^{L1} is —C₁-C₃ alkyl optionally substituted with one or more halo.

Embodiment A12: The compound of Embodiment A10, or a pharmaceutically acceptable salt thereof, wherein at least one instance of R^{L2} is —C₁-C₃ alkyl optionally substituted with one or more halo.

Embodiment A13: The compound of Embodiment A10, or a pharmaceutically acceptable salt thereof, wherein each instance of R^{L1} and R^{L2} is H.

Embodiment A14: The compound of Embodiment A10, or a pharmaceutically acceptable salt thereof, wherein group of formula (ii) is:

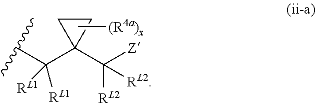

Embodiment A15: The compound of Embodiment A14, or a pharmaceutically acceptable salt thereof, wherein group of formula (ii-a) is:

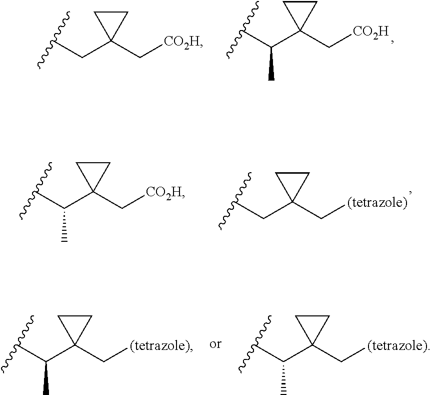

Embodiment A16: The compound of any one of Embodiments A1-A4, or a pharmaceutically acceptable salt thereof, wherein R⁴ is -(L₁)ₚ-(C₃-C₁₂ cycloalkyl)-, -(L₁)ₚ-(3- to 12-membered heterocyclyl)-, -(L₂)ₚ-(C₆-C₁₀ aryl)-, or -(L₂)ₚ-(5- to 10-membered heteroaryl)-, wherein the cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; p is 0; n is 0; and Z is a -(L₂)ₙ-(carboxylic acid) or -(L₂)ₙ-tetrazole; such that the least number of consecutively covalently bonded atoms between the NR⁷ amide nitrogen to the terminal carboxylic acid or tetrazole group is 3 consecutively covalently bonded atoms.

Embodiment A17: The compound of Embodiment A16, or a pharmaceutically acceptable salt thereof, wherein —R⁴—Z is a group of formula (iii):

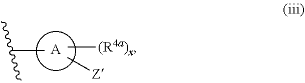

wherein Z′ is tetrazole or carboxylic acid; Ring A is C₃-C₅ cycloalkyl or 4- to 5-membered heterocyclyl; each R^{4a} is independently halo, C₁-C₃ alkyl, C₁-C₃ haloalkyl, or C₃ cycloalkyl; and x is 0, 1, 2, or 3.

Embodiment A18: The compound of Embodiment A17, or a pharmaceutically acceptable salt thereof, wherein the group of formula (iii) is:

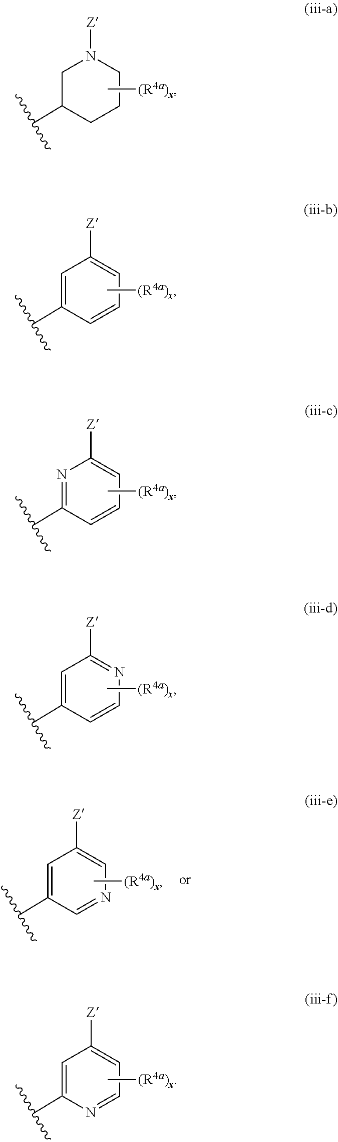

Embodiment A19: The compound of Embodiment A18, or a pharmaceutically acceptable salt thereof, wherein the group of formula (iii-a) is:

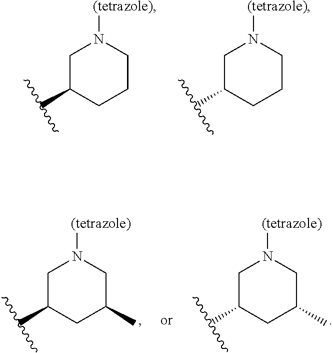

Embodiment A20: The compound of any one of Embodiments A1-A4, or a pharmaceutically acceptable salt thereof, wherein R⁴ is -(n-propyl)- optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; n is 0; and Z is a -(L₂)ₙ-(carboxylic acid) or -(L₂)ₙ-tetrazole.

Embodiment A21: The compound of Embodiment A20, or a pharmaceutically acceptable salt thereof, wherein —R⁴—Z is a group of formula (iv):

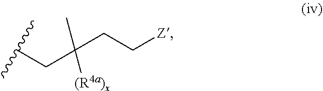

wherein Z′ is tetrazole or carboxylic acid; each R^{4a} is independently halo, C₁-C₃ alkyl, C₁-C₃ haloalkyl, or C₃ cycloalkyl; and x is 0, 1, 2, or 3.

Embodiment A22: The compound of Embodiment A21, or a pharmaceutically acceptable salt thereof, wherein the group of formula (iv) is:

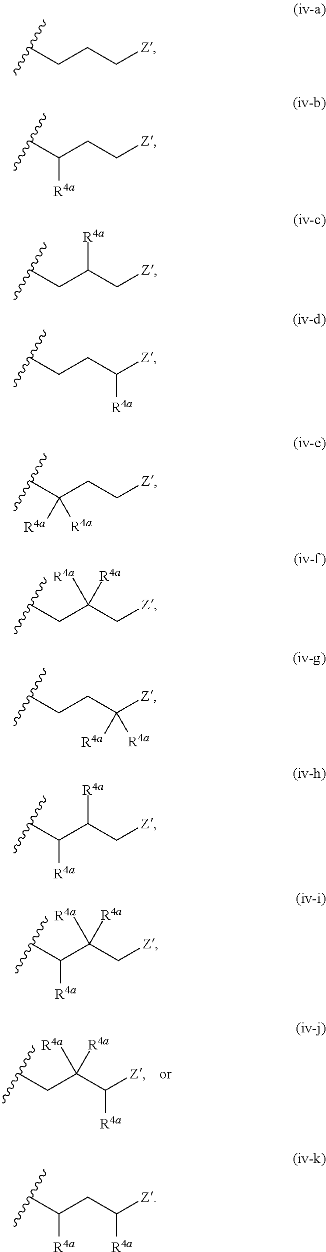

Embodiment A23: The compound of Embodiment A21, or a pharmaceutically acceptable salt thereof, wherein the group of formula (iv) is:

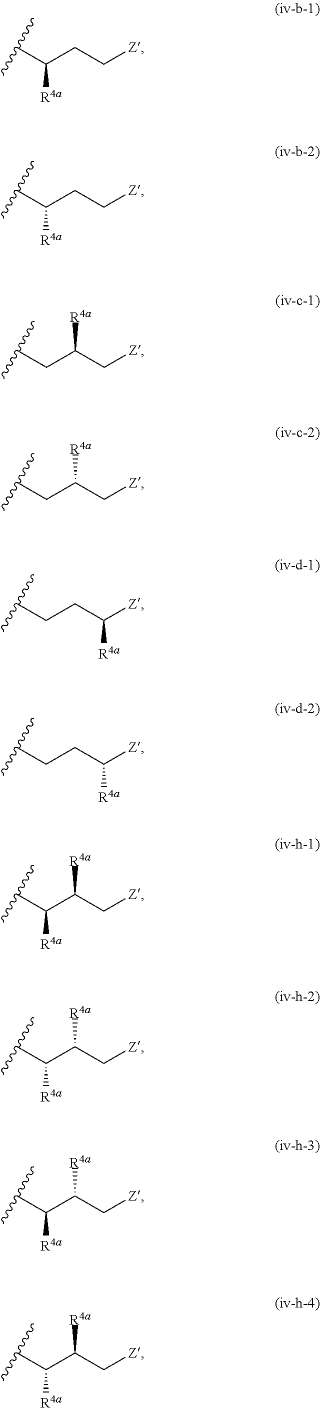

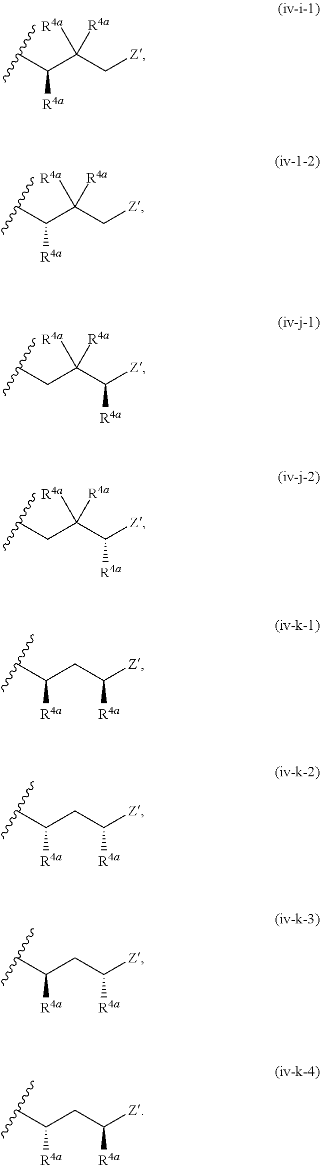

Embodiment A24: The compound of any one of Embodiments A21-A23, or a pharmaceutically acceptable salt thereof, wherein each instance of R⁴ is independently —CH₃, —CF₃, C₃ cycloalkyl, or fluoro.

Embodiment A25: The compound of Embodiment A21, or a pharmaceutically acceptable salt thereof, wherein the group of formula (iv) is:

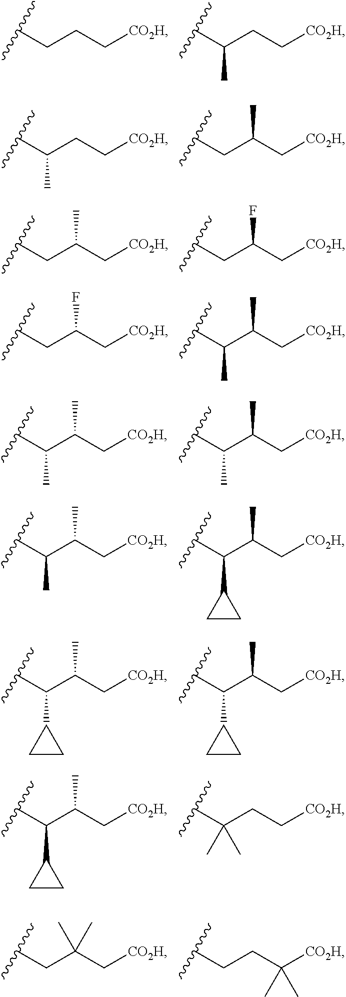

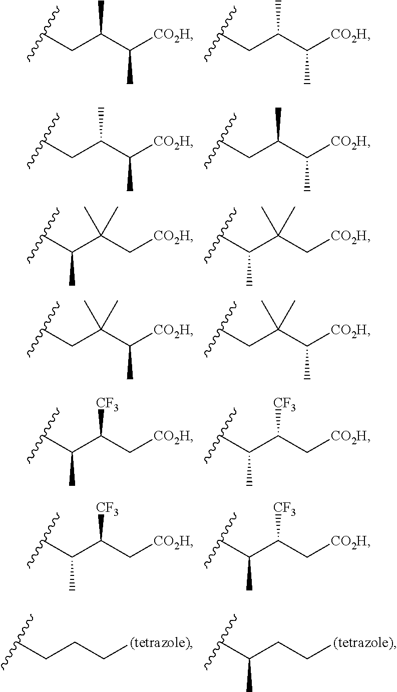

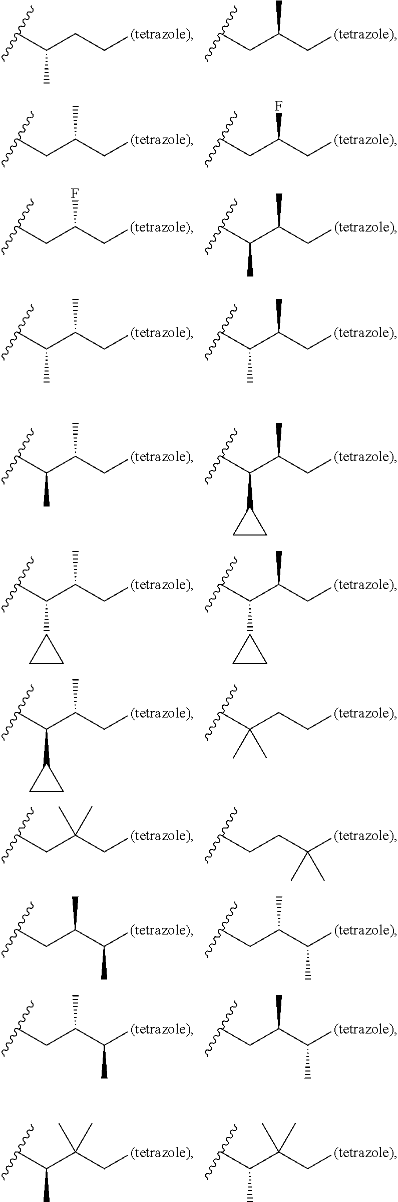

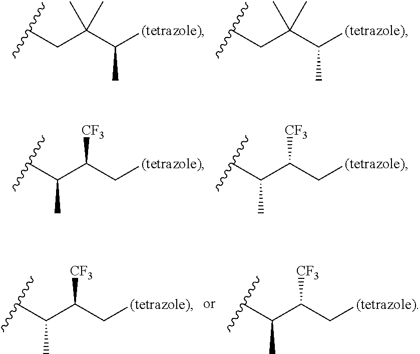

Embodiment A26: The compound of any one of Embodiments A1-A4, or a pharmaceutically acceptable salt thereof, wherein p is 1; n is 0; Z is a -(L₂)ₙ-(carboxylic acid) or -(L₂)ₙ-tetrazole; and R⁴ is -(L₁)ₚ-(C₆-C₁₀ aryl)- or -(L₁)ₚ-(5- to 10-membered heteroaryl), wherein the aryl or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; such that the least number of consecutively covalently bonded atoms between the NR⁷ amide nitrogen to the terminal carboxylic acid or tetrazole group is 3 consecutively covalently bonded atoms.

Embodiment A27: The compound of Embodiment A26, or a pharmaceutically acceptable salt thereof, wherein —R⁴—Z group of formula:

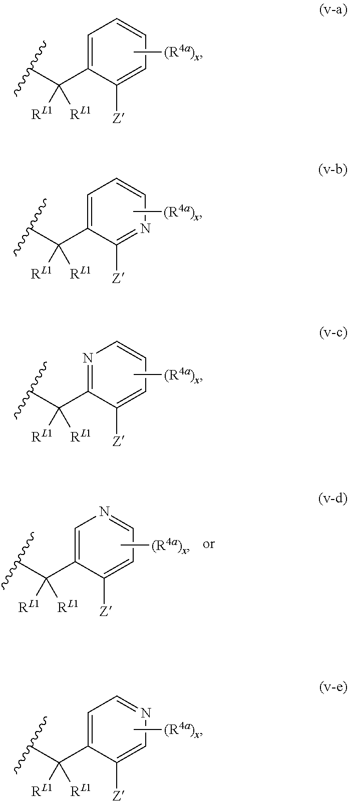

wherein Z′ is tetrazole or carboxylic acid; each R^{4a} is independently halo, C₁-C₃ alkyl, C₁-C₃ haloalkyl; and x is 0, 1, 2, or 3.

Embodiment A28: The compound of any one of Embodiments A1-A4, or a pharmaceutically acceptable salt thereof, wherein p is 0; n is 0; Z is a -(L₂)ₙ-(carboxylic acid) or -(L₂)ₙ-tetrazole; and R⁴ is -(L₁)ₚ-(C₃-C₁₂ cycloalkyl)-, -(L₁)ₚ-(3- to 12-membered heterocyclyl)-, -(L₁)ₚ-(C₆-C₁₀ aryl)-, or -(L₁)ₚ-(5- to 10-membered heteroaryl), wherein the cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; such that the least number of consecutively covalently bonded atoms between the NR⁷ amide nitrogen to the terminal carboxylic acid or tetrazole group is 2 consecutively covalently bonded atoms.

Embodiment A29: The compound of Embodiment A28, or a pharmaceutically acceptable salt thereof, wherein —R⁴—Z group of formula:

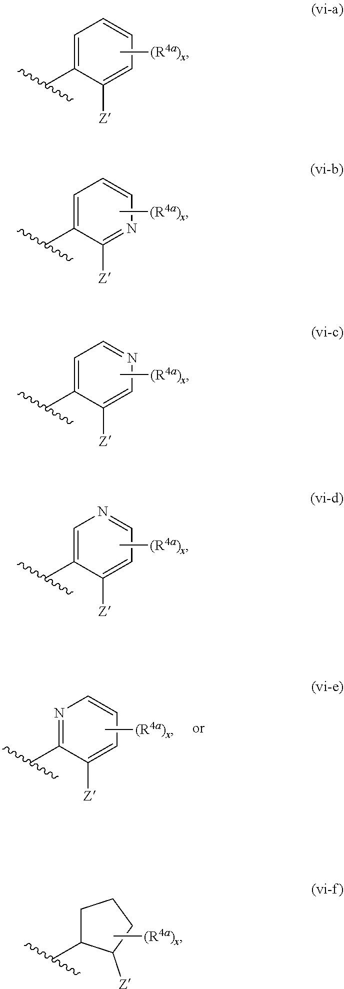

wherein Z′ is tetrazole or carboxylic acid; each R^{4a} is independently halo, C₁-C₃ alkyl, C₁-C₃ haloalkyl; and x is 0, 1, 2, or 3.

Embodiment A30: The compound of Embodiment A28, or a pharmaceutically acceptable salt thereof, wherein —R⁴—Z group is of formula:

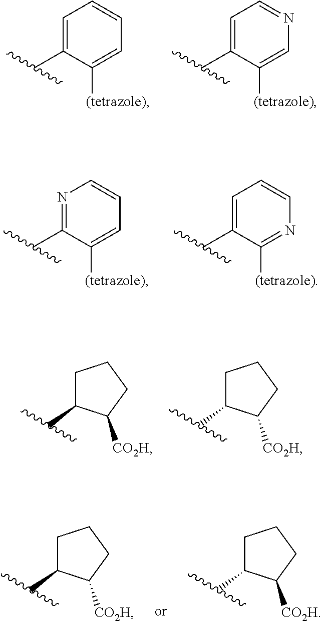

Embodiment A31: The compound of any one of Embodiments A1-A4, or a pharmaceutically acceptable salt thereof, wherein p is 0; n is 0; Z is a -(L₂)ₙ-(carboxylic acid) or -(L₂)ₙ-tetrazole; and R⁴ is -(L₁)ₚ-(C₆-C₁₀ aryl)-, or -(L₁)ₚ-(5- to 10-membered heteroaryl)- wherein the aryl or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl; such that the least number of consecutively covalently bonded atoms between the NR⁷ amide nitrogen to the terminal carboxylic acid or tetrazole group is 4 consecutively covalently bonded atoms.

Embodiment A32: The compound of Embodiment A31, or a pharmaceutically acceptable salt thereof, wherein —R⁴—Z is a group of formula:

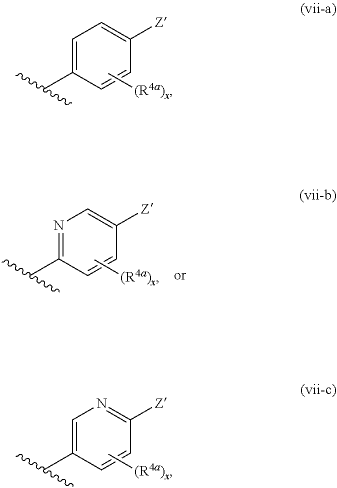

wherein Z′ is tetrazole or carboxylic acid; and each R^{4a} is independently halo, C₁-C₃ alkyl, C₁-C₃ haloalkyl, and x is 0, 1, 2, or 3.

Embodiment A33: The compound of any one of Embodiments A1-A32, or a pharmaceutically acceptable salt thereof, wherein each instance of R⁵ is selected from the group consisting of fluoro and C₁-C₆ alkyl, or both R⁵ cyclize, together with the atom to which they are attached, to form a C₃ cycloalkyl.

Embodiment A34: The compound of Embodiment A33, or a pharmaceutically acceptable salt thereof, wherein each instance of R⁵ is the same, and is selected from the group consisting of fluoro and C₁-C₆ alkyl.

Embodiment A35: The compound of Embodiment A34, or a pharmaceutically acceptable salt thereof, wherein each instance of R⁵ is fluoro or —CH₃.

Embodiment A36: The compound of any one of Embodiments A1-A35, or a pharmaceutically acceptable salt thereof, wherein R¹ is halo, C₁-C₃ alkyl, C₁-C₃ haloalkyl, or C₃-C₅ cycloalkyl.

Embodiment A37: The compound of any one of Embodiments A1-A36, or a pharmaceutically acceptable salt thereof, wherein R⁶ is H, halo, or —OH.

Embodiment A38: The compound of any one of Embodiments A1-A37, or a pharmaceutically acceptable salt thereof, wherein R^{6′} is H, halo, —OH, C₁-C₃ alkyl, C₁-C₃ haloalkyl, or C₁-C₃ alkoxy.

Embodiment A39: The compound of any one of Embodiments A1-A38, or a pharmaceutically acceptable salt thereof, wherein R^{6″} is H or halo.

Embodiment A40: The compound of any one of Embodiments A1-A39, or a pharmaceutically acceptable salt thereof, wherein the 6,5-bicyclic core of formula (viii):

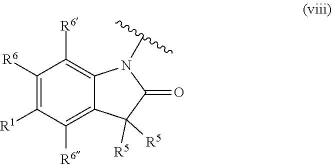

is of formula:

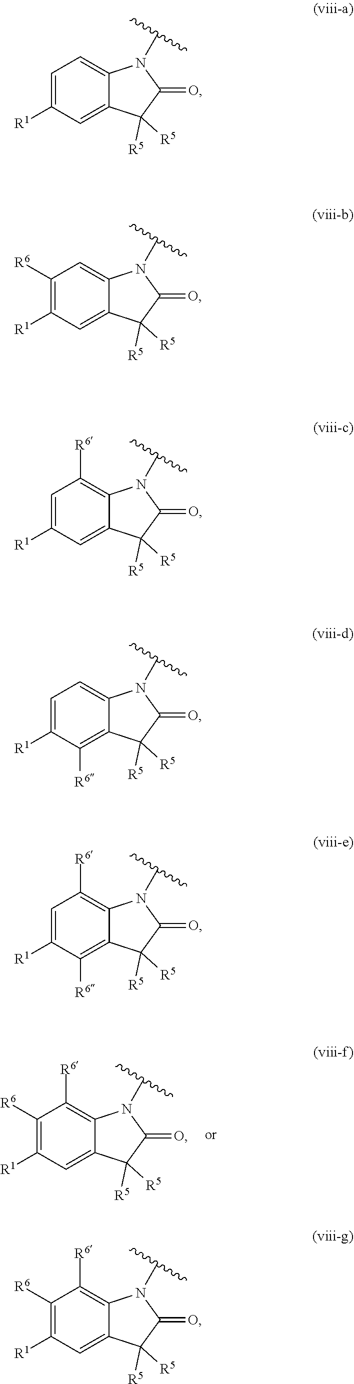

wherein at least one of R⁶, R^{6′} and R^{6″} is not H.

Embodiment A41: The compound of any one of Embodiments A1-A40, or a pharmaceutically acceptable salt thereof, wherein the 6,5-bicyclic core is of formula:

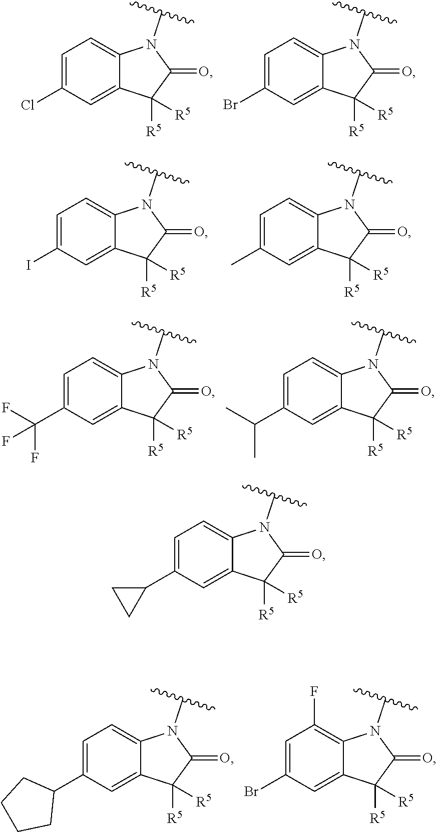

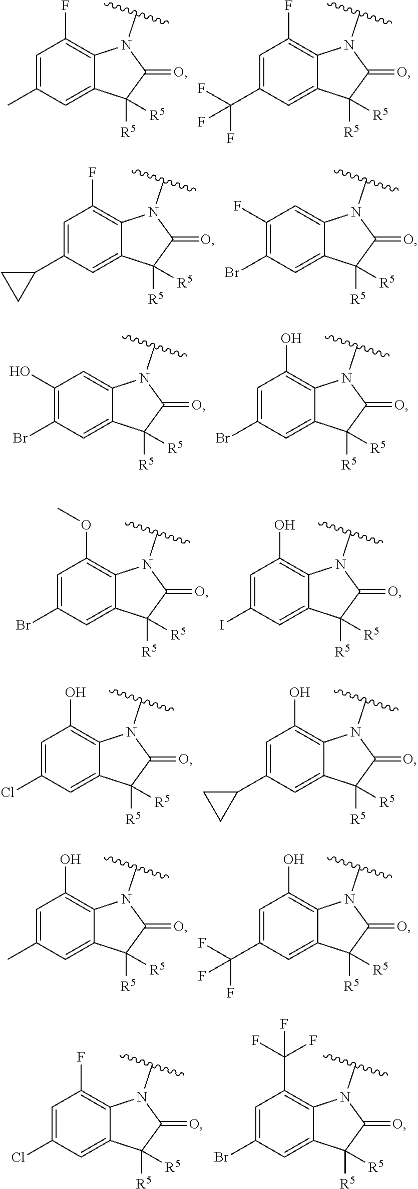

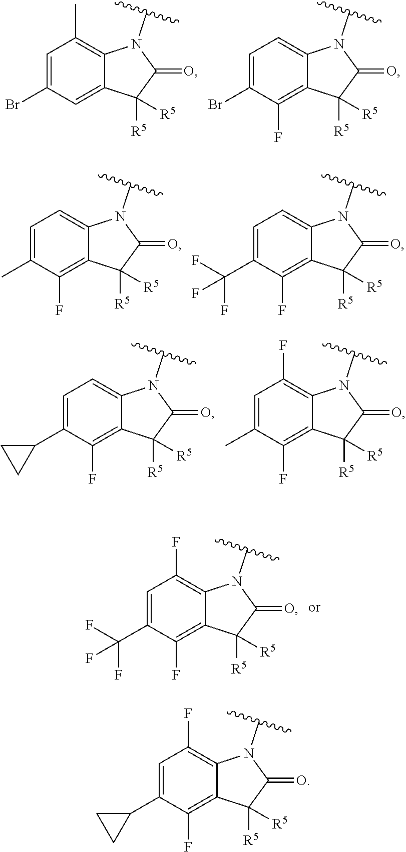

Embodiment A42: The compound of any one of Embodiments A1-A40, wherein the compound of Formula (I) is of Formula:

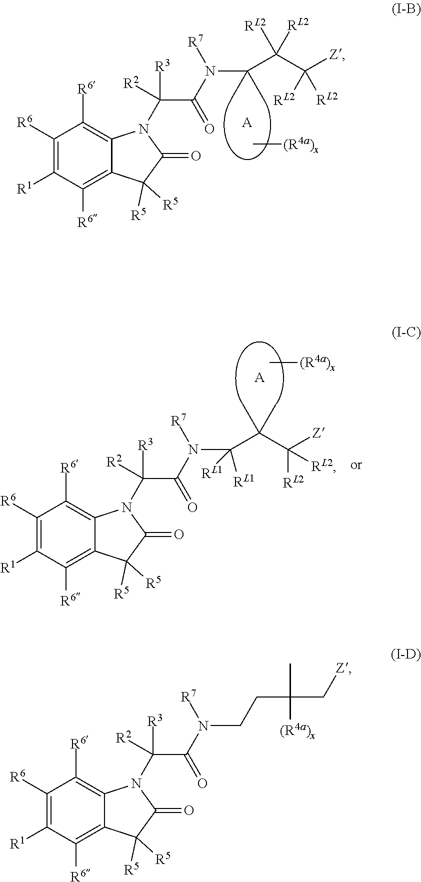

or a pharmaceutically acceptable salt thereof, wherein:
Z′ is tetrazole or carboxylic acid;
Ring A is C₃-C₅ cycloalkyl or 4- to 5-membered heterocyclyl;
each R^{4a} is independently halo, C₁-C₃ alkyl, C₁-C₃ haloalkyl, or C₃ cycloalkyl; and
x is 0, 1, 2, or 3.

Embodiment A43: The compound of any one of Embodiments A1-A40, wherein the compound of Formula (I) is of Formula:

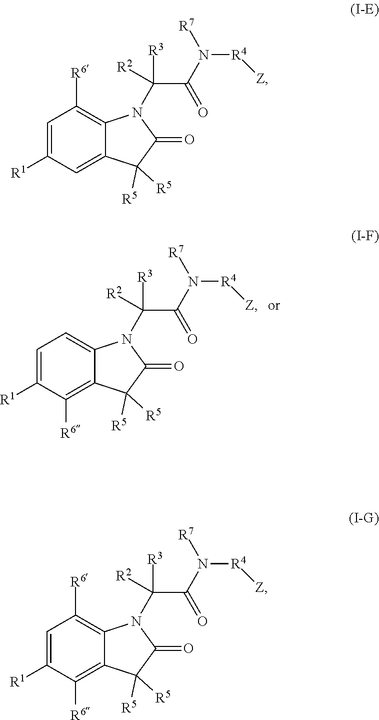

or a pharmaceutically acceptable salt thereof, wherein at least one of R⁶, R^{6′} and R^{6″} is not H.

Embodiment A44: The compound of Embodiment A1, selected from any one of the compounds of Table 1 or Table 2, or a pharmaceutically acceptable salt thereof.

Embodiment A45: A pharmaceutical composition comprising a compound of any one of Embodiments A1-A44, or a pharmaceutically acceptable salt thereof, and one or more pharmaceutically acceptable excipients.

Embodiment A46: A method of treating a disease or disorder in a subject in need thereof, comprising administering to the subject a compound of any one of Embodiments A1-A44, or a pharmaceutically acceptable salt thereof.

Embodiment A47: The method of Embodiment A46, wherein the disease or disorder is an NLRP3-related disease or disorder.

Embodiment A48: The method of Embodiment A46 or A47, wherein the subject is a human.

Embodiment A49: The method of any one of Embodiments A46-A48, wherein the disease or disorder is a disease or disorder of the central nervous system (CNS), a disease or disorder of the peripheral nervous system (PNS), a primary neurological disease of the muscles, an inflammatory disorder, an autoimmune disorder, cancer, an infection, a metabolic disease, a cardiovascular disease, a respiratory disease, a kidney disease, a liver disease, an ocular disease, a skin disease, a lymphatic disease, a rheumatic disease, a psychological disease, graft versus host disease, pain (including disorders related to pain management), or an NLRP3-related disease in a subject that has been determined to carry a germline or somatic non-silent mutation in NLRP3.

Embodiment A50: The method of Embodiment A49, wherein the disease or disorder of the central nervous system is dementia, Alzheimer's disease (“AD”) epilepsy, traumatic brain injury (“TBI”), multiple sclerosis (“MS”), developmental disturbances, acute disseminated encephalopathy, transverse myelitis, Parkinson's disease (“PD”), amyotrophic lateral sclerosis (“ALS”), Huntington's disease (“HD”), or spinal cord injury.

Embodiment A51: The method of Embodiment A49, wherein the primary neurological disease of the muscles is a dystrophy or spinal muscular atrophy.

Embodiment A52: The method of Embodiment A49, wherein the inflammatory disorder is gout or anemia of inflammation.

Embodiment A53: The method of Embodiment A49, wherein the autoimmune disease is ulcerative colitis.

Embodiment A54: The method of Embodiment A49, wherein the cancer is skin cancer or colon cancer.

Embodiment A55: The method of Embodiment A49, wherein the infection is a neuro-infection.

Embodiment A56: The method of Embodiment A49, wherein the metabolic disease is diabetes.

Embodiment A57: The method of Embodiment A49, wherein the cardiovascular disease is stroke.

Embodiment A58: The method of Embodiment A49, wherein the respiratory disease is asthma or chronic obstructive pulmonary disease.

Embodiment A59: The method of Embodiment A49, wherein the kidney disease is acute kidney disease, a chronic kidney disease, or a rare kidney disease.

Embodiment A60: The method of Embodiment A49, wherein the liver disease is nonalcoholic fatty liver disease (NAFLD) or non-alcoholic steatohepatitis (NASH).

Embodiment A61: The method of Embodiment A49, wherein the ocular disease is optic neuritis or macular degeneration.

Embodiment A62: The method of Embodiment A49, wherein the skin disease is psoriasis, hidradenitis suppurativa (HS), or atopic dermatitis.

Embodiment A63: The method of Embodiment A49, wherein the rheumatic disease is osteoarthritis, dermatomyositis, Still's disease, or juvenile idiopathic arthritis.

Embodiment A64: The method of Embodiment A49, wherein the psychological disease is a neuropsychiatric condition selected from the group consisting of depression, major depressive disorder, and refractory depression.

Embodiment A65: The method of Embodiment A49, wherein the pain is pain management addiction, osteoarthritis pain, or allodynia.

Embodiment A66: The method of Embodiment A49, wherein the NLRP3-related disease in a subject that has been determined to carry a germline or somatic non-silent mutation in NLRP3 is cryopyrin-associated autoinflammatory syndrome.

Embodiment A67: The method of Embodiment A49, wherein the disease or disorder is dementia, Alzheimer's disease (“AD”), epilepsy, traumatic brain injury (“TBI”), multiple sclerosis (“MS”), a developmental disturbance, acute disseminated encephalopathy, transverse myelitis, Parkinson's disease (“PD”), amyotrophic lateral sclerosis (“ALS”), spinal muscular atrophy, Huntington's disease (“HD”), spinal cord injury, a dystrophy, a neuro-infection, a pain management addiction, a neuropsychiatric condition, neonatal onset multisystem inflammatory disease (“NOMID”), asthma, osteoarthritis, ulcerative colitis, gout, anemia of inflammation, Still's disease, chronic obstructive pulmonary disease (“COPD”), osteoarthritis pain, or hidradenitis suppurativa.

Embodiment A68: A method of inhibiting NLRP3 activity in a cell, comprising contacting the cell with an effective amount of a compound of any one of Embodiments A1-A44, or a pharmaceutically acceptable salt thereof.

Embodiment A69: The method of Embodiment A68, wherein the method is an in vitro method.

Embodiment A70: A method of preparing a compound of Formula (I), wherein Z is -(L₂)ₙ-tetrazole, the method comprising coupling of a substituted indolinone(acetic acid) (i):

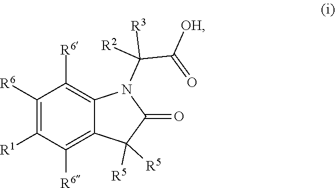

or salt thereof;
with an aminotetrazole (ii):

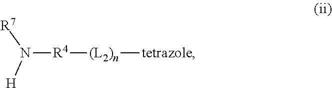

or salt thereof,
to provide the tetrazole analog:

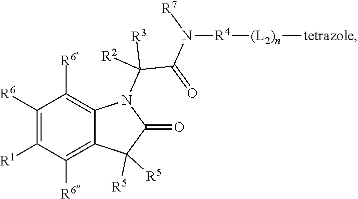

or salt thereof.

Embodiment A71: A method of preparing a compound of Formula (I), wherein Z is -(L₂)ₙ-carboxylic acid, the method comprising deprotecting a compound of Formula (v-o):

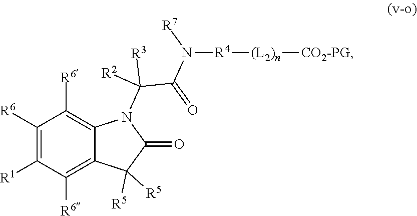

or salt thereof,
wherein PG is C₁₋₆ alkyl, C₆ aryl, or aryl-C₁₋₆ alkyl, wherein the alkyl and aryl are optionally substituted with one or more halo, C₁₋₆ alkyl, or C₁₋₆ alkoxy.

Embodiment A72: The method of Embodiment A71, wherein the compound of Formula (v-o), or salt thereof, is prepared by coupling of a substituted indolinone(acetic acid) (i):

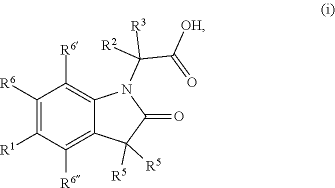

or salt thereof;
with an aminoester (iv):

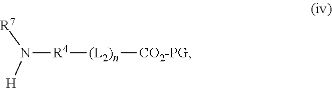

or salt thereof,
to provide the carboxylic acid analog (v-a):

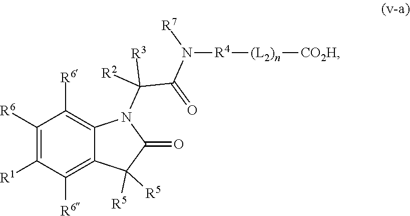

or salt thereof.

Embodiment A73: A compound of Formula (v-o):

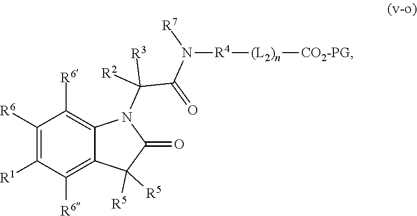

or salt thereof,
wherein n, R¹, R², R³, R⁴, L₂, R⁵, R⁶, R^{6′}, R^{6″}, and R⁷ are as defined in Embodiment A1, and PG is C₁₋₆ alkyl, C₆ aryl, or aryl-C₁₋₆ alkyl, wherein the alkyl and aryl are optionally substituted with one or more halo, C₁₋₆ alkyl, or C₁₋₆ alkoxy.

Embodiment A74: The compound of Embodiment A73, or salt thereof, wherein PG is methyl, ethyl, or t-butyl.

Embodiment A75: The compound, composition or method of any one of Embodiments A1-A74, wherein the compound of Formula (I) is an isotopic derivative, or a pharmaceutically acceptable salt thereof.

Embodiment A76: The compound of Embodiment A73 selected from any one of the compounds of Table 3, or a salt thereof.

### Exemplification

In order that this disclosure may be more fully understood, the following Examples are set forth. It should be understood that these Examples are for illustrative purposes only and are not to be construed as limiting this disclosure in any manner.

### Analytical Methods

Nuclear magnetic resonance (NMR) spectra were recorded at 400 megahertz (MHz) as stated and at 300.3 K unless otherwise stated; the chemical shifts (6) are reported in parts per million (ppm). Spectra were recorded using a Bruker Avance 400 instrument with 8, 16 or 32 scans. Exemplary NMR solvents include deuterated dimethylsulfoxide (DMSO-d₆), deuterated methanol (CD₃OD), and deuterated chloroform (CDCl₃). Other abbreviations: s=singlet; d=doublet; t=triplet; m=multiplet; br=broad.

Liquid Chromatography-Mass Spectrometry (LCMS) and spectra were recorded using a Shimadzu LCMS-2020. Injection volumes were 0.7-8.0 μl and the flow rates were typically 0.8 or 1.2 ml/min. Detection methods were diode array (DAD) or evaporative light scattering (ELSD) as well as positive ion electrospray ionization (ESI). MS range was 100-1000 Da. Solvents were gradients of water (H₂O) and/or acetonitrile (MeCN) may contain a modifier (typically 0.01-0.04%) such as formic acid (FA), trifluoroacetic acid (TFA) or ammonium carbonate (NH₄HCO₃). ESI or ES=electrospray ionization; m/z=mass/charge; RT=retention time (minutes).

Gas Chromatography-Mass Spectrometry (GCMS) chromatograms and spectra were recorded using Agilent GCMS 8890-5977 and Detector Channel FID. GC Parameters: DB-5MS, 12m×0.20 mm×0.33 um; Column Oven Temp: 50.0; Injection volume: 0.5 μL; Column Flow: 1.2 ml/min; Injection temperature: 300° C.; Injection Mode: Split; Split Ratio: 20:1; Detector temperature: 300° C.; Initial temperature: 50° C. for 1 min then 40° C./min to 300° C. for 1.75 min. Makeup Gas: He; Makeup Flow: 25.0 mL/min; H2; Flow: 30.0 mL/min; Air Flow: 400.0 mL/min; Final temperature: 300° C. The MS detector of acquisition mode: Start Time: 2.00 min; End Time: 9.00 min; Acquisition Mode: Scan; Interface Type: EI Threshold: 150; Scan Speed: 1562; Start m/z: 50.00; End m/z: 550.00; MS Source: 230.00° C.; MS Quad: 150.00° C.; Solvent Cut Time: 2.00 min.

Purification/Separation Methods. The Synthetic methods describe purification and/or separation chromatographic methods which have been employed in the purification and/or isolation of the exemplified compounds. Rf=retention factor; RT=retention time (minutes); Prep-HPLC=Preparative High-performance liquid chromatography; Prep-SFC=Preparative Supercritical Fluid Chromatography; TLC=thin layer chromatography.

### Synthetic Methods

### Example 1: N-(3-(1H-tetrazol-5-yl)propyl)-2-(5-bromo-3,3-dimethyl-2-oxoindolin-1-yl)acetamide (tautomer 1) and N-(3-(2H-tetrazol-5-yl)propyl)-2-(5-bromo-3,3-dimethyl-2-oxoindolin-1-yl)acetamide (tautomer 2) (Compound 1)

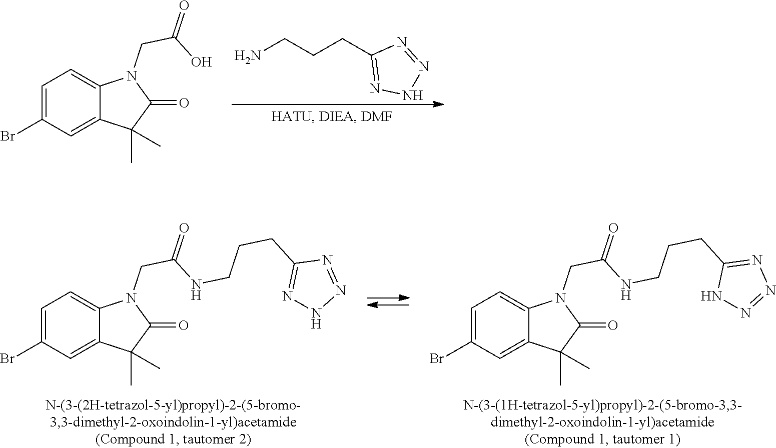 [SMILES: CC1(C)C(=O)N(CC(=O)O)c2ccc(Br)cc21 ; NCCCc1nn[nH]n1 ; CC1(C)C(=O)N(CC(=O)NCCCc2nn[nH]n2)c2ccc(Br)cc21 ; CC1(C)C(=O)N(CC(=O)NCCCc2nnn[nH]2)c2ccc(Br)cc21]

A solution of 2-(5-bromo-3,3-dimethyl-2-oxo-indolin-1-yl)acetic acid (1 eq, 54 mg, 0.181 mmol) and [bis(dimethylamino)methylene]-1H-1,2,3-triazolo[4,5-b]pyridinium 3-oxide hexafluorophosphate (HATU) (1.1 eq, 72 mg, 0.190 mmol) in N,N-dimethyl formamide (DMF) (0.8 mL) was treated with N,N-diisopropylethylamine (DIPEA) (2.2 eq, 0.069 mL, 0.398 mmol) and NaOH (5 eq, 0.43 mL, 0.906 mmol) and stirred at room temperature for 20 min. The mixture was directly injected onto a C18 reverse phase chromatographic column, eluting with 0-100% MeCN/H₂O, to afford N-(3-(1H-tetrazol-5-yl)propyl)-2-(5-bromo-3,3-dimethyl-2-oxoindolin-1-yl)acetamide (tautomer 1) and N-(3-(2H-tetrazol-5-yl)propyl)-2-(5-bromo-3,3-dimethyl-2-oxoindolin-1-yl)acetamide (tautomer 2) (Compound 1). LCMS (ES, m/z): RT=1.97 min, m/z=409.23[M+1]+. ¹H NMR (400 MHz, DMSO-d₆) δ 8.30 (t, J=5.7 Hz, 1H), 7.62 (d, J=2.0 Hz, 1H), 7.41 (dd, J=8.3, 2.1 Hz, 1H), 6.86 (d, J=8.3 Hz, 1H), 4.30 (s, 2H), 3.23-3.08 (m, 3H), 2.95-2.78 (m, 2H), 1.86 (p, J=7.2 Hz, 2H), 1.30 (s, 6H).

### Example 2: 4-(2-(5-bromo-3,3-dimethyl-2-oxoindolin-1-yl)acetamido)butanoic acid (Compound 2) and methyl 4-(2-(5-bromo-3,3-dimethyl-2-oxoindolin-1-yl)acetamido)butanoate (Compound 2-OMe)

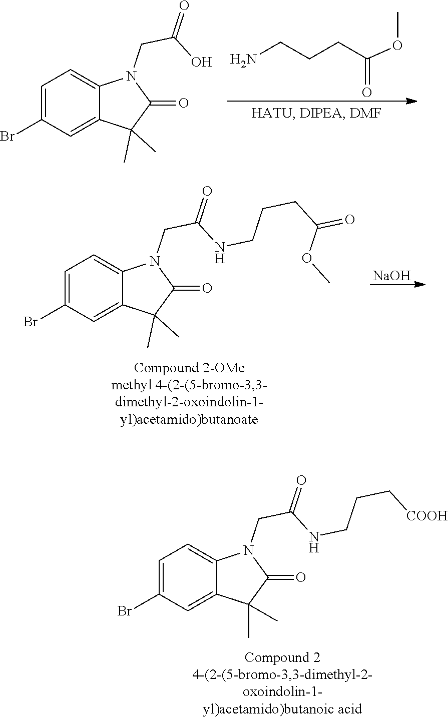

A solution of methyl 4-aminobutanoate hydrochloride (1.1 eq, 31 mg, 0.203 mmol) and 2-(5-bromo-3,3-dimethyl-2-oxo-indolin-1-yl)acetic acid (1 eq, 55 mg, 0.184 mmol) in N,N-dimethyl formamide (DMF) (0.8 mL) was treated with N,N-diisopropylethylamine (DIPEA) (3 eq, 0.096 mL, 0.553 mmol) and [bis(dimethylamino)methylene]-1H-1,2,3-triazolo[4,5-b]pyridinium 3-oxide hexafluorophosphate (HATU) (1.1 eq, 74 mg, 0.194 mmol) and stirred at room temperature for 20 min. Methyl 4-(2-(5-bromo-3,3-dimethyl-2-oxoindolin-1-yl)acetamido)butanoate (Compound 2-OMe) was formed in situ. The mixture was then treated with NaOH (5 eq, 0.43 mL, 0.922 mmol) and stirred for an extra 2 hours. The mixture was quenched upon addition of 6N HCl (0.23 mL). The residue was purified using a C18 reverse phase chromatographic column, eluting with 0-100% MeCN/H₂O, to afford 4-(2-(5-bromo-3,3-dimethyl-2-oxoindolin-1-yl)acetamido)butanoic acid (Compound 2). LCMS (ES, m/z): RT=1.99 min, m/z=385.33[M+1]⁺. ¹H NMR (400 MHz, DMSO-d₆) δ 8.20 (t, J=5.6 Hz, 1H), 7.60 (d, J=2.1 Hz, 1H), 7.40 (dd, J=8.3, 2.1 Hz, 1H), 6.85 (d, J=8.3 Hz, 1H), 4.28 (s, 2H), 3.08 (q, J=6.6 Hz, 2H), 2.23 (t, J=7.4 Hz, 2H), 1.63 (p, J=7.2 Hz, 2H), 1.29 (s, 6H).

### Example 3: 4-(2-(5′-bromo-2′-oxospiro[cyclopentane-1,3′-indolin]-1′-yl)acetamido)butanoic acid (Compound 5) and methyl 4-(2-(5′-bromo-2′-oxospiro[cyclopentane-1,3′-indolin]-1′-yl)acetamido)butanoate (Compound 5-OMe)

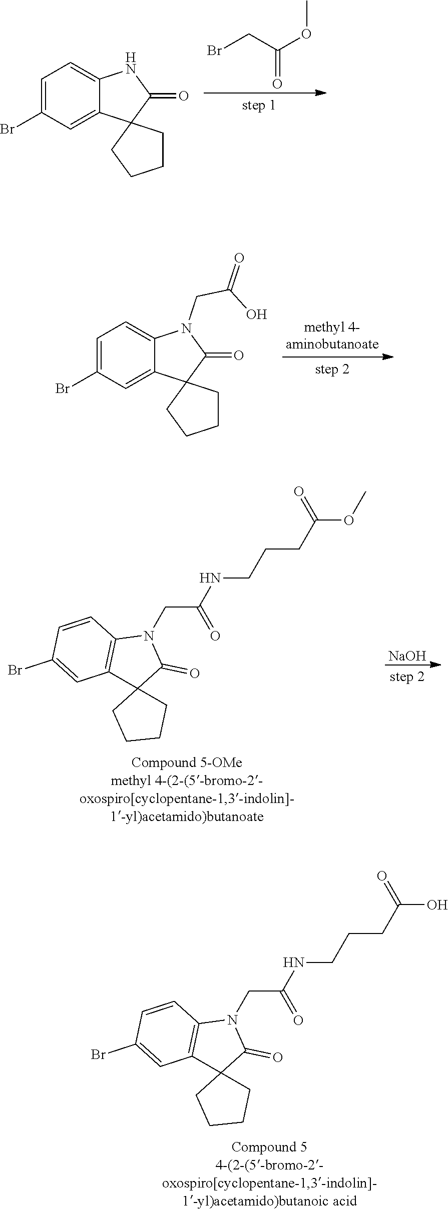

Step 1: A solution of 5′-bromospiro[cyclopentane-1,3′-indoline]-2′-one (1 eq, 100 mg, 0.376 mmol) in N,N-dimethyl formamide (DMF) (3 mL) was cooled to 0° C. and treated with NaH (1.10 eq, 17 mg, 0.413 mmol) and stirred for 20 min, then methyl 2-bromoacetate was added (1.1 eq, 0.039 mL, 0.413 mmol), and the reaction was brought up to room temperature and stirred for 40 min. The mixture was then treated with NaOH (1.20 eq, 0.18 mL, 0.451 mmol) and stirred for 1 h. The mixture was then neutralized using 6N HCl and directly injected onto a C18 reverse phase chromatographic column, eluting with 0-100% MeCN/H₂O, to afford 2-(5′-bromo-2′-oxospiro[cyclopentane-1,3′-indolin]-1′-yl)acetic acid. LCMS (ES, m/z): RT=2.34 min, m/z=324.31[M+1]⁺.

Step 2: A solution of 2-(5′-bromo-2′-oxo-spiro[cyclopentane-1,3′-indoline]-1′-yl)acetic acid (1 eq, 90 mg, 0.278 mmol) and methyl 4-aminobutanoate hydrochloride (1.1 eq, 47 mg, 0.305 mmol) in 0.8 mL N,N-dimethyl formamide (DMF) was treated with N,N-diisopropylethylamine (DIPEA) (3 eq, 0.15 mL, 0.833 mmol) and [bis(dimethylamino)methylene]-1H-1,2,3-triazolo[4,5-b]pyridinium 3-oxide hexafluorophosphate (HATU) (1.1 eq, 116 mg, 0.305 mmol) and stirred at room temperature for 20 min. Methyl 4-(2-(5′-bromo-2′-oxospiro[cyclopentane-1,3′-indolin]-1′-yl)acetamido)butanoate (Compound 5-OMe) was formed in situ. The mixture was then treated with a 2N solution of NaOH (5 eq, 0.56 mL, 1.4 mmol) and stirred for an extra 2 hours. The mixture was quenched upon addition of 6N HCl (0.23 mL). The residue was purified using reverse phase chromatography eluting with 0-100% MeCN/H₂O to afford 4-(2-(5′-bromo-2′-oxospiro[cyclopentane-1,3′-indolin]-1′-yl)acetamido)butanoic acid (Compound 5). LCMS (ES, m/z): RT=2.20 min, m/z=411.36[M+1]⁺. ¹H NMR (400 MHz, DMSO-d₆) δ 12.09 (s, 1H), 8.20 (t, J=5.6 Hz, 1H), 7.48 (d, J=2.0 Hz, 1H), 7.39 (dd, J=8.3, 2.0 Hz, 1H), 6.82 (d, J=8.4 Hz, 1H), 4.27 (s, 2H), 3.17 (s, 1H), 3.08 (q, J=6.6 Hz, 2H), 2.23 (t, J=7.4 Hz, 2H), 2.07-1.90 (m, 5H), 1.87-1.77 (m, 2H), 1.63 (p, J=7.2 Hz, 2H).

### Example 4: 4-(2-(3,3-dimethyl-2-oxo-5-(trifluoromethyl)indolin-1-yl)acetamido)-4-methylpentanoic acid (Compound 8) and methyl 4-(2-(3,3-dimethyl-2-oxo-5-(trifluoromethyl)indolin-1-yl)acetamido)-4-methylpentanoate (Compound 8-OMe)

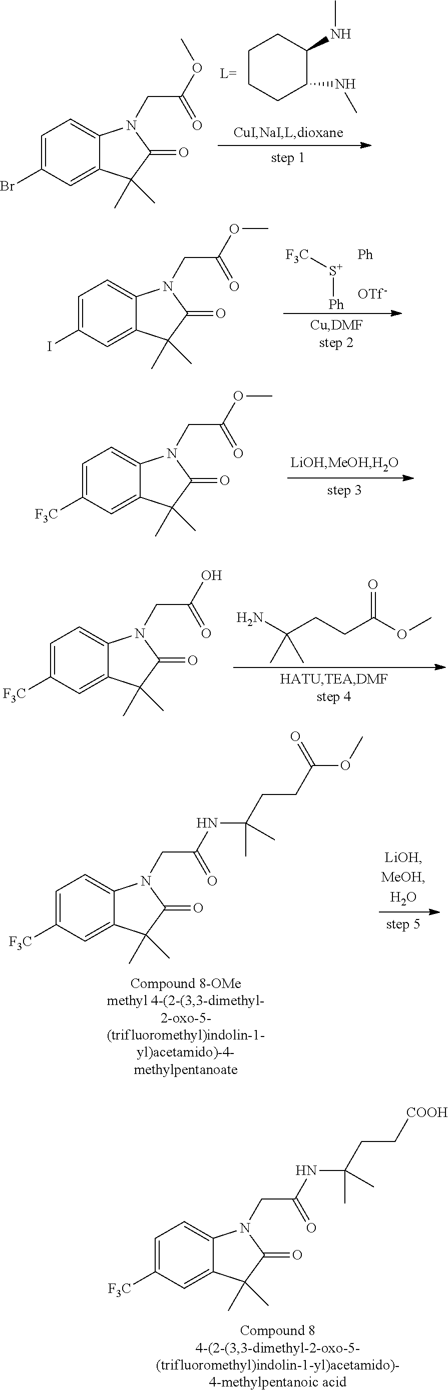

Step 1: Into a 20 mL vial was added methyl 2-(5-bromo-3,3-dimethyl-2-oxoindol-1-yl)acetate (280 mg, 0.897 mmol, 1 equiv), copper(I) iodide (342 mg, 1.79 mmol, 2 equiv), (1R,2R)—N1,N2-dimethylcyclohexane-1,2-diamine (153 mg, 1.08 mmol, 1.2 equiv), iodosodium (161 mg, 1.08 mmol, 1.2 equiv), dioxane (5 mL) at 100° C. The resulting mixture was stirred for 12 h at 100° C. under nitrogen atmosphere. The reaction was monitored by LCMS. The resulting mixture was filtered, the filter cake was washed with methanol (MeOH) (3×5 mL). The filtrate was concentrated under reduced pressure. The resulting mixture was concentrated under reduced pressure. The residue was purified by silica gel column chromatography, eluting with DCM/MeOH (10:1), to afford methyl 2-(5-iodo-3,3-dimethyl-2-oxoindol-1-yl)acetate (240 mg, 75% yield) as a red oil. LCMS: (ES, m/z): RT=1.15 min, m/z=360.2 [M+H]⁺.

Step 2: Into a 20 mL vial was added methyl 2-(5-iodo-3,3-dimethyl-2-oxoindol-1-yl)acetate (230 mg, 0.640 mmol, 1 equiv), copper (407 mg, 6.40 mmol, 10 equiv), diphenyl(trifluoromethyl)sulfanium (490 mg, 1.92 mmol, 3 equiv) and N,N-dimethyl formamide (DMF) (3 mL) at 80° C. The resulting mixture was stirred for 2 h at 80° C. under nitrogen atmosphere. The reaction was monitored by LCMS. The resulting mixture was filtered, and the filter cake was washed with methanol (MeOH) (2×4 mL). The filtrate was concentrated under reduced pressure. The resulting mixture was concentrated under reduced pressure. The residue was purified by silica gel column chromatography, eluting with DCM/MeOH (10:1), to afford methyl 2-[3,3-dimethyl-2-oxo-5-(trifluoromethyl)indol-1-yl]acetate (150 mg, 78% yield) as a red oil. LCMS: (ES, m/z): RT=1.15 min, m/z=302.1[M+H]⁺.

Step 3: Into a 8 mL vial was added methyl 2-[3,3-dimethyl-2-oxo-5-(trifluoromethyl)indol-1-yl]acetate (130 mg, 0.432 mmol, 1 equiv) and lithium hydroxide (31.0 mg, 1.30 mmol, 3 equiv), methanol (MeOH) (2 mL) and water (2 mL) at room temperature. The resulting mixture was stirred for 2 h at room temperature. The reaction was monitored by LCMS. The resulting mixture was diluted with water (10 mL). The mixture was acidified to pH 3 with HCl (2 aq.). The aqueous layer was extracted with ethyl acetate (EtOAc) (3×10 mL). The resulting mixture was concentrated under reduced pressure. This resulted in [3,3-dimethyl-2-oxo-5-(trifluoromethyl)indol-1-yl]acetic acid (100 mg, 81% yield) as a white solid. LCMS: (ES, m/z): RT=0.68 min, m/z=288.1[M+H]⁺.

Step 4: Into a 8 mL vial was added [3,3-dimethyl-2-oxo-5-(trifluoromethyl)indol-1-yl]acetic acid (80 mg, 0.279 mmol, 1 equiv), methyl 4-amino-4-methylpentanoate (48.5 mg, 0.335 mmol, 1.2 equiv), [bis(dimethylamino)methylene]-1H-1,2,3-triazolo[4,5-b]pyridinium 3-oxide hexafluorophosphate (HATU) (127 mg, 0.335 mmol, 1.2 equiv), triethylamine (TEA) (84.5 mg, 0.837 mmol, 3 equiv) and N,N-dimethyl formamide (DMF) (2 mL) at room temperature. The resulting mixture was stirred for 1 h at room temperature. The reaction was monitored by LCMS. The resulting mixture was extracted with ethyl acetate (EtOAc) (3×8 mL). The combined organic layers were washed with water (4×10 mL), dried over anhydrous Na₂SO₄. After filtration, the filtrate was concentrated under reduced pressure. The resulting mixture was concentrated under reduced pressure. The residue was purified by silica gel column chromatography, eluting with petroleum ether/ethyl acetate (1:1), to afford methyl 4-{2-[3,3-dimethyl-2-oxo-5-(trifluoromethyl)indol-1-yl]acetamido}-4-methylpentanoate (also referred to herein as methyl 4-(2-(3,3-dimethyl-2-oxo-5-(trifluoromethyl)indolin-1-yl)acetamido)-4-methylpentanoate; Compound 8-OMe) (80 mg, 69% yield) as a yellow oil. LCMS: (ES, m/z): RT=1.09 min, m/z=415.1[M+H]⁺.

Step 5: Into a 8 mL vial was added methyl 4-{2-[3,3-dimethyl-2-oxo-5-(trifluoromethyl)indol-1-yl]acetamido}-4-methylpentanoate (60 mg, 0.145 mmol, 1 equiv) and lithium hydroxide (10.4 mg, 0.435 mmol, 3 equiv), methanol (MeOH) (1 mL) and water (1 mL) at room temperature. The reaction was monitored by LCMS. The resulting mixture was diluted with water (8 mL). The mixture was acidified to pH 3 with HCl (2 aq.). The resulting mixture was extracted with ethyl acetate (EtOAc) (3×8 mL). The combined organic layers were washed with water (4×7 mL), dried over anhydrous Na₂SO₄. After filtration, the filtrate was concentrated under reduced pressure. The resulting mixture was concentrated under reduced pressure. The crude product (30 mg) was purified by Prep-HPLC (XBridge Prep OBD C18 Column, 30*150 mm, 5 μm; Mobile Phase A: water (10 mmol/L NH₄HCO₃), Mobile Phase B: acetonitrile (MeCN); Flow rate: 60 mL/min; Gradient: 19% B to 29% B in 8 min, 29% B; Wave Length: 254 nm; HPLC RT(min): 7) to afford 4-(2-(3,3-dimethyl-2-oxo-5-(trifluoromethyl)indolin-1-yl)acetamido)-4-methylpentanoic acid (Compound 8) (19.5 mg) as a white solid. LCMS: (ES, m/z): RT=0.67 min, m/z=401.0[M+H]⁺. ¹H NMR (400 MHz, DMSO-d₆) δ 8.16 (s, 1H), 7.77 (d, J=1.9 Hz, 1H), 7.64-7.57 (m, 1H), 7.01 (d, J=8.2 Hz, 1H), 4.31 (s, 2H), 2.18 (t, J=8.0 Hz, 2H), 1.84 (m, J=9.1, 6.9 Hz, 2H), 1.34 (s, 6H), 1.23 (s, 6H).

### Example 5: 4-(2-(5-cyclopropyl-3,3-dimethyl-2-oxoindolin-1-yl)acetamido)-4-methylpentanoic acid (Compound 9) and methyl 4-(2-(5-cyclopropyl-3,3-dimethyl-2-oxoindolin-1-yl)acetamido)-4-methylpentanoate (Compound 9-OMe)

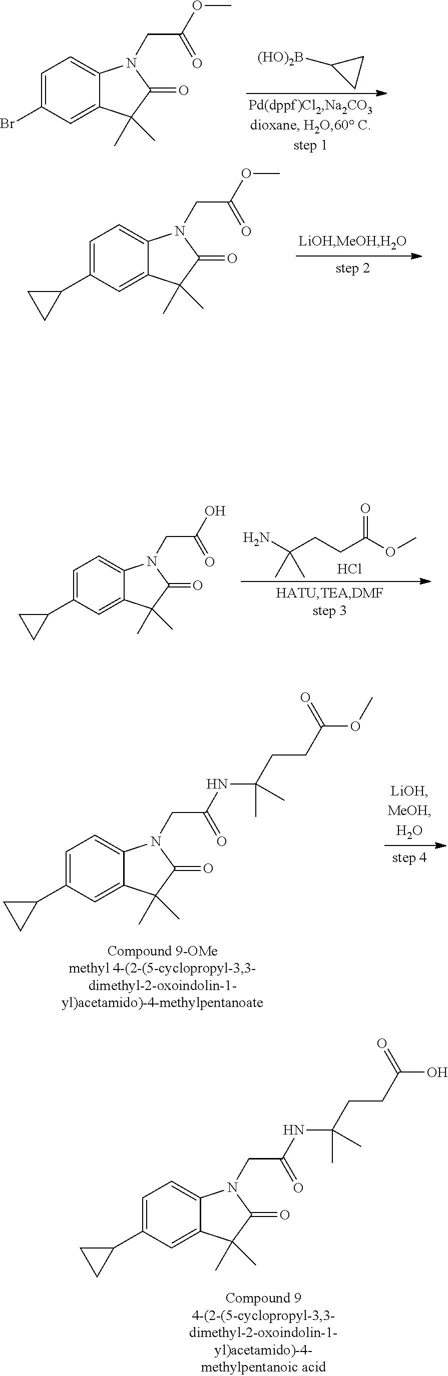

Step 1: Into a 8 mL sealed tube was added methyl 2-(5-bromo-3,3-dimethyl-2-oxoindol-1-yl)acetate (150 mg, 0.48 mmol, 1 equiv), cyclopropylboronic acid (206 mg, 2.4 mmol, 5 equiv), [1,1′-bis(diphenylphosphino)ferrocene]dichloropalladium(II) (Pd(dppf)Cl₂) (70.3 mg, 0.09 mmol, 0.2 equiv), Na₂CO₃ (153 mg, 1.44 mmol, 3 equiv), dioxane (1.5 mL), and H₂O (0.3 mL). The resulting mixture was stirred for 2 h at 60° C. under nitrogen atmosphere. The reaction was monitored by LCMS. The resulting mixture was extracted with ethyl acetate (EtOAc) (3×3 mL). The combined organic layers were washed with water (3×2 mL), and dried over anhydrous Na₂SO₄. After filtration, the filtrate was concentrated under reduced pressure. The residue was purified by silica gel column chromatography, eluting with petroleum ether/ethyl acetate (5:1), to afford methyl 2-(5-cyclopropyl-3,3-dimethyl-2-oxoindol-1-yl)acetate (95 mg, 43% yield) as a yellow oil. LCMS: (ES, m/z): RT=1.57 min, m/z=274[M+1]⁺.

Step 2: Into a 8-mL vial was placed methyl 2-(5-cyclopropyl-3,3-dimethyl-2-oxoindol-1-yl)acetate (140 mg, 0.51 mmol, 1 equiv), methanol (MeOH) (1.5 mL), H₂O (0.30 mL), and LiOH (24.5 mg, 1.02 mmol, 2 equiv). The resulting solution was stirred for 2 h at 25° C. The reaction was monitored by LCMS. The resulting mixture was concentrated under reduced pressure. The crude product, (5-cyclopropyl-3,3-dimethyl-2-oxoindol-1-yl)acetic acid, was used in the next step directly without further purification. LCMS: (ES, m/z): RT=0.77 min, m/z=260 [M+1]⁺.

Step 3: Into a 25 mL sealed tube was added (5-cyclopropyl-3,3-dimethyl-2-oxoindol-1-yl)acetic acid (150 mg, 0.57 mmol, 1 equiv), methyl 4-amino-4-methylpentanoate (101 mg, 0.69 mmol, 1.2 equiv), [bis(dimethylamino)methylene]-1H-1,2,3-triazolo[4,5-b]pyridinium 3-oxide hexafluorophosphate (HATU) (264 mg, 0.69 mmol, 1.20 equiv), N,N-dimethyl formamide (DMF) (1.50 mL), and triethylamine (TEA) (176 mg, 1.73 mmol, 3 equiv). The resulting mixture was stirred for 2 h at room temperature. The reaction was monitored by LCMS. The reaction was quenched by the addition of water (3 mL) at room temperature. The resulting mixture was extracted with ethyl acetate (EtOAc) (3×5 mL). The combined organic layers were washed with water (3×5 mL), and dried over anhydrous Na₂SO₄. After filtration, the filtrate was concentrated under reduced pressure. The residue was purified by Prep-TLC (petroleum ether/ethyl acetate 1:1) to afford methyl 4-[2-(5-cyclopropyl-3,3-dimethyl-2-oxoindol-1-yl)acetamido]-4-methylpentanoate (also referred to herein as methyl 4-(2-(5-cyclopropyl-3,3-dimethyl-2-oxoindolin-1-yl)acetamido)-4-methylpentanoate; Compound 9-OMe) (130 mg, 41% yield) as a light yellow oil. LCMS: (ES, m/z): RT=0.92 min, m/z=387 [M+1]⁺.

Step 4: Into a 8 mL vial was added methyl 4-[2-(5-cyclopropyl-3,3-dimethyl-2-oxoindol-1-yl)acetamido]-4-methylpentanoate (120 mg, 0.31 mmol, 1 equiv), LiOH (14.9 mg, 0.62 mmol, 2 equiv), H²O (0.20 mL), and methanol (MeOH) (1 mL). The resulting mixture was stirred for 2 h at room temperature. The reaction was monitored by LCMS. The resulting mixture was extracted with ethyl acetate (EtOAc) (3×4 mL). The combined organic layers were washed with water (3×4 mL), dried over anhydrous Na₂SO₄. After filtration, the filtrate was concentrated under reduced pressure. The residue was purified by Prep-TLC (DCM/MeOH (9:1)) to afford 4-[2-(5-cyclopropyl-3,3-dimethyl-2-oxoindol-1-yl)acetamido]-4-methylpentanoic acid (120 mg, 62% purity) as a yellow oil. The crude product was purified by Prep-HPLC (2 #SHIMADZU (HPLC-01): XBridge Prep OBD C18 Column, 19*250 mm, 5 μm; Mobile Phase A: water (10 mmol/L NH₄HCO₃), Mobile Phase B: methanol (MeOH); Flow rate: 20 mL/min; Gradient: 54% B to 54% B in 12 min, 54% B; Wave Length: 254/220 nm) to provide 4-(2-(5-cyclopropyl-3,3-dimethyl-2-oxoindolin-1-yl)acetamido)-4-methylpentanoic acid (Compound 9) (9.1 mg, 8% yield) as a white solid. LCMS: (ES, m/z): RT=0.80 min, m/z=373[M+1]⁺. ¹H NMR (400 MHz, Methanol-d₄) δ 7.05 (d, J=1.9 Hz, 1H), 7.00 (d, J=8.1 Hz, 1H), 6.77 (d, J=8.0 Hz, 1H), 4.33 (s, 2H), 2.26 (t, J=7.8 Hz, 2H), 1.99 (t, J=7.9 Hz, 2H), 1.92 (d, J=8.7 Hz, 1H), 1.37 (d, J=15.5 Hz, 12H), 0.98-0.89 (m, 2H), 0.69-0.60 (m, 2H).

### Example 6: 4-(1-(5-bromo-3,3-dimethyl-2-oxoindolin-1-yl)cyclopropane-1-carboxamido)butanoic acid (Compound 15) and methyl 4-(1-(5-bromo-3,3-dimethyl-2-oxoindolin-1-yl)cyclopropane-1-carboxamido)butanoate (Compound 15-OMe)

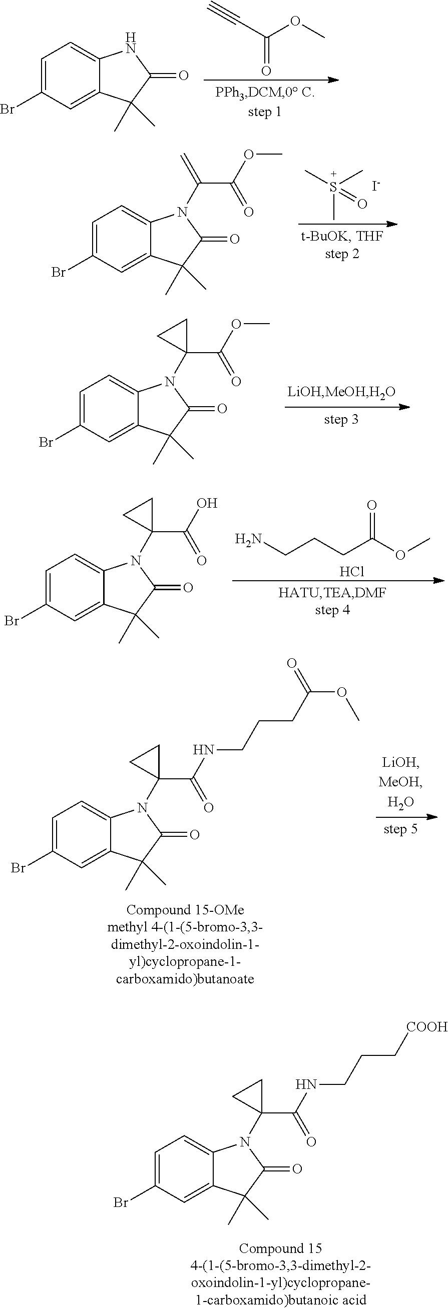

Step 1: Into a 25-mL 2-necked round-bottom flask, was placed 5-bromo-3,3-dimethyl-1H-indol-2-one (120 mg, 0.5 mmol, 1 equiv) (Compound 24a of Fensome et al., J. Med. Chem. (2008) 1861-1873), triphenyl phosphine (PPh₃) (262.18 mg, 1 mmol, 2 equiv), and dichloromethane (DCM) (3 mL). This was followed by the addition of propiolic acid methyl ester (50.4 mg, 0.6 mmol, 1.2 equiv) dropwise with stirring at 0° C. The resulting solution was stirred for 2 h at 25° C. The reaction progress was monitored by LCMS. The resulting mixture was concentrated under vacuum. The residue was purified by silica gel column chromatography, eluting with petroleum ether/ethyl acetate (2:1), to afford methyl 2-(5-bromo-3,3-dimethyl-2-oxoindol-1-yl)prop-2-enoate (120 mg, 67% yield) as a brown yellow oil. LCMS: (ES, m/z): RT=0.92, m/z=324.0[M+H]⁺.

Step 2: Into a 50-mL 3-necked round-bottom flask purged and maintained with an inert atmosphere of nitrogen, was placed sodium tert-butoxide (t-BuONa) (119 mg, 1.2 mmol, 2 equiv), and dimethyl sulfoxide (DMSO) (5 mL). This was followed by the addition of trimethylsulfoxonium iodide (271 mg, 1.234 mmol, 2 equiv) dropwise with stirring at 0° C. To this was added methyl 2-(5-bromo-3,3-dimethyl-2-oxoindol-1-yl)prop-2-enoate (200 mg, 0.617 mmol, 1 equiv) in tetrahydrofuran (THF) (1 mL) at 0° C. in 1 h. The resulting solution was stirred for 6 h at 0° C. The reaction progress was monitored by LCMS. The resulting mixture was extracted with ethyl acetate (EtOAc) (3×3 mL). The combined organic layers were washed with water (3×3 mL), and dried over anhydrous MgSO₄. After filtration, the filtrate was concentrated under reduced pressure. The residue was purified by silica gel column chromatography, eluting with petroleum ether/ethyl acetate (1:1), to afford methyl 1-(5-bromo-3,3-dimethyl-2-oxoindol-1-yl)cyclopropane-1-carboxylate (10 mg, 4.3% yield) as a white solid. LCMS: (ES, m/z): RT=1.11, m/z=338.0[M+H]⁺.

Step 3: Into a 8-mL vial was placed methyl 1-(5-bromo-3,3-dimethyl-2-oxoindol-1-yl)cyclopropane-1-carboxylate (20 mg, 0.059 mmol, 1 equiv), methanol (MeOH) (0.5 mL), and H₂O (0.1 mL). The resulting solution was stirred for 2 h at 25° C. The reaction progress was monitored by LCMS. The resulting mixture was concentrated under vacuum. The crude product, 1-(5-bromo-3,3-dimethyl-2-oxoindol-1-yl)cyclopropane-1-carboxylic acid, was used in the next step directly without further purification. LCMS: (ES, m/z): RT=0.82, m/z=323.9[M+H]⁺.

Step 4: Into a 8-mL vial, was placed 1-(5-bromo-3,3-dimethyl-2-oxoindol-1-yl)cyclopropane-1-carboxylic acid (30 mg, 0.093 mmol, 1 equiv), methyl 4-aminobutanoate hydrochloride (17.1 mg, 0.112 mmol, 1.2 equiv), [bis(dimethylamino)methylene]-1H-1,2,3-triazolo[4,5-b]pyridinium 3-oxide hexafluorophosphate (HATU) (42.2 mg, 0.112 mmol, 1.2 equiv), triethylamine (TEA) (18.7 mg, 0.186 mmol, 2 equiv), N,N-dimethyl formamide (DMF) (1 mL). The resulting solution was stirred for 2 h at 25° C. The reaction progress was monitored by LCMS. The resulting mixture was extracted with ethyl acetate (EtOAc) (3×3 mL). The combined organic layers were washed with water (3×3 mL), dried over anhydrous MgSO₄. After filtration, the filtrate was concentrated under reduced pressure. The residue was purified by Prep-TLC (petroleum ether/ethyl acetate (1:1)) to afford methyl 4-{[1-(5-bromo-3,3-dimethyl-2-oxoindol-1-yl)cyclopropyl]formamido}butanoate (also referred to herein as methyl 4-(1-(5-bromo-3,3-dimethyl-2-oxoindolin-1-yl)cyclopropane-1-carboxamido)butanoate; Compound 15-OMe) (13 mg, 32% yield) as a white solid. LCMS: (ES, m/z): RT=1.12, m/z=423.1[M+H]⁺.

Step 5: Into a 8-mL vial was placed methyl 4-{[1-(5-bromo-3,3-dimethyl-2-oxoindol-1-yl)cyclopropyl]formamido}butanoate (10 mg, 0.024 mmol, 1 equiv), LiOH (2.83 mg, 0.120 mmol, 5 equiv), methanol (MeOH) (0.5 mL), and H₂O (0.1 mL). The resulting solution was stirred for 2 h at 25° C. The reaction progress was monitored by LCMS. The resulting mixture was concentrated under vacuum. The residue was acidified/basified/neutralized to pH 5 with conc. HCl. The resulting mixture was extracted with ethyl acetate (EtOAc) (3×3 mL). The combined organic layers were washed with water (1×3 mL) and dried over anhydrous MgSO₄. After filtration, the filtrate was concentrated under reduced pressure. This resulted in 4-{[1-(5-bromo-3,3-dimethyl-2-oxoindol-1-yl)cyclopropyl]formamido}butanoic acid (7.2 mg), which was purified by reverse phase chromatography (Xselect CSH C18 OBD Column 30*150 mm 5 μm, n; Mobile Phase A: acetonitrile (MeCN), Mobile Phase B: water (0.05% TFA); Flow rate: 60 mL/min; Gradient: 34% B to 44% B in 10 min, 44% B; Wave Length: 254/220 nm; RT1 (min): 7.17; Number Of Runs: 0) to afford the trifluoroacetic acid (TFA) salt of 4-(1-(5-bromo-3,3-dimethyl-2-oxoindolin-1-yl)cyclopropane-1-carboxamido)butanoic acid (Compound 15) (also referred to herein as 4-{[1-(5-bromo-3,3-dimethyl-2-oxoindol-1-yl)cyclopropyl]formamido} butanoic acid) (7.2 mg, 58% yield) as a white solid. LCMS: (ES, m/z): RT=0.68, m/z=409.0[M+H]⁺. ¹H NMR (400 MHz, DMSO-d₆) δ 12.00 (s, 1H), 7.68 (t, J=5.9 Hz, 1H), 7.60 (d, J=2.1 Hz, 1H), 7.44 (dd, J=8.3, 2.1 Hz, 1H), 6.85 (d, J=8.3 Hz, 1H), 3.04 (q, J=6.7 Hz, 2H), 2.13 (t, J=7.4 Hz, 2H), 1.58 (dd, J=14.5, 7.4 Hz, 4H), 1.21 (d, J=75.7 Hz, 8H).

### Example 7: 4-(2-(5-bromo-7-hydroxy-3,3-dimethyl-2-oxoindolin-1-yl)acetamido)butanoic acid (Compound 16) and 4-(2-(5-bromo-7-hydroxy-3,3-dimethyl-2-oxoindolin-1-yl)acetamido)butanoic acid (Compound 16-OMe)

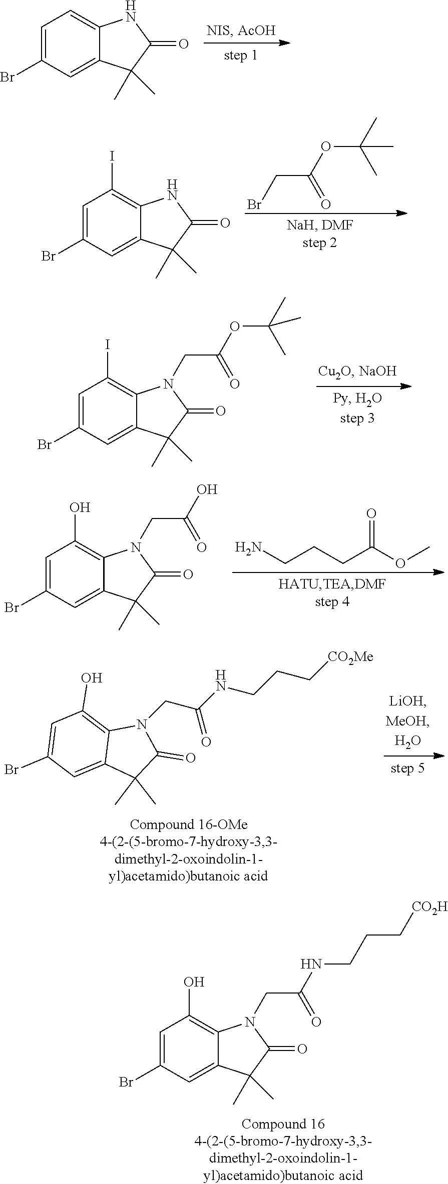

Step 1: To an ice cold solution of 5-bromo-3,3-dimethyl-1H-indol-2-one (400 mg, 1.66 mmol, 1 equiv) (Compound 24a of Fensome et al., J. Med. Chem. (2008) 1861-1873) in acetic acid (AcOH) (4 mL) was added N-iodosuccinimide (NIS) (450 mg, 1.99 mmol, 1.2 equiv) portion wise followed by stirring for 3 h at 50° C. Then conc. H₂SO₄ (49.01 mg, 0.50 mmol, 0.3 equiv) was added at room temperature. The resulting mixture was stirred for additional 2 h at 50° C. The reaction mixture was quenched by ice/water (10 mL). The resulting mixture was extracted with ethyl acetate (EtOAc) (5×10 mL). The combined organic layers were dried over anhydrous Na₂SO₄. After filtration, the filtrate was concentrated under reduced pressure. The residue was purified by silica gel column chromatography, eluting with petroleum ether/ethyl acetate (5:1), to afford 5-bromo-7-iodo-3,3-dimethyl-1H-indol-2-one (500 mg, 82% yield) as a brown solid. LCMS:(ES, m/z): RT=0.85 min, m/z=366 [M+H]⁺. ¹H NMR (400 MHz, DMSO-d₆) δ 10.49 (s, 1H), 7.70 (d, J=1.9 Hz, 1H), 7.57 (d, J=1.9 Hz, 1H), 1.27 (s, 6H).

Step 2: To a solution of 5-bromo-7-iodo-3,3-dimethyl-1H-indol-2-one (500 mg, 1.36 mmol, 1 equiv) in N,N-dimethyl formamide (DMF) (10 mL) was added NaH (60% in mineral oil) (164 mg, 4.09 mmol, 3 equiv) at 0° C. The resulting mixture was stirred for 30 min at 0° C. Then tert-butyl 2-bromoacetate (320 mg, 1.63 mmol, 1.2 equiv) was added dropwise to the above solution at 0° C. The resulting mixture was stirred for 2 h at room temperature. The reaction was quenched with sat. NH₄Cl (20 mL) at room temperature. The resulting mixture was extracted with ethyl acetate (EtOAc) (5×20 mL). The combined organic layers were dried over anhydrous Na₂SO₄. After filtration, the filtrate was concentrated under reduced pressure. The residue was purified by silica gel column chromatography, eluting with petroleum ether/ethyl acetate (5:1), to afford tert-butyl 2-(5-bromo-7-iodo-3,3-dimethyl-2-oxoindol-1-yl)acetate (500 mg, 76% yield) as a yellow green solid. LCMS:(ES, m/z): RT=0.92 min, m/z=480 [M+H]⁺. ¹H NMR (400 MHz, DMSO-d₆) δ 7.83 (d, J=2.0 Hz, 1H), 7.70 (d, J=2.0 Hz, 1H), 4.74 (s, 1H), 4.02 (s, 1H), 1.43 (d, J=2.5 Hz, 9H), 1.31 (s, 6H).

Step 3: To a stirred solution of tert-butyl 2-(5-bromo-7-iodo-3,3-dimethyl-2-oxoindol-1-yl)acetate (500 mg, 1.04 mmol, 1 equiv) in pyridine (4 mL) and H₂O (8 mL) was added NaOH (208 mg, 5.20 mmol, 5 equiv) followed by copper (I) oxide (Cu₂O) (29.8 mg, 0.20 mmol, 0.2 equiv) at room temperature. The resulting mixture was stirred for 12 h at 110° C. The resulting mixture was filtered, the filtrate was neutralized to pH 2 with 1N HCl aq. The resulting mixture was extracted with ethyl acetate (EtOAc) (5×20 mL). The combined organic layers were dried over anhydrous Na₂SO₄. After filtration, the filtrate was concentrated under reduced pressure. The crude product was purified by reverse phase flash chromatography to afford (5-bromo-7-hydroxy-3,3-dimethyl-2-oxoindol-1-yl)acetic acid (200 mg, 52% yield) as a brown solid. LCMS: (ES, m/z): RT=0.70 min, m/z=314 [M+H]⁺. ¹H NMR (400 MHz, DMSO-d₆) δ 12.12 (s, 1H), 7.02 (d, J=1.9 Hz, 1H), 6.87-6.82 (m, 1H), 4.37 (s, 1H), 4.04 (d, J=7.1 Hz, 1H), 1.25 (s, 6H).

Step 4: To a stirred solution of (5-bromo-7-hydroxy-3,3-dimethyl-2-oxoindol-1-yl)acetic acid (100 mg, 0.31 mmol, 1 equiv) and methyl 4-aminobutanoate (44.8 mg, 0.38 mmol, 1.2 equiv) in N,N-dimethyl formamide (DMF) (2.5 mL) was added triethylamine (TEA) (96.6 mg, 0.95 mmol, 3 equiv) followed by [bis(dimethylamino)methylene]-1H-1,2,3-triazolo[4,5-b]pyridinium 3-oxide hexafluorophosphate (HATU) (145.25 mg, 0.38 mmol, 1.2 equiv) at room temperature. The resulting mixture was stirred for 2 h at room temperature. The reaction was monitored by LCMS. The resulting mixture was extracted with ethyl acetate (EtOAc) (5×20 mL). The combined organic layers were dried over anhydrous Na₂SO₄. After filtration, the filtrate was concentrated under reduced pressure. The residue was purified by silica gel column chromatography, eluting with petroleum ether/ethyl acetate (1:1), to afford 2-(5-bromo-7-hydroxy-3,3-dimethyl-2-oxoindol-1-yl)-N-(4-methoxy-4,4-dioxobutyl)acetamide (also referred to herein as 4-(2-(5-bromo-7-hydroxy-3,3-dimethyl-2-oxoindolin-1-yl)acetamido)butanoic acid; Compound 16-OMe) (65 mg, 48% yield) as an off-white solid. LCMS:(ES, m/z): RT=0.52 min, m/z=413 [M+H]⁺.

Step 5: To a solution of 2-(5-bromo-7-hydroxy-3,3-dimethyl-2-oxoindol-1-yl)-N-(4-methoxy-4,4-dioxobutyl)acetamide (50 mg, 0.116 mmol, 1 equiv) in methanol (MeOH) (2 mL) and H₂O (0.5 mL) was added LiOH (8.37 mg, 0.35 mmol, 3 equiv). The resulting mixture was stirred for 3 h at room temperature. The reaction was monitored by LCMS. The mixture was acidified to pH 2 with 1N HCl aq. The resulting mixture was extracted with ethyl acetate (EtOAc) (5×20 mL). The combined organic layers were dried over anhydrous Na₂SO₄. After filtration, the filtrate was concentrated under reduced pressure. The crude product was purified by Prep-HPLC (YMC-Actus Triart C18 ExRS, 30*150 mm, 5 μm; mobile phase, water (10 mmol/L NH₄HCO₃) and acetonitrile (MeCN) (10% up to 22% in 10 min); Detector, UV 254 nm) to provide 4-(2-(5-bromo-7-hydroxy-3,3-dimethyl-2-oxoindolin-1-yl)acetamido)butanoic acid (Compound 16) (26.3 mg, 55% yield) as a white solid. LCMS: (ES, m/z): RT=0.67 min, m/z=399.00 [M+H]⁺. ¹H NMR (400 MHz, DMSO-d₆) δ 7.95 (s, 1H), 6.99 (s, 1H), 6.80 (s, 1H), 4.42 (s, 2H), 3.05 (d, J=5.9 Hz, 2H), 2.17 (s, 2H), 1.62 (d, J=7.0 Hz, 2H), 1.26 (s, 6H).

### Example 8: 4-(2-(5-bromo-7-fluoro-3,3-dimethyl-2-oxoindolin-1-yl)acetamido)butanoic acid (Compound 24) and methyl 4-(2-(5-bromo-7-fluoro-3,3-dimethyl-2-oxoindolin-1-yl)acetamido)butanoate (Compound 24-OMe)

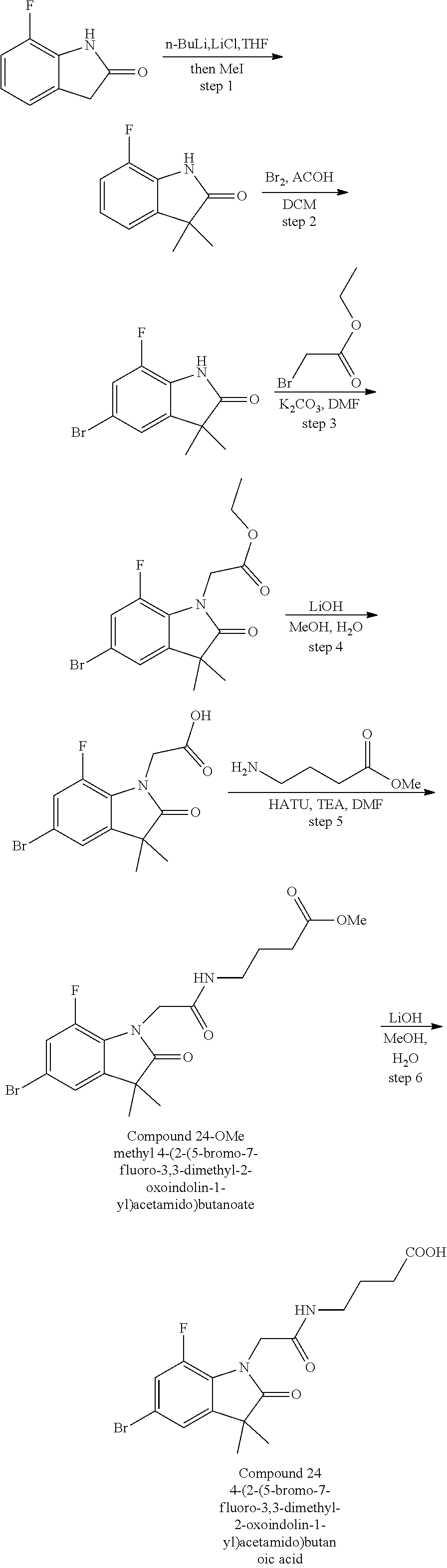

Step 1: To a solution of 7-fluoro-1,3-dihydroindol-2-one (2 g, 13.23 mmol, 1 equiv) and lithium chloride (1.40 g, 33.08 mmol, 2.5 equiv) in tetrahydrofuran (THF) (40 mL) at 0° C. was added dropwise added n-butyl lithium (n-BuLi) (2.5M in hexanes, 10.5 ml, 26.46 mmol, 2 equiv) in portions under nitrogen atmosphere. The resulting mixture was stirred for 30 min at 0° C. Then methyl iodide (Mel) (4.69 g, 33.08 mmol, 2.5 equiv) was added slowly and the mixture was stirred at 0° C. for 2 h, and then further stirred at ambient temperature for 16 h. The reaction was monitored by LCMS. The reaction was quenched with sat. NH₄Cl (40 mL). The aqueous layer was extracted with ethyl acetate (EtOAc) (4×50 mL). The combined organic layers were washed with brine (1×50 mL) and dried over anhydrous Na₂SO₄. After filtration, the filtrate was concentrated under reduced pressure. The residue was purified by reverse flash chromatography (C18 silica gel; mobile phase, MeCN in water, 10% to 50% gradient in 30 min; detector, UV 254 nm) to provide 7-fluoro-3,3-dimethyl-1H-indol-2-one (1.3 g, 54% yield) as a brown solid. LCMS: (ES, m/z): RT=0.73 min, m/z=180[M+H]⁺. ¹H NMR (400 MHz, DMSO-d₆) δ 10.81 (s, 1H), 7.15 (dd, J=7.3, 1.1 Hz, 1H), 7.08 (m, J=10.4, 8.5, 1.1 Hz, 1H), 6.98 (m, J=8.4, 7.3, 4.8 Hz, 1H), 1.27 (s, 6H).

Step 2: To a solution of 7-fluoro-3,3-dimethyl-1H-indol-2-one (1 g, 5.58 mmol, 1 equiv) in acetic acid (AcOH) (4 mL) and dichloromethane (DCM) (40 mL), was added bromine (Br₂) (892 mg, 5.58 mmol, 1 equiv) was added by dropwise over 4 min at 0° C. The resulting mixture was stirred for additional 2 h at room temperature. The reaction was monitored by LCMS. The reaction was quenched with saturated NaHSO₃ aq (20 mL) at 0° C. The aqueous layer was extracted with ethyl acetate (EtOAc) (3×50 mL). The combined organic layers were washed with brine (1×50 mL), dried over anhydrous Na₂SO₄. After filtration, the filtrate was concentrated under reduced pressure. The residue was purified by silica gel column chromatography, eluting with petroleum ether/ethyl acetate (4:1), to afford 5-bromo-7-fluoro-3,3-dimethyl-1H-indol-2-one (1.10 g, 73% yield) as a yellow solid. LCMS: (ES, m/z): RT=0.83 min, m/z=258[M+H]⁺. ¹H NMR (400 MHz, DMSO-d₆) δ 10.97 (s, 1H), 7.45 (d, J=1.8 Hz, 1H), 7.41 (dd, J=9.8, 1.8 Hz, 1H), 1.28 (s, 6H).

Step 3: To a solution of 5-bromo-7-fluoro-3,3-dimethyl-1H-indol-2-one (500 mg, 1.93 mmol, 1 equiv) in N,N-dimethyl formamide (DMF) (30 mL) was added K₂CO₃ (535 mg, 3.87 mmol, 2 equiv) and ethyl bromoacetate (388 mg, 2.32 mmol, 1.2 equiv) at 0° C. The resulting mixture was stirred for additional 2 h at room temperature. The reaction progress was monitored by LCMS. The resulting solution was diluted with water (200 mL), and extracted with ethyl acetate (EtOAc) (4×50 mL). The combined organic layers were washed with brine (1×50 mL) and dried over anhydrous Na₂SO₄. After filtration, the filtrate was concentrated under reduced pressure. The residue was purified by reverse flash chromatography (C18 silica gel; mobile phase, MeCN in water, 10% to 50% gradient in 10 min; detector, UV 254 nm) to provide ethyl 2-(5-bromo-7-fluoro-3,3-dimethyl-2-oxoindol-1-yl)acetate (550 mg, 78% yield) as a light yellow solid. LCMS: (ES, m/z): RT=1.03 min, m/z=344[M+H]⁺.

Step 4: To a solution of ethyl 2-(5-bromo-7-fluoro-3,3-dimethyl-2-oxoindol-1-yl) acetate (100 mg, 0.20 mmol, 1 equiv) in methanol (MeOH) (5 mL) and water (1 mL) was added LiOH (13.9 mg, 0.58 mmol, 2 equiv). The resulting mixture was stirred for 1 h at room temperature. The reaction was monitored by LCMS. The resulting mixture was concentrated under reduced pressure and dissolved by water (5 mL). The mixture residue was acidified to pH 5 with 1N HCl aq. The precipitated solids were collected by filtration and washed with H₂O (20 mL). This resulted in (5-bromo-7-fluoro-3,3-dimethyl-2-oxoindol-1-yl) acetic acid (85 mg, 88% yield) as a white solid. LCMS: (ES, m/z): RT=0.84 min, m/z=316[M+H]⁺.

Step 5: To a solution of (5-bromo-7-fluoro-3,3-dimethyl-2-oxoindol-1-yl)acetic acid (100 mg, 0.31 mmol, 1 equiv) in N,N-dimethyl formamide (DMF) (3 mL) was added methyl 4-aminobutanoate (44.5 mg, 0.37 mmol, 1.2 equiv), triethylamine (TEA) (96 mg, 0.94 mmol, 3 equiv) and [bis(dimethylamino)methylene]-1H-1,2,3-triazolo[4,5-b]pyridinium 3-oxide hexafluorophosphate (HATU) (132 mg, 0.34 mmol, 1.1 equiv). The resulting mixture was stirred for 2 h at room temperature. The reaction progress was monitored by LCMS. The resulting mixture was diluted with H₂O (40 mL). The aqueous layer was extracted with ethyl acetate (EtOAc) (3×50 mL). The combined organic layers were washed with brine (1×50 mL) and dried over anhydrous Na₂SO₄. After filtration, the filtrate was concentrated under reduced pressure. The residue was purified by silica gel column chromatography, eluting with petroleum ether/ethyl acetate (3:2), to afford methyl 4-[2-(5-bromo-7-fluoro-3,3-dimethyl-2-oxoindol-1-yl)acetamido]butanoate (also referred to as methyl 4-(2-(5-bromo-7-fluoro-3,3-dimethyl-2-oxoindolin-1-yl)acetamido)butanoate; Compound 24-OMe) (120 mg, 87% yield) as a light yellow solid. LCMS: (ES, m/z): RT=0.87 min, m/z=415[M+H]⁺.

Step 6: To a solution of methyl 4-[2-(5-bromo-7-fluoro-3,3-dimethyl-2-oxoindol-1-yl)acetamido]butanoate (150 mg, 0.36 mmol, 1 equiv) in H₂O (0.40 mL) and methanol (MeOH) (2 mL) was added LiOH (17.3 mg, 0.72 mmol, 2 equiv) at room temperature. The resulting mixture was stirred for 2 h at room temperature. The reaction was monitored by LCMS. The resulting mixture was concentrated under reduced pressure. The mixture residue was acidified to pH 4 with 1N HCl aq. The aqueous layer was extracted with ethyl acetate (EtOAc) (4×20 mL). The combined organic layers were washed with brine (1×20 mL) and dried over anhydrous Na₂SO₄. After filtration, the filtrate was concentrated under reduced pressure. The crude product was purified by Prep-HPLC (2 #SHIMADZU (HPLC-01)): Column: XSelect CSH C18 OBD Column 30*150 mm 5 μm; Mobile Phase A: acetonitrile (MeCN), Mobile Phase B: water (0.05% TFA); Flow rate: 60 mL/min; Gradient: 30% B to 38% B in 10 min, 38% B; Wave Length: 254/220 nm) to provide 4-(2-(5-bromo-7-fluoro-3,3-dimethyl-2-oxoindolin-1-yl)acetamido)butanoic acid (Compound 24) (53.2 mg, 37% yield) as a white solid. LCMS: (ES, m/z): RT=0.73 min, m/z=401[M+H]⁺. ¹H NMR (400 MHz, DMSO-d₆) δ 8.17 (t, J=5.6 Hz, 1H), 7.53 (d, J=1.7 Hz, 1H), 7.43 (dd, J=11.0, 1.8 Hz, 1H), 4.35 (d, J=1.8 Hz, 2H), 3.09 (m, 2H), 2.22 (t, J=7.4 Hz, 2H), 1.63 (m, 2H), 1.32 (s, 6H).

### Example 9: 4-(2-(5-bromo-7-methoxy-3,3-dimethyl-2-oxoindolin-1-yl)acetamido)butanoic acid (Compound 27) and methyl 4-(2-(5-bromo-7-methoxy-3,3-dimethyl-2-oxoindolin-1-yl)acetamido)butanoate (Compound 27-OMe)

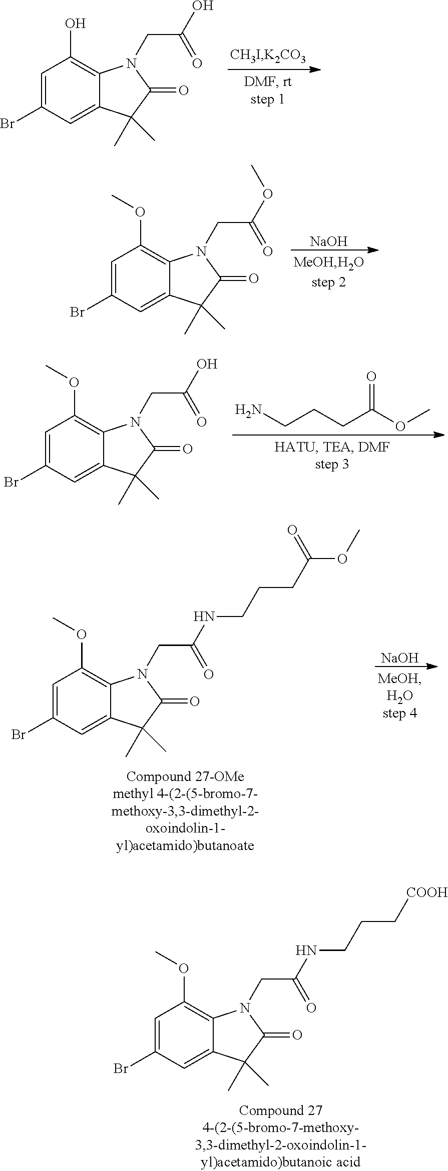

Step 1: To a solution of (5-bromo-7-hydroxy-3,3-dimethyl-2-oxoindol-1-yl)acetic acid (product of Step 3 of Example 7) (120 mg, 0.38 mmol, 1 equiv) in N,N-dimethyl formamide (DMF) (4 mL) was added K₂CO₃ (158 mg, 1.15 mmol, 3 equiv) and CH₃I (271 mg, 1.91 mmol, 5 equiv) at room temperature. The resulting mixture was stirred for 12 h at room temperature under air atmosphere. The reaction progress was monitored by LCMS. The resulting mixture was diluted with H₂O (20 mL). The aqueous layer was extracted with ethyl acetate (EtOAc) (3×30 mL). The combined organic layers were washed with brine (1×20 mL), and dried over anhydrous Na₂SO₄. After filtration, the filtrate was concentrated under reduced pressure. The residue was purified by silica gel column chromatography, eluting with petroleum ether/ethyl acetate (7:1), to afford methyl 2-(5-bromo-7-methoxy-3,3-dimethyl-2-oxoindol-1-yl)acetate (125 mg, 96% yield) as a white solid. LCMS: (ES, m/z): RT=0.92 min, m/z=342[M+H]⁺. ¹H NMR (400 MHz, DMSO-d₆) δ 7.29 (d, J=1.6 Hz, 1H), 7.17 (d, J=1.6 Hz, 1H), 4.61 (s, 2H), 3.77 (s, 3H), 3.68 (s, 3H), 1.29 (s, 6H).

Step 2: To a solution of methyl 2-(5-bromo-7-methoxy-3,3-dimethyl-2-oxoindol-1-yl)acetate (120 mg, 0.35 mmol, 1 equiv) in methanol (MeOH) (5 mL) and H₂O (1 mL) was added NaOH (28.1 mg, 0.70 mmol, 2 equiv) at room temperature. The resulting mixture was stirred for 2 h at room temperature under air atmosphere. The reaction progress was monitored by LCMS. After the reaction was completed, the residue was concentrated under reduced pressure and re-dissolved with water (5 mL), then acidified to pH=4 with 1N HCl aq. The resulting mixture was extracted with ethyl acetate (EtOAc) (5×20 mL). The combined organic layers were washed with brine (1×10 mL), dried over anhydrous Na₂SO₄. After filtration, the filtrate was concentrated under reduced pressure. This resulted in (5-bromo-7-methoxy-3,3-dimethyl-2-oxoindol-1-yl) acetic acid (110 mg, 96% yield) as a white solid. LCMS: (ES, m/z): RT=1.20 min, m/z=328[M+H]⁺.

Step 3: To a solution of (5-bromo-7-methoxy-3,3-dimethyl-2-oxoindol-1-yl) acetic acid (90 mg, 0.27 mmol, 1 equiv) and methyl 4-aminobutanoate (38.6 mg, 0.33 mmol, 1.2 equiv) in N,N-dimethyl formamide (DMF) (3 mL) was added triethylamine (TEA) (55.5 mg, 0.55 mmol, 2 equiv) followed by [bis(dimethylamino)methylene]-1H-1,2,3-triazolo[4,5-b]pyridinium 3-oxide hexafluorophosphate (HATU) (125 mg, 0.33 mmol, 1.2 equiv) at room temperature. The resulting mixture was stirred for 2 h at room temperature under air atmosphere. The reaction progress was monitored by LCMS. After the reaction was completed, the resulting mixture was diluted with H₂O (10 mL), and the resulting mixture was extracted with ethyl acetate (EtOAc) (3×20 mL). The combined organic layers were washed with brine (1×15 mL) and dried over anhydrous Na₂SO₄. After filtration, the filtrate was concentrated under reduced pressure. The residue was purified by silica gel column chromatography, eluting with petroleum ether/ethyl acetate (5:1), to afford methyl 4-[2-(5-bromo-7-methoxy-3,3-dimethyl-2-oxoindol-1-yl)acetamido]butanoate (also referred to as methyl 4-(2-(5-bromo-7-methoxy-3,3-dimethyl-2-oxoindolin-1-yl)acetamido)butanoate, Compound 27-OMe) (90 mg, 77% yield) as a white solid. LCMS: (ES, m/z): RT=1.25 min, m/z=427[M+H]⁺. ¹H NMR (400 MHz, DMSO-d₆) δ 8.01 (t, J=5.6 Hz, 1H), 7.24 (d, J=1.6 Hz, 1H), 7.12 (d, J=1.6 Hz, 1H), 4.40 (s, 2H), 3.75 (s, 3H), 3.59 (s, 3H), 3.08 (q, J=6.4 Hz, 2H), 2.32 (t, J=7.2 Hz, 2H), 1.67 (p, J=7.2 Hz, 2H), 1.28 (s, 6H).

Step 4: To a solution of methyl 4-[2-(5-bromo-7-methoxy-3,3-dimethyl-2-oxoindol-1-yl)acetamido]butanoate (85 mg, 0.20 mmol, 1 equiv) in methanol (MeOH) (2 mL) and H₂O (0.5 mL) was added NaOH (15.9 mg, 0.40 mmol, 2 equiv) at room temperature. The resulting mixture was stirred for 12 h at room temperature. The reaction progress was monitored by LCMS. After the reaction was completed, the residue was concentrated under reduced pressure and re-dissolved with water (10 mL), then acidified to pH=4 with 1N HCl aq. The resulting mixture was extracted with ethyl acetate (EtOAc) (5×20 mL). The combined organic layers were washed with brine (1×10 mL) and dried over anhydrous Na₂SO₄. After filtration, the filtrate was concentrated under reduced pressure. The crude product was purified by Prep-HPLC (Column: XSelect CSH Prep C18 OBD Column, 19*250 mm, 5 μm; Mobile Phase A: water (0.05% FA), Mobile Phase B: acetonitrile (MeCN); Flow rate: 25 mL/min; Gradient: 42% B to 42% B in 8 min, 42% B; Wave Length: 254 nm; HPLC RT(min): 7.6) to afford 4-(2-(5-bromo-7-methoxy-3,3-dimethyl-2-oxoindolin-1-yl)acetamido)butanoic acid (Compound 27) (43.0 mg, 54% yield) as a white solid. LCMS: (ES, m/z): RT=1.41 min, m/z=413[M+H]⁺. ¹H NMR (400 MHz, DMSO-d₆) δ 12.04 (s, 1H), 8.02 (t, J=5.6 Hz, 1H), 7.24 (d, J=1.6 Hz, 1H), 7.12 (d, J=1.6 Hz, 1H), 4.39 (s, 2H), 3.75 (s, 3H), 3.08 (q, J=6.4 Hz, 2H), 2.23 (t, J=7.2 Hz, 2H), 1.63 (d, J=7.2 Hz, 2H), 1.28 (s, 6H).

### Example 10: 4-(2-(7-hydroxy-5-iodo-3,3-dimethyl-2-oxoindolin-1-yl)acetamido)butanoic acid (Compound 28) and methyl 4-(2-(7-hydroxy-5-iodo-3,3-dimethyl-2-oxoindolin-1-yl)acetamido)butanoate (Compound 28-OMe)

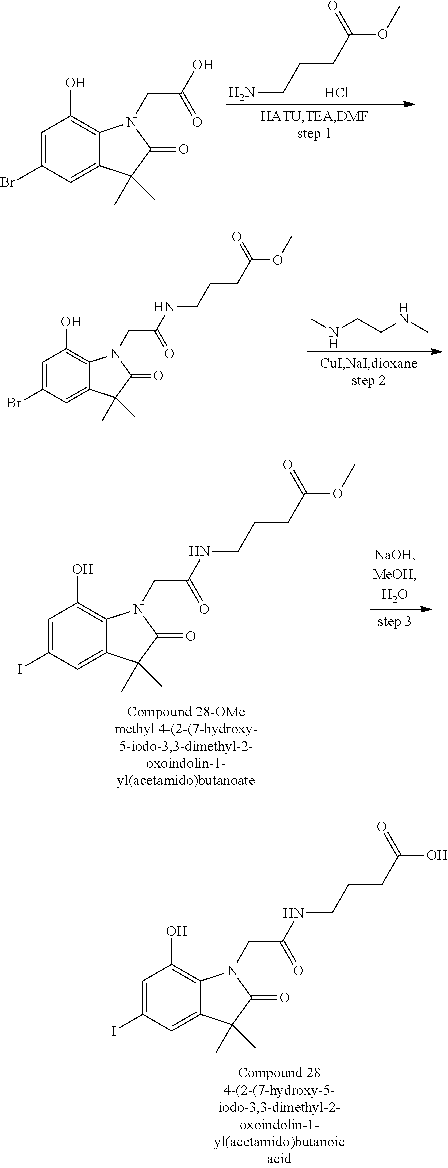

Step 1: To a solution of (5-bromo-7-hydroxy-3,3-dimethyl-2-oxoindol-1-yl)acetic acid (product of Step 3 of Example 7) (240 mg, 0.76 mmol, 1 equiv), methyl 4-aminobutanoate hydrochloride (141 mg, 0.91 mmol, 1.2 equiv), and triethylamine (TEA) (232 mg, 2.29 mmol, 3 equiv) in N,N-dimethyl formamide (DMF) (5 mL), was added [bis(dimethylamino)methylene]-1H-1,2,3-triazolo[4,5-b]pyridinium 3-oxide hexafluorophosphate (HATU) (349 mg, 0.91 mmol, 1.20 equiv), and the resulting solution was stirred for 1 h at room temperature. The reaction progress was monitored by LCMS. The reaction was quenched by the addition of water (15 mL) at room temperature. The resulting mixture was extracted with ethyl acetate (EtOAc) (3×30 mL) and dried over anhydrous Na₂SO₄. After filtration, the filtrate was concentrated under reduced pressure. The residue was purified by reverse flash chromatography (C18 silica gel; mobile phase, MeCN in water (0.1% TFA), 10% to 50% gradient in 10 min; detector, UV 254 nm) to provide methyl 4-[2-(5-bromo-7-hydroxy-3,3-dimethyl-2-oxoindol-1-yl) acetamido] butanoate (190 mg, 60% yield) as a yellow oil. LCMS: (ES, m/z): RT=0.762 min, m/z=413.0[M+H]⁺.

Step 2: A solution of methyl 4-[2-(5-bromo-7-hydroxy-3,3-dimethyl-2-oxoindol-1-yl) acetamido] butanoate (150 mg, 0.36 mmol, 1 equiv), methyl[2-(methylamino)ethyl]amine (64.0 mg, 0.72 mmol, 2 equiv), and NaI (435 mg, 2.90 mmol, 8 equiv) in dioxane (8 mL) was stirred for 2 h at 120° C. under nitrogen atmosphere. The reaction progress was monitored by LCMS. The reaction was quenched by the addition of water (30 mL) at room temperature. The resulting mixture was extracted with ethyl acetate (EtOAc) (3×20 mL) and dried over anhydrous Na₂SO₄. After filtration, the filtrate was concentrated under reduced pressure. The residue was purified by silica gel column chromatography, eluting with petroleum ether/ethyl acetate (3:2), to afford methyl 4-[2-(7-hydroxy-5-iodo-3,3-dimethyl-2-oxoindol-1-yl) acetamido] butanoate (also referred to as methyl 4-(2-(7-hydroxy-5-iodo-3,3-dimethyl-2-oxoindolin-1-yl)acetamido)butanoate, Compound 28-OMe) (100 mg, 60% yield) as a yellow oil. LCMS: (ES, m/z): RT=0.778 min, m/z=460.9[M+H]⁺.

Step 3: A solution of methyl 4-[2-(7-hydroxy-5-iodo-3,3-dimethyl-2-oxoindol-1-yl) acetamido] butanoate (80.0 mg, 0.17 mmol, 1 equiv) and NaOH (104 mg, 2.61 mmol, 15.0 equiv) in methanol (3 mL) and water (3 mL) was stirred for 1 h at room temperature. The reaction progress was monitored by LCMS. The reaction solution was concentrated under reduced pressure. The reaction was dissolved by the addition of water (20 mL) at room temperature. The residue was acidified to pH 6 with 2N HCl(aq). The aqueous layer was extracted with ethyl acetate (EtOAc) (3×20 mL) and dried over anhydrous Na₂SO₄. After filtration, the filtrate was concentrated under reduced pressure. The residue was purified by silica gel column chromatography, eluting with petroleum ether/ethyl acetate (3:7), to afford 4-(2-(7-hydroxy-5-iodo-3,3-dimethyl-2-oxoindolin-1-yl)acetamido)butanoic acid (80 mg, 85% purity) as a yellow oil which was further purified by Prep-HPLC (Column: XBridge Prep Phenyl OBD Column, 19*250 mm, 5 μm; Mobile Phase A: water (0.05% TFA), Mobile Phase B: acetonitrile (MeCN); Flow rate: 25 mL/min; Gradient: 30% B to 40% B in 10 min, 40% B; Wave Length: 254 nm; HPLC RT(min): 9) to afford 4-(2-(7-hydroxy-5-iodo-3,3-dimethyl-2-oxoindolin-1-yl)acetamido)butanoic acid (Compound 28) (39.9 mg, 66% yield) as a white solid. LCMS: (ES, m/z): RT=1.295 min, m/z=446.9[M+H]⁺. ¹H NMR (400 MHz, DMSO-d₆) δ 12.03 (s, 1H), 9.96 (s, 1H), 7.97 (t, J=5.6 Hz, 1H), 7.18 (d, J=1.6 Hz, 1H), 7.01 (d, J=1.6 Hz, 1H), 4.43 (s, 2H), 3.07 (q, J=6.6 Hz, 2H), 2.22 (t, J=7.4 Hz, 2H), 1.62 (p, J=7.3 Hz, 2H), 1.26 (s, 6H).

### Example 11: N-(3-(1H-tetrazol-5-yl)propyl)-2-(5-cyclopropyl-7-hydroxy-3,3-dimethyl-2-oxoindolin-1-yl)acetamide (tautomer 1) and N-(3-(2H-tetrazol-5-yl)propyl)-2-(5-cyclopropyl-7-hydroxy-3,3-dimethyl-2-oxoindolin-1-yl)acetamide (tautomer 2) (Compound 29)

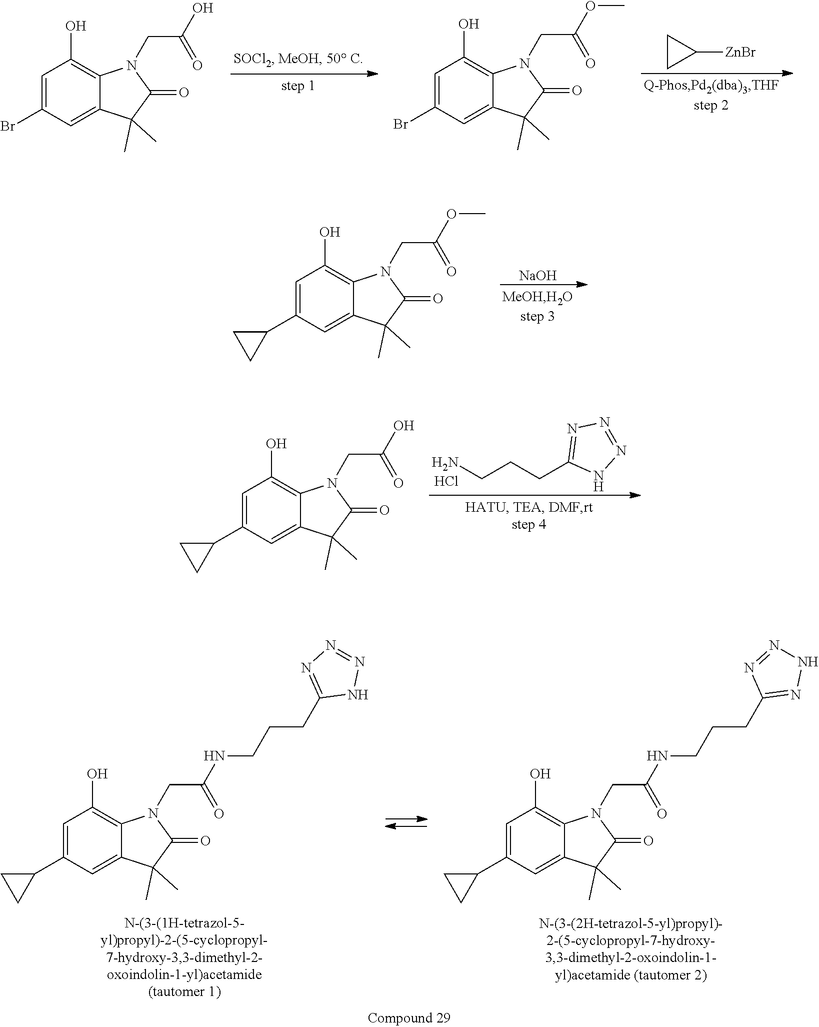

Step 1: To a solution of (5-bromo-7-hydroxy-3,3-dimethyl-2-oxoindol-1-yl)acetic acid (product of Step 3 of Example 7) (120 mg, 0.30 mmol, 1 equiv) in methanol (MeOH) (5 mL) was added SOCl₂ (13.6 mg, 0.12 mmol, 0.3 equiv) at room temperature. The resulting mixture was stirred for 2 h at 70° C. The reaction progress was monitored by LCMS. After the reaction was completed, the residue was concentrated under reduced pressure. The crude product was purified by silica gel column chromatography, eluting with petroleum ether/ethyl acetate (7:1), to afford methyl 2-(5-bromo-7-hydroxy-3,3-dimethyl-2-oxoindol-1-yl) acetate (125 mg, 100% yield) as a white solid. LCMS: (ES, m/z): RT=0.92 min, m/z=328[M+H]⁺. ¹H NMR (400 MHz, DMSO-d₆) δ 10.19 (s, 1H), 7.11 (d, J=1.6 Hz, 1H), 6.86 (d, J=2.4 Hz, 1H), 3.75 (d, J=3.2 Hz, 3H), 4.64 (s, 2H), 1.28 (s, 6H).

Step 2: To a solution of methyl 2-(5-bromo-7-hydroxy-3,3-dimethyl-2-oxoindol-1-yl)acetate (125 mg, 0.52 mmol, 1 equiv) in tetrahydrofuran (THF) (6 mL) was added 1,2,3,4,5-pentaphenyl-1′-(di-tert-butylphosphino)ferrocene (Q-Phos) (90.0 mg, 0.45 mmol, 0.80 equiv), bromo(cyclopropyl)zinc (0.5M in THF) (3 mL, 1.50 mmol, 3 equiv) and tris(dibenzylideneacetone)dipalladium(0) (Pd₂(dba)₃) (140 mg, 0.16 mmol, 0.30 equiv). The resulting mixture was stirred for 1 h at 25° C. under nitrogen atmosphere. The reaction progress was monitored by LCMS. The resulting mixture was diluted with H₂O (50 mL). The aqueous layer was extracted with ethyl acetate (EtOAc) (3×50 mL). The combined organic layers were washed with brine (1×50 mL) and dried over anhydrous Na₂SO₄. After filtration, the filtrate was concentrated under reduced pressure. The residue was purified by reverse flash chromatography (C18 silica gel; mobile phase, MeCN in water (0.1% FA), 10% to 50% gradient in 10 min; detector, UV 254 nm) to afford methyl 2-(5-cyclopropyl-7-hydroxy-3,3-dimethyl-2-oxoindolin-1-yl)acetate (105 mg, 62% yield) as a brown solid. LCMS: (ES, m/z): RT=1.20 min, m/z=290[M+H]⁺. ¹H NMR (400 MHz, DMSO-d₆) δ 9.55 (s, 1H), 6.60 (d, J=1.6 Hz, 1H), 6.40 (d, J=1.6 Hz, 1H), 3.67 (d, J=11.2 Hz, 4H), 1.81 (m, 1H), 1.67-1.42 (m, 2H), 1.32 (d, J=7.2 Hz, 3H), 1.25 (s, 6H).

Step 3: To a solution of methyl 2-(5-cyclopropyl-7-hydroxy-3,3-dimethyl-2-oxoindol-1-yl) acetate (105 mg, 0.38 mmol, 1 equiv) in methanol (MeOH) (5 mL) and H₂O (1 mL) was added NaOH (30.4 mg, 0.76 mmol, 2 equiv) at room temperature. The resulting mixture was stirred for 2 h at room temperature. The reaction was monitored by LCMS. After the reaction was completed, the residue was concentrated under reduced pressure and re-dissolved with water (5 mL), then acidified to pH=4 with 1N HCl aq. The resulting mixture was extracted with ethyl acetate (EtOAc) (5×20 mL). The combined organic layers were washed with brine (1×10 mL) and dried over anhydrous Na₂SO₄. After filtration, the filtrate was concentrated under reduced pressure to provide (5-cyclopropyl-7-hydroxy-3,3-dimethyl-2-oxoindol-1-yl) acetic acid (95 mg, 91% yield) as a white solid. LCMS: (ES, m/z): RT=1.25 min, m/z=276[M+H]⁺.

Step 4: To a solution of (5-cyclopropyl-7-hydroxy-3,3-dimethyl-2-oxoindol-1-yl) acetic acid (80 mg, 0.31 mmol, 1 equiv) and 3-(1H-1,2,3,4-tetrazol-5-yl)propan-1-amine (46.7 mg, 0.37 mmol, 1.2 equiv) in N,N-dimethyl formamide (DMF) (3 mL) was added triethylamine (TEA) (62 mg, 0.61 mmol, 2 equiv), followed by [bis(dimethylamino)methylene]-1H-1,2,3-triazolo[4,5-b]pyridinium 3-oxide hexafluorophosphate (HATU) (128.1 mg, 0.34 mmol, 1.1 equiv) at room temperature. The resulting mixture was stirred for 2 h at 25° C. The reaction was monitored by LCMS. After the reaction was completed, the residue was purified by reverse flash chromatography (C18 silica gel; mobile phase, acetonitrile (MeCN) in water (0.1% FA), 10% to 50% gradient in 10 min; detector, UV 254 nm) to afford N-(3-(1H-tetrazol-5-yl)propyl)-2-(5-cyclopropyl-7-hydroxy-3,3-dimethyl-2-oxoindolin-1-yl)acetamide (tautomer 1) and N-(3-(2H-tetrazol-5-yl)propyl)-2-(5-cyclopropyl-7-hydroxy-3,3-dimethyl-2-oxoindolin-1-yl)acetamide (tautomer 2) (Compound 29) (9.3 mg, 62% yield) as a white solid. LCMS: (ES, m/z): RT=1.26 min, m/z=385[M+H]⁺. ¹H NMR (400 MHz, DMSO-d₆) δ 8.04 (t, J=5.6 Hz, 1H), 6.54 (d, J=1.6 Hz, 1H), 6.43 (d, J=1.6 Hz, 1H), 4.42 (s, 2H), 3.08 (q, J=6.4 Hz, 2H), 2.74 (t, J=7.2 Hz, 2H), 1.79 (m, J₁=8.4, J₂=4.4 Hz, 1H), 1.71 (q, J=7.2, 6.8 Hz, 2H), 1.24 (s, 6H), 0.85 (m, 2H), 0.54 (m, 2H).

### Example 12: 4-(2-(5-bromo-6-hydroxy-3,3-dimethyl-2-oxoindolin-1-yl)acetamido)butanoic acid (Compound 36) and methyl 4-(2-(5-bromo-6-hydroxy-3,3-dimethyl-2-oxoindolin-1-yl)acetamido)butanoate (Compound 36-OMe)

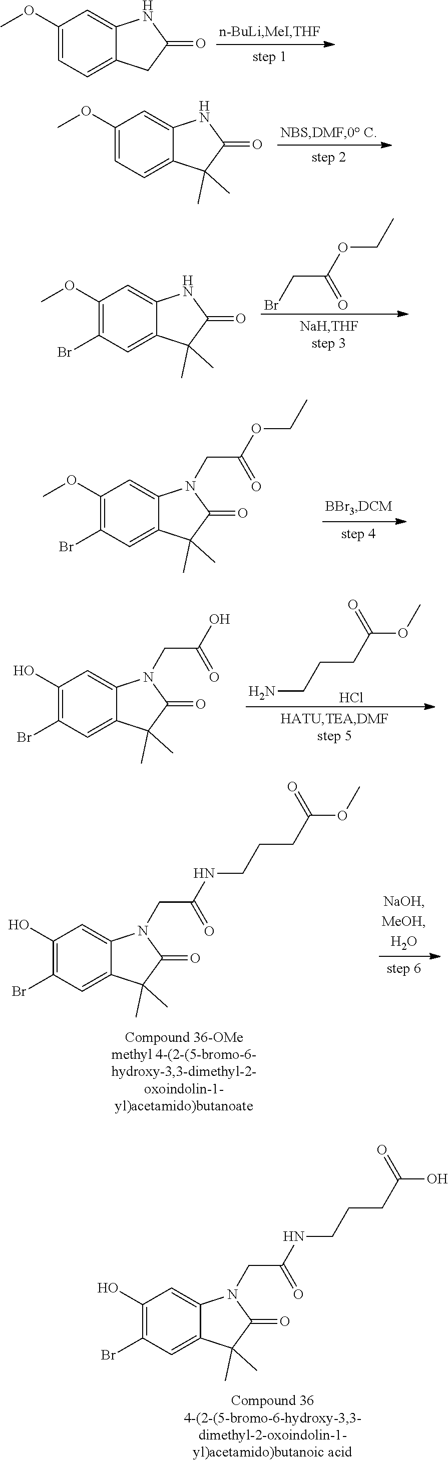

Step 1: To a stirred solution of 6-methoxy-1,3-dihydroindol-2-one (1 g, 6.12 mmol, 1 equiv) and LiCl (649 mg, 15.32 mmol, 2.5 equiv) in tetrahydrofuran (THF) (100 mL) was added n-butyl lithium (n-BuLi) (2.5N) (6.13 mL, 15.32 mmol, 2.5 equiv) dropwise at 0° C. under nitrogen atmosphere. The resulting mixture was stirred for 30 min at 0° C. under nitrogen atmosphere. To the above mixture was added Mel (1739.70 mg, 12.25 mmol, 2 equiv) dropwise over 20 min at 0° C. The resulting mixture was stirred for additional 1 h at 0° C. The reaction was quenched by the addition of water/ice (20 mL) at 0° C. The aqueous layer was extracted with ethyl acetate (EtOAc) (3×200 mL) and dried over anhydrous Na₂SO₄. After filtration, the filtrate was concentrated under reduced pressure. The residue was purified by silica gel column chromatography, eluting with petroleum ether/ethyl acetate (8:1), to afford 6-methoxy-3,3-dimethyl-1H-indol-2-one (200 mg, 17% yield) as a yellow solid. ¹H NMR (400 MHz, DMSO-d₆) δ 10.24 (s, 1H), 7.16 (d, J=8.2 Hz, 1H), 6.51 (dd, J=8.2, 2.4 Hz, 1H), 6.41 (d, J=2.3 Hz, 1H), 3.73 (s, 3H), 1.21 (s, 6H).

Step 2: Into a 20 mL vial was added 6-methoxy-3,3-dimethyl-1H-indol-2-one (200 mg, 1.04 mmol, 1 equiv), N,N-dimethyl formamide (DMF) (8 mL), and N-bromosuccinimide (NBS) (186 mg, 1.046 mmol, 1 equiv) at room temperature. The resulting mixture was stirred for 2 h at room temperature under nitrogen atmosphere. The reaction was quenched by the addition of water/ice (20 mL) at 0° C. The aqueous layer was extracted with ethyl acetate (EtOAc) (3×20 mL) and dried over anhydrous Na₂SO₄. After filtration, the filtrate was concentrated under reduced pressure. The residue was purified by silica gel column chromatography, eluting with petroleum ether/ethyl acetate (2:1), to afford 5-bromo-6-methoxy-3,3-dimethyl-1H-indol-2-one (190 mg, 67% yield) as a yellow solid. LCMS (LCMS, ESI): RT=0.735 min, m/z 270[M+H]. ¹H NMR (400 MHz, DMSO-d₆) δ 10.42 (s, 1H), 7.50 (s, 1H), 6.59 (s, 1H), 3.83 (s, 3H), 1.23 (s, 6H).

Step 3: A solution of 5-bromo-6-methoxy-3,3-dimethyl-1H-indol-2-one (150 mg, 0.55 mmol, 1 equiv) in tetrahydrofuran (THF) (10 mL) was treated with NaH (60% dispersion in mineral oil) (26.65 mg, 1.11 mmol, 2 equiv) for 30 min at 0° C. under nitrogen atmosphere, followed by the addition of ethyl bromoacetate (185 mg, 1.11 mmol, 2 equiv) dropwise at 0° C. The resulting mixture was stirred for 1 h at room temperature under nitrogen atmosphere. The reaction progress was monitored by LCMS. The reaction was quenched by the addition of water/ice (20 mL) at 0° C. The aqueous layer was extracted with ethyl acetate (EtOAc) (3×50 mL) and dried over anhydrous Na₂SO₄. The resulting mixture was concentrated under reduced pressure, and the residue was purified by silica gel column chromatography, eluting with petroleum ether/ethyl acetate (5:1), to afford ethyl 2-(5-bromo-6-methoxy-3,3-dimethyl-2-oxoindol-1-yl)acetate (170 mg, 86% yield) as a yellow solid. LCMS (LCMS, ESI): RT=928 min, m/z=356 [M+H]. ¹H NMR (400 MHz, DMSO-d₆) δ 7.59 (s, 1H), 6.99 (s, 1H), 4.60 (s, 2H), 4.15 (q, J=7.1 Hz, 2H), 3.85 (s, 3H), 1.28 (s, 6H), 1.21 (d, J=7.2 Hz, 3H).

Step 4: To a stirred solution of ethyl 2-(5-bromo-6-methoxy-3,3-dimethyl-2-oxoindol-1-yl)acetate (100 mg, 0.28 mmol, 1 equiv) in dichloromethane (DCM) was added BBr₃ (1N in DCM) (0.56 mL, 0.56 mmol, 2 equiv) dropwise at 0° C. under nitrogen atmosphere. The resulting mixture was stirred for 1 h at room temperature under nitrogen atmosphere. The reaction progress was monitored by LCMS. The reaction was quenched with water/ice at 0° C. The aqueous layer was extracted with ethyl acetate (EtOAc) (3×50 mL) and dried over anhydrous Na₂SO₄. After filtration, the filtrate was concentrated under reduced pressure, and the residue was purified by silica gel column chromatography, eluting with petroleum ether/ethyl acetate (1:1), to afford (5-bromo-6-hydroxy-3,3-dimethyl-2-oxoindol-1-yl)acetic acid (70 mg, 79% yield) as a yellow solid. LCMS (LCMS, ESI): RT=667 min, m/z=314 [M+H]⁺.

Step 5: Into a 8 mL vial was added (5-bromo-6-hydroxy-3,3-dimethyl-2-oxoindol-1-yl)acetic acid (60 mg, 0.19 mmol, 1 equiv), methyl 4-aminobutanoate (26.8 mg, 0.23 mmol, 1.2 equiv), triethylamine (TEA) (58.0 mg, 0.57 mmol, 3 equiv), and N,N-dimethyl formamide (DMF) (3 mL), and [bis(dimethylamino)methylene]-1H-1,2,3-triazolo[4,5-b]pyridinium 3-oxide hexafluorophosphate (HATU) (87.2 mg, 0.23 mmol, 1.20 equiv) was added at room temperature. The resulting mixture was stirred for 1 h at room temperature under nitrogen atmosphere. The reaction progress was monitored by LCMS. The residue was purified by reverse flash chromatography (C18 silica gel; mobile phase, acetonitrile (MeCN) in water (60% to 70% gradient in 10 min); detector, UV 254 nm) to provide methyl 4-[2-(5-bromo-6-hydroxy-3,3-dimethyl-2-oxoindol-1-yl)acetamido]butanoate (also referred to as methyl 4-(2-(5-bromo-6-hydroxy-3,3-dimethyl-2-oxoindolin-1-yl)acetamido)butanoate, Compound 36-OMe) (50 mg, 63% yield) as a yellow solid. LCMS (LCMS, ESI): RT=705 min, m/z=413 [M+H]⁺.

Step 6: Into a 8 mL vial was added methyl 4-[2-(5-bromo-6-hydroxy-3,3-dimethyl-2-oxoindol-1-yl)acetamido]butanoate (50.0 mg, 0.12 mmol, 1 equiv), NaOH (9.68 mg, 0.24 mmol, 2 equiv), methanol (MeOH) (1 mL) and H₂O (1 mL) at room temperature. The resulting mixture was stirred for 2 h at room temperature under air atmosphere. The reaction progress was monitored by LCMS. After the reaction was completed, the residue was concentrated under reduced pressure and re-dissolved with water (5 mL). The residue was acidified to pH 6 with 2N HCl (aq.), and the aqueous layer was extracted with ethyl acetate (EtOAc) (3×20 mL), and then dried over anhydrous Na₂SO₄. After filtration, the filtrate was concentrated under reduced pressure, and the residue was purified by reverse flash chromatography (C18 silica gel; mobile phase, acetonitrile (MeCN) in water (10% to 30% gradient in 10 min); detector, UV 254 nm) to provide 4-(2-(5-bromo-6-hydroxy-3,3-dimethyl-2-oxoindolin-1-yl)acetamido)butanoic acid (Compound 36) (20.7 mg, 43% yield) as a white solid. LCMS (LCMS, ESI): RT=1.211 min, m/z=398.95 [M+H]⁺. ¹H NMR (400 MHz, DMSO-d₆) δ 8.70 (s, 1H), 7.04 (s, 1H), 6.00 (s, 1H), 4.03 (s, 2H), 3.06 (q, J=6.0 Hz, 2H), 1.98 (t, J=6.9 Hz, 2H), 1.63 (dt, J=12.0, 6.0 Hz, 2H), 1.18 (s, 6H).

### Example 13: 4-(2-(5-cyclopropyl-4-fluoro-3,3-dimethyl-2-oxoindolin-1-yl)acetamido)butanoic acid (Compound 56) and methyl 4-(2-(5-cyclopropyl-4-fluoro-3,3-dimethyl-2-oxoindolin-1-yl)acetamido)butanoate (Compound 56-OMe)

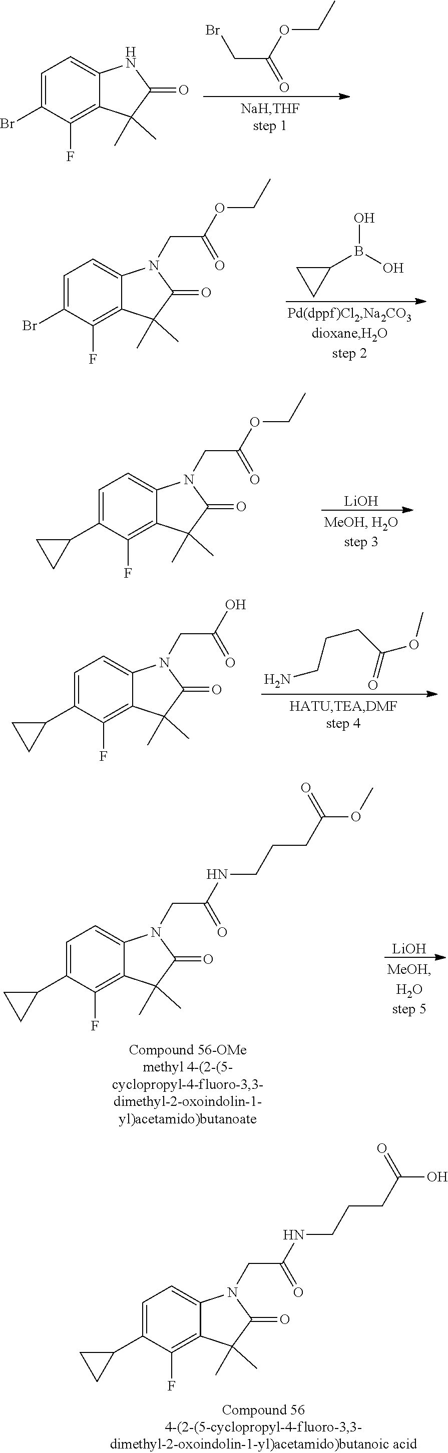

Step 1: A solution of ethyl bromoacetate (1.29 g, 7.75 mmol, 2 equiv) in tetrahydrofuran (THF) (10 mL) was treated with NaH (186 mg, 7.75 mmol, 2 equiv) for 30 min at 0° C. under nitrogen atmosphere followed by the addition of 5-bromo-4-fluoro-3,3-dimethyl-1H-indol-2-one (1.0 g, 3.88 mmol, 1 equiv) (Compound 36a of Fensome et al., J. Med. Chem. (2008) 1861-1873) dropwise at room temperature. The resulting mixture was stirred for 1 h at room temperature under nitrogen atmosphere. The reaction progress was monitored by LCMS. The reaction was quenched by the addition of water (100 mL) at 0° C. The resulting mixture was extracted with ethyl acetate (EtOAc) (3×100 mL). The combined organic layers were washed with water (3×100 mL) and dried over anhydrous Na₂SO₄. After filtration, the filtrate was concentrated under reduced pressure. The residue was purified by silica gel column chromatography, eluting with petroleum ether/ethyl acetate (1:1), to afford ethyl 2-(5-bromo-4-fluoro-3,3-dimethyl-2-oxoindol-1-yl)acetate (900 mg, 54% yield) as a white solid. LCMS: (ES, m/z): RT=0.996 min, m/z=344[M+1]⁺.

Step 2: To a stirred solution of ethyl 2-(5-bromo-4-fluoro-3,3-dimethyl-2-oxoindol-1-yl)acetate (200 mg, 0.58 mmol, 1 equiv) and cyclopropylboronic acid (250 mg, 2.9 mmol, 5 equiv) in dioxane (5 mL) and H₂O (1 mL) was added [1,1′-bis(diphenylphosphino)ferrocene]dichloropalladium(II) complex with dichloromethane (Pd(dppf)Cl₂—DCM) (85.0 mg, 0.11 mmol, 0.2 equiv) and Na₂CO₃ (185 mg, 1.74 mmol, 3 equiv). The resulting mixture was stirred for 2 h at 80° C. under nitrogen atmosphere. The reaction was monitored by LCMS. The resulting mixture was diluted with water (20 mL). The aqueous layer was extracted with ethyl acetate (EtOAc) (3×20 mL). The combined organic layers were washed with brine (1×20 mL) and dried over anhydrous Na₂SO₄. After filtration, the filtrate was concentrated under reduced pressure. The residue was purified by silica gel column chromatography, eluting with petroleum ether/ethyl acetate (5:1), to afford ethyl 2-(5-cyclopropyl-4-fluoro-3,3-dimethyl-2-oxoindol-1-yl)acetate (90 mg, 48% yield) as an off-white solid. LCMS: (ES, m/z): RT=1.03 min, m/z=306[M+H]⁺.

Step 3: To a stirred solution of ethyl 2-(5-cyclopropyl-4-fluoro-3,3-dimethyl-2-oxoindol-1-yl)acetate (100 mg, 0.32 mmol, 1 equiv) in MeOH (5 mL) and H₂O (1 mL) was added LiOH (39.2 mg, 1.63 mmol, 5 equiv). The resulting solution was stirred for 2 h at room temperature. The reaction progress was monitored by LCMS. The resulting mixture was concentrated under reduced pressure. The resulting mixture was diluted with water (4 mL) and the mixture was adjusted to pH=5 with HCl (aq. 1M). The precipitated solids were collected by filtration and washed with water (3×5 mL) to provide (5-cyclopropyl-4-fluoro-3,3-dimethyl-2-oxoindol-1-yl)acetic acid (80 mg, 88% yield) as an off white solid. LCMS: (ES, m/z): RT=1.03 min, m/z=278[M+H]⁺.

Step 4: To a stirred solution of (5-cyclopropyl-4-fluoro-3,3-dimethyl-2-oxoindol-1-yl)acetic acid (100 mg, 0.36 mmol, 1 equiv) and methyl 4-aminobutanoate (50.7 mg, 0.433 mmol, 1.2 equiv) in N,N-dimethyl formamide (DMF) (2 mL) was added triethylamine (TEA) (73.0 mg, 0.72 mmol, 2 equiv) and [bis(dimethylamino)methylene]-1H-1,2,3-triazolo[4,5-b]pyridinium 3-oxide hexafluorophosphate (HATU) (165 mg, 0.43 mmol, 1.20 equiv). The resulting solution was stirred for 2 h at room temperature. The reaction progress was monitored by LCMS. The resulting mixture was quenched with water (20 mL). The aqueous layer was extracted with ethyl acetate (EtOAc) (3×20 mL). The combined organic layers were washed with brine (1×20 mL) and dried over anhydrous Na₂SO₄. After filtration, the filtrate was concentrated under reduced pressure, and the residue was purified by silica gel column chromatography, eluting with petroleum ether/ethyl acetate (3:1), to afford methyl 4-[2-(5-cyclopropyl-4-fluoro-3,3-dimethyl-2-oxoindol-1-yl)acetamido]butanoate (also referred to as methyl 4-(2-(5-cyclopropyl-4-fluoro-3,3-dimethyl-2-oxoindolin-1-yl)acetamido)butanoate; Compound 56-OMe) (120 mg, 71% yield) as a yellow oil. LCMS: (ES, m/z): RT=0.98 min, m/z=377[M+H]⁺.

Step 5: To a stirred solution of 4-[2-(5-cyclopropyl-4-fluoro-3,3-dimethyl-2-oxoindol-1-yl)acetamido]butanoate (100 mg, 0.27 mmol, 1 equiv) in MeOH (2.50 mL) and H₂O (0.50 mL) was added LiOH (32.4 mg, 1.35 mmol, 5 equiv). The resulting mixture was stirred for 2 h at room temperature. The reaction progress was monitored by LCMS. The resulting mixture was concentrated under vacuum. The resulting mixture was diluted with water (4 mL) and acidified to pH=5 with HCl (aq. 1M). The aqueous layer was extracted with ethyl acetate (EtOAc) (3×10 mL), and the combined organic layers were washed with brine (1×20 mL) and dried over anhydrous Na₂SO₄. After filtration, the filtrate was concentrated under reduced pressure, and the crude product was purified by Prep-HPLC (Xselect CSH C18 OBD Column 30*150 mm 5 μm, n; Mobile Phase A: acetonitrile (MeCN), Mobile Phase B: water (0.05% TFA); Flow rate: 60 mL/min; Gradient: 31% B to 41% B in 10 min; Wave Length: 254/220 nm; HPLC RT(min): 8.53) to provide 4-(2-(5-cyclopropyl-4-fluoro-3,3-dimethyl-2-oxoindolin-1-yl)acetamido)butanoic acid (Compound 56) (27.1 mg, 28% yield) as a white solid. LCMS: (ES, m/z): RT=0.78 min, m/z=363[M+H]⁺. ¹H NMR (400 MHz, DMSO-d₆) δ 8.17 (t, J=5.6 Hz, 1H), 6.89 (t, J=7.9 Hz, 1H), 6.64 (d, J=8.1 Hz, 1H), 4.27 (s, 2H), 3.08 (q, J=6.6 Hz, 2H), 2.23 (t, J=7.4 Hz, 2H), 1.98 (m, 1H), 1.64 (m, 2H), 1.38 (s, 6H), 0.96-0.85 (m, 2H), 0.69-0.61 (m, 2H).

### Example 14: 4-(2-(5-cyclopropyl-4,7-difluoro-3,3-dimethyl-2-oxoindolin-1-yl)acetamido)butanoic acid (Compound 57) and tert-butyl 4-(2-(5-cyclopropyl-4,7-difluoro-3,3-dimethyl-2-oxoindolin-1-yl)acetamido)butanoate (Compound 57-OtBu)

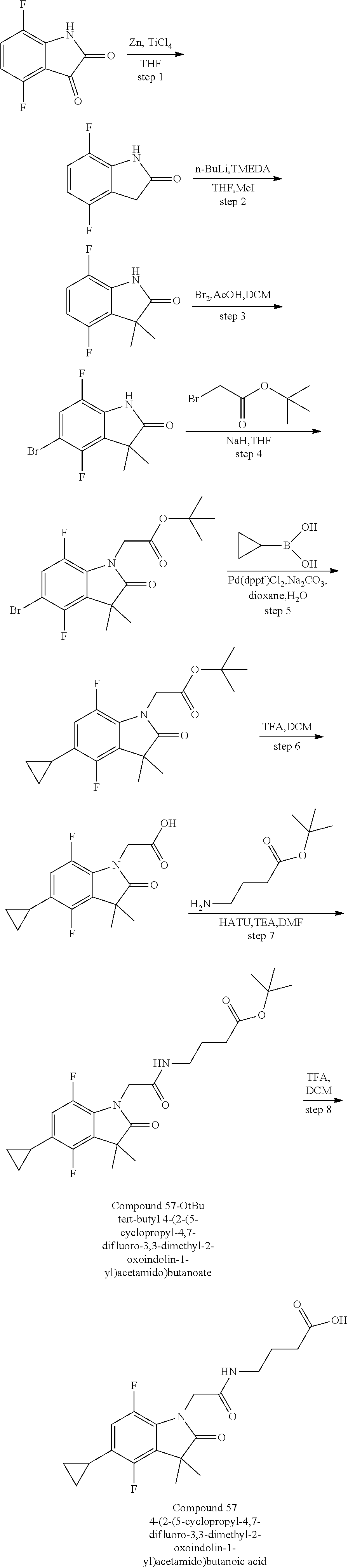

Step 1: To a stirred mixture of Zn powder (3.21 g, 49.1 mmol, 9 equiv) in THF (100 mL) was added TiCl₄ (3.79 mL, 27.3 mmol, 5 equiv) in portions at 0° C. under nitrogen atmosphere. The resulting mixture was stirred for 2 h at 70° C. under nitrogen atmosphere. To the above mixture was added 4,7-difluoro-1H-indole-2,3-dione (1 g, 5.46 mmol, 1 equiv) by dropwise at room temperature. The resulting mixture was stirred for additional 12 h at room temperature. The reaction was monitored by LCMS. The reaction was quenched with water (150 mL). The resulting mixture was extracted with CH₂Cl₂ (3×300 mL) and dried over anhydrous Na₂SO₄. After filtration, the filtrate was concentrated under reduced pressure. The residue was purified by silica gel column chromatography, eluting with petroleum ether/ethyl acetate (9:1), to afford 4,7-difluoro-1,3-dihydroindol-2-one (450 mg, 27% yield) as a red solid. LCMS (LCMS, ESI): RT=0.72 min, m/z=168 [M−H]⁻; ¹H NMR (400 MHz, DMSO-d₆) δ 11.05 (s, 1H), 7.15 (td, J=9.5, 4.2 Hz, 1H), 7.03 (s, 1H), 3.42 (td, J=6.3, 5.0 Hz, 2H).

Step 2: A solution of 4,7-difluoro-1,3-dihydroindol-2-one (450 mg, 3.25 mmol, 1 equiv) and tetramethylethylenediamine (TMEDA) (805 mg, 8.26 mmol, 2.5 equiv) in tetrahydrofuran (THF) (50 mL) was treated with n-butyl lithium (n-BuLi) (3.3 mL, 8.26 mmol, 2.5 equiv, 2.5M in THF) for 30 min at 0° C. under nitrogen atmosphere followed by the addition of methyl iodide (Mel) (785 mg, 6.52 mmol, 2 equiv) by dropwise at room temperature. The resulting mixture was stirred for 1 h at room temperature under nitrogen atmosphere. The reaction was monitored by LCMS. The reaction was quenched by the addition of water (200 mL) at room temperature. The resulting mixture was extracted with ethyl acetate (EtOAc) (3×150 mL) and dried over anhydrous Na₂SO₄. After filtration, the filtrate was concentrated under reduced pressure and the residue was purified by silica gel column chromatography, eluting with petroleum ether/ethyl acetate (6:1), to afford 4,7-difluoro-3,3-dimethyl-1H-indol-2-one (230 mg, 41% yield) as a yellow solid. LCMS (ESI): RT=0.92 min, m/z=196 [M−H]⁻; ¹H NMR (400 MHz, DMSO-d₆) δ 11.07 (s, 1H), 7.16 (t, J=9.4 Hz, 1H), 6.80 (t, J=9.0 Hz, 1H), 1.36 (s, 6H).

Step 3: To a stirred mixture of 4,7-difluoro-3,3-dimethyl-1H-indol-2-one (230 mg, 1.31 mmol, 1 equiv) and acetic acid (AcOH) (2 mL) in dichloromethane (DCM) (10 mL) was added Br₂ (1.05 g, 6.59 mmol, 5 equiv, in 3 mL DCM) by dropwise at 0° C. The resulting mixture was stirred for 1 h at room temperature under nitrogen atmosphere. The reaction was monitored by LCMS, and upon completion, was quenched by the addition of sat. NaHCO₃ (aq.) (50 mL) at room temperature. The resulting mixture was extracted with ethyl acetate (EtOAc) (3×200 mL) and dried over anhydrous Na₂SO₄. After filtration, the filtrate was concentrated under reduced pressure to provide a residue, which was purified by silica gel column chromatography, eluting with petroleum ether/ethyl acetate (5:1), to afford 5-bromo-4,7-difluoro-3,3-dimethyl-1H-indol-2-one (300 mg, 69% yield) as an off-white solid. LCMS (LCMS, ESI): RT=1.05 min, m/z=274 [M−H]⁻; ¹H NMR (400 MHz, DMSO-d₆) δ 11.23 (s, 1H), 7.63 (d, J=9.1 Hz, 1H), 1.37 (s, 6H).

Step 4: A solution of 5-bromo-4,7-difluoro-3,3-dimethyl-1H-indol-2-one (320 mg, 1.15 mmol, 1 equiv) in tetrahydrofuran (THF) (15 mL) was treated with NaH (dispersion in mineral oil) (83.5 mg, 3.47 mmol, 3 equiv) for 30 min at 0° C. under nitrogen atmosphere followed by the addition of tert-butyl 2-bromoacetate (678 mg, 3.47 mmol, 3 equiv) by dropwise at 0° C. The resulting mixture was stirred for 1 h at room temperature. The reaction was monitored by LCMS. The reaction was quenched by the addition of water (100 mL) at room temperature. The resulting mixture was extracted with ethyl acetate (EtOAc) (3×100 mL) and dried over anhydrous Na₂SO₄. After filtration, the filtrate was concentrated under reduced pressure to provide a residue, which was purified by silica gel column chromatography, eluting with petroleum ether/ethyl acetate (6:1), to afford tert-butyl 2-(5-bromo-4,7-difluoro-3,3-dimethyl-2-oxoindol-1-yl)acetate (400 mg, 88% yield) as an off-white solid. LCMS (LCMS, ESI): RT=1.35 min, m/z=388 [M−H]⁻.

Step 5: Into a 20 mL sealed tube was added tert-butyl 2-(5-bromo-4,7-difluoro-3,3-dimethyl-2-oxoindol-1-yl)acetate (300 mg, 0.76 mmol, 1 equiv), cyclopropylboronic acid (250 mg, 2.9 mmol, 5 equiv), [1,1′-bis(diphenylphosphino)ferrocene] dichloropalladium(II) complex with dichloromethane (Pd(dppf)Cl₂—DCM) (113 mg, 0.15 mmol, 0.2 equiv), Na₂CO₃ (245 mg, 2.30 mmol, 3 equiv), dioxane (7 mL) and H₂O (1 mL) at room temperature. The resulting mixture was stirred for 12 h at 80° C. under nitrogen atmosphere. The reaction was monitored by LCMS. The reaction was quenched by the addition of water (100 mL) at room temperature. The resulting mixture was extracted with ethyl acetate (EtOAc) (3×150 mL) and dried over anhydrous Na₂SO₄. After filtration, the filtrate was concentrated under reduced pressure, and the residue was purified by silica gel column chromatography, eluting with petroleum ether/ethyl acetate (7:1), to afford tert-butyl 2-(5-cyclopropyl-4,7-difluoro-3,3-dimethyl-2-oxoindol-1-yl)acetate (250 mg, 79% yield) as a brown solid. LCMS (LCMS, ESI): RT=1.34 min, m/z=296 [M−H]⁻.

Step 6: Into a 8 mL sealed tube was added tert-butyl 2-(5-cyclopropyl-4,7-difluoro-3,3-dimethyl-2-oxoindol-1-yl)acetate (200 mg, 0.58 mmol, 1 equiv), trifluoroacetic acid (TFA) (2 mL) and dichloromethane (DCM) (5 mL) at room temperature. The resulting mixture was stirred for 1 h at room temperature. The reaction was monitored by LCMS. The resulting mixture was concentrated under reduced pressure. The reaction was quenched by the addition of water (100 mL) at room temperature. The resulting mixture was extracted with ethyl acetate (EtOAc) (3×150 mL) and dried over anhydrous Na₂SO₄. After filtration, the filtrate was concentrated under reduced pressure to 2-(5-cyclopropyl-4,7-difluoro-3,3-dimethyl-2-oxoindolin-1-yl)acetic acid (180 mg, 68% yield) as a white solid. LCMS (LCMS, ESI): RT=0.89 min, m/z=296 [M+H]⁺; ¹H NMR (400 MHz, DMSO-d₆) δ 13.17 (s, 1H), 6.84 (d, J=12.2 Hz, 1H), 4.56-4.43 (m, 2H), 1.99 (q, J=4.5, 3.3 Hz, 1H), 1.40 (s, 6H), 0.94 (d, J=8.4 Hz, 2H), 0.76-0.68 (m, 2H).

Step 7: Into a 8 mL sealed tube was added 2-(5-cyclopropyl-4,7-difluoro-3,3-dimethyl-2-oxoindolin-1-yl)acetic acid (180 mg, 0.67 mmol, 1 equiv), tert-butyl 4-aminobutanoate hydrogen chloride (396 mg, 2.03 mmol, 3 equiv), [bis(dimethylamino)methylene]-1H-1,2,3-triazolo[4,5-b]pyridinium 3-oxide hexafluorophosphate (HATU) (309 mg, 0.81 mmol, 1.20 equiv), triethylamine (TEA) (343 mg, 3.38 mmol, 5 equiv) and N,N-dimethyl formamide (DMF) (4 mL) at room temperature. The resulting mixture was stirred for 1 h at room temperature. The reaction was monitored by LCMS. The reaction was quenched by the addition of water (50 mL) at room temperature. The resulting mixture was extracted with ethyl acetate (EtOAc) (3×50 mL) and dried over anhydrous Na₂SO₄. After filtration, the filtrate was concentrated under reduced pressure to provide a residue, which was purified by reverse flash chromatography (C18 silica gel; mobile phase, acetonitrile (MeCN) in water, 30% to 45% gradient in 10 min; detector, UV 254 nm/220 nm) to provide tert-butyl 4-[2-(5-cyclopropyl-4,7-difluoro-3,3-dimethyl-2-oxoindol-1-yl)acetamido]butanoate (also referred to as tert-butyl 4-(2-(5-cyclopropyl-4,7-difluoro-3,3-dimethyl-2-oxoindolin-1-yl)acetamido)butanoate; Compound 57-OtBu) (200 mg, 68% yield) as a white solid. LCMS (LCMS, ESI): RT=1.03 min, m/z=437 [M+H]⁺.

Step 8: Into a 20 mL sealed tube was added tert-butyl 4-[2-(5-cyclopropyl-4,7-difluoro-3,3-dimethyl-2-oxoindol-1-yl)acetamido]butanoate (150 mg, 0.34 mmol, 1 equiv), trifluoroacetic acid (TFA) (1.3 mL) and dichloromethane (DCM) (5 mL) at room temperature. The resulting mixture was stirred for 1 h at room temperature. The reaction was monitored by LCMS. The resulting mixture was concentrated under reduced pressure. The reaction was quenched by the addition of water (50 mL) at room temperature. The resulting mixture was extracted with ethyl acetate (EtOAc) (3×50 mL), dried over anhydrous Na₂SO₄. After filtration, the filtrate was concentrated under reduced pressure. The crude product (100 mg, 80% purity) was purified by Prep-HPLC (XBridge Shield RP18 OBD Column, 30*150 mm, 5 μm; Mobile Phase A: water (10 mmol/L NH₄HCO₃), Mobile Phase B: acetonitrile (MeCN); Flow rate: 60 mL/min; Gradient: 20% B to 30% B in 10 min, 30% B; Wave Length: 254 nm; RT1 (min): 8.5) to provide 4-(2-(5-cyclopropyl-4,7-difluoro-3,3-dimethyl-2-oxoindolin-1-yl)acetamido)butanoic acid (Compound 57) (70 mg, 38% yield) as a white solid. LCMS (ES, m/z): RT=0.72 min, m/z=381 [M+H]⁺; ¹H NMR (400 MHz, DMSO-d₆) δ 8.32 (s, 1H), 6.78 (d, J=12.0 Hz, 1H), 4.32 (d, J=1.9 Hz, 2H), 3.06 (q, J=6.5 Hz, 2H), 2.11 (t, J=7.3 Hz, 2H), 1.99 (d, J=13.5 Hz, 1H), 1.61 (d, J=7.2 Hz, 2H), 1.40 (s, 6H), 0.98-0.89 (m, 2H), 0.74-0.66 (m, 2H).

### Example 15: 4-(2-(5-cyclopropyl-4-fluoro-3,3-dimethyl-2-oxoindolin-1-yl)acetamido)-3-methylpentanoic acid (Compound 58, rac-58), (3S,4R)-4-(2-(5-cyclopropyl-4-fluoro-3,3-dimethyl-2-oxoindolin-1-yl)acetamido)-3-methylpentanoic acid (Compound 58A*), (3R,4S)-4-(2-(5-cyclopropyl-4-fluoro-3,3-dimethyl-2-oxoindolin-1-yl)acetamido)-3-methylpentanoic acid (Compound 58B*), (3S,4S)-4-(2-(5-cyclopropyl-4-fluoro-3,3-dimethyl-2-oxoindolin-1-yl)acetamido)-3-methylpentanoic acid (Compound 58C*), (3R,4R)-4-(2-(5-cyclopropyl-4-fluoro-3,3-dimethyl-2-oxoindolin-1-yl)acetamido)-3-methylpentanoic acid (Compound 58D*), tert-butyl (3S,4R)-4-(2-(5-cyclopropyl-4-fluoro-3,3-dimethyl-2-oxoindolin-1-yl)acetamido)-3-methylpentanoate (Compound 58A*-OtBu), tert-butyl (3R,4S)-4-(2-(5-cyclopropyl-4-fluoro-3,3-dimethyl-2-oxoindolin-1-yl)acetamido)-3-methylpentanoate (Compound 58B*-OtBu), tert-butyl (3S,4S)-4-(2-(5-cyclopropyl-4-fluoro-3,3-dimethyl-2-oxoindolin-1-yl)acetamido)-3-methylpentanoate (Compound 58C*-OtBu), and tert-butyl (3R,4R)-4-(2-(5-cyclopropyl-4-fluoro-3,3-dimethyl-2-oxoindolin-1-yl)acetamido)-3-methylpentanoate (Compound 58D*-OtBu)

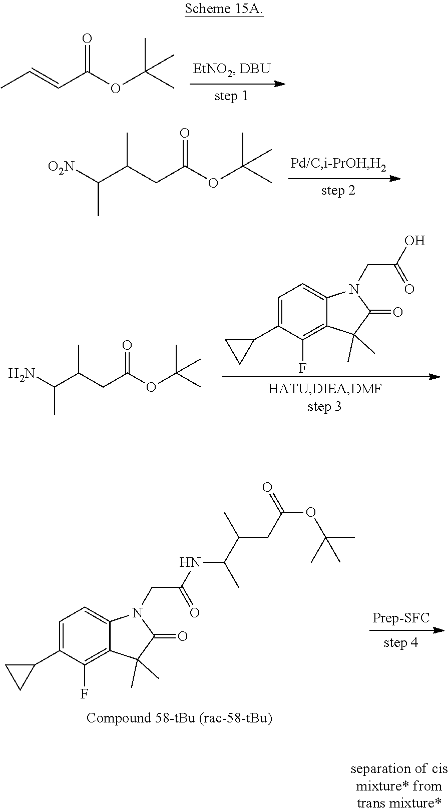

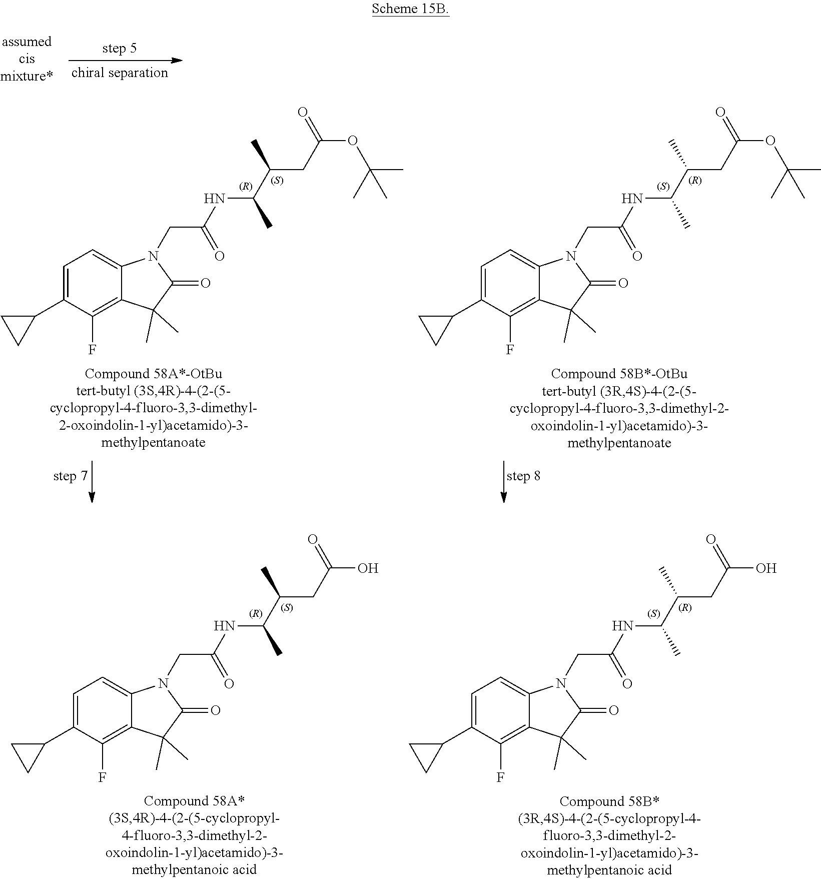

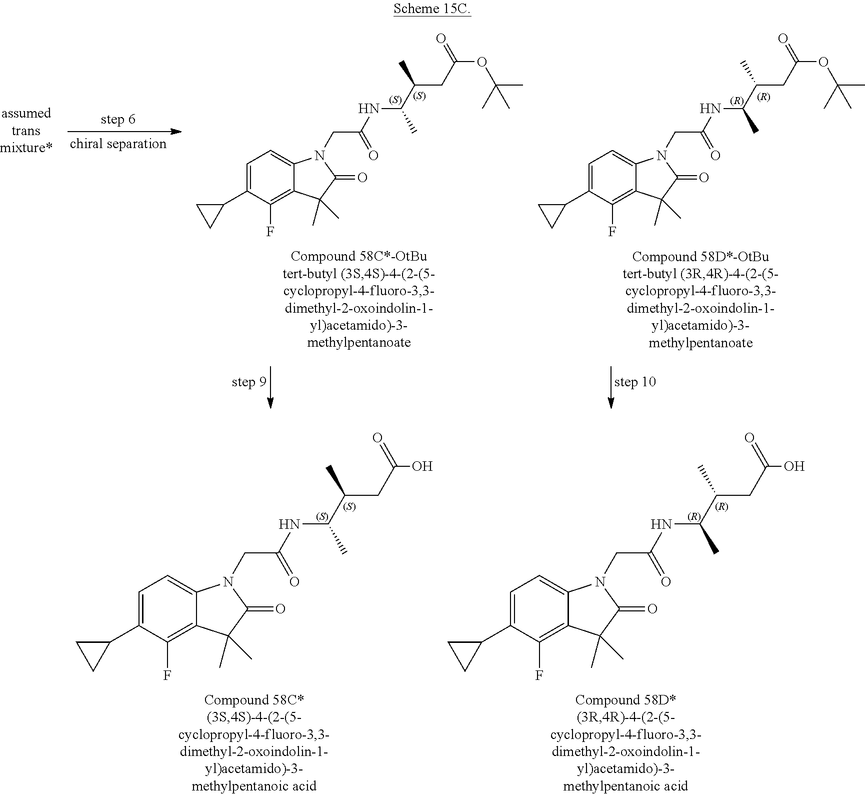

Step 1: Into a 40 mL round-bottom flask was added tert-butyl (2E)-but-2-enoate (8 g, 56.3 mmol, 1 equiv), 1,8-diazabicyclo(5.4.0)undec-7-ene (DBU) (12.9 g, 84.4 mmol, 1.5 equiv), acetonitrile (MeCN) (20 mL) and nitroethane (EtNO₂) (21.1 g, 281 mmol, 5 equiv) at room temperature. The mixture was stirred for 1 h at 50° C. The reaction progress was monitored by LCMS. The reaction was quenched by the addition of water (30 mL) at room temperature. The aqueous layer was extracted with ethyl acetate (EtOAc) (3×20 mL). The organic phase was concentrated under reduced pressure. The residue was purified by reversed-phase flash chromatography (C18 silica gel; mobile phase, MeCN in Water, 10% to 50% gradient in 10 min; detector, UV 254/220 nm) to provide tert-butyl 3-methyl-4-nitropentanoate (5.0 g, 49% yield) as a colorless oil. LCMS: (ES, m/z): RT=0.61 min, m/z=162[M+1]⁺. ¹H NMR (400 MHz, DMSO-d₆) δ 3.21 (d, J=6.5 Hz, 1H), 2.55-2.26 (m, 2H), 2.22-2.05 (m, 1H), 1.61-1.50 (m, 3H), 1.41 (s, 9H), 0.90 (d, J=6.7 Hz, 3H).

Step 2: Into a 500 mL 3-necked round-bottom flask was added tert-butyl 3-methyl-4-nitropentanoate (6 g, 27.6 mmol, 1 equiv), isopropanol (i-PrOH) (250 mL) and Pd/C (8.14 g) at room temperature, and the mixture was stirred for overnight at room temperature under hydrogen (H₂) atmosphere. The reaction progress was monitored by LCMS. The resulting mixture was filtered, the filter cake was washed with i-PrOH (3×10 mL). To the filtrate was added hydrogen chloride (2M, 1 mL), and the reaction was concentrated under reduced pressure to provide tert-butyl 4-amino-3-methylpentanoate hydrochloride (4 g, 65% yield) as a colorless oil. LCMS: (ES, m/z): RT=0.83 min, m/z=132[M+1]⁺.

Step 3: Into a 250 mL round-bottom flask was added tert-butyl 4-amino-3-methylpentanoate hydrochloride (4 g, 21.4 mmol, 1 equiv), [bis(dimethylamino)methylene]-1H-1,2,3-triazolo[4,5-b]pyridinium 3-oxide hexafluorophosphate (HATU) (12.2 g, 32.0 mmol, 1.5 equiv), N,N-diisopropylethylamine (DIPEA) (8.28 g, 64.1 mmol, 3 equiv), N,N-dimethyl formamide (DMF) (20 mL) and (5-cyclopropyl-4-fluoro-3,3-dimethyl-2-oxoindol-1-yl)acetic acid (product of Step 3, Example 13) (3.20 g, 11.5 mmol, 0.54 equiv) at room temperature. The reaction progress was monitored by LCMS. The reaction was quenched by the addition of water (30 mL) at room temperature. The aqueous layer was extracted with ethyl acetate (EtOAc) (3×30 mL), and the organic phase was concentrated under reduced pressure to provide a residue, which was purified by Prep-HPLC (XBridge Prep Phenyl Column, 19*250 mm, 5 μm; Mobile Phase A: water (10 mmol/L NH₄HCO₃), Mobile Phase B: acetonitrile (MeCN); Flow rate: 25 mL/min; Gradient: 25% B to 35% B in 9 min; Wave Length: 254 nm/220 nm; HPLC RT(min): 9) to provide tert-butyl 4-[2-(5-cyclopropyl-4-fluoro-3,3-dimethyl-2-oxoindol-1-yl)acetamido]-3-methylpentanoate (Compound 58-OtBu, rac-58-OtBu) (2.9 g, 30% yield) as a colorless oil, and comprising a mixture of cis and trans isomers. LCMS: (ES, m/z): RT=0.98 min, m/z=391[M+1]⁺.

Step 4: Compound 58-OtBu (2.9 g, 98% purity) was purified by Prep-SFC with the following conditions ((S, S)-Whelk-O 1 5 μm Kromasil, 3*25 cm, 5 μm; Mobile Phase A: CO₂, Mobile Phase B: isopropanol (IPA); Flow rate: 100 mL/min; Gradient: isocratic 28% B; Column Temperature (° C.): 35; Back Pressure (bar): 100; Wave Length: 220 nm) to provide a cis mixture* of tert-butyl 4-(2-(5-cyclopropyl-4-fluoro-3,3-dimethyl-2-oxoindolin-1-yl)acetamido)-3-methylpentanoate (Compound 58A-OtBu-cis) (Prep-SFC RT(min): 5.97; 0.93 g; 96% purity; 32% yield; LCMS: (ES, m/z): RT=0.64 min, m/z=391[M+1]⁺) as a white solid and to provide a trans mixture* of tert-butyl 4-(2-(5-cyclopropyl-4-fluoro-3,3-dimethyl-2-oxoindolin-1-yl)acetamido)-3-methylpentanoate (Compound 58-OtBu-trans) (Prep-SFC RT(min): 7.17; 1.1 g; 96% purity; 38% yield; LCMS: (ES, m/z): RT=0.67 min, m/z=391[M+1]⁺) as a white solid. Cis and trans stereochemistry arbitrarily assigned.

Step 5: Compound 58A-OtBu-cis (0.93 g) from Step 4 was purified by CHIRAL-Prep-HPLC (Column: CHIRAL ART Cellulose-SB, 2*25 cm, 5 μm; Mobile Phase A: Hexanes (0.1% TFA), Mobile Phase B: isopropanol (IPA); Flow rate: 20 mL/min; Gradient: 5% B to 5% B in 14 min; Wave Length: 220/254 nm) to provide tert-butyl (3S,4R)-4-(2-(5-cyclopropyl-4-fluoro-3,3-dimethyl-2-oxoindolin-1-yl)acetamido)-3-methylpentanoate (Compound 58A*-OtBu) (Chiral HPLC RT(min): 12.76; 513 mg; 55% yield; LCMS: (ES, m/z): RT=0.66 min, m/z=391[M+H]⁺) as a white solid, and tert-butyl (3R,4S)-4-(2-(5-cyclopropyl-4-fluoro-3,3-dimethyl-2-oxoindolin-1-yl)acetamido)-3-methylpentanoate (Compound 58B*-OtBu) (Chiral HPLC RT(min): 12.0; 96 mg; 10% yield; LCMS: (ES, m/z): RT=0.65 min, m/z=391.1[M+H]⁺) as a white solid. Stereochemistry was arbitrarily assigned.

Step 6: Compound 58A-OtBu-trans (1.1 g) from Step 4 was purified by CHIRAL-Prep-HPLC (CHIRAL ART Cellulose-SC, 2*25 cm, 5 μm; Mobile Phase A: Hexanes (0.1% TFA), Mobile Phase B: isopropanol (IPA); Flow rate: 20 mL/min; Gradient: 15% B to 15% B in 23 min; Wave Length: 220/254 nm) to provide tert-butyl (3S,4S)-4-(2-(5-cyclopropyl-4-fluoro-3,3-dimethyl-2-oxoindolin-1-yl)acetamido)-3-methylpentanoate (Compound 58C*-OtBu) (Chiral HPLC RT(min): 16.03; 790 mg; 72% yield; LCMS: (ES, m/z): RT=0.66 min, m/z=391.1[M+H]⁺) as a white solid and tert-butyl (3R,4R)-4-(2-(5-cyclopropyl-4-fluoro-3,3-dimethyl-2-oxoindolin-1-yl)acetamido)-3-methylpentanoate (Compound 58D*-OtBu) (Chiral HPLC RT(min): 18.95; 166 mg; 15% yield; LCMS: (ES, m/z): RT=0.65 min, m/z=391.1[M+H]⁺) as a white solid. Stereochemistry was arbitrarily assigned.

Step 7: Into a 40 mL round-bottom flask was added Compound 58A*-OtBu (513 mg, 1.15 mmol, 1 equiv), trifluoroacetic acid (TFA) (0.5 mL), and dichloromethane (DCM) (2 mL) at room temperature. The mixture was stirred for 1 h at room temperature. The reaction progress was monitored by LCMS. The resulting mixture was concentrated under reduced pressure, and further dried by lyophilization, to afford (3S,4R)-4-(2-(5-cyclopropyl-4-fluoro-3,3-dimethyl-2-oxoindolin-1-yl)acetamido)-3-methylpentanoic acid (Compound 58A*) (419 mg, 93% yield) as a white solid. LCMS: (ES, m/z): RT=1.65 min, m/z=391.1[M+H]⁺. ¹H NMR (400 MHz, Methanol-d₄) δ 6.90 (t, J=7.8 Hz, 1H), 6.64 (d, J=8.0 Hz, 1H), 4.44-4.34 (m, 2H), 4.03-3.94 (m, 1H), 2.49-2.37 (m, 1H), 2.20-2.07 (m, 2H), 2.02 (d, J=13.8 Hz, 1H), 1.50 (s, 6H), 1.16 (d, J=6.8 Hz, 3H), 1.02-0.89 (m, 5H), 0.71-0.63 (m, 2H).

Step 8: Into a 40 mL round-bottom flask was added Compound 58B*-OtBu (96 mg, 0.48 mmol, 1 equiv), trifluoroacetic acid (TFA) (0.5 mL), and dichloromethane (DCM) (2 mL) at room temperature. The mixture was stirred for 1 h at room temperature. The reaction progress was monitored by LCMS. The resulting mixture was concentrated under reduced pressure, and further dried by lyophilization, to provide (3R,4S)-4-(2-(5-cyclopropyl-4-fluoro-3,3-dimethyl-2-oxoindolin-1-yl)acetamido)-3-methylpentanoic acid (Compound 58B*) (25 mg, 26% yield) as a white solid. LCMS: (ES, m/z): RT=1.546 min, m/z=391.1[M+H]⁺. ¹H NMR (400 MHz, Methanol-d₄) δ 6.91 (t, J=7.8 Hz, 1H), 6.64 (d, J=8.0 Hz, 1H), 4.47-4.32 (m, 2H), 3.98 (t, J=6.4 Hz, 1H), 2.48-2.37 (m, 1H), 2.18-1.97 (m, 3H), 1.50 (s, 6H), 1.16 (d, J=6.8 Hz, 3H), 1.02-0.91 (m, 5H), 0.71-0.63 (m, 2H).

Step 9: Into a 40 mL round-bottom flask was added Compound 58C*-OtBu (790 mg, 1.99 mmol, 1 equiv), trifluoroacetic acid (TFA) (0.5 mL), and dichloromethane (DCM) (2 mL) at room temperature. The mixture was stirred for 1 h at room temperature. The reaction progress was monitored by LCMS. The resulting mixture was concentrated under reduced pressure, and further dried by lyophilization, to provide (3S,4S)-4-(2-(5-cyclopropyl-4-fluoro-3,3-dimethyl-2-oxoindolin-1-yl)acetamido)-3-methylpentanoic acid (Compound 59C*) (686.9 mg, 87% yield) as a white solid. LCMS: (ES, m/z): RT=1.70 min, m/z=391.1[M+H]⁺. ¹H NMR (400 MHz, Methanol-d₄) δ 6.90 (t, J=7.8 Hz, 1H), 6.64 (d, J=8.0 Hz, 1H), 4.46-4.32 (m, 2H), 4.01-3.94 (m, 1H), 2.49-2.36 (m, 1H), 2.19-2.07 (m, 2H), 2.06-1.96 (m, 1H), 1.50 (s, 6H), 1.16 (d, J=6.8 Hz, 3H), 1.02-0.89 (m, 5H), 0.71-0.63 (m, 2H).

Step 10: Into a 40 mL round-bottom flask was added Compound 58D*-OtBu (166 mg, 0.48 mmol, 1 equiv), trifluoroacetic acid (TFA) (0.5 mL), and dichloromethane (DCM) (2 mL) at room temperature. The mixture was stirred for 1 h at room temperature. The reaction progress was monitored by LCMS. The resulting mixture was concentrated under reduced pressure, and further dried by lyophilization, to provide (3R,4R)-4-(2-(5-cyclopropyl-4-fluoro-3,3-dimethyl-2-oxoindolin-1-yl)acetamido)-3-methylpentanoic acid (Compound 58D*) (136.4 mg, 82% yield) as a white solid. LCMS: (ES, m/z): RT=1.22 min, m/z=391.1[M+H]⁺. ¹H NMR (400 MHz, Methanol-d₄) δ 6.91 (t, J=7.8 Hz, 1H), 6.64 (d, J=8.0 Hz, 1H), 4.38 (d, J=3.6 Hz, 2H), 3.83 (t, J=6.4 Hz, 1H), 2.46 (d, J=8.4 Hz, 1H), 2.13-1.97 (m, 3H), 1.50 (s, 6H), 1.16 (d, J=6.4 Hz, 3H), 1.04-0.91 (m, 5H), 0.71-0.62 (m, 2H).

### Example 16: (S)-4-(2-(5-cyclopropyl-4,7-difluoro-3,3-dimethyl-2-oxoindolin-1-yl)acetamido)pentanoic acid (Compound 59A) and (R)-4-(2-(5-cyclopropyl-4,7-difluoro-3,3-dimethyl-2-oxoindolin-1-yl)acetamido)pentanoic acid (Compound 59B), methyl (S)-4-(2-(5-cyclopropyl-4,7-difluoro-3,3-dimethyl-2-oxoindolin-1-yl)acetamido)pentanoate (Compound 59A-OMe) and methyl (R)-4-(2-(5-cyclopropyl-4,7-difluoro-3,3-dimethyl-2-oxoindolin-1-yl)acetamido)pentanoate (Compound 59B-OMe)

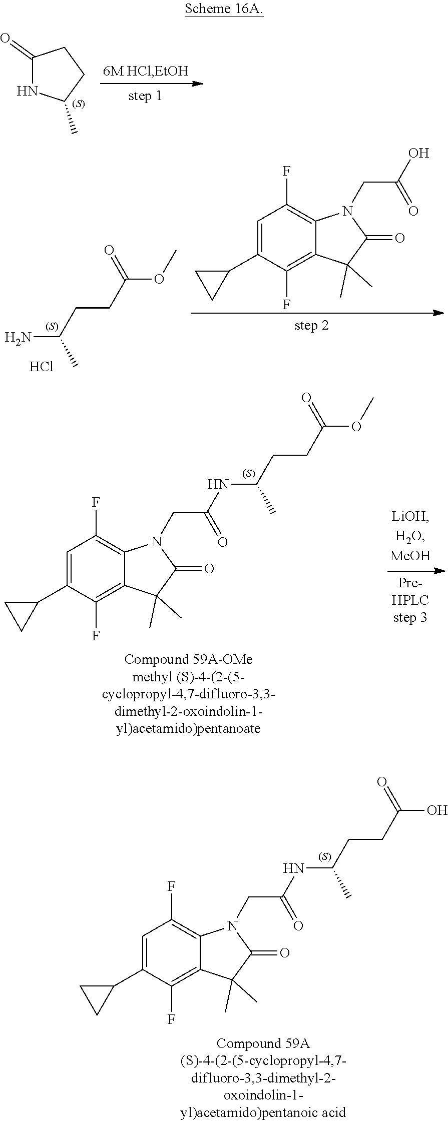

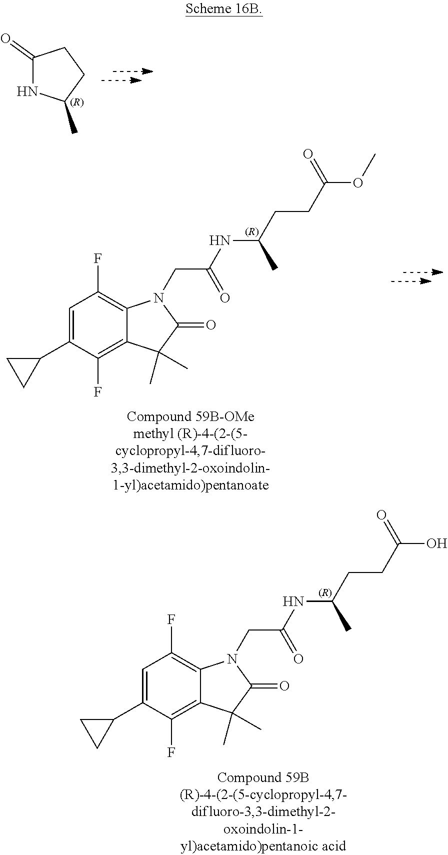

Step 1: Into a 25 mL sealed tube was added (5S)-5-methylpyrrolidin-2-one (1 g, 10.1 mmol, 1 equiv), HCl (aq, 6M) (10 mL) and MeOH (10 mL), The solution was stirred for 12 h at 100° C. The reaction progress was monitored by LCMS, and upon completion, the resulting mixture was concentrated under reduced pressure to provide methyl (4S)-4-aminopentanoate hydrogen chloride (900 mg, 68% yield) as a yellow oil. LCMS:(ES, m/z): RT=0.74 min, m/z=132[M+H]⁺.

Step 2: Into a 50 mL round-bottom flask was added methyl (4S)-4-aminopentanoate hydrogen chloride (850 mg, 6.48 mmol, 1 equiv), 2-(5-cyclopropyl-4,7-difluoro-3,3-dimethyl-2-oxoindolin-1-yl)acetic acid (product of step 6, Example 14) (1.90 g, 6.48 mmol, 1 equiv), [bis(dimethylamino)methylene]-1H-1,2,3-triazolo[4,5-b]pyridinium 3-oxide hexafluorophosphate (HATU) (3.20 g, 8.42 mmol, 1.3 equiv), N,N-diisopropylethylamine (DIPEA) (2.51 g, 19.44 mmol, 3 equiv) and N,N-dimethyl formamide (DMF) (10 mL). The solution was stirred for 1 h at room temperature. The reaction progress was monitored by LCMS. The reaction was quenched by the addition of water (10 mL) at room temperature. The aqueous layer was extracted with ethyl acetate (EtOAc) (3×10 mL). The resulting mixture was concentrated under reduced pressure. The residue was purified by silica gel column chromatography, eluting with petroleum ether/ethyl acetate (5:1), to afford methyl (4S)-4-[2-(5-cyclopropyl-4,7-difluoro-3,3-dimethyl-2-oxoindol-1-yl)acetamido]pentanoate (also referred to as methyl (S)-4-(2-(5-cyclopropyl-4,7-difluoro-3,3-dimethyl-2-oxoindolin-1-yl)acetamido)pentanoate, Compound 59A-OMe) (750 mg, 28% yield) as a yellow oil. LCMS:(ES, m/z): RT=0.84 min, m/z=409[M+H]⁺. ¹H NMR (400 MHz, DMSO-d₆) δ 8.06-7.94 (m, 1H), 6.79 (d, J=12.1 Hz, 1H), 4.32 (s, 2H), 3.74 (d, J=7.1 Hz, 1H), 3.58 (s, 3H), 2.90 (s, 2H), 2.34-2.25 (m, 2H), 1.98 (d, J=8.6 Hz, 1H), 1.40 (s, 6H), 1.05 (d, J=6.6 Hz, 3H), 0.99-0.88 (m, 2H), 0.75-0.66 (m, 2H).

Step 3: Into a 25 mL round-bottom flask was added methyl (4S)-4-[2-(5-cyclopropyl-4,7-difluoro-3,3-dimethyl-2-oxoindol-1-yl)acetamido]pentanoate (700 mg, 1.71 mmol, 1 equiv) in methanol (MeOH) (6 mL) was added LiOH (61.6 mg, 2.57 mmol, 1.50 equiv) in H₂O (2 mL) at 0° C. The solution was stirred for 1 h at room temperature. The reaction progress was monitored by LCMS. The mixture was adjusted to pH 6 with HCl (aq, 1M). The aqueous layer was extracted with ethyl acetate (EtOAc) (3×10 mL), and then concentrated under reduced pressure to provide (4S)-4-[2-(5-cyclopropyl-4,7-difluoro-3,3-dimethyl-2-oxoindol-1-yl)acetamido]pentanoic acid (500 mg, 80% purity) as a white solid. The crude product was purified by Prep-HPLC (XBridge Prep OBD C18 Column, 30*150 mm, 5 μm; Mobile Phase A: water (10 mmol/L NH₄HCO₃), Mobile Phase B: acetonitrile (MeCN); Flow rate: 60 mL/min; Gradient: isocratic 15-25; Wave Length: 254 nm/220 nm; HPLC RT(min): 11.8) to afford (S)-4-(2-(5-cyclopropyl-4,7-difluoro-3,3-dimethyl-2-oxoindolin-1-yl)acetamido)pentanoic acid (Compound 59A) (371 mg, 77% yield) as a white solid. LCMS:(ES, m/z): RT=1.42 min, m/z=395[M+H]⁺. ¹H NMR (400 MHz, Methanol-d₄) δ 6.69 (d, J=12.0 Hz, 1H), 4.56-4.41 (m, 2H), 3.88 (q, J=6.8 Hz, 1H), 2.31-2.21 (m, 2H), 2.24-2.14 (m, 1H), 2.03 (d, J=13.8 Hz, 2H), 1.51 (s, 6H), 1.17 (d, J=6.5 Hz, 3H), 1.03-0.94 (m, 2H), 0.69 (t, J=6.4 Hz, 2H).

(R)-4-(2-(5-cyclopropyl-4,7-difluoro-3,3-dimethyl-2-oxoindolin-1-yl)acetamido)pentanoic acid (Compound 59B) and methyl (R)-4-(2-(5-cyclopropyl-4,7-difluoro-3,3-dimethyl-2-oxoindolin-1-yl)acetamido)pentanoate (Compound 59B-OMe) may be prepared following steps 1-3 of Example 16 using (5R)-5-methylpyrrolidin-2-one instead of (5S)-5-methylpyrrolidin-2-one as the starting material in Step 1.

### Example 17: (3S,4S)-4-cyclopropyl-4-(2-(5-cyclopropyl-4-fluoro-3,3-dimethyl-2-oxoindolin-1-yl)acetamido)-3-methylbutanoic acid (Compound 60A*), (3R,4R)-4-cyclopropyl-4-(2-(5-cyclopropyl-4-fluoro-3,3-dimethyl-2-oxoindolin-1-yl)acetamido)-3-methylbutanoic acid (Compound 60B*), (3S,4R)-4-cyclopropyl-4-(2-(5-cyclopropyl-4-fluoro-3,3-dimethyl-2-oxoindolin-1-yl)acetamido)-3-methylbutanoic acid (Compound 60C*), (3R,4S)-4-cyclopropyl-4-(2-(5-cyclopropyl-4-fluoro-3,3-dimethyl-2-oxoindolin-1-yl)acetamido)-3-methylbutanoic acid (Compound 60D*), tert-butyl (3S,4S)-4-cyclopropyl-4-(2-(5-cyclopropyl-4-fluoro-3,3-dimethyl-2-oxoindolin-1-yl)acetamido)-3-methylbutanoate (Compound 60A*-OtBu), tert-butyl (3R,4R)-4-cyclopropyl-4-(2-(5-cyclopropyl-4-fluoro-3,3-dimethyl-2-oxoindolin-1-yl)acetamido)-3-methylbutanoate (Compound 60B*-OtBu), tert-butyl (3S,4R)-4-cyclopropyl-4-(2-(5-cyclopropyl-4-fluoro-3,3-dimethyl-2-oxoindolin-1-yl)acetamido)-3-methylbutanoate (Compound 60C*-OtBu), and tert-butyl (3R,4S)-4-cyclopropyl-4-(2-(5-cyclopropyl-4-fluoro-3,3-dimethyl-2-oxoindolin-1-yl)acetamido)-3-methylbutanoate (Compound 60D*-OtBu)

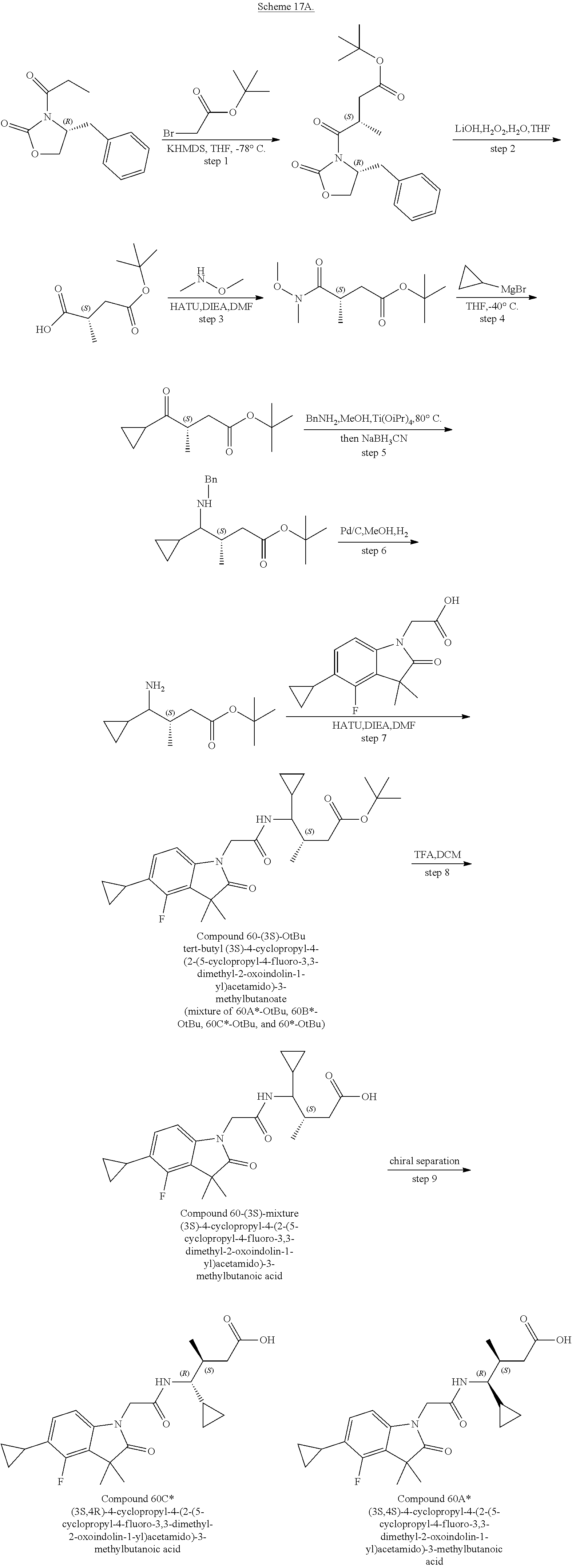

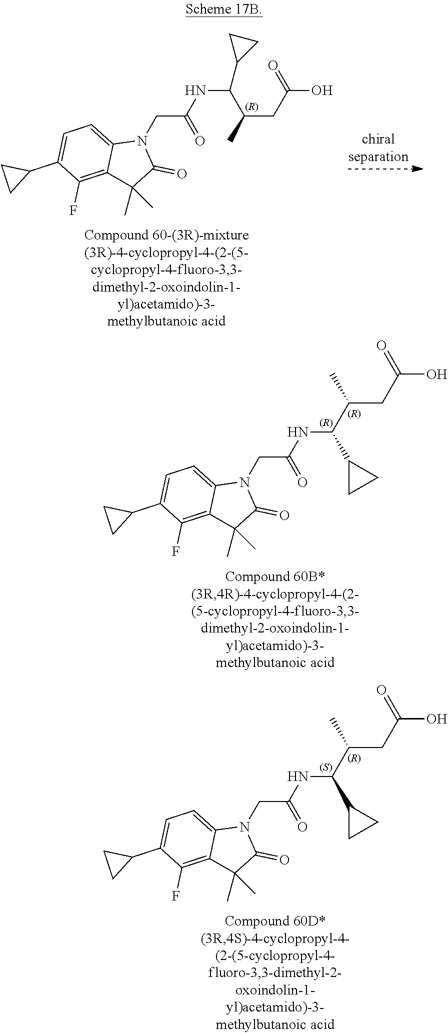

Step 1: Into a 250 mL 3-necked round-bottom flask was added (4R)-4-benzyl-3-propanoyl-1,3-oxazolidin-2-one (5 g, 21.4 mmol, 1 equiv) and tetrahydrofuran (THF) (50 mL) at room temperature. Then added potassium bis(trimethylsilyl)amide (KHMDS) (32.0 mL, 32.2 mmol, 1.5 equiv, 1M in THF) at −78° C. The resulting mixture was stirred for 30 min at −78° C. under nitrogen atmosphere, and then tert-butyl 2-bromoacetate (6.27 g, 32.2 mmol, 1.5 equiv) was added at −78° C., and the reaction further stirred for 30 min at room temperature under nitrogen atmosphere. The reaction was monitored by LCMS, and upon completion, quenched by the addition of water (30 mL) at 0° C. The resulting mixture was extracted with ethyl acetate (EtOAc) (3×20 mL), the combined organic layers were dried over anhydrous Na₂SO₄, filtered, and the filtrate was concentrated under reduced pressure to provide a residue which was purified by silica gel column chromatography, eluting with petroleum ether/ethyl acetate (5:1), to afford tert-butyl (3S)-4-[(4R)-4-benzyl-2-oxo-1,3-oxazolidin-3-yl]-3-methyl-4-oxobutanoate (5.17 g, 69% yield) as a colorless oil. LCMS: (ES, m/z): RT=0.96 min, m/z=292[M+1]⁺. ¹H NMR (400 MHz, DMSO-d₆) δ 7.32 (d, J=6.9 Hz, 2H), 7.30-7.24 (m, 2H), 7.24-7.17 (m, 1H), 4.65 (t, J=8.0 Hz, 1H), 4.43-4.22 (m, 1H), 4.10-3.91 (m, 1H), 3.09-2.76 (m, 2H), 2.67 (d, J=16.6 Hz, 1H), 2.54-2.33 (m, 2H), 1.37 (s, 9H), 1.21-1.01 (m, 3H).

Step 2: Into a 250 mL round-bottom flask was added tert-butyl (3S)-4-[(4R)-4-benzyl-2-oxo-1,3-oxazolidin-3-yl]-3-methyl-4-oxobutanoate (4.80 g, 3.45 mmol, 1 equiv), H₂O (10 mL), tetrahydrofuran (THF) (20 mL) and H₂O₂ (30% in water) (3.75 g, 27.6 mmol, 8 equiv) at 0° C. The resulting mixture was stirred for 5 min at 0° C., then LiOH (248 mg, 10.4 mmol, 3 equiv) was added at 0° C. The mixture was stirred for overnight at room temperature. The reaction progress was monitored by GCMS. The reaction was quenched by the addition of Na₂S₂O₄ (aq, 10 mL) at 0° C. The organic phase was concentrated under reduced pressure. To the reaction was added water (20 mL) at room temperature. The resulting mixture was extracted with ethyl acetate (EtOAc) (2×10 mL). The water layers were then combined and adjusted to pH 6 with HCl (aq. 1M). The resulting water mixture was extracted with EtOAc (3×10 mL). The combined EtOAc organic layers were then washed with water (10 mL), dried over anhydrous Na₂SO₄, and concentrated under reduced pressure to provide a residue, which was purified by silica gel column chromatography, eluting with petroleum ether/ethyl acetate (5:1), to afford (2S)-4-(tert-butoxy)-2-methyl-4-oxobutanoic acid (2 g, 77% yield) as a colorless oil. GCMS: (ES, m/z): RT=4.19 min, m/z=188[M]. ¹H NMR (300 MHz, DMSO-d₆) δ 12.20 (s, 1H), 2.75-2.52 (m, 1H), 2.45 (t, J=8.1 Hz, 1H), 2.37-2.17 (m, 1H), 1.39 (s, 9H), 1.09 (d, J=7.2 Hz, 3H).

Step 3: Into a 40 mL round-bottom flask was added (2S)-4-(tert-butoxy)-2-methyl-4-oxobutanoic acid (2 g, 2.65 mmol, 1 equiv), [bis(dimethylamino)methylene]-1H-1,2,3-triazolo[4,5-b]pyridinium 3-oxide hexafluorophosphate (HATU) (6.06 g, 3.98 mmol, 1.5 equiv), N,N-diisopropylethylamine (DIPEA) (4.12 g, 7.96 mmol, 3 equiv), N,N-dimethyl formamide (DMF) (40 mL) and N,O-dimethylhydroxylamine hydrochloride (788 mg, 7.96 mmol, 3 equiv) at room temperature. The mixture was stirred for 1 h at room temperature. The reaction progress was monitored by LCMS. The reaction was quenched by the addition of water (20 mL) at room temperature. The aqueous layer was extracted with ethyl acetate (EtOAc) (3×20 mL), and the organic phase was concentrated under reduced pressure to provide a residue, which was purified by reversed-phase flash chromatography (C18 silica gel; mobile phase, acetonitrile (MeCN) in water, 10% to 50% gradient in 10 min; detector, UV 254/220 nm) to provide tert-butyl (3S)-3-[methoxy(methyl)carbamoyl]-3-methylpropanoate (2.0 g, 81% yield) as a colorless oil. LCMS: (ES, m/z): RT=0.71 min, m/z=176[M+1]⁺. ¹H NMR (400 MHz, DMSO-d₆) δ 3.72 (s, 3H), 3.18-3.13 (m, 1H), 3.10 (s, 3H), 2.58-2.44 (m, 1H), 2.25 (d, J=16.3 Hz, 1H), 1.37 (s, 9H), 1.01 (d, J=7.0 Hz, 3H).

Step 4. Into a 250 mL round-bottom flask was added tert-butyl (3S)-3-[methoxy(methyl)carbamoyl]-3-methylpropanoate (2 g, 25.9 mmol, 1 equiv), tetrahydrofuran (THF) (50 mL) and bromo(cyclopropyl)magnesium (130 mL, 130 mmol, 5 equiv, 1M in THF) at −40° C. The mixture was stirred for 2 h at room temperature. The reaction progress was monitored by LCMS. The reaction was quenched by the addition of water (30 mL) at room temperature. The aqueous layer was extracted with ethyl acetate (EtOAc) (3×20 mL). The organic phase was concentrated under reduced pressure. The residue was purified by silica gel column chromatography, eluting with petroleum ether/ethyl acetate (5:1), to afford tert-butyl (3S)-4-cyclopropyl-3-methyl-4-oxobutanoate (1.7 g, 30% yield) as a colorless oil. LCMS: (ES, m/z): RT=0.903 min, m/z=157[M+1]⁺. ¹H NMR (300 MHz, DMSO-d₆) δ 3.04 (d, J=8.5 Hz, 1H), 2.59-2.45 (m, 1H), 2.27 (d, J=16.3 Hz, 1H), 2.21-2.04 (m, 1H), 1.37 (s, 9H), 1.10 (d, J=7.2 Hz, 3H), 0.97-0.80 (m, 2H), 0.80-0.69 (m, 2H).

Step 5: Into a 250 mL round-bottom flask was added tert-butyl (3S)-4-cyclopropyl-3-methyl-4-oxobutanoate (1.25 g, 11.8 mmol, 1 equiv), benzylamine (1.26 g, 23.66 mmol, 2 equiv), titanium isopropoxide (Ti(Oi-Pr)₄) (0.18 g, 5.88 mmol, 0.5 equiv) and methanol (MeOH) (50 mL) at room temperature. The mixture was stirred for 30 min at 80° C., and then sodium cyanoborohydride (NaBH₃CN) (1.11 g, 35.3 mmol, 3 equiv) was added at room temperature and stirred for 1 h at room temperature. The reaction progress was monitored by LCMS. The reaction was quenched by the addition of water (10 mL) at 0° C. The resulting mixture was extracted with ethyl acetate (EtOAc) (3×20 mL), dried over anhydrous Na₂SO₄, and after filtration, the filtrate was concentrated under reduced pressure. The residue was purified by reversed-phase flash chromatography (C18 silica gel; mobile phase, acetonitrile (MeCN) in water, 10% to 50% gradient in 10 min; detector, UV 254/220 nm) to afford tert-butyl (3S)-4-(benzylamino)-4-cyclopropyl-3-methylbutanoate (0.9 g, 50% yield) as a colorless oil. LCMS: (ES, m/z): RT=0.575 min, m/z=304[M+1]⁺. ¹H NMR (400 MHz, DMSO-d₆) δ 7.43-7.15 (m, 5H), 3.94-3.78 (m, 1H), 3.77-3.65 (m, 2H), 2.49-2.33 (m, 1H), 2.23-2.07 (m, 2H), 1.65 (d, J=9.0 Hz, 1H), 1.34 (s, 9H), 1.27-1.07 (m, 1H), 1.07-0.76 (m, 3H), 0.76-0.53 (m, 2H), 0.17-0.05 (m, 2H).

Step 6: Into a 100 mL round-bottom flask was added tert-butyl (3S)-4-(benzylamino)-4-cyclopropyl-3-methylbutanoate (500 mg, 1.64 mmol, 1 equiv), methanol (MeOH) (20 mL) and Pd/C (877 mg, 8.24 mmol, 5 equiv) at room temperature. The mixture was stirred for 3 h at room temperature under hydrogen (H₂) atmosphere. The reaction progress was monitored by LCMS. The resulting mixture was filtered, the filter cake was washed with MeOH (3×10 mL). The filtrate was concentrated under reduced pressure to provide tert-butyl (3S)-4-amino-4-cyclopropyl-3-methylbutanoate (260 mg, 74% yield) as a colorless oil. LCMS: (ES, m/z): RT=0.50 min, m/z=214[M+1]⁺.

Step 7: Into a 40 mL round-bottom flask was added tert-butyl (3S)-4-amino-4-cyclopropyl-3-methylbutanoate (130 mg, 0.61 mmol, 1 equiv), [bis(dimethylamino)methylene]-1H-1,2,3-triazolo[4,5-b]pyridinium 3-oxide hexafluorophosphate (HATU) (348 mg, 0.91 mmol, 1.50 equiv), N,N-diisopropylethylamine (DIPEA) (234 mg, 1.83 mmol, 3 equiv), N,N-dimethyl formamide (DMF) (4 mL) and (5-cyclopropyl-4-fluoro-3,3-dimethyl-2-oxoindol-1-yl)acetic acid (product of Step 3, Example 13) (253.48 mg, 0.91 mmol, 1.5 equiv) at room temperature. The mixture was stirred for 1 h at room temperature. The reaction progress was monitored by LCMS, and upon completion, the reaction was quenched by the addition of water (5 mL) at room temperature. The aqueous layer was extracted with ethyl acetate (EtOAc) (3×10 mL) and the organic phase was concentrated under reduced pressure to provide a residue which was purified by reversed-phase flash chromatography (C18 silica gel; mobile phase, acetonitrile (MeCN) in water, 10% to 50% gradient in 10 min; detector, UV 254 nm) to provide tert-butyl (3S)-4-cyclopropyl-4-(2-(5-cyclopropyl-4-fluoro-3,3-dimethyl-2-oxoindolin-1-yl)acetamido)-3-methylbutanoate (Compound 60-(3S)-OtBu) (100 mg, 35% yield), which comprises a mixture of 2 stereoisomers, Compound 60A*-OtBu and Compound 60C*-OtBu, provided as a colorless oil. LCMS: (ES, m/z): RT=1.05 min, m/z=417[M+1]⁺.

Step 8: Into a 50 mL round-bottom flask was added Compound 60-(3S)-OtBu (100 mg, 0.275 mmol, 1 equiv), trifluoroacetic acid (TFA) (1 mL), and dichloromethane (DCM) (5 mL) at room temperature, and the mixture was stirred for 1 h at room temperature. The reaction progress was monitored by LCMS. The resulting mixture was concentrated under reduced pressure to provide a crude product (80 mg, 85% purity) which was purified by Prep-HPLC (XBridge Prep OBD C18 Column, 30*150 mm, 5 μm; Mobile Phase A: Water (10 mmol/L NH₄HCO₃), Mobile Phase B: acetonitrile (MeCN); Flow rate: 60 mL/min; Gradient: 19% B to 29% B in 7.8 min; Wave Length: 254 nm/220 nm; HPLC RT (min): 11.08) to provide (3S)-4-cyclopropyl-4-[2-(5-cyclopropyl-4-fluoro-3,3-dimethyl-2-oxoindol-1-yl)acetamido]-3-methylbutanoic acid (Compound 60-(3S)-mixture) (50 mg, 57% yield) as a colorless oil, comprising a mixture of 3S4R and 3S4S stereoisomers. LCMS: (ES, m/z): RT=0.79 min, m/z=417[M+1]⁺.

Step 9: The Compound 60-(3S)-mixture (50 mg) was separated by Prep-CHIRAL-HPLC (CHIRALPAK IE 2*25 cm, 5 um; Mobile Phase A: Hexanes (0.2% TFA), Mobile Phase B: isopropanol (IPA): DCM=1:1; Flow rate: 20 mL/min; Gradient: 15% B to 15% B in 22 min; Wave Length: 220/254 nm) to provide (3S,4R)-4-cyclopropyl-4-(2-(5-cyclopropyl-4-fluoro-3,3-dimethyl-2-oxoindolin-1-yl)acetamido)-3-methylbutanoic acid (Compound 60C*) (Chiral HPLC RT (min): 13.73; 26.2 mg; 20% yield) as a white solid, and (3S,4S)-4-cyclopropyl-4-(2-(5-cyclopropyl-4-fluoro-3,3-dimethyl-2-oxoindolin-1-yl)acetamido)-3-methylbutanoic acid (Compound 60A*) (Chiral HPLC RT (min): 19.46; 2.8 mg, 2% yield) as a white solid. Stereochemistry was arbitrarily assigned.

Compound 60C*: LCMS: (ES, m/z): RT=0.72 min, m/z=417[M+1]⁺. ¹H NMR (400 MHz, DMSO-d₆) δ 11.96 (s, 1H), 8.08 (d, J=9.1 Hz, 1H), 6.89 (t, J=7.8 Hz, 1H), 6.60 (d, J=8.0 Hz, 1H), 4.37-4.23 (m, 2H), 3.12 (d, J=19.9 Hz, 1H), 2.49-2.35 (m, 1H), 2.16-1.92 (m, 3H), 1.38 (s, 6H), 1.02-0.82 (m, 6H), 0.73-0.61 (m, 2H), 0.49 (d, J=8.7 Hz, 1H), 0.43-0.33 (m, 1H), 0.27-0.10 (m, 2H).

Compound 60A*: LCMS: (ES, m/z): RT=0.71 min, m/z=417[M+1]⁺. ¹H NMR (400 MHz, DMSO-d₆) δ 11.20 (s, 1H), 8.07 (d, J=9.1 Hz, 1H), 6.89 (t, J=7.8 Hz, 1H), 6.60 (d, J=8.1 Hz, 1H), 4.30 (d, J=1.6 Hz, 2H), 3.14 (t, J=8.9 Hz, 1H), 2.40 (d, J=10.9 Hz, 1H), 2.08-1.92 (m, 3H), 1.38 (s, 6H), 0.97-0.82 (m, 6H), 0.70-0.61 (m, 2H), 0.48 (d, J=8.8 Hz, 1H), 0.38 (d, J=8.3 Hz, 1H), 0.18 (d, J=20.0 Hz, 2H).

(3R,4R)-4-cyclopropyl-4-(2-(5-cyclopropyl-4-fluoro-3,3-dimethyl-2-oxoindolin-1-yl)acetamido)-3-methylbutanoic acid (Compound 60B*), (3R,4S)-4-cyclopropyl-4-(2-(5-cyclopropyl-4-fluoro-3,3-dimethyl-2-oxoindolin-1-yl)acetamido)-3-methylbutanoic acid (Compound 60D*), Compound 60B*-OtBu and Compound 60D*-OtBu may be prepared following steps 1-9 of Example 17 using (4S)-4-benzyl-3-propanoyl-1,3-oxazolidin-2-one instead of (4R)-4-benzyl-3-propanoyl-1,3-oxazolidin-2-one as the starting material.

### Example 18: (R)-4-(2-(5-cyclopropyl-4,7-difluoro-3,3-dimethyl-2-oxoindolin-1-yl)acetamido)-3-methylbutanoic acid (Compound 61A), (S)-4-(2-(5-cyclopropyl-4,7-difluoro-3,3-dimethyl-2-oxoindolin-1-yl)acetamido)-3-methylbutanoic acid (Compound 61B), methyl (R)-4-(2-(5-cyclopropyl-4,7-difluoro-3,3-dimethyl-2-oxoindolin-1-yl)acetamido)-3-methylbutanoate (Compound 61A-OMe) and ethyl (S)-4-(2-(5-cyclopropyl-4,7-difluoro-3,3-dimethyl-2-oxoindolin-1-yl)acetamido)-3-methylbutanoate (Compound 61B-OEt)

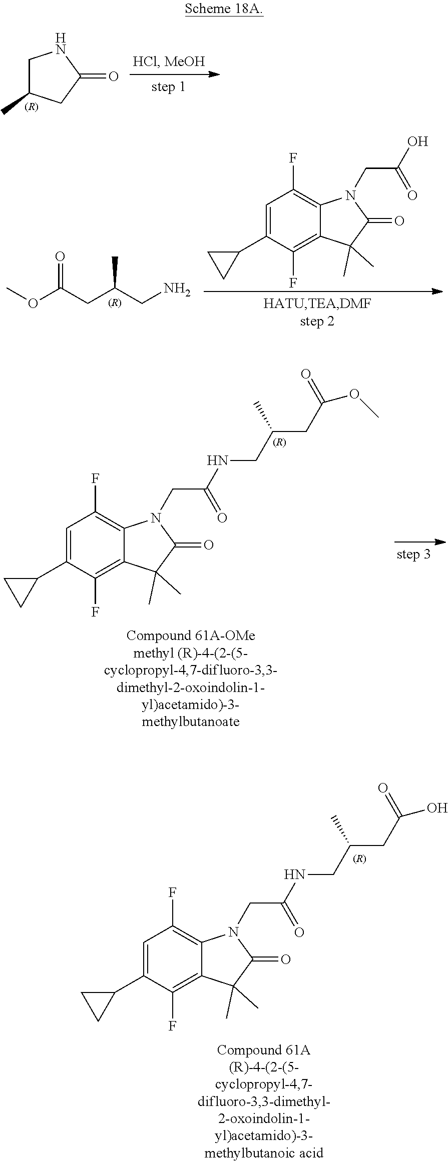

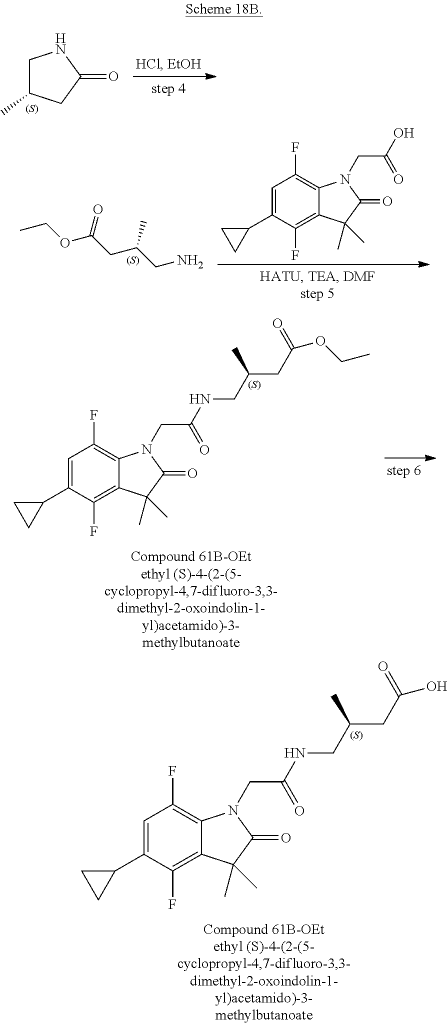

Step 1: A solution of (4R)-4-methylpyrrolidin-2-one (1 g, 10.1 mmol, 1 equiv) and HCl (5 mL, 6 M) in methanol (MeOH) (5 mL) was stirred for overnight at 100° C. The reaction was monitored by LCMS. The resulting mixture was concentrated under reduced pressure. To the above mixture was added 2,2-dimethoxypropane (20 mL) dropwise over 3 min at room temperature. The resulting mixture was stirred for additional 1 h at room temperature. The reaction was monitored by LCMS. The resulting mixture was concentrated under reduced pressure to provide methyl (3R)-4-amino-3-methylbutanoate (1.20 g, 91% yield) as a yellow oil. LCMS (LCMS, ESI): RT=0.25 min, m/z=132 [M+H]⁺.

Step 2: Into a 20 mL vial was added 2-(5-cyclopropyl-4,7-difluoro-3,3-dimethyl-2-oxoindolin-1-yl)acetic acid (product of Step 6, Example 14) (600 mg, 2.03 mmol, 1 equiv) and methyl (R)-4-amino-3-methylbutanoate hydrochloride (501.6 mg, 3.45 mmol, 1.7 equiv), [bis(dimethylamino)methylene]-1H-1,2,3-triazolo[4,5-b]pyridinium 3-oxide hexafluorophosphate (HATU) (1.15 mg, 3.04 mmol, 1.5 equiv), triethylamine (TEA) (1.02 g, 10.2 mmol, 5 equiv), and N,N-dimethyl formamide (DMF) (6 mL) at room temperature. The resulting mixture was stirred for 1 h at room temperature. The reaction was monitored by LCMS. The reaction was quenched by the addition of water (10 mL) at room temperature. The resulting mixture was extracted with ethyl acetate (EtOAc) (3×80 mL). The combined organic layers were washed with water (3×60 mL), dried over anhydrous Na₂SO₄. After filtration, the filtrate was concentrated under reduced pressure. The residue was purified by reversed-phase flash chromatography (C18 silica gel; mobile phase, acetonitrile (MeCN) in water, 40% to 50% gradient in 12 min; detector, UV 254/220 nm) to provide methyl (3R)-4-[2-(5-cyclopropyl-4,7-difluoro-3,3-dimethyl-2-oxoindol-1-yl)acetamido]-3-methylbutanoate (Compound 61A-OMe) (200 mg, 23% yield) as a yellow oil. LCMS (LCMS, ESI): RT=0.85 min, m/z=409 [M+H]⁺; ¹H NMR (400 MHz, DMSO-d₆) δ 8.17 (t, J=5.7 Hz, 1H), 6.79 (d, J=12.1 Hz, 1H), 4.36 (d, J=1.8 Hz, 2H), 3.59 (s, 3H), 3.08-2.63 (m, 2H), 2.36 (d, J=15.0 Hz, 1H), 2.13-2.04 (m, 1H), 2.04-1.95 (m, 2H), 1.40 (s, 6H), 1.37-0.89 (m, 4H), 0.85 (d, J=6.5 Hz, 3H).

Step 3: Into a 20 mL vial was added Compound 61A-OMe (200 mg, 0.49 mmol, 1 equiv), LiOH (35.2 mg, 1.47 mmol, 3 equiv), H₂O (3 mL), and methanol (MeOH) (3 mL) at room temperature. The resulting mixture was stirred for 1 h at room temperature. The reaction was monitored by LCMS. The reaction was quenched with water (10 mL) at room temperature, and the pH of the mixture was adjusted to pH 4 with HCl (aq. 4 M). The resulting mixture was extracted with ethyl acetate (EtOAc) (3×40 mL). The combined organic layers were washed with water (3×40 mL), dried over anhydrous Na₂SO₄. After filtration, the filtrate was concentrated under reduced pressure to provide a crude product (200 mg, 80% purity), which was purified by Prep-HPLC (Xselect CSH OBD Column 30*150 mm, 5 umn; Mobile Phase A: water (0.05% TFA), Mobile Phase B: acetonitrile (MeCN); Flow rate: 60 mL/min; Gradient: 35% B to 45% B in 10 min; Wave Length: 254 nm/220 nm; HPLC RT (min): 8.43) to afford (R)-4-(2-(5-cyclopropyl-4,7-difluoro-3,3-dimethyl-2-oxoindolin-1-yl)acetamido)-3-methylbutanoic acid (Compound 61A) (90.6 mg, 47% yield) as a white solid. LCMS (LCMS, ESI): RT=1.52 min, m/z=395 [M+H]⁺; ¹H NMR (400 MHz, Methanol-d₄) δ 6.68 (d, J=12.0 Hz, 1H), 4.52 (d, J=1.7 Hz, 2H), 3.17 (d, J=6.3 Hz, 2H), 2.46-2.30 (m, 1H), 2.27-1.91 (m, 3H), 1.51 (s, 6H), 1.10-0.89 (m, 5H), 0.74-0.59 (m, 2H).

Step 4: A solution of (4S)-4-methylpyrrolidin-2-one (500 mg, 5.04 mmol, 1 equiv) and 4M HCl (10 mL) in EtOH (10 mL) was stirred for overnight at 100° C. The reaction was monitored by LCMS. The resulting mixture was concentrated under reduced pressure to provide ethyl (3S)-4-amino-3-methylbutanoate (300 mg, 41% yield) as a yellow oil. LCMS: (ES, m/z): RT=0.24 min, m/z=146.0[M+H]⁺.

Step 5: A solution of 2-(5-cyclopropyl-4,7-difluoro-3,3-dimethyl-2-oxoindolin-1-yl)acetic acid (product of Step 6, Example 14) (300 mg, 1.01 mmol, 1 equiv), ethyl (3S)-4-amino-3-methylbutanoate (177 mg, 1.21 mmol, 1.2 equiv), [bis(dimethylamino)methylene]-1H-1,2,3-triazolo[4,5-b]pyridinium 3-oxide hexafluorophosphate (HATU) (502 mg, 1.32 mmol, 1.3 equiv), and triethylamine (308 mg, 3.04 mmol, 3 equiv) in N,N-dimethyl formamide (DMF) (6 mL) was stirred for 1 h at room temperature. The reaction was monitored by LCMS. The reaction was quenched with water (50 mL) at room temperature. The resulting mixture was extracted with ethyl acetate (EtOAc) (3×50 mL). The combined organic layers were washed with water (2×50 mL), dried over anhydrous Na₂SO₄, and after filtration, the filtrate was concentrated under reduced pressure to provide a residue, which was purified by reversed-phase flash chromatography (C18 silica gel; mobile phase, acetonitrile (MeCN) in water, 55% to 65% gradient in 10 min; detector, UV 254/220 nm) to provide ethyl (S)-4-(2-(5-cyclopropyl-4,7-difluoro-3,3-dimethyl-2-oxoindolin-1-yl)acetamido)-3-methylbutanoate (Compound 61B-OEt)(160 mg, 39% yield) as a yellow solid. LCMS: (ES, m/z): RT=0.86 min, m/z=409.3[M+H]⁺.

Step 6: A solution of Compound 61B-OEt (160 mg, 0.39 mmol, 1 equiv), LiOH (28.2 mg, 1.17 mmol, 3 equiv), H₂O (0.5 mL), and tetrahydrofuran (THF) (4 mL) was stirred for 1 h at room temperature. The reaction was monitored by LCMS. The mixture was adjusted to pH=3 with HCl (aq. 4M). The resulting mixture was extracted with ethyl acetate (EtOAc) (3×30 mL). The combined organic layers were washed with water (2×10 mL), dried over anhydrous Na₂SO₄. After filtration, the filtrate was concentrated under reduced pressure. The residue was purified by reversed-phase flash chromatography (C18 silica gel; mobile phase, acetonitrile (MeCN) in water, 40% to 50% gradient in 10 min; detector, UV 254/220 nm) to provide a crude product (130 mg, 80% purity), which was further purified by Prep-HPLC (Xselect CSH OBD Column 30*150 mm, 5 umn; Mobile Phase A: Water (0.1% FA), Mobile Phase B: acetonitrile (MeCN); Flow rate: 60 mL/min; Gradient: 37% B to 47% B in 10 min; Wave Length: 254 nm/220 nm; HPLC RT (min): 7.02) to afford (S)-4-(2-(5-cyclopropyl-4,7-difluoro-3,3-dimethyl-2-oxoindolin-1-yl)acetamido)-3-methylbutanoic acid (Compound 61B) (118 mg, 76% yield) as a white solid. LCMS: (ES, m/z): RT=1.64 min, m/z=395.2[M+H]; ¹H NMR (400 MHz, Methanol-d₄) δ 6.69 (d, J=12.0 Hz, 1H), 4.52 (d, J=1.7 Hz, 2H), 3.17 (d, J=6.4 Hz, 2H), 2.44-2.35 (m, 1H), 2.22-1.98 (m, 3H), 1.51 (s, 6H), 1.04-0.95 (m, 5H), 0.68 (d, J=6.5 Hz, 2H).

### Example 19: (R)-4-(2-(5-cyclopropyl-4,7-difluoro-3,3-dimethyl-2-oxoindolin-1-yl)acetamido)-3-fluorobutanoic acid (Compound 62A) and (S)-4-(2-(5-cyclopropyl-4,7-difluoro-3,3-dimethyl-2-oxoindolin-1-yl)acetamido)-3-fluorobutanoic acid (Compound 62B), methyl (R)-4-(2-(5-cyclopropyl-4,7-difluoro-3,3-dimethyl-2-oxoindolin-1-yl)acetamido)-3-fluorobutanoate (Compound 62A-OMe) and methyl (S)-4-(2-(5-cyclopropyl-4,7-difluoro-3,3-dimethyl-2-oxoindolin-1-yl)acetamido)-3-fluorobutanoate (Compound 62B-OMe)

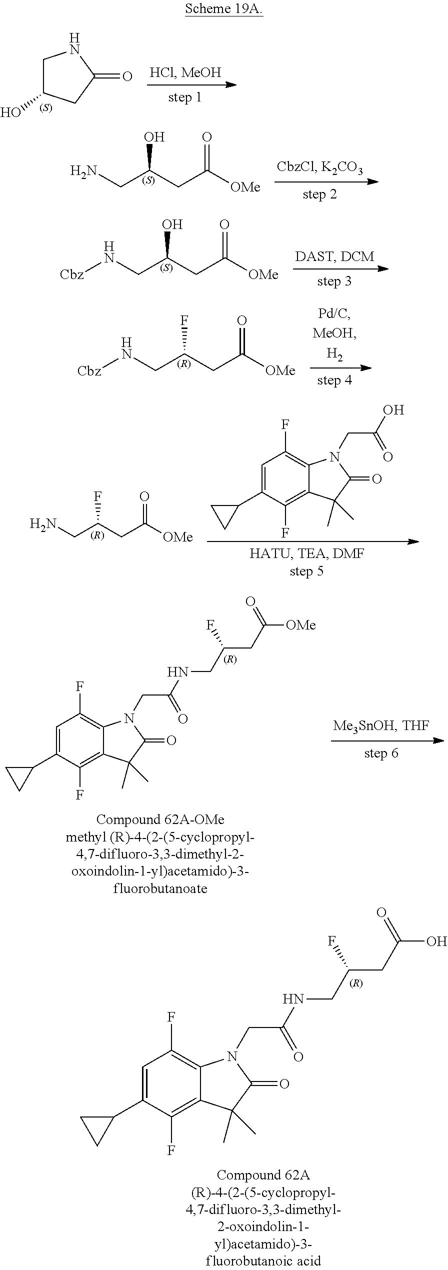

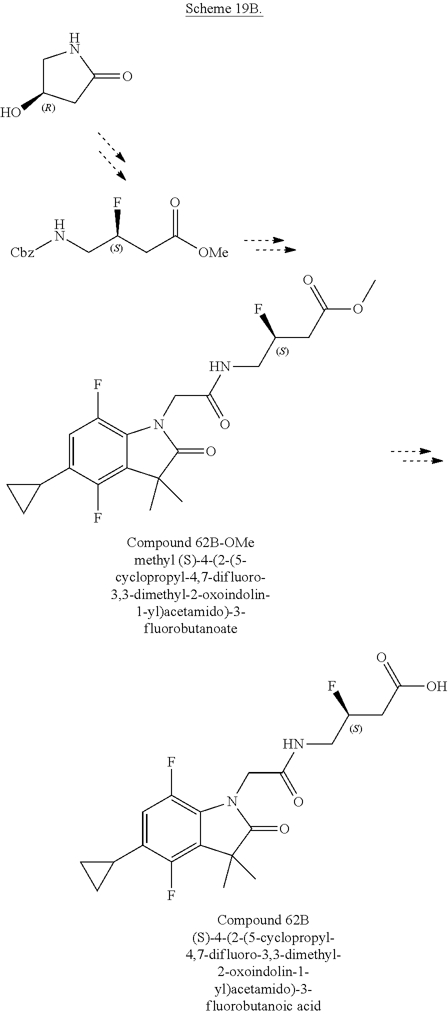

Step 1: Into a 20 mL vial was added (4S)-4-hydroxypyrrolidin-2-one (500 mg, 4.95 mmol, 1 equiv) and methanol (MeOH) (5 mL), and HCl (6M, 5 mL). The resulting mixture was stirred for 12 h at 80° C. The reaction was monitored by LCMS. The resulting mixture was concentrated under reduced pressure to afford methyl (S)-4-amino-3-hydroxybutanoate hydrochloride (800 mg, 99% yield) as colorless oil. LCMS: (ES, m/z): RT=0.10 min, m/z=134.0[M+H]⁺.

Step 2: Into a 20 mL vial was added methyl (3S)-4-amino-3-hydroxybutanoate (500 mg, 3.76 mmol, 1 equiv), acetonitrile (MeCN) (8 mL), K₂CO₃ (1.04 g, 7.51 mmol, 2 equiv), and benzyl chloroformate (832 mg, 4.88 mmol, 1.3 equiv). The resulting mixture was stirred for 2 h at 80° C. The reaction was monitored by LCMS. The reaction was quenched by the addition of water (10 mL) at room temperature. The resulting mixture was extracted with ethyl acetate (EtOAc) (3×20 mL). The combined organic layers were washed with brine (20 mL) and dried over anhydrous Na₂SO₄. After filtration, the filtrate was concentrated under reduced pressure to provide a residue, which was purified by reversed-phase flash chromatography (C18 silica gel; mobile phase, acetonitrile (MeCN) in water, 40% to 60% gradient in 10 min; detector, UV 220/254 nm) to provide methyl (3S)-4-{[(benzyloxy)carbonyl]amino}-3-hydroxybutanoate (200 mg, 20% yield) as a yellow oil. LCMS: (ES, m/z): RT=0.53 min, m/z=268.0[M+H]⁺. ¹H NMR (400 MHz, DMSO-d₆) δ 7.36-7.27 (m, 5H), 7.23 (t, J=6.0 Hz, 1H), 5.01 (s, 1H), 4.98 (d, J=14.1 Hz, 2H), 3.96-3.87 (m, 1H), 3.59 (s, 3H), 3.11-3.01 (m, 1H), 3.04-2.94 (m, 1H), 2.51-2.43 (m, 1H), 2.30-2.19 (m, 1H).

Step 3: Into a 20 mL vial was added methyl (3S)-4-{[(benzyloxy)carbonyl]amino}-3-hydroxybutanoate (270 mg, 1.01 mmol, 1 equiv) and dichloromethane (DCM) (3 mL). To the above mixture was added diethylaminosulfur trifluoride (DAST) (977 mg, 6.06 mmol, 6 equiv) by dropwise at 0° C. The resulting mixture was stirred for additional 1 h at room temperature. The reaction was monitored by LCMS. The reaction was quenched by the addition of sat. NaHCO₃ (aq.) (10 mL) at 0° C. The resulting mixture was extracted with DCM (30 mL). The combined organic layers were washed with brine (15 mL), dried over anhydrous Na₂SO₄, and after filtration, the filtrate was concentrated under reduced pressure to provide a residue, which was purified by reversed-phase flash chromatography (C18 silica gel; mobile phase, acetonitrile (MeCN) in water, 0% to 30% gradient in 10 min; detector, UV 220/254 nm) to provide methyl (3R)-4-{[(benzyloxy)carbonyl]amino}-3-fluorobutanoate (130 mg, 48% yield) as a yellow oil. LCMS: (ES, m/z): RT=0.61 min, m/z=270.0[M+H]⁺.

Step 4: Into a 50 mL round-bottom flask was added methyl (3R)-4-{[(benzyloxy)carbonyl]amino}-3-fluorobutanoate (110 mg, 0.41 mmol, 1 equiv), methanol (MeOH) (5 mL), Pd/C (435 mg, 4.09 mmol, 10 equiv), and HCl (12M, 0.1 mL). The resulting mixture was stirred for 12 h at 50° C. under hydrogen (H₂) atmosphere. The reaction was monitored by LCMS. The resulting mixture was filtered, the filter cake was washed with MeOH (30 mL). The filtrate was concentrated under reduced pressure to afford methyl (R)-4-amino-3-fluorobutanoate hydrochloride (70 mg, 80% purity) as a white solid. LCMS: (ES, m/z): RT=0.10 min, m/z=136.0[M+H]⁺.

Step 5: Into a 8 mL vial was 2-(5-cyclopropyl-4,7-difluoro-3,3-dimethyl-2-oxoindolin-1-yl)acetic acid (product of Step 6, Example 14) (50 mg, 0.17 mmol, 1 equiv), N,N-dimethylformamide (DMF) (2 mL), methyl (R)-4-amino-3-fluorobutanoate hydrochloride (91.5 mg, 0.68 mmol, 4 equiv), [bis(dimethylamino)methylene]-1H-1,2,3-triazolo[4,5-b]pyridinium 3-oxide hexafluorophosphate (HATU) (77.3 mg, 0.20 mmol, 1.2 equiv), and triethylamine (TEA) (51.4 mg, 0.51 mmol, 3 equiv). The resulting mixture was stirred for 2 h at room temperature. The reaction was monitored by LCMS. The reaction was quenched by the addition of water (5 mL) at room temperature. The resulting mixture was extracted with ethyl acetate (EtOAc) (3×15 mL). The combined organic layers were washed with brine (10 mL) and dried over anhydrous Na₂SO₄. After filtration, the filtrate was concentrated under reduced pressure to provide a residue, which was purified by reversed-phase flash chromatography (C18 silica gel; mobile phase, acetonitrile (MeCN) in water, 25% to 40% gradient in 10 min; detector, UV 220/254 nm) to provide methyl (R)-4-(2-(5-cyclopropyl-4,7-difluoro-3,3-dimethyl-2-oxoindolin-1-yl)acetamido)-3-fluorobutanoate (Compound 62A-OMe) (30 mg, 43% yield) as a yellow oil. LCMS: (ES, m/z): RT=0.82 min, m/z=413.0[M+H]⁺.

Step 6: Into a 8 mL vial was added Compound 62A-OMe (20 mg, 0.05 mmol, 1 equiv), tetrahydrofuran (THF) (1 mL), and trimethyltin hydroxide (35.1 mg, 0.19 mmol, 4 equiv). The resulting mixture was stirred for 12 h at 60° C. The reaction was monitored by LCMS. The reaction was quenched with water (10 mL) at room temperature. The resulting mixture was extracted with ethyl acetate (EtOAc) (30 mL). The combined organic layers were washed with brine (5 mL) and dried over anhydrous Na₂SO₄. After filtration, the filtrate was concentrated under reduced pressure to afford a crude product (20 mg, 90% purity), which was purified by Prep-HPLC (XBridge Prep Phenyl Column, 19*250 mm, 5 μm; Mobile Phase A: Water (0.1% TFA), Mobile Phase B: acetonitrile (MeCN); Flow rate: 25 mL/min; Gradient: 43% B to 53% B in 9 min; Wave Length: 254 nm/220 nm; HPLC RT (min): 9.0) to afford (R)-4-(2-(5-cyclopropyl-4,7-difluoro-3,3-dimethyl-2-oxoindolin-1-yl)acetamido)-3-fluorobutanoic acid (Compound 62A) (3.6 mg, 19% yield) as a white solid. LCMS: (ES, m/z): RT=1.40 min, m/z=399.0[M+H]⁺. ¹H NMR (400 MHz, Methanol-d₄) δ 6.74-6.65 (m, 1H), 5.08-4.89 (m, 1H), 4.55 (d, J=1.7 Hz, 2H), 3.63-3.53 (m, 1H), 3.53-3.43 (m, 1H), 2.75-2.64 (m, 1H), 2.66-2.59 (m, 1H), 2.10-1.98 (m, 1H), 1.51 (s, 6H), 1.04-0.93 (m, 2H), 0.73-0.64 (m, 2H).

(S)-4-(2-(5-cyclopropyl-4,7-difluoro-3,3-dimethyl-2-oxoindolin-1-yl)acetamido)-3-fluorobutanoic acid (Compound 62B) and methyl (S)-4-(2-(5-cyclopropyl-4,7-difluoro-3,3-dimethyl-2-oxoindolin-1-yl)acetamido)-3-fluorobutanoate (Compound 62B-OMe) may be synthesized following steps 1-6 of Example 19 using (4R)-4-hydroxypyrrolidin-2-one instead of (4S)-4-hydroxypyrrolidin-2-one as the starting material.

### Example 20: (R)-4-(2-(5-cyclopropyl-4-fluoro-3,3-dimethyl-2-oxoindolin-1-yl)acetamido)-3-fluorobutanoic acid (Compound 63A), (S)-4-(2-(5-cyclopropyl-4-fluoro-3,3-dimethyl-2-oxoindolin-1-yl)acetamido)-3-fluorobutanoic acid (Compound 63B), methyl (R)-4-(2-(5-cyclopropyl-4-fluoro-3,3-dimethyl-2-oxoindolin-1-yl)acetamido)-3-fluorobutanoate (Compound 63A-OMe) and methyl (S)-4-(2-(5-cyclopropyl-4-fluoro-3,3-dimethyl-2-oxoindolin-1-yl)acetamido)-3-fluorobutanoate (Compound 63B-OMe)

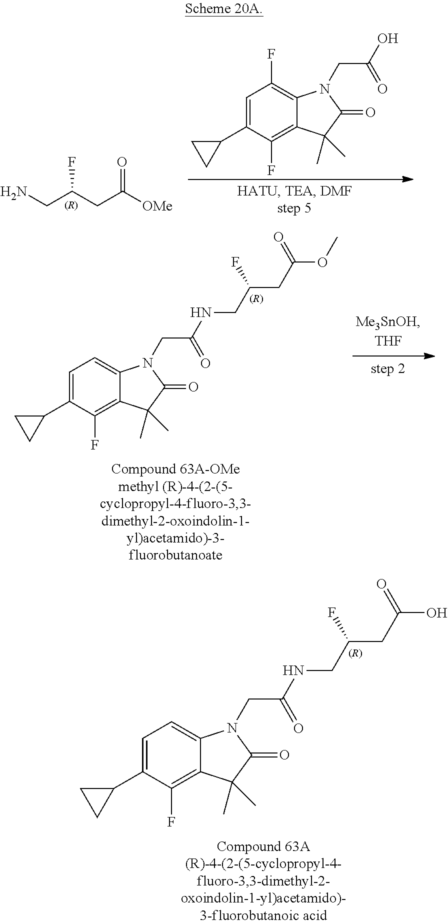

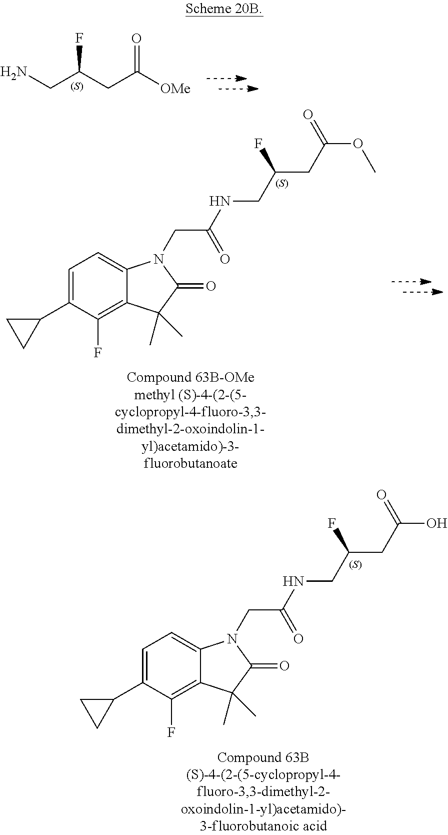

Step 1: Into a 8 mL vial was added (5-cyclopropyl-4-fluoro-3,3-dimethyl-2-oxoindol-1-yl)acetic acid (product of Step 3, Example 13) (40 mg, 0.15 mmol, 1 equiv), N,N-dimethylformamide (DMF) (1 mL), methyl (R)-4-amino-3-fluorobutanoate HCl salt (product of Step 4, Example 19) (99.0 mg, 0.58 mmol, 4 equiv), [bis(dimethylamino)methylene]-1H-1,2,3-triazolo[4,5-b]pyridinium 3-oxide hexafluorophosphate (HATU) (41.7 mg, 0.17 mmol, 1.20 equiv), and triethylamine (TEA) (43.8 mg, 0.43 mmol, 3 equiv). The resulting mixture was stirred for 2 h at room temperature. The reaction was monitored by LCMS. The reaction was quenched with water (5 mL) at room temperature. The resulting mixture was extracted with ethyl acetate (EtOAc) (3×15 mL). The combined organic layers were washed with brine (2×5 mL) and dried over anhydrous Na₂SO₄. After filtration, the filtrate was concentrated under reduced pressure to provide a residue, which was purified by reversed-phase flash chromatography (C18 silica gel; mobile phase, acetonitrile (MeCN) in water, 40% to 50% gradient in 10 min; detector, UV 220/254 nm) to provide methyl (R)-4-(2-(5-cyclopropyl-4-fluoro-3,3-dimethyl-2-oxoindolin-1-yl)acetamido)-3-fluorobutanoate (Compound 63A-OMe) (50 mg, 88% yield) as a yellow oil. LCMS: (ES, m/z): RT=1.04 min, m/z=395.0[M+H]⁺.

Step 2: Into a 8 mL vial was added Compound 63A-OMe (40 mg, 0.10 mmol, 1 equiv), tetrahydrofuran (THF) (1 mL), and trimethyltin hydroxide (55.0 mg, 0.30 mmol, 3 equiv), and the resulting mixture was stirred for 12 h at 60° C. The reaction was monitored by LCMS. The reaction was quenched by the addition of water (5 mL) at room temperature. The resulting mixture was extracted with ethyl acetate (EtOAc) (3×10 mL). The combined organic layers were washed with brine (2×5 mL) and dried over anhydrous Na₂SO₄. After filtration, the filtrate was concentrated under reduced pressure to afford a crude product (40 mg, 80% yield), which was purified by Prep-HPLC (Xselect CSH OBD Column 30*150 mm, 5 umn; Mobile Phase A: Water (0.05% TFA), Mobile Phase B: acetonitrile (MeCN); Flow rate: 60 mL/min; Gradient: 35% B to 45% B in 10 min; Wave Length: 254 nm/220 nm; HPLC RT (min): 9.4) to provide (R)-4-(2-(5-cyclopropyl-4-fluoro-3,3-dimethyl-2-oxoindolin-1-yl)acetamido)-3-fluorobutanoic acid (Compound 63A) (13.1 mg, 34% yield) as a white solid. LCMS: (ES, m/z): RT=1.52 min, m/z=381.0[M+H]⁺. ¹H NMR (400 MHz, DMSO-d₆) δ 12.46-12.42 (m, 1H), 8.45 (t, J=5.7 Hz, 1H), 6.89 (t, J=7.8 Hz, 1H), 6.65 (d, J=8.1 Hz, 1H), 5.00-4.78 (m, 1H), 4.33 (s, 2H), 3.48-3.32 (m, 2H), 2.74-2.62 (m, 1H), 2.60-2.51 (m, 1H), 2.03-1.92 (m, 1H), 1.38 (s, 6H), 0.96-0.87 (m, 2H), 0.69-0.61 (m, 2H).

(S)-4-(2-(5-cyclopropyl-4-fluoro-3,3-dimethyl-2-oxoindolin-1-yl)acetamido)-3-fluorobutanoic acid (Compound 63B) and methyl (S)-4-(2-(5-cyclopropyl-4-fluoro-3,3-dimethyl-2-oxoindolin-1-yl)acetamido)-3-fluorobutanoate (Compound 63B-OMe) may be synthesized following steps 1-4 of Example 19 using (4R)-4-hydroxypyrrolidin-2-one instead of (4S)-4-hydroxypyrrolidin-2-one as the starting material to provide methyl (S)-4-amino-3-fluorobutanoate HCl salt, and steps 1-2 of Example 20.

### Example 21: (R)-4-(2-(4,7-difluoro-3,3-dimethyl-2-oxo-5-(trifluoromethyl)indolin-1-yl)acetamido)-3-fluorobutanoic acid (Compound 64A), (S)-4-(2-(4,7-difluoro-3,3-dimethyl-2-oxo-5-(trifluoromethyl)indolin-1-yl)acetamido)-3-fluorobutanoic acid (Compound 64B), methyl (R)-4-(2-(4,7-difluoro-3,3-dimethyl-2-oxo-5-(trifluoromethyl)indolin-1-yl)acetamido)-3-fluorobutanoate (Compound 64A-OMe), and methyl (S)-4-(2-(4,7-difluoro-3,3-dimethyl-2-oxo-5-(trifluoromethyl)indolin-1-yl)acetamido)-3-fluorobutanoate (Compound 64B-OMe)

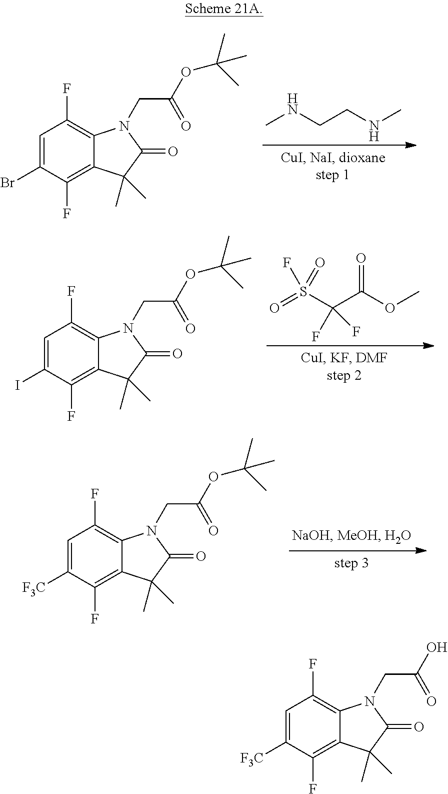

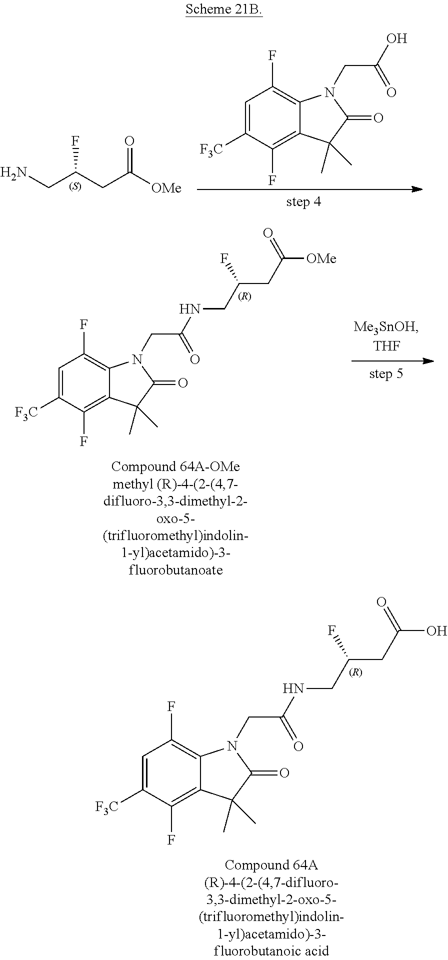

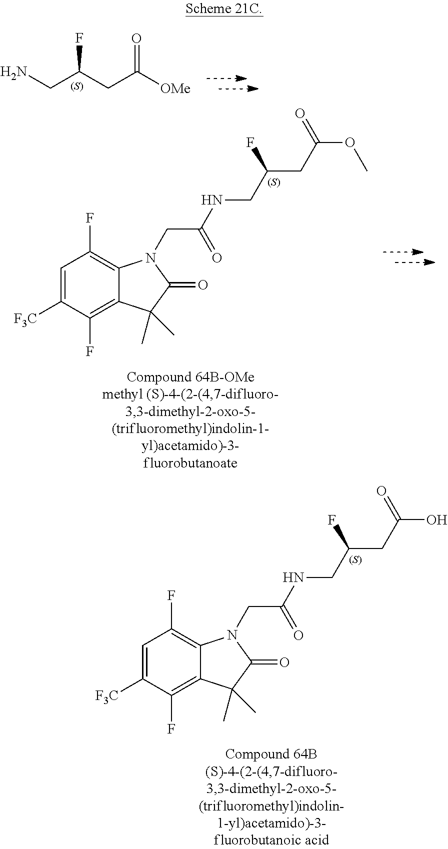

Step 1: Into a 30 mL sealed tube was added tert-butyl 2-(5-bromo-4,7-difluoro-3,3-dimethyl-2-oxoindol-1-yl)acetate (500 mg, 1.28 mmol, 1 equiv) (product of Step 4, Example 14), CuI (488 mg, 2.56 mmol, 2 equiv), NaI (1540 mg, 10.24 mmol, 8 equiv), 1,4-dioxane (7.5 mL) and methyl[2-(methylamino)ethyl]amine (226 mg, 2.56 mmol, 2 equiv) at room temperature. The resulting mixture was stirred for 2 h at 120° C. under nitrogen atmosphere. The reaction was monitored by LCMS. The resulting mixture was concentrated under reduced pressure. The residue was purified by reversed-phase flash chromatography (C18 silica gel; mobile phase, MeCN in water, 0% to 100% gradient in 30 min; detector, UV 254 nm) to provide tert-butyl 2-(4,7-difluoro-5-iodo-3,3-dimethyl-2-oxoindol-1-yl)acetate (190 mg, 34% yield) as a brown solid. LCMS: (ES, m/z): RT=1.027 min, m/z=438 [M+1]⁺. ¹H NMR (400 MHz, DMSO-d₆) δ 7.79-7.71 (m, 1H), 4.51-4.44 (m, 2H), 1.45 (s, 6H), 1.40 (d, J=1.4 Hz, 9H).

Step 2: Into a 8 mL sealed tube was added tert-butyl 2-(4,7-difluoro-5-iodo-3,3-dimethyl-2-oxoindol-1-yl)acetate (180 mg, 0.39 mmol, 1 equiv), CuI (157 mg, 0.81 mmol, 2 equiv), KF (71.8 mg, 1.23 mmol, 3 equiv), N,N-dimethyl formamide (DMF) (2 mL) and methyl 2,2-difluoro-2-sulfoacetate (158 mg, 0.81 mmol, 2 equiv) at room temperature. The resulting mixture was stirred for 2 h at 100° C. under nitrogen atmosphere. The reaction was monitored by LCMS. The reaction was quenched with water (2 mL) at room temperature. The resulting mixture was extracted with dichloromethane (DCM) (3×8 mL). The combined organic layers were washed with water (3×5 mL), dried over anhydrous Na₂SO₄. After filtration, the filtrate was concentrated under reduced pressure. The residue was purified by reversed-phase flash chromatography (C18 silica gel; mobile phase, MeCN in water, 0% to 100% gradient in 30 min; detector, UV 254 nm) to provide tert-butyl 2-[4,7-difluoro-3,3-dimethyl-2-oxo-5-(trifluoromethyl)indol-1-yl]acetate (100 mg, 64% yield) as a brown oil. LCMS: (ES, m/z): RT=1.011 min, m/z=380 [M+1]⁺. ¹H NMR (400 MHz, DMSO-d₆) δ 7.83-7.77 (m, 1H), 4.54 (d, J=2.3 Hz, 2H), 1.45 (s, 6H), 1.41 (s, 9H).

Step 3: Into a 8 mL vial was added isopropyl 2-[4,7-difluoro-3,3-dimethyl-2-oxo-5-(trifluoromethyl)indol-1-yl]acetate (100 mg, 0.06 mmol, 1 equiv), methanol (MeOH) (1 mL), H₂O (0.5 mL) and NaOH (219 mg, 1.08 mmol, 20 equiv) at room temperature. The resulting mixture was stirred for 1 h at room temperature. The reaction was monitored by LCMS. The mixture was acidified to pH 5 with HCl (1 M, 2 mL). The resulting mixture was extracted with dichloromethane (DCM) (3×5 mL). The combined organic layers were washed with water (3×5 mL), dried over anhydrous Na₂SO₄. After filtration, the filtrate was concentrated under reduced pressure. This resulted in [4,7-difluoro-3,3-dimethyl-2-oxo-5-(trifluoromethyl)indol-1-yl]acetic acid (60 mg, 70% yield) as a white solid. LCMS: (ES, m/z): RT=0.752 min, m/z=322 [M−1]⁺. ¹H NMR (400 MHz, DMSO-d₆) δ 7.82-7.74 (m, 1H), 4.54 (d, J=2.3 Hz, 2H), 1.45 (s, 6H).

Step 4: Into a 25 mL round-bottom flask was added [4,7-difluoro-3,3-dimethyl-2-oxo-5-(trifluoromethyl)indol-1-yl]acetic acid (50 mg, 0.15 mmol, 1 equiv), methyl (R)-4-amino-3-fluorobutanoate HCl salt (product of Step 4, Example 19) (31.4 mg, 0.23 mmol, 1.5 equiv), [bis(dimethylamino)methylene]-1H-1,2,3-triazolo[4,5-b]pyridinium 3-oxide hexafluorophosphate (HATU) (88 mg, 0.23 mmol, 1.5 equiv), N,N-diisopropylethylamine (DIPEA) (60.0 mg, 0.46 mmol, 3 equiv) and N,N-dimethyl formamide (DMF) (3 mL). The solution was stirred for 1 h at 60° C. The reaction progress was monitored by LCMS. The reaction was quenched by the addition of water (5 mL) at room temperature. The aqueous layer was extracted with ethyl acetate (EtOAc) (3×10 mL). The organic phase was concentrated under reduced pressure. The residue was purified by reversed-phase flash chromatography (C18 silica gel; mobile phase, acetonitrile (MeCN) in water, 10% to 50% gradient in 10 min; detector, UV 254/220 nm) to provide methyl (R)-4-(2-(4,7-difluoro-3,3-dimethyl-2-oxo-5-(trifluoromethyl)indolin-1-yl)acetamido)-3-fluorobutanoate (Compound 64A-OMe) (40 mg, 59% yield) as a yellow oil. LCMS:(ES, m/z): RT=0.74 min, m/z=441.0[M+H]⁺. ¹H NMR (400 MHz, Methanol-d₄) δ 7.49 (d, J=10.8 Hz, 1H), 4.58 (d, J=1.9 Hz, 1H), 4.27 (d, J=4.9 Hz, 2H), 3.55-3.43 (m, 2H), 3.42 (s, 3H), 2.35-2.13 (m, 2H), 1.35 (s, 6H).

Step 5: Into a 25 mL round-bottom flask was added Compound 64A-OMe (30 mg, 0.06 mmol, 1 equiv), trimethyltin hydroxide (36.96 mg, 0.20 mmol, 3 equiv). 1 equiv) and tetrahydrofuran (THF) (2 mL). The solution was stirred for 1 h at 60° C. The reaction progress was monitored by LCMS. The resulting mixture was filtered, the filter cake was washed with acetonitrile (2×10 mL). The filtrate was concentrated under reduced pressure to provide a crude product (25 mg, 85% yield), which was purified by Prep-HPLC (Xselect CSH OBD Column 30*150 mm, 5 um; Mobile Phase A: Water (0.1% FA), Mobile Phase B: acetonitrile (MeCN); Flow rate: 60 mL/min; Gradient: 36% B to 46% B in 10 min; Wave Length: 254 nm/220 nm; HPLC RT (min): 8.15) to provide (R)-4-(2-(4,7-difluoro-3,3-dimethyl-2-oxo-5-(trifluoromethyl)indolin-1-yl)acetamido)-3-fluorobutanoic acid (Compound 64A) (13.6 mg, 27% yield) as an off-white solid. LCMS: (ES, m/z): RT=1.33 min, m/z=427.2 [M+H]⁺. ¹H NMR (400 MHz, Methanol-d₄) δ 7.49 (d, J=10.8 Hz, 1H), 5.06 (q, J=5.7 Hz, 1H), 4.63 (d, J=1.9 Hz, 2H), 3.65-3.54 (m, 1H), 3.54-3.44 (m, 1H), 2.74-2.65 (m, 1H), 2.67-2.59 (m, 1H), 1.55 (s, 6H).

(S)-4-(2-(4,7-difluoro-3,3-dimethyl-2-oxo-5-(trifluoromethyl)indolin-1-yl)acetamido)-3-fluorobutanoic acid (Compound 64B) and methyl (S)-4-(2-(4,7-difluoro-3,3-dimethyl-2-oxo-5-(trifluoromethyl)indolin-1-yl)acetamido)-3-fluorobutanoate (Compound 64B-OMe) may be synthesized following steps 1-4 of Example 19 using (4R)-4-hydroxypyrrolidin-2-one instead of (4S)-4-hydroxypyrrolidin-2-one as the starting material to provide methyl (S)-4-amino-3-fluorobutanoate HCl salt, and steps 1-2 of Example 21.

### Example 22: 4-(2-(5-cyclopropyl-7-fluoro-3,3-dimethyl-2-oxoindolin-1-yl)acetamido)butanoic acid (Compound 66) and tert-butyl 4-(2-(5-cyclopropyl-7-fluoro-3,3-dimethyl-2-oxoindolin-1-yl)acetamido)butanoate (Compound 66-OtBu)

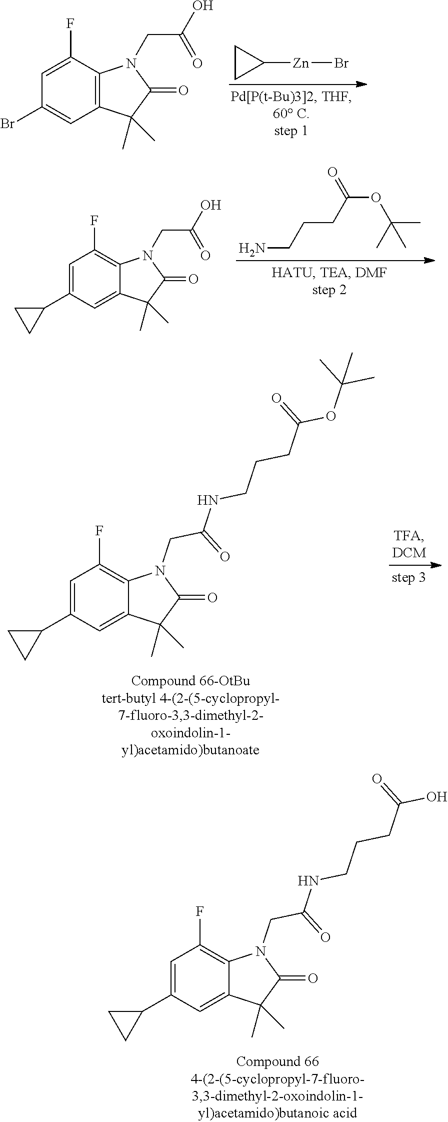

Step 1: Into a 40 mL sealed tube was added (5-bromo-7-fluoro-3,3-dimethyl-2-oxoindol-1-yl)acetic acid (200 mg, 0.633 mmol, 1 equiv) (product of Step 4, Example 8), bis(tri-tert-butylphosphine)palladium(0) (Pd[P(t-Bu)₃)]2) (97 mg, 0.19 mmol, 0.30 equiv), bromo(cyclopropyl)zinc (354 mg, 1.90 mmol, 3 equiv) and tetrahydrofuran (THF) (5 mL) at room temperature. The resulting mixture was stirred for 2 h at 60° C. under nitrogen atmosphere. Desired product could be detected by LCMS. The crude product was directly purified by C18 reverse phase flash chromatography (water:acetonitrile=8:2) to afford (5-cyclopropyl-7-fluoro-3,3-dimethyl-2-oxoindol-1-yl)acetic acid (180 mg, 92% yield) as a white solid. LCMS (LCMS, ESI): RT=0.84 min, m/z=277 [M+H]⁺.

Step 2: Into a 20 mL sealed tube was added (5-cyclopropyl-7-fluoro-3,3-dimethyl-2-oxoindol-1-yl)acetic acid (170 mg, 0.613 mmol, 1 equiv), tert-butyl 4-aminobutanoate (147 mg, 0.919 mmol, 1.5 equiv), [bis(dimethylamino)methylene]-1H-1,2,3-triazolo[4,5-b]pyridinium 3-oxide hexafluorophosphate (HATU) (280 mg, 0.736 mmol, 1.2 equiv), triethylamine (TEA) (310 mg, 3.07 mmol, 5 equiv) and N,N-dimethyl formamide (DMF) (3 mL) at room temperature. The resulting mixture was stirred for 1 h at room temperature under air atmosphere. The residue was directly purified by reverse flash chromatography (C18 silica gel; mobile phase, MeCN in water, 40% to 50% gradient in 15 min; detector, UV 254 nm and 220 nm) to provide tert-butyl 4-[2-(5-cyclopropyl-7-fluoro-3,3-dimethyl-2-oxoindol-1-yl)acetamido]butanoate (also referred to herein as tert-butyl 4-(2-(5-cyclopropyl-7-fluoro-3,3-dimethyl-2-oxoindolin-1-yl)acetamido)butanoate; Compound 66-OtBu) (200 mg, 78% yield) as an off-white solid. LCMS (LCMS, ESI): RT=1.01 min, m/z=418 [M+H]⁺.

Step 3: Into a 8 mL sealed tube was added tert-butyl 4-[2-(5-cyclopropyl-7-fluoro-3,3-dimethyl-2-oxoindol-1-yl)acetamido]butanoate (100 mg, 0.239 mmol, 1 equiv), trifluoroacetic acid (TFA) (2 mL, 26.9 mmol, 113 equiv) and dichloromethane (DCM) (4 mL) at room temperature. The resulting mixture was stirred for 1 h at room temperature under air atmosphere. The resulting mixture was concentrated under reduced pressure. The crude product was purified by C18 reverse phase flash chromatography (water:acetonitrile=6:4) to afford 4-(2-(5-cyclopropyl-7-fluoro-3,3-dimethyl-2-oxoindolin-1-yl)acetamido)butanoic acid (Compound 66) (76.1 mg, 88% yield) as a white solid. LCMS (LCMS, ESI): RT=1.01 min, m/z=418 [M+H]⁺. ¹H NMR (400 MHz, DMSO-d₆) δ 12.00 (s, 1H), 8.12 (t, J=5.7 Hz, 1H), 6.99 (d, J=1.6 Hz, 1H), 6.78 (dd, J=13.0, 1.6 Hz, 1H), 4.33 (d, J=1.7 Hz, 2H), 3.08 (q, J=6.6 Hz, 2H), 2.22 (t, J=7.4 Hz, 2H), 1.91 (tt, J=8.5, 5.1 Hz, 1H), 1.63 (p, J=7.2 Hz, 2H), 1.29 (s, 6H), 0.97-0.86 (m, 2H), 0.71-0.62 (m, 2H).

### Example 23: 4-(2-(4,7-difluoro-3,3-dimethyl-2-oxo-5-(trifluoromethyl)indolin-1-yl)acetamido)-3-methylpentanoic acid (Compound 67, rac-67), (3S,4R)-4-(2-(4,7-difluoro-3,3-dimethyl-2-oxo-5-(trifluoromethyl)indolin-1-yl)acetamido)-3-methylpentanoic acid (Compound 67A*), (3R,4S)-4-(2-(4,7-difluoro-3,3-dimethyl-2-oxo-5-(trifluoromethyl)indolin-1-yl)acetamido)-3-methylpentanoic acid (Compound 67B*), (3S,4S)-4-(2-(4,7-difluoro-3,3-dimethyl-2-oxo-5-(trifluoromethyl)indolin-1-yl)acetamido)-3-methylpentanoic acid (Compound 67C*), (3R,4R)-4-(2-(4,7-difluoro-3,3-dimethyl-2-oxo-5-(trifluoromethyl)indolin-1-yl)acetamido)-3-methylpentanoic acid (Compound 67D*), tert-butyl (3S,4R)-4-(2-(4,7-difluoro-3,3-dimethyl-2-oxo-5-(trifluoromethyl)indolin-1-yl)acetamido)-3-methylpentanoate (Compound 67A*-OtBu), tert-butyl (3R,4S)-4-(2-(4,7-difluoro-3,3-dimethyl-2-oxo-5-(trifluoromethyl)indolin-1-yl)acetamido)-3-methylpentanoate (Compound 67B*-OtBu), tert-butyl (3S,4S)-4-(2-(4,7-difluoro-3,3-dimethyl-2-oxo-5-(trifluoromethyl)indolin-1-yl)acetamido)-3-methylpentanoate (Compound 67C*-OtBu), and tert-butyl (3R,4R)-4-(2-(4,7-difluoro-3,3-dimethyl-2-oxo-5-(trifluoromethyl)indolin-1-yl)acetamido)-3-methylpentanoate (Compound 67D*-OtBu)

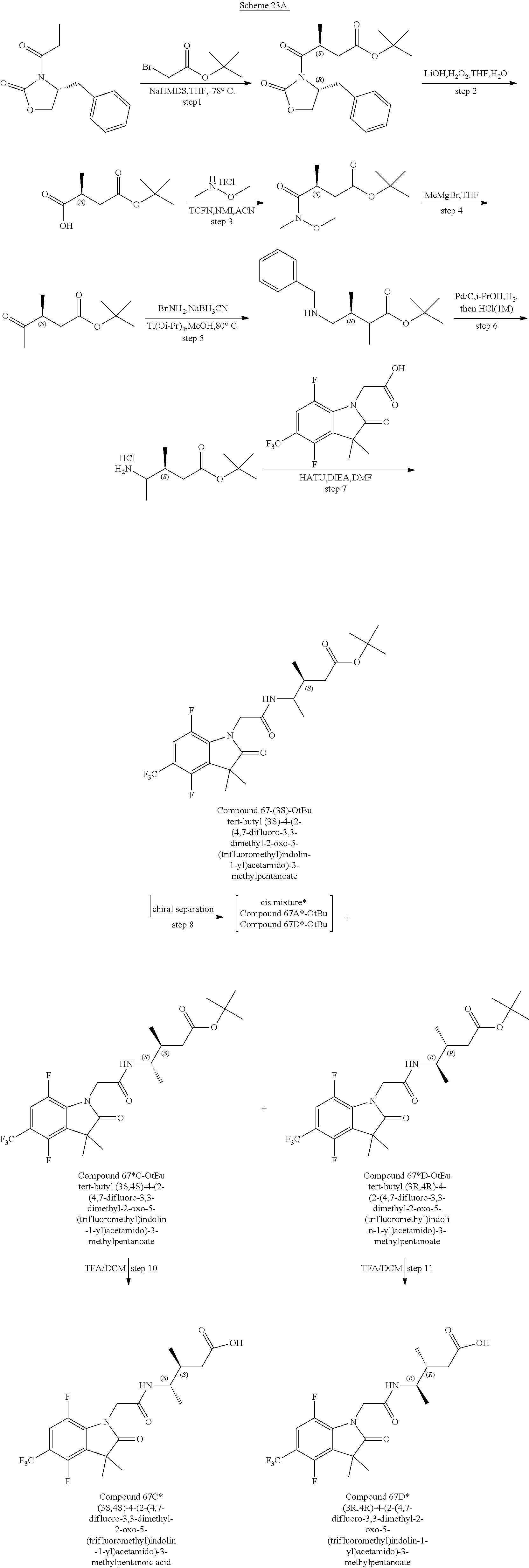

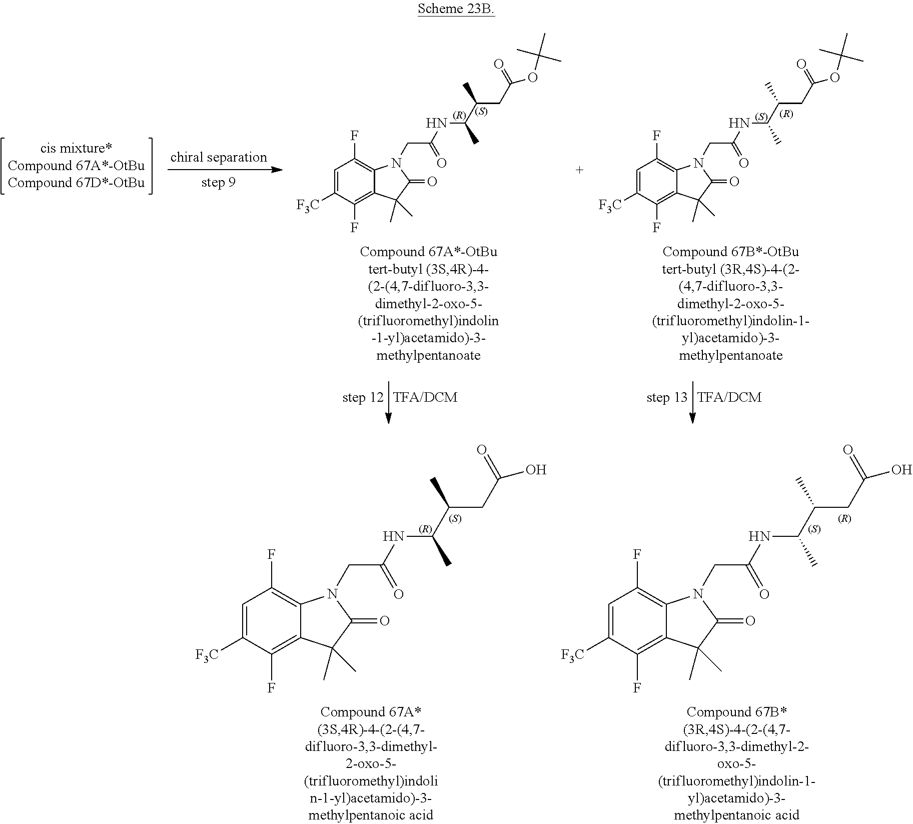

Step 1: To a solution of (4R)-4-benzyl-3-propanoyl-1,3-oxazolidin-2-one (13.6 g, 58.3 mmol, 1 equiv) in tetrahydrofuran (THF) (10 mL) was added dropwise sodium hexamethyldisilazide (NaHMDS) (70 mL, 70 mmol, 1.2 equiv, 1 M in THF) at −78° C. under nitrogen atmosphere. The reaction mixture was stirred at −78° C. for 30 min. Then to the mixture was added tert-butyl 2-bromoacetate (17.1 g, 87.5 mmol, 1.5 equiv). The resulting mixture was stirred for additional 2 h at −78° C., then warmed slowly to room temperature. The reaction was monitored by LCMS. The reaction was quenched with sat. NH₄Cl (100 mL), and then the mixture was extracted with ethyl acetate (EtOAc) (3×40 mL), dried over anhydrous Na₂SO₄, the resulting mixture was concentrated under reduced pressure to provide a residue, which was purified by silica gel column chromatography, eluting with petroleum ether/ethyl acetate (4:1) to afford tert-butyl (3S)-4-[(4R)-4-benzyl-2-oxo-1,3-oxazolidin-3-yl]-3-methyl-4-oxobutanoate (19 g, 80% yield) as a white solid. LCMS:(ES, m/z): RT=1.21 min, m/z=348.2 [M+H]⁺; ¹H NMR (300 MHz, DMSO-d6) δ 7.30 (t, J=5.0 Hz, 5H), 4.65 (s, 1H), 4.43-4.28 (m, 1H), 4.17 (d, J=9.5 Hz, 1H), 3.97 (d, J=10.1 Hz, 1H), 3.08-2.82 (m, 2H), 2.76-2.60 (m, 1H), 2.48-2.36 (m, 1H), 1.41 (t, J=2.5 Hz, 9H), 1.09 (dd, J=6.5, 3.3 Hz, 3H).

Step 2: To a solution of tert-butyl (3S)-4-[(4R)-4-benzyl-2-oxo-1,3-oxazolidin-3-yl]-3-methyl-4-oxobutanoate (19 g, 54.7 mmol, 1 equiv) in tetrahydrofuran (THF) (100 mL) was added H₂O₂ (49.8 g, 1460 mmol, 26.8 equiv, 30% in water) at 0° C. The resulting solution was stirred for 5 min at 0° C. Then a solution of LiOH (3.93 g, 164 mmol, 3 equiv) in H₂O (20 mL) was added to the above solution dropwise at 0° C. The resulting mixture was stirred for additional 2 h at room temperature. The reaction was monitored by LCMS. The reaction was quenched with sat. sodium bicarbonate (100 mL) at room temperature. The aqueous layer was extracted with ethyl acetate (EtOAc) (3×40 mL). The water phase was adjusted to pH 4 with 1M HCl (aq.). The resulting water phase was then extracted with EtOAc (3×40 mL), and the organic phase was dried over anhydrous Na₂SO₄. After filtration, the filtrate was concentrated under reduced pressure to provide (2S)-4-(tert-butoxy)-2-methyl-4-oxobutanoic acid (7.54 g, 73% yield) as a colorless oil. LCMS (ES, m/z): RT=0.73 min, m/z=187.1 [M−H]⁻.

Step 3: To a solution of (2S)-4-(tert-butoxy)-2-methyl-4-oxobutanoic acid (7.5 g, 39.9 mmol, 1 equiv), N,O-dimethylhydroxylamine hydrochloride (2.92 g, 47.8 mmol, 1.2 equiv) in acetonitrile (MeCN) (40 mL) was added chloro- N, N, N′, N′-tetramethylformamidinium hexafluorophosphate (TCFH) (13.4 g, 47.8 mmol, 1.2 equiv) and N-methylimidazole (NMI) (9.81 g, 120 mmol, 3 equiv). The resulting mixture was stirred for 2 h at room temperature. The reaction was monitored by LCMS. The resulting mixture was concentrated under reduced pressure. The reaction was quenched with water (50 mL) at room temperature. The aqueous layer was extracted with ethyl acetate (EtOAc) (3×30 mL). The resulting mixture was concentrated under reduced pressure. The residue was purified by reversed-phase flash chromatography (C18 silica gel; mobile phase, MeCN in water, 45% to 55% gradient in 10 min; detector, UV 254 nm) to provide tert-butyl (3S)-3-[methoxy(methyl)carbamoyl]-3-methylpropanoate (5.54 g, 60% yield) as a colorless oil. LCMS:(ES, m/z): RT=0.93 min, m/z=232.1 [M+H]⁺. ¹H NMR (300 MHz, DMSO-d₆) δ 3.72 (s, 3H), 3.10 (s, 3H), 2.71-2.59 (m, 1H), 2.36-2.18 (m, 2H), 1.38 (s, 9H), 1.02 (d, J=7.0 Hz, 3H).

Step 4: Into a 50 mL 3-necked round-bottom flask was added tert-butyl (3S)-3-[methoxy(methyl)carbamoyl]-3-methylpropanoate (1.00 g, 4.32 mmol, 1 equiv) and tetrahydrofuran (THF) (15 mL) at −78° C. under nitrogen atmosphere. To the above mixture was added methyl magnesium bromide (MeMgBr) (13 mL, 113 mmol, 26.1 equiv, 1 M in THF) dropwise at −78° C. The resulting mixture was stirred for additional 2 h at −40° C. The reaction was monitored by GCMS. The reaction was quenched with sat. NH₄Cl (50 mL). The aqueous layer was extracted with CH₂Cl₂ (3×20 mL). The resulting mixture was concentrated under reduced pressure. The residue was purified by silica gel column chromatography, eluted with CH₂Cl₂/petroleum ether (1:3) to afford tert-butyl (3S)-3-methyl-4-oxopentanoate (790 mg, 59% yield) as a yellow oil. GCMS: RT=3.58 min, m/z=186.1 [M].

Step 5: Into a 20 mL vial was added tert-butyl (3S)-3-methyl-4-oxopentanoate (550 mg, 1.77 mmol, 1 equiv), benzylamine (380 mg, 3.54 mmol, 2 equiv), Ti(Oi-Pr)₄ (252 mg, 0.89 mmol, 0.5 equiv) and methanol (MeOH) (5 mL). The resulting mixture was stirred for 1 h at 80° C. To the above mixture was added sodium cyanoborohydride (NaBH₃CN) (334 mg, 5.32 mmol, 3 equiv) at room temperature. The resulting mixture was stirred for additional overnight at room temperature. The reaction was quenched with water (20 mL) at room temperature. The resulting mixture was filtered, the filter cake was washed with MeOH (3×5 mL). The aqueous layer was extracted with CH₂Cl₂ (3×20 mL). The filtrate was concentrated under reduced pressure. The residue was purified by reversed-phase flash chromatography (C18 silica gel; mobile phase, MeCN in water, 50% to 60% gradient in 10 min; detector, UV 254 nm) to provide tert-butyl (3S)-4-(benzylamino)-3-methylpentanoate (320 mg, 46% yield) as a yellow oil. LCMS (ES, m/z): RT=1.29 min, m/z=278.2 [M+H]⁺. ¹H NMR (400 MHz, DMSO-d₆) δ 7.36-7.26 (m, 4H), 7.24-7.17 (m, 1H), 3.77-3.59 (m, 2H), 2.45 (dd, J=14.7, 4.4 Hz, 1H), 2.05-1.93 (m, 1H), 1.87 (dd, J=14.7, 9.5 Hz, 2H), 1.38 (d, J=7.7 Hz, 9H), 0.90 (t, J=7.4 Hz, 3H), 0.83 (d, J=6.7 Hz, 3H).

Step 6: Into a 8 mL vial was added tert-butyl (3S)-4-(benzylamino)-3-methylpentanoate (300 mg, 1.08 mmol, 1 equiv), Pd/C (750 mg, wet, 10% on carbon) and isopropanol (iPrOH) (4 mL). The resulting mixture was stirred for 2 h at room temperature under hydrogen atmosphere. The reaction was monitored by LCMS. To the above mixture was added HCl (1M, 0.1 mL) and was stirred for additional 10 min. The resulting mixture was filtered, the filter cake was washed with methanol (MeOH) (3×5 mL). The filtrate was concentrated under reduced pressure to afford tert-butyl (3S)-4-amino-3-methylpentanoate hydrochloride (180 mg, 44% yield) as a brown oil. LCMS ES, m/z): RT=0.77 min, m/z=188.2 [M+H]⁺.

Step 7: Into a 8 mL vial was added tert-butyl (3S)-4-amino-3-methylpentanoate hydrochloride (175 mg, 0.93 mmol, 1.2 equiv), [4,7-difluoro-3,3-dimethyl-2-oxo-5-(trifluoromethyl)indol-1-yl]acetic acid (product of step 3, Example 21) (151 mg, 0.47 mmol, 1 equiv), [bis(dimethylamino)methylene]-1H-1,2,3-triazolo[4,5-b]pyridinium 3-oxide hexafluorophosphate (HATU) (213 mg, 0.56 mmol, 1.2 equiv), N,N-diisopropylethylamine (DIPEA) (181 mg, 1.40 mmol, 3 equiv) and N,N-dimethyl formamide (DMF) (2 mL). The resulting mixture was stirred for 2 h at room temperature. The reaction was monitored by LCMS. The reaction was quenched with water (30 mL). The aqueous layer was extracted with ethyl acetate (EtOAc) (4×20 mL). The resulting mixture was concentrated under reduced pressure. The residue was purified by silica gel column chromatography, eluted with petroleum ether/ethyl acetate (9:1) to afford tert-butyl (3S)-4-(2-[4,7-difluoro-3,3-dimethyl-2-oxo-5-(trifluoromethyl)indol-1-yl]acetamido-3-methylpentanoate (220 mg, 58% yield) as a yellow oil. The crude product (220 mg, 80% purity) was purified by Prep-HPLC (XBridge Prep OBD C18 Column, 30*150 mm, 5 μm; Mobile Phase A: water (10 mmol/L NH₄HCO₃), Mobile Phase B: acetonitrile (MeCN); Flow rate: 60 mL/min; Gradient: 55% B to 65% B in 10 min; Wave Length: 254 nm/220 nm; HPLC RT(min): 8.8) to afford tert-butyl (3S)-4-(2-(4,7-difluoro-3,3-dimethyl-2-oxo-5-(trifluoromethyl)indolin-1-yl)acetamido)-3-methylpentanoate (Compound 67-(3S)-OtBu) (100 mg, 35% yield) as a white solid.

Step 8: The Compound 67-(3S)-OtBu mixture of stereoisomers was separated by Prep-Chiral-HPLC (Lux 5 um Cellulose-4, 2.12*25 cm, 5 μm; Mobile Phase A: Hexanes (0.5% 2M NH₃-MeOH), Mobile Phase B: isopropanol (IPA); Flow rate: 20 mL/min; Gradient: 5% B to 5% B in 21.5 min; Wave Length: 220/254 nm) to afford an assumed cis mixture of tert-butyl 4-{2-[4,7-difluoro-3,3-dimethyl-2-oxo-5-(trifluoromethyl)indol-1-yl]acetamido}-3-methylpentanoate isomers (Compound 67A*-OtBu and Compound 67B*-OtBu) (HPLC RT(min): 12.56; 30 mg, 13% yield); tert-butyl (3S,4S)-4-{2-[4,7-difluoro-3,3-dimethyl-2-oxo-5-(trifluoromethyl)indol-1-yl]acetamido}-3-methylpentanoate (Compound 67C*-OtBu) (HPLC RT(min): 16.37; 25 mg, 11% yield) and tert-butyl (3R,4R)-4-{2-[4,7-difluoro-3,3-dimethyl-2-oxo-5-(trifluoromethyl)indol-1-yl]acetamido}-3-methylpentanoate (Compound 67D*-OtBu) (HPLC RT(min): 19.38; 25 mg, 110% yield). Stereochemistry arbitrarily assigned.

Compound 67C*-OtBu: LCMS:(ES, m/z): RT=1.67 min, m/z=493.2 [M+H]⁺. ¹H NMR (400 MHz, DMSO-d₆) δ 8.08 (d, J=8.6 Hz, 1H), 7.71 (dd, J=10.8, 5.6 Hz, 1H), 4.43 (s, 2H), 3.71 (q, J=6.8 Hz, 1H), 2.37-2.26 (m, 1H), 1.98-1.87 (m, 2H), 1.42 (d, J=13.7 Hz, 15H), 1.01 (d, J=6.8 Hz, 3H), 0.85 (d, J=5.8 Hz, 3H).

Compound 67D*-OtBu: LCMS:(ES, m/z): RT=1.67 min, m/z=493.2 [M+H]⁺. ¹H NMR (400 MHz, DMSO-d₆) δ 8.04 (d, J=8.6 Hz, 1H), 7.69 (dd, J=10.8, 5.6 Hz, 1H), 4.44 (d, J=2.0 Hz, 2H), 3.86-3.73 (m, 1H), 2.26 (q, J=8.8 Hz, 1H), 1.99-1.88 (m, 2H), 1.44 (s, 6H), 1.37 (s, 9H), 1.02 (d, J=6.8 Hz, 3H), 0.86 (d, J=6.2 Hz, 3H).

Step 9: The assumed cis mixture of tert-butyl 4-{2-[4,7-difluoro-3,3-dimethyl-2-oxo-5-(trifluoromethyl)indol-1-yl]acetamido}-3-methylpentanoate isomers (Compound 67A*—OtBu and Compound 67B*-OtBu) (30 mg) was purified by Prep-SFC ((S, S)-Whelk-O 1 5 μm Kromasil, 3*25 cm, 5 μm; Mobile Phase A: CO₂, Mobile Phase B: isopropanol (IPA); Flow rate: 90 mL/min; Gradient: isocratic 10% B; Column Temperature (° C.): 35; Back Pressure (bar): 100; Wave Length: 220 nm) to afford tert-butyl (3S,4R)-4-(2-[4,7-difluoro-3,3-dimethyl-2-oxo-5-(trifluoromethyl)indol-1-yl]acetamido-3-methylpentanoate (Compound 67A*-OtBu) (HPLC RT (min): 9.05; 12 mg, 40% yield) and tert-butyl (3R,4S)-4-{2-[4,7-difluoro-3,3-dimethyl-2-oxo-5-(trifluoromethyl)indol-1-yl]acetamido}-3-methylpentanoate (Compound 67B*-OtBu) (HPLC RT (min): 10.63; 10 mg, 33% yield). Stereochemistry arbitrarily assigned.

Compound 67A*-OtBu: LCMS:(ES, m/z): RT=1.32 min, m/z=493.2 [M+H]⁺. ¹H NMR (400 MHz, DMSO-d₆) δ 8.03 (d, J=8.6 Hz, 1H), 7.69 (dd, J=10.8, 5.5 Hz, 1H), 4.44 (d, J=2.0 Hz, 2H), 3.80 (q, J=7.0, 5.8 Hz, 1H), 2.26 (q, J=8.8 Hz, 1H), 2.00-1.87 (m, 2H), 1.44 (s, 6H), 1.37 (s, 9H), 1.02 (d, J=6.8 Hz, 3H), 0.86 (d, J=6.0 Hz, 3H).

Compound 67B*-OtBu: LCMS:(ES, m/z): RT=1.31 min, m/z=493.2 [M+H]⁺. ¹H NMR (400 MHz, DMSO-d₆) δ 8.05 (dd, J=16.2, 8.6 Hz, 1H), 7.75-7.64 (m, 1H), 4.44 (s, 2H), 3.86-3.65 (m, 1H), 2.35-2.21 (m, 1H), 1.99-1.86 (m, 2H), 1.44 (s, 6H), 1.39 (d, J=13.6 Hz, 9H), 1.02 (dd, J=6.8, 4.4 Hz, 3H), 0.86 (dd, J=6.4, 2.7 Hz, 3H).

Step 10: Into a 8 mL vial was added Compound 67C*-OtBu (15 mg, 0.03 mmol, 1 equiv), trifluoroacetic acid (TFA) (0.2 mL) and dichloromethane (DCM) (1 mL). The resulting mixture was stirred for 1 h at room temperature. The reaction was monitored by LCMS. The resulting mixture was concentrated under reduced pressure. The resulting solid was dried under vacuum to afford (3S,4S)-4-(2-(4,7-difluoro-3,3-dimethyl-2-oxo-5-(trifluoromethyl)indolin-1-yl)acetamido)-3-methylpentanoic acid (Compound 67C*) (11 mg, 84% yield) as a white solid. LCMS:(ES, m/z): RT=1.21 min, m/z=437.1 [M+H]⁺. ¹H NMR (400 MHz, Methanol-d₄) δ 7.48 (dd, J=10.8, 5.5 Hz, 1H), 4.58 (t, J=1.6 Hz, 2H), 3.85 (p, J=6.6 Hz, 1H), 2.56-2.36 (m, 1H), 2.12-1.99 (m, 2H), 1.55 (s, 6H), 1.16 (d, J=6.8 Hz, 3H), 1.00 (d, J=6.0 Hz, 3H).

Step 11: Into a 8 mL vial was added Compound 67D*-OtBu (15 mg, 0.03 mmol, 1 equiv), trifluoroacetic acid (TFA) (0.2 mL) and dichloromethane (DCM) (1 mL). The resulting mixture was stirred for 1 h at room temperature. The reaction was monitored by LCMS. The resulting mixture was concentrated under reduced pressure. The crude product (12 mg) was purified by Prep-HPLC (Xselect CSH OBD Column 30*150 mm, 5 μm; Mobile Phase A: water (0.05% TFA), Mobile Phase B: acetonitrile (MeCN); Flow rate: 60 mL/min; Gradient: 5% B to 5% B in 2 min, 40% B to 50% B in 10 min; Wave Length: 254 nm/220 nm, HPLC RT (min): 7.7) to afford (3R,4R)-4-(2-(4,7-difluoro-3,3-dimethyl-2-oxo-5-(trifluoromethyl)indolin-1-yl)acetamido)-3-methylpentanoic acid (Compound 67D*) (8.9 mg, 67% yield) as a white solid. LCMS:(ES, m/z): RT=0.75 min, m/z=437.1 [M+H]⁺. ¹H NMR (400 MHz, DMSO-d₆) δ 12.01 (s, 1H), 8.05 (d, J=8.6 Hz, 1H), 7.70 (dd, J=10.8, 5.6 Hz, 1H), 4.44 (d, J=2.0 Hz, 2H), 3.88-3.63 (m, 1H), 2.37-2.22 (m, 1H), 2.01-1.86 (m, 2H), 1.44 (s, 6H), 1.03 (d, J=6.8 Hz, 3H), 0.87 (d, J=6.0 Hz, 3H).

Step 12: Into a 8 mL vial was added Compound 67A*-OtBu (12 mg, 0.02 mmol, 1 equiv), trifluoroacetic acid (TFA) (0.1 mL) and dichloromethane (DCM) (0.5 mL). The resulting mixture was stirred for 1 h at room temperature. The reaction was monitored by LCMS. The resulting mixture was concentrated under reduced pressure. The resulting mixture was diluted with water (15 mL). The resulting solution was dried by lyophilization. This resulted in (3S,4R)-4-(2-(4,7-difluoro-3,3-dimethyl-2-oxo-5-(trifluoromethyl)indolin-1-yl)acetamido)-3-methylpentanoic acid (Compound 67A*) (9.2 mg, 87% yield) as a white solid. LCMS:(ES, m/z): RT=1.62 min, m/z=437.2 [M+H]⁺. ¹H NMR (400 MHz, Methanol-d₄) δ 7.47 (dd, J=10.8, 5.5 Hz, 1H), 4.67-4.50 (m, 2H), 4.04-3.90 (m, 1H), 2.44 (q, J=8.9 Hz, 1H), 2.15-2.05 (m, 2H), 1.55 (d, J=1.4 Hz, 6H), 1.17 (d, J=6.9 Hz, 3H), 0.99 (d, J=6.2 Hz, 3H).

Step 13: Into a 8 mL vial was added Compound 67B*-OtBu (10 mg, 0.02 mmol, 1 equiv), trifluoroacetic acid (TFA) (0.2 mL) and dichloromethane (DCM) (1 mL). The resulting mixture was stirred for 1 h at room temperature. The reaction was monitored by LCMS. The resulting mixture was concentrated under reduced pressure to afford a crude product (10 mg) which was purified by Prep-HPLC (Xselect CSH OBD Column 30*150 mm, 5 μm; Mobile Phase A: water (0.05% TFA), Mobile Phase B: acetonitrile (MeCN); Flow rate: 60 mL/min; Gradient: 5% B to 5% B in 2 min, 36% B to 46% B in 10 min; Wave Length: 254 nm/220 nm; HPLC RT (min): 9.2) to afford (3R,4S)-4-(2-(4,7-difluoro-3,3-dimethyl-2-oxo-5-(trifluoromethyl)indolin-1-yl)acetamido)-3-methylpentanoic acid (Compound 67B*-OtBu) (2.7 mg, 30% yield) as a white solid. LCMS:(ES, m/z): RT=1.18 min, m/z=437.1 [M+H]⁺. ¹H NMR (400 MHz, Methanol-d₄) δ 7.47 (dd, J=11.2, 5.4 Hz, 1H), 4.59 (dd, J=4.8, 1.9 Hz, 2H), 4.05-3.80 (m, 1H), 2.55-2.35 (m, 1H), 2.15-2.03 (m, 2H), 1.55 (s, 6H), 1.16 (dd, J=6.8, 1.2 Hz, 3H), 1.00 (d, J=6.0 Hz, 3H).

### Example 24: 2-(1-(1-(2-(5-cyclopropyl-4,7-difluoro-3,3-dimethyl-2-oxoindolin-1-yl)acetamido)ethyl)cyclopropyl)acetic acid (Compound 69, rac-69), (R)-2-(1-(1-(2-(5-cyclopropyl-4,7-difluoro-3,3-dimethyl-2-oxoindolin-1-yl)acetamido)ethyl)cyclopropyl)acetic acid (Compound 69A*), (S)-2-(1-(1-(2-(5-cyclopropyl-4,7-difluoro-3,3-dimethyl-2-oxoindolin-1-yl)acetamido)ethyl)cyclopropyl)acetic acid (Compound 69B*), methyl (R)-2-(1-(1-(2-(5-cyclopropyl-4,7-difluoro-3,3-dimethyl-2-oxoindolin-1-yl)acetamido)ethyl)cyclopropyl)acetate (Compound 69A*-OMe) and methyl (S)-2-(1-(1-(2-(5-cyclopropyl-4,7-difluoro-3,3-dimethyl-2-oxoindolin-1-yl)acetamido)ethyl)cyclopropyl)acetate (Compound 69B*-OMe)

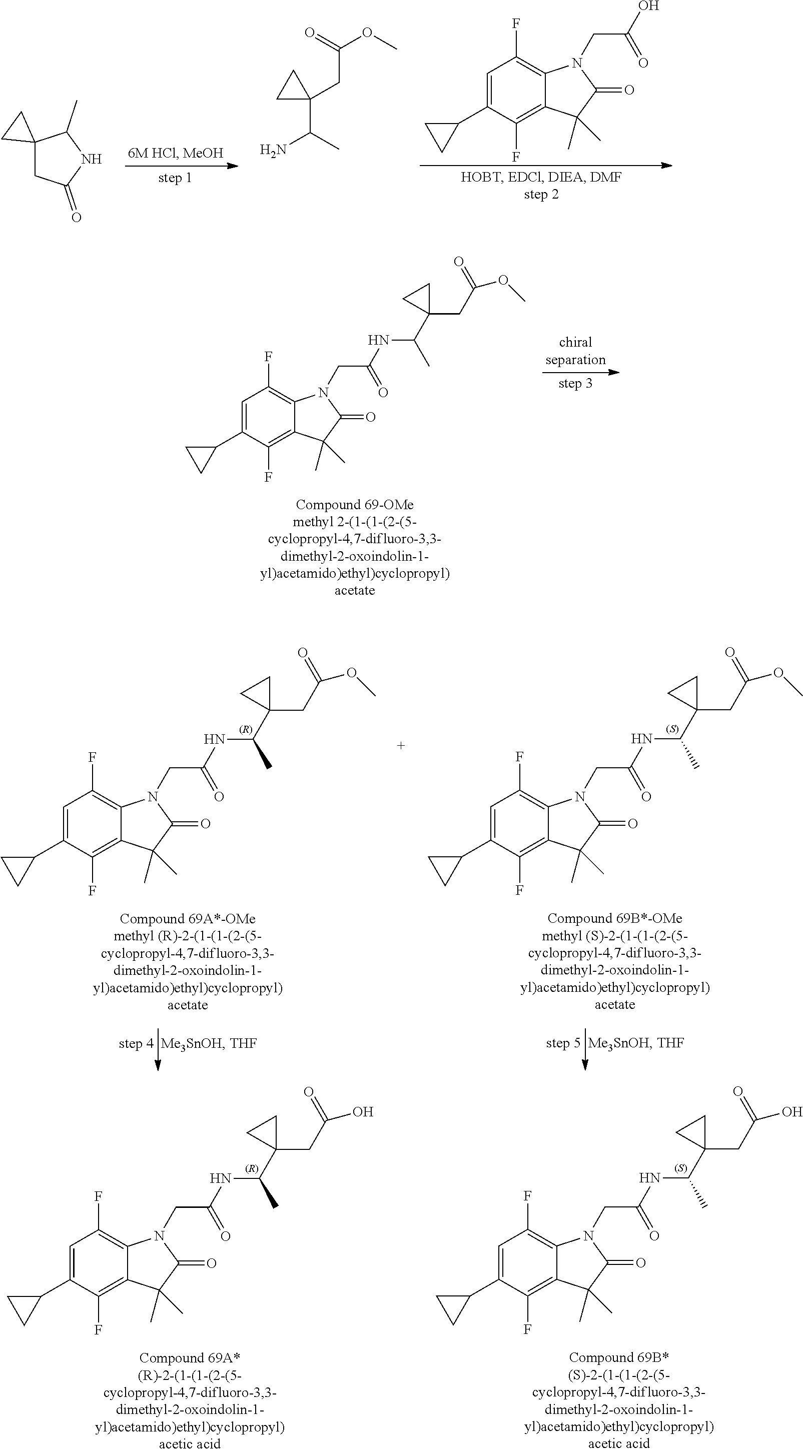

Step 1: Into a 8 mL vial was added 4-methyl-5-azaspiro[2.4]heptan-6-one (200 mg, 1.59 mmol, 1 equiv) and 6M HCl (1 mL), and methanol (MeOH) (1 mL) at room temperature. The mixture was stirred for overnight at 80° C., and the reaction was monitored by LCMS. The resulting mixture was concentrated under reduced pressure to provide methyl 2-[1-(1-aminoethyl)cyclopropyl]acetate hydrochloride (310 mg, 90% yield) as a yellow oil. LCMS:(ES, m/z): RT=0.31 min, m/z=158.1[M+H]⁺.

Step 2: Into a 20 mL vial was added methyl 2-[1-(1-aminoethyl)cyclopropyl]acetate hydrochloride (300 mg, 1.54 mmol, 1.2 equiv) and 2-(5-cyclopropyl-4,7-difluoro-3,3-dimethyl-2-oxoindolin-1-yl)acetic acid (product of Step 6, Example 14) (381 mg, 1.29 mmol, 1 equiv), hydroxybenzotriazle (HOBT) (349 mg, 2.58 mmol, 2 equiv), 1-ethyl-3-(3-dimethylaminopropyl)carbodiimide (EDCI) (495 mg, 2.58 mmol, 2 equiv), N,N-diisopropylethylamine (DIPEA) (500 mg, 3.87 mmol, 3 equiv) and N,N-dimethyl formamide (DMF) (4 mL) at room temperature. The mixture was stirred for 1 h at room temperature, and the reaction monitored by LCMS. The reaction was quenched with water (10 mL) at room temperature. The resulting mixture was extracted with ethyl acetate (EtOAc) (3×20 mL). The combined organic layers were washed with brine (20 mL) and dried over anhydrous Na₂SO₄. After filtration, the filtrate was concentrated under reduced pressure. The residue was purified by reversed-phase flash chromatography (C18 silica gel; mobile phase, MeCN in water (0.1% TFA), 50% to 60% gradient in 20 min; detector, UV 220/254 nm) to afford methyl 2-(1-(1-(2-(5-cyclopropyl-4,7-difluoro-3,3-dimethyl-2-oxoindolin-1-yl)acetamido)ethyl)cyclopropyl)acetate (Compound 69-OMe) (180 mg, 27% yield) as a white solid. LCMS:(ES, m/z): RT=0.96 min, m/z=435.1[M+H]⁺. ¹H NMR (400 MHz, Methanol-d₄) δ 6.70 (dd, J=12.0, 6.0 Hz, 1H), 4.48 (t, J=2.4 Hz, 2H), 3.79-3.69 (m, 1H), 3.59 (s, 3H), 2.68 (dd, J=16.4, 1.2 Hz, 1H), 2.15 (d, J=16.4 Hz, 1H), 2.09-1.99 (m, 1H), 1.53 (d, J=2.6 Hz, 6.0H), 1.13 (d, J=6.8 Hz, 3H), 1.06-0.96 (m, 2H), 0.73-0.61 (m, 3H), 0.60-0.54 (m, 1H), 0.52-0.43 (m, 2H).

Step 3: Compound 69-OMe was purified by Chiral-Prep-HPLC (Lux 5 um Cellulose-4, 2.12*25 cm, 5 μm; Mobile Phase A: Hexanes (0.5% 2M NH₃-MeOH), Mobile Phase B: MeOH:EtOH=1:1—HPLC; Flow rate: 20 mL/min; Gradient: 45% B to 45% B in 14 min; Wave Length: 220/254 nm) to provide methyl (R)-2-(1-(1-(2-(5-cyclopropyl-4,7-difluoro-3,3-dimethyl-2-oxoindolin-1-yl)acetamido)ethyl)cyclopropyl)acetate (Compound 69A*-OMe) (HPLC RT (min): 11.79; 35 mg; 99% purity; LCMS:(ES, m/z): RT=0.92 min, m/z=435.1[M+H]⁺) as a white solid and methyl (S)-2-(1-(1-(2-(5-cyclopropyl-4,7-difluoro-3,3-dimethyl-2-oxoindolin-1-yl)acetamido)ethyl)cyclopropyl)acetate (Compound 69B*-OMe) (HPLC RT (min): 13.01; 32 mg; 98% purity; LCMS:(ES, m/z): RT=0.92 min, m/z=435.1[M+H]⁺) as a white solid. Stereochemistry arbitrarily assigned.

Step 4: Into a 8 mL vial was added Compound 69A*-OMe (30 mg, 0.06 mmol, 1 equiv), trimethylstannanol (37.5 mg, 0.20 mmol, 3 equiv), and tetrahydrofuran (THF) (0.5 mL) at room temperature. The mixture was stirred for 16 h at 60° C. The reaction was monitored by LCMS. The reaction was quenched with water (10 mL) at room temperature. The resulting mixture was extracted with ethyl acetate (EtOAc) (3×30 mL). The combined organic layers were washed with brine (20 mL), and dried over anhydrous Na₂SO₄. After filtration, the filtrate was concentrated under reduced pressure.

The residue was purified by reversed-phase flash chromatography (C18 silica gel; mobile phase, MeCN in water (5 mmol/L NH₄HCO₃), 50% to 60% gradient in 20 min; detector, UV 220/254 nm) to provide (R)-2-(1-(1-(2-(5-cyclopropyl-4,7-difluoro-3,3-dimethyl-2-oxoindolin-1-yl)acetamido)ethyl)cyclopropyl)acetic acid (Compound 69A*) (11.7 mg, 40% yield) as a white solid. LCMS:(ES, m/z): RT=1.53 min, m/z=421.1[M+H]. ¹H NMR (400 MHz, Methanol-d₄) δ 6.69 (dd, J=12.0, 6.0 Hz, 1H), 4.55-4.42 (m, 2H), 3.61 (s, 1H), 2.67 (d, J=16.4 Hz, 1H), 2.10-1.98 (m, 2H), 1.52 (d, J=1.6 Hz, 6H), 1.18 (d, J=6.8 Hz, 3H), 1.03-0.93 (m, 2H), 0.73-0.63 (m, 2H), 0.62-0.55 (m, 1H), 0.55-0.43 (m, 3H).

Step 5: Into a 8 mL vial was added Compound 69B*-OMe (30 mg, 0.06 mmol, 1 equiv), trimethylstannanol (37.46 mg, 0.2 mmol, 3 equiv), and tetrahydrofuran (THF) (0.5 mL) at room temperature. The mixture was stirred for 16 h at 60° C. The reaction was monitored by LCMS. The reaction was quenched with water (10 mL) at room temperature. The resulting mixture was extracted with ethyl acetate (EtOAc) (3×30 mL). The combined organic layers were washed with brine (20 mL), and dried over anhydrous Na₂SO₄. After filtration, the filtrate was concentrated under reduced pressure. The residue was purified by reversed-phase flash chromatography (C18 silica gel; mobile phase, MeCN in water (5 mmol/L NH₄HCO₃), 45% to 60% gradient in 20 min; detector, UV 220/254 nm) to provide This resulted in (S)-2-(1-(1-(2-(5-cyclopropyl-4,7-difluoro-3,3-dimethyl-2-oxoindolin-1-yl)acetamido)ethyl)cyclopropyl)acetic acid (Compound 69B*) (11.5 mg, 40% yield) as a white solid. LCMS:(ES, m/z): RT=1.49 min, m/z=421.1[M+H]⁺. ¹H NMR (400 MHz, Methanol-d₄) δ 6.68 (dd, J=12.0, 6.0 Hz, 1H), 4.51 (dd, J=6.6, 1.8 Hz, 2H), 3.68-3.37 (m, 1H), 2.72-2.68 (m, 1H), 2.11-1.92 (m, 2H), 1.52 (s, 6H), 1.19 (d, J=6.8 Hz, 3H), 1.03-0.95 (m, 2H), 0.72-0.66 (m, 2H), 0.61-0.41 (m, 4H).

### Example 25: 4-(2-(5-cyclopropyl-4,7-difluoro-3,3-dimethyl-2-oxoindolin-1-yl)acetamido)-2,3-dimethylbutanoic acid (Compound 70, rac-70), (2S,3S)-4-(2-(5-cyclopropyl-4,7-difluoro-3,3-dimethyl-2-oxoindolin-1-yl)acetamido)-2,3-dimethylbutanoic acid (Compound 70A*), (2R,3R)-4-(2-(5-cyclopropyl-4,7-difluoro-3,3-dimethyl-2-oxoindolin-1-yl)acetamido)-2,3-dimethylbutanoic acid (Compound 70B*), (2S,3R)-4-(2-(5-cyclopropyl-4,7-difluoro-3,3-dimethyl-2-oxoindolin-1-yl)acetamido)-2,3-dimethylbutanoic acid (Compound 70C*), (2R,3S)-4-(2-(5-cyclopropyl-4,7-difluoro-3,3-dimethyl-2-oxoindolin-1-yl)acetamido)-2,3-dimethylbutanoic acid (Compound 70D*), methyl (2S,3S)-4-(2-(5-cyclopropyl-4,7-difluoro-3,3-dimethyl-2-oxoindolin-1-yl)acetamido)-2,3-dimethylbutanoate (Compound 70A*-OMe), methyl (2R,3R)-4-(2-(5-cyclopropyl-4,7-difluoro-3,3-dimethyl-2-oxoindolin-1-yl)acetamido)-2,3-dimethylbutanoate (Compound 70B*-OMe), methyl (2S,3R)-4-(2-(5-cyclopropyl-4,7-difluoro-3,3-dimethyl-2-oxoindolin-1-yl)acetamido)-2,3-dimethylbutanoate (Compound 70C*-OMe), and methyl (2R,3S)-4-(2-(5-cyclopropyl-4,7-difluoro-3,3-dimethyl-2-oxoindolin-1-yl)acetamido)-2,3-dimethylbutanoate (Compound 70D*-OMe)

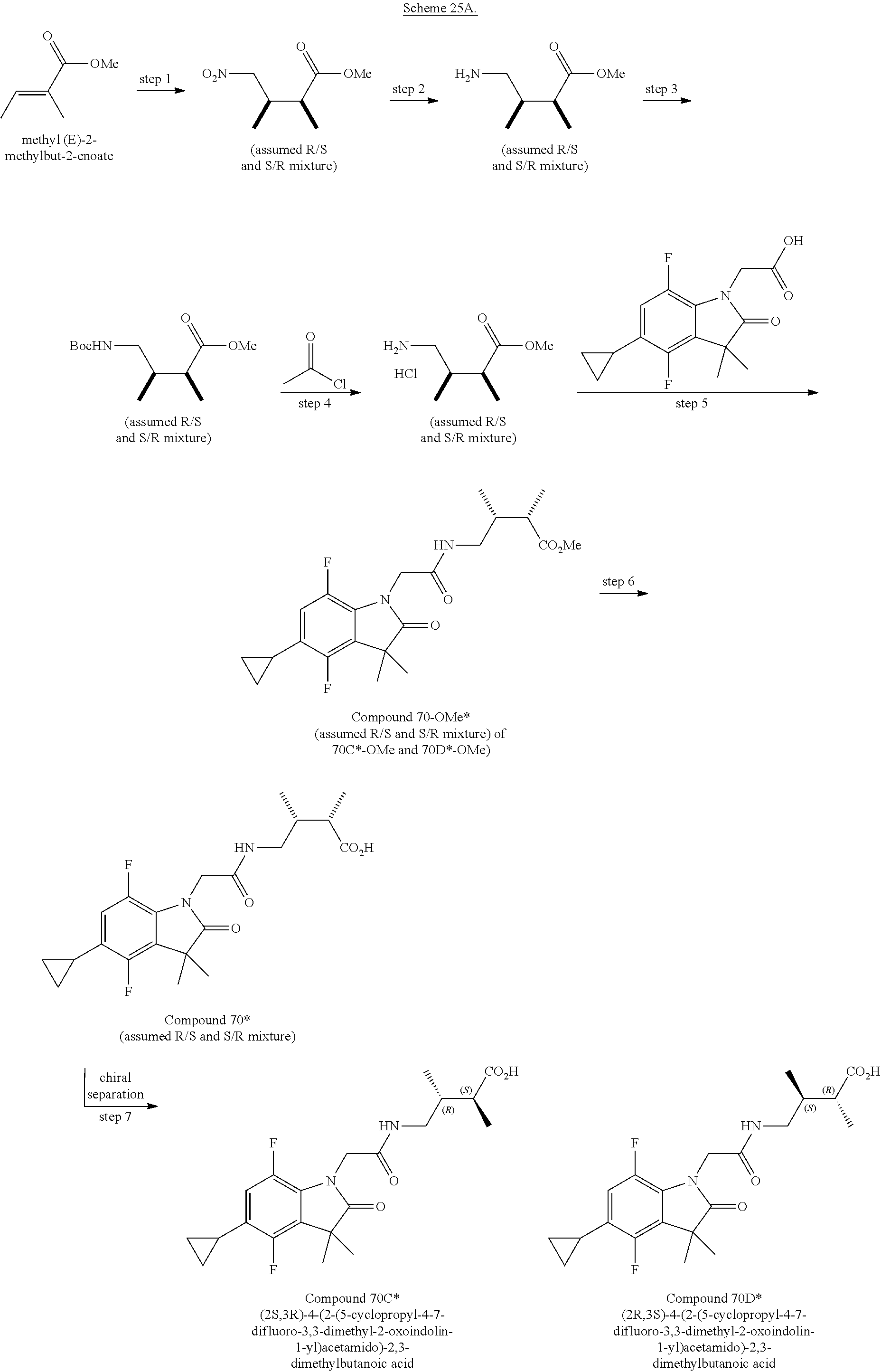

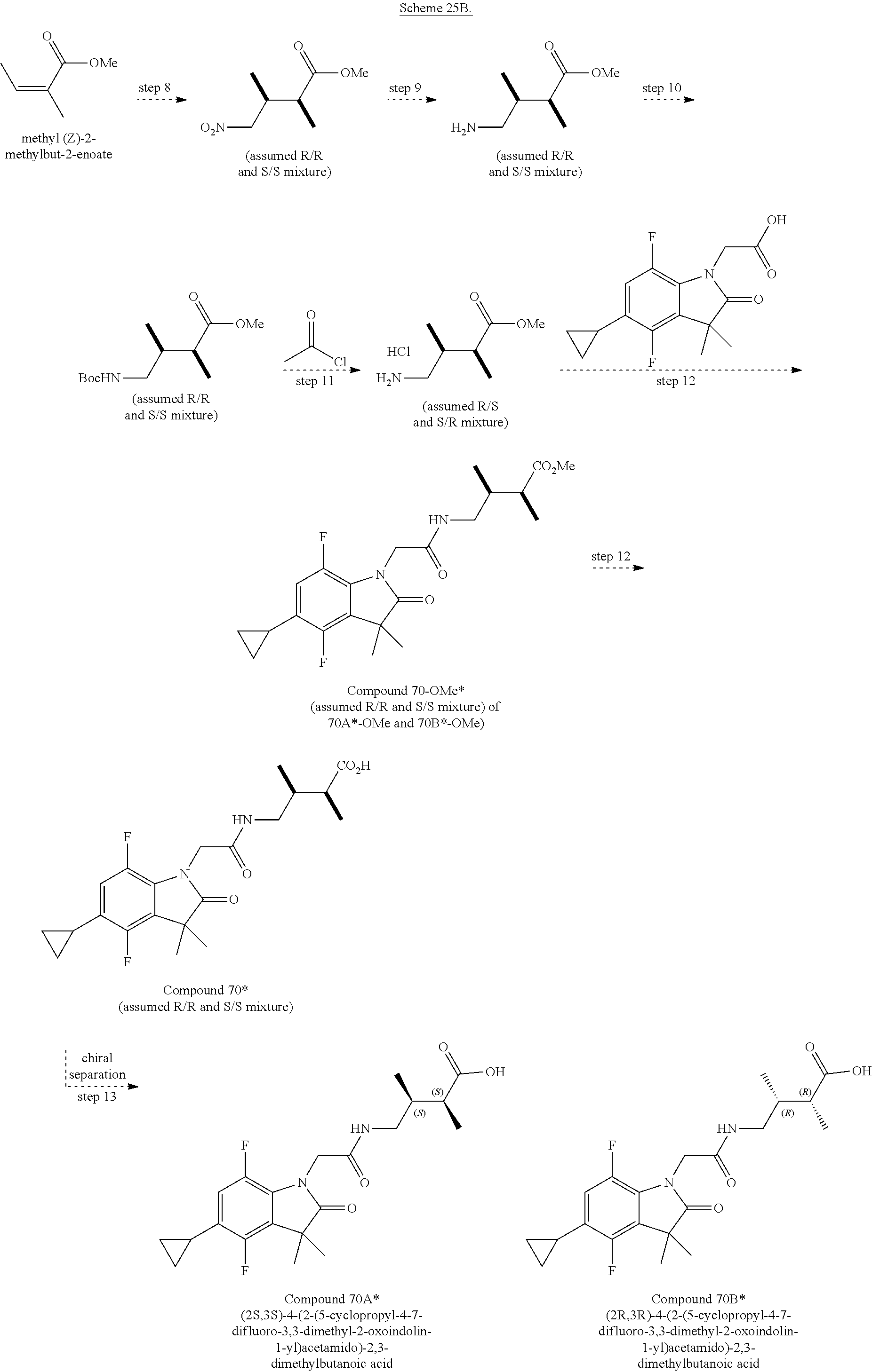

Step 1: Into a 40 mL vial was added methyl (E)-2-methylbut-2-enoate (1 g, 8.76 mmol, 1 equiv), K₂CO₃ (1210 mg, 8.76 mmol, 1 equiv), nitromethane (535 mg, 8.76 mmol, 1 equiv) and methyltrioctylazanium chloride (283 mg, 0.7 mmol, 0.08 equiv) at room temperature. The resulting mixture was stirred for 16 h at room temperature. The reaction was monitored by LCMS. The resulting mixture was filtered, the filter cake was washed with acetonitrile (5 mL). The filtrate was concentrated under reduced pressure. This resulted in crude methyl 2,3-dimethyl-4-nitrobutanoate (assumed S/R and R/S mixture*) (1 g, 49% yield) as a yellow oil. LCMS: (ES, m/z): RT=0.479 min, m/z=176.2[M+H]⁺.

Step 2: Into a 250 mL round-bottom flask was added crude methyl 2,3-dimethyl-4-nitrobutanoate (assumed S/R and R/S mixture*) (1 g, 5.70 mmol, 1 equiv), Fe (3.18 g, 57.1 mmol, 10 equiv), NH₄Cl (3.05 g, 57.1 mmol, 10 equiv), H₂O (4 mL) and methanol (MeOH) (20 mL) at room temperature. The resulting mixture was stirred for 2 h at 80° C. under nitrogen atmosphere. The reaction was monitored by LCMS. The resulting mixture was filtered, the filter cake was washed with MeOH (3×20 mL). The filtrate was concentrated under reduced pressure. This resulted in methyl 4-amino-2,3-dimethylbutanoate (assumed S/R and R/S mixture*) (800 mg, 68% yield) as a brown yellow solid. LCMS: (ES, m/z): RT=0.190 min, m/z=146.2[M+H]⁺.

Step 3: Into a 40 mL vial was added methyl 4-amino-2,3-dimethylbutanoate (assumed S/R and R/S mixture*) (500 mg, 3.44 mmol, 1 equiv), methanol (MeOH) (5 mL), H₂O (1 mL), ditertbutyl decarbonate ((Boc)₂O) (1.12 g, 5.16 mmol, 1.5 equiv) and NaHCO₃ (868 mg, 10.32 mmol, 3 equiv) at room temperature. The resulting mixture was stirred for 1 h at room temperature. The reaction was quenched by the addition of water (50 mL) at room temperature. The resulting mixture was extracted with CH₂Cl₂ (3×50 mL), and the organic phase was dried over anhydrous Na₂SO₄. After filtration, the filtrate was concentrated under reduced pressure. The residue was purified by silica gel column chromatography, eluted with petroleum ether/ethyl acetate (1:1) to afford methyl 4-[(tert-butoxycarbonyl)amino]-2,3-dimethylbutanoate (assumed S/R and R/S mixture*) (300 mg, 28% yield) as a yellow oil. LCMS: (ES, m/z): RT=0.783 min, m/z=246.2[M+H]⁺. ¹H NMR (400 MHz, DMSO-d₆) δ 6.77 (d, J=6.4 Hz, 1H), 3.59 (d, J=2.4 Hz, 3H), 3.02-2.96 (m, 1H), 2.85-2.75 (m, 1H), 2.37 (q, J=6.8 Hz, 1H), 1.81-1.73 (m, 1H), 1.37 (s, 9H), 1.06 (d, J=7.2 Hz, 3H), 0.79 (d, J=6.8 Hz, 3H).

Step 4: Into a 8 mL vial was added methyl (2S,3R)-4-[(tert-butoxycarbonyl)amino]-2,3-dimethylbutanoate (assumed S/R and R/S mixture*) (200 mg, 0.81 mmol, 1 equiv) and methanol (MeOH) (3 mL), and acetyl chloride (96.0 mg, 1.22 mmol, 1 equiv) was added by dropwise at room temperature. The resulting mixture was stirred for 1 h at room temperature. The reaction was monitored by LCMS. The resulting mixture was concentrated under reduced pressure. This resulted in methyl 4-amino-2,3-dimethylbutanoate hydrochloride salt (assumed S/R and R/S mixture*) (250 mg, 90% yield) as a white solid. LCMS: (ES, m/z): RT=0.265 min, m/z=146.2[M+H]⁺.

Step 5: Into a 8 mL vial was added 2-(5-cyclopropyl-4,7-difluoro-3,3-dimethyl-2-oxoindolin-1-yl)acetic acid (product of Step 6, Example 14) (160 mg, 0.54 mmol, 1 equiv), methyl 4-amino-2,3-dimethylbutanoate hydrochloride salt (assumed S/R and R/S mixture*) (246 mg, 1.35 mmol, 2.5 equiv), acetonitrile (1.5 mL), chloro- N, N, N′, N′-tetramethylformamidinium hexafluorophosphate (TCFH) (182 mg, 0.65 mmol, 1.2 equiv) and N-methylimidazole (NMI) (133 mg, 1.62 mmol, 3 equiv) at room temperature. The resulting mixture was stirred for 1 h at room temperature. The reaction was monitored by LCMS. The reaction was quenched by the addition of water (10 mL) at room temperature. The resulting mixture was extracted with CH₂Cl₂ (3×10 mL) and the organic phase was dried over anhydrous Na₂SO₄. After filtration, the filtrate was concentrated under reduced pressure. The residue was purified by reversed-phase flash chromatography (C18 silica gel; mobile phase, MeCN in water, 0% to 100% gradient in 30 min; detector, UV 254/220 nm) to provide methyl 4-[2-(5-cyclopropyl-4,7-difluoro-3,3-dimethyl-2-oxoindol-1-yl)acetamido]-2,3-dimethylbutanoate (Compound 70-OMe*) (assumed S/R and R/S mixture*; Compounds 70C*-OMe and 70D*-OMe) (40 mg, 17% yield) as a colorless oil. LCMS: (ES, m/z): RT=1.394 min, m/z=423.1[M+H]⁺. ¹H NMR (400 MHz, Methanol-d₄) δ 6.69 (dd, J=12.0, Hz, 1H), 4.51 (d, J=1.6 Hz, 2H), 3.68 (d, J=0.8 Hz, 3H), 3.30-3.14 (m, 2H), 2.53-2.44 (m, 1H), 2.07-2.01 (m, 1H), 1.94 (p, J=6.4 Hz, 1H), 1.51 (s, 6H), 1.17 (dd, J=7.2 Hz, 3H), 1.05-0.96 (m, 2H), 0.93 (dd, J=7.2 Hz, 3H), 0.72-0.64 (m, 2H).

Step 6: Into a 8 mL vial was added Compound 70-OMe* (assumed S/R and R/S mixture*; Compounds 70C*-OMe and 70D*-OMe) (30 mg, 0.07 mmol, 1 equiv), tetrahydrofuran (THF) (1 mL), H₂O (0.20 mL) and LiOH (8.50 mg, 0.35 mmol, 5 equiv) at room temperature. The resulting mixture was stirred for 1 h at room temperature. The reaction was monitored by LCMS. The mixture was adjusted to pH 3 with HCl (1M, 2 mL). The resulting mixture was extracted with ethyl acetate (EtOAc) (3×5 mL), and the organic phase was dried over anhydrous Na₂SO₄. After filtration, the filtrate was concentrated under reduced pressure to provide 4-[2-(5-cyclopropyl-4,7-difluoro-3,3-dimethyl-2-oxoindol-1-yl)acetamido]-2,3-dimethylbutanoic acid (Compound 70*) (assumed S/R and R/S mixture*; Compounds 70C* and 70D*) (25 mg, 40% purity) as a light yellow solid, which was purified by Prep-HPLC (XBridge Prep OBD C18 Column, 30*150 mm, 5 μm; Mobile Phase A: Water (10 mmol/L NH₄HCO₃), Mobile Phase B: acetonitrile; Flow rate: 60 mL/min; Gradient: 22% B to 32% B in 10 min; Wave Length: 254/220 nm; HPLC RT (min): 11.83) to provide (Compound 70*) (assumed S/R and R/S mixture*; Compounds 70C* and 70D*) (8 mg, 8% yield) as a light yellow solid. LCMS: (ES, m/z): RT=1.612 min, m/z=409.2[M+H]⁺¹H NMR (400 MHz, Methanol-d₄) δ 6.68 (dd, J=12.0 Hz, 1H), 4.52 (d, J=1.6 Hz, 2H), 3.30-3.24 (m, 1H), 3.18 (dd, J=13.2 Hz, 1H), 2.25-2.17 (m, 1H), 2.06-2.01 (m, 1H), 1.87 (p, J=6.8 Hz, 1H), 1.51 (s, 6H), 1.12 (d, J=6.8 Hz, 3H), 1.02-0.92 (m, 5H), 0.72-0.63 (m, 2H).

Step 7: The diastereomeric mixture of Compound 70* (assumed S/R and R/S mixture*; Compounds 70C* and 70D*) (8 mg, 99% purity) was separated by Chiral-Prep-HPLC (Lux 5 um Cellulose-2 2.12*25 cm, 5 μm; Mobile Phase A: Hexanes (0.1% TFA), Mobile Phase B: isopropanol (IPA); Flow rate: 20 mL/min; Gradient: 15% B to 15% B in 25 min; Wave Length: 220/254 nm) to provide (2S,3R)-4-(2-(5-cyclopropyl-4,7-difluoro-3,3-dimethyl-2-oxoindolin-1-yl)acetamido)-2,3-dimethylbutanoic acid (Compound 70C*) (HPLC RT (min): 16.04; 1 mg; 10% yield) as a white solid, and (2R,3S)-4-(2-(5-cyclopropyl-4,7-difluoro-3,3-dimethyl-2-oxoindolin-1-yl)acetamido)-2,3-dimethylbutanoic acid (Compound 70D*) (HPLC RT (min): 20.75; 0.9 mg; 10% yield) as a white solid. Stereochemistry arbitrarily assigned.

Compound 70C*: LCMS: (ES, m/z): RT=1.306 min, m/z=409.2[M+H]⁺. ¹H NMR (400 MHz, DMSO-d₆) δ 8.12 (t, J=5.6 Hz, 1H), 7.26 (s, 1H), 6.79 (dd, J=12.0 Hz, 1H), 4.36 (d, J=2.0 Hz, 2H), 3.12 (s, 1H), 3.07-2.97 (m, 1H), 2.27 (p, J=6.8 Hz, 1H), 2.01-1.95 (m, 1H), 1.77 (p, J=7.2 Hz, 1H), 1.40 (s, 6H), 1.04 (d, J=7.2 Hz, 3H), 0.98-0.91 (m, 2H), 0.84 (d, J=6.8 Hz, 3H), 0.75-0.64 (m, 2H).

Compound 70D*: LCMS: (ES, m/z): RT=1.319 min, m/z=409.2[M+H]⁺. ¹H NMR (400 MHz, DMSO-d₆) δ 8.12 (t, J=5.6 Hz, 1H), 6.79 (dd, J=12.0 Hz, 1H), 4.36 (s, 2H), 3.12 (s, 1H), 3.03 (d, J=7.2 Hz, 1H), 2.26 (q, J=6.8 Hz, 1H), 2.02-1.96 (m, 1H), 1.84-1.67 (m, 1H), 1.40 (s, 6H), 1.04 (d, J=7.2 Hz, 3H), 0.99-0.89 (m, 2H), 0.84 (d, J=6.8 Hz, 3H), 0.71 (t, J=5.2 Hz, 2H).

Steps 8-13: (2S,3S)-4-(2-(5-cyclopropyl-4,7-difluoro-3,3-dimethyl-2-oxoindolin-1-yl)acetamido)-2,3-dimethylbutanoic acid (Compound 70A*), (2R,3R)-4-(2-(5-cyclopropyl-4,7-difluoro-3,3-dimethyl-2-oxoindolin-1-yl)acetamido)-2,3-dimethylbutanoic acid (Compound 70B*), and Compounds 70A*-OMe and 70B*-OMe may be synthesized following Steps 1-7 of Example 25, using methyl (Z)-2-methylbut-2-enoate as the Step 1 starting material instead of methyl (E)-2-methylbut-2-enoate.

### Example 26: 4-(2-(5-cyclopropyl-4,7-difluoro-3,3-dimethyl-2-oxoindolin-1-yl)acetamido)-3,3-dimethylpentanoic acid (Compound 71, rac-71), (R)-4-(2-(5-cyclopropyl-4,7-difluoro-3,3-dimethyl-2-oxoindolin-1-yl)acetamido)-3,3-dimethylpentanoic acid (Compound 71A*), (S)-4-(2-(5-cyclopropyl-4,7-difluoro-3,3-dimethyl-2-oxoindolin-1-yl)acetamido)-3,3-dimethylpentanoic acid (Compound 71B*), ethyl (R)-4-(2-(5-cyclopropyl-4,7-difluoro-3,3-dimethyl-2-oxoindolin-1-yl)acetamido)-3,3-dimethylpentanoate (Compound 71A*-OEt) and ethyl (S)-4-(2-(5-cyclopropyl-4,7-difluoro-3,3-dimethyl-2-oxoindolin-1-yl)acetamido)-3,3-dimethylpentanoate (Compound 71B*-OEt)

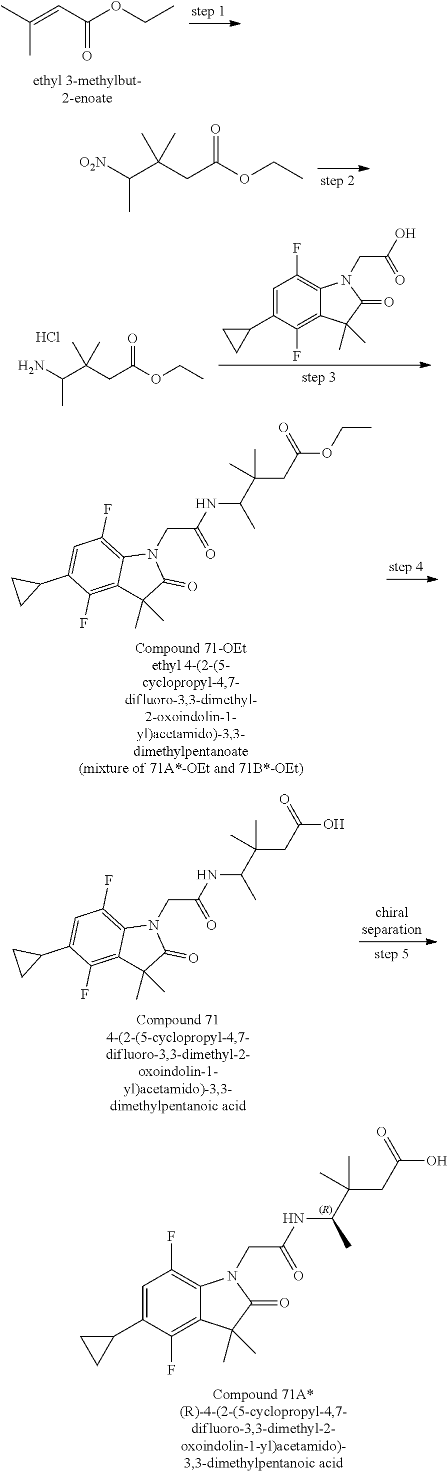

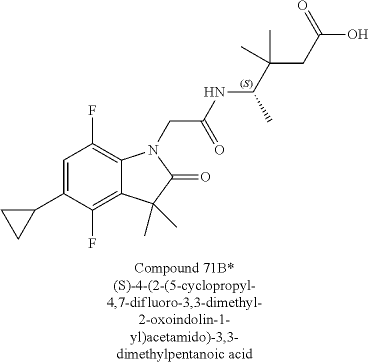

Step 1: Into a 40 mL round-bottom flask was added ethyl 3-methylbut-2-enoate (10 g, 87.6 mmol, 1.00 equiv), tetra n-butylammonium fluoride (TBAF) (1M in THF, 87 mL, 87.6 mmol, 1 equiv) and nitroethane (6.58 g, 87.6 mmol, 1 equiv) at room temperature. The mixture was stirred for 48 h at 60° C. The reaction was monitored by LCMS. The residue was purified by silica gel column chromatography, eluted with petroleum ether/ethyl acetate (6:1) to afford ethyl 3,3-dimethyl-4-nitropentanoate (5 g, 30% yield) as a yellow oil. LCMS: (ES, m/z): RT=0.68 min, m/z=204.1[M+H]⁺.

Step 2: Into a 100 mL round-bottom flask was added ethyl 3,3-dimethyl-4-nitropentanoate (5 g, 24.6 mmol, 1 equiv) and Zn (3.22 g, 49.2 mmol, 2 equiv), HCl (6M) (20 mL), tetrahydrofuran (THF) (20 mL) at room temperature. The mixture was stirred for 2 h at room temperature. The reaction was monitored by LCMS. The resulting mixture was filtered, and the filtrate was concentrated under reduced pressure to provide ethyl 4-amino-3,3-dimethylpentanoate hydrogen chloride (1 g, 24% yield) as a colorless oil. LCMS: (ES, m/z): RT=0.41 min, m/z=174.1[M+H]⁺.

Step 3: A solution of ethyl 4-amino-3,3-dimethylpentanoate hydrogen chloride (141 mg, 0.812 mmol, 1.2 equiv), 2-(5-cyclopropyl-4,7-difluoro-3,3-dimethyl-2-oxoindolin-1-yl)acetic acid (product of Step 6, Example 14) (200 mg, 0.677 mmol, 1 equiv), bis(2-oxo-3-oxazolidinyl)phosphinic chloride (BOPCl) (344 mg, 1.35 mmol, 2.0 equiv) and triethylamine (TEA) (137 mg, 1.35 mmol, 2 equiv) in dichloromethane (DCM) (3 mL) was stirred for 2 h at room temperature. The reaction was monitored by LCMS. The resulting mixture was concentrated under reduced pressure. The residue was purified by reversed-phase flash chromatography (C18 silica gel; mobile phase, MeCN in water (0.1% TFA), 10% to 50% gradient in 10 min; detector, UV 254 nm) to provide ethyl 4-[2-(5-cyclopropyl-4,7-difluoro-3,3-dimethyl-2-oxoindol-1-yl)acetamido]-3,3-dimethylpentanoate (Compound 71-OEt), comprising a mixture of two enantiomers, Compound 71A*-OEt and Compound 71B*-OEt (100 mg, 33% yield) as a yellow oil. LCMS: (ES, m/z): RT=1.01 min, m/z=451.2[M+H]⁺.

Step 4: A solution of Compound 71-OEt (50 mg, 0.11 mmol, 1 equiv), HCl (3M, 1 mL), and acetic acid (AcOH) (2 mL) was stirred for 1 h at 80° C. The reaction was monitored by LCMS. The resulting mixture was concentrated under reduced pressure. The residue was purified by reversed-phase flash chromatography (C18 silica gel; mobile phase, MeCN in water (0.1% TFA), 10% to 50% gradient in 10 min; detector, UV 254 nm) to provide a crude residue (30 mg) which was purified by Prep-HPLC (XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 17% B to 27% B in 7.8 min; Wave Length: 254/272 nm; HPLC RT (min): 14.67) to provide 4-[2-(5-cyclopropyl-4,7-difluoro-3,3-dimethyl-2-oxoindol-1-yl)acetamido]-3,3-dimethylpentanoic acid (Compound 71). LCMS: (ES, m/z): RT=0.67 min, m/z=423.2 [M+H]⁺.

Step 5: The stereoisomers of Compound 71 may be separated using Chiral Prep-HPLC to provide (R)-4-(2-(5-cyclopropyl-4,7-difluoro-3,3-dimethyl-2-oxoindolin-1-yl)acetamido)-3,3-dimethylpentanoic acid (Compound 71A*), and (S)-4-(2-(5-cyclopropyl-4,7-difluoro-3,3-dimethyl-2-oxoindolin-1-yl)acetamido)-3,3-dimethylpentanoic acid (Compound 71B*). Stereochemistry arbitrarily assigned.

### Example 27: 4-(2-(5-cyclopropyl-4,7-difluoro-3,3-dimethyl-2-oxoindolin-1-yl)acetamido)-3-(trifluoromethyl)pentanoic acid (Compound 72, rac-72), (3S,4R)-4-(2-(5-cyclopropyl-4,7-difluoro-3,3-dimethyl-2-oxoindolin-1-yl)acetamido)-3-(trifluoromethyl)pentanoic acid (Compound 72A*), (3R,4S)-4-(2-(5-cyclopropyl-4,7-difluoro-3,3-dimethyl-2-oxoindolin-1-yl)acetamido)-3-(trifluoromethyl)pentanoic acid (Compound 72B*), (3S,4S)-4-(2-(5-cyclopropyl-4,7-difluoro-3,3-dimethyl-2-oxoindolin-1-yl)acetamido)-3-(trifluoromethyl)pentanoic acid (Compound 72C*), (3R,4R)-4-(2-(5-cyclopropyl-4,7-difluoro-3,3-dimethyl-2-oxoindolin-1-yl)acetamido)-3-(trifluoromethyl)pentanoic acid (Compound 72D*), ethyl (3S,4R)-4-(2-(5-cyclopropyl-4,7-difluoro-3,3-dimethyl-2-oxoindolin-1-yl)acetamido)-3-(trifluoromethyl)pentanoate (Compound 72A*-OEt), ethyl (3R,4S)-4-(2-(5-cyclopropyl-4,7-difluoro-3,3-dimethyl-2-oxoindolin-1-yl)acetamido)-3-(trifluoromethyl)pentanoate (Compound 72B*-OEt), ethyl (3S,4S)-4-(2-(5-cyclopropyl-4,7-difluoro-3,3-dimethyl-2-oxoindolin-1-yl)acetamido)-3-(trifluoromethyl)pentanoate (Compound 72C*-OEt), and ethyl (3R,4R)-4-(2-(5-cyclopropyl-4,7-difluoro-3,3-dimethyl-2-oxoindolin-1-yl)acetamido)-3-(trifluoromethyl)pentanoate (Compound 72D*-OEt)

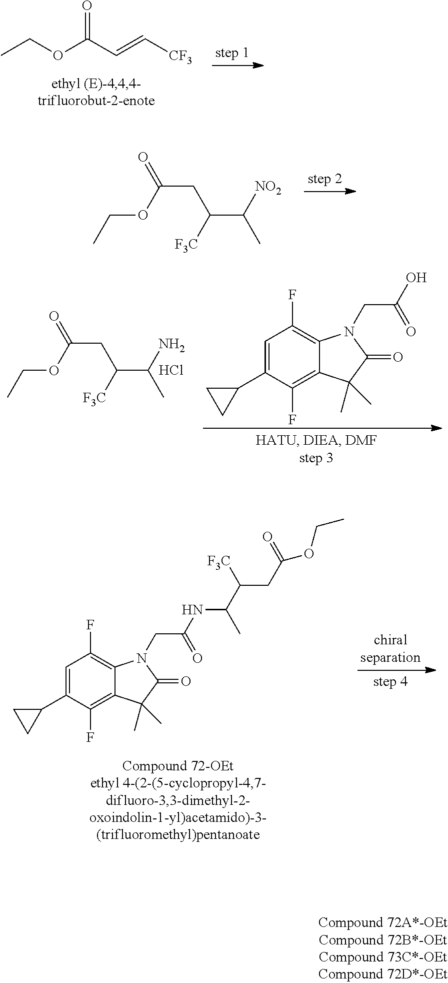 [SMILES: CCOC(=O)/C=C/C(F)(F)F ; CCOC(=O)CC(C(C)[N+](=O)[O-])C(F)(F)F ; CCOC(=O)CC(C(C)N)C(F)(F)F ; Cl ; CC1(C)C(=O)N(CC(=O)O)c2c(F)cc(C3CC3)c(F)c21 ; CCOC(=O)CC(C(C)NC(=O)CN1C(=O)C(C)(C)c2c(F)c(C3CC3)cc(F)c21)C(F)(F)F]

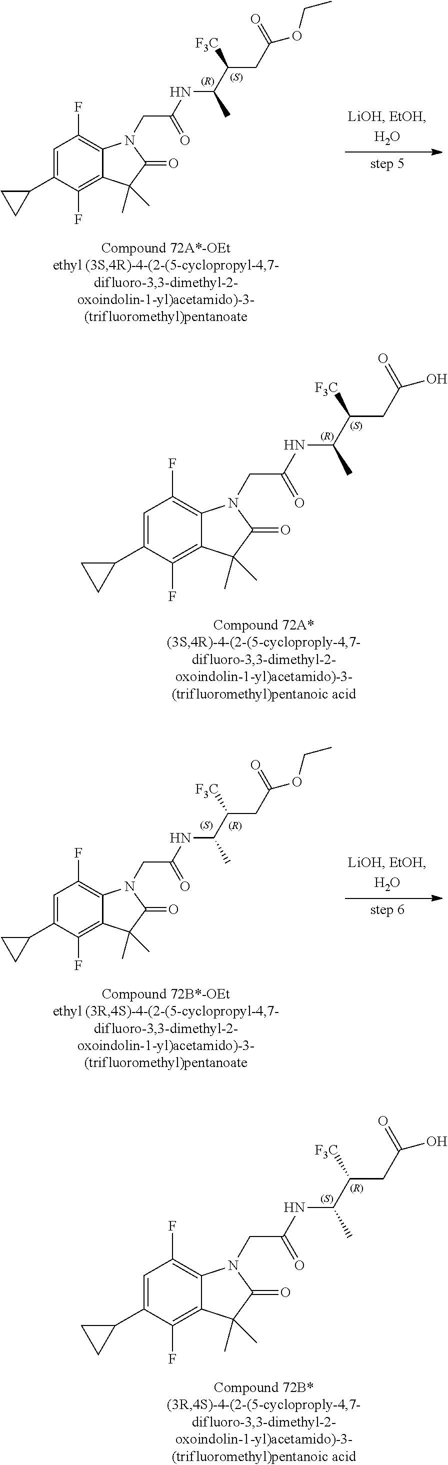

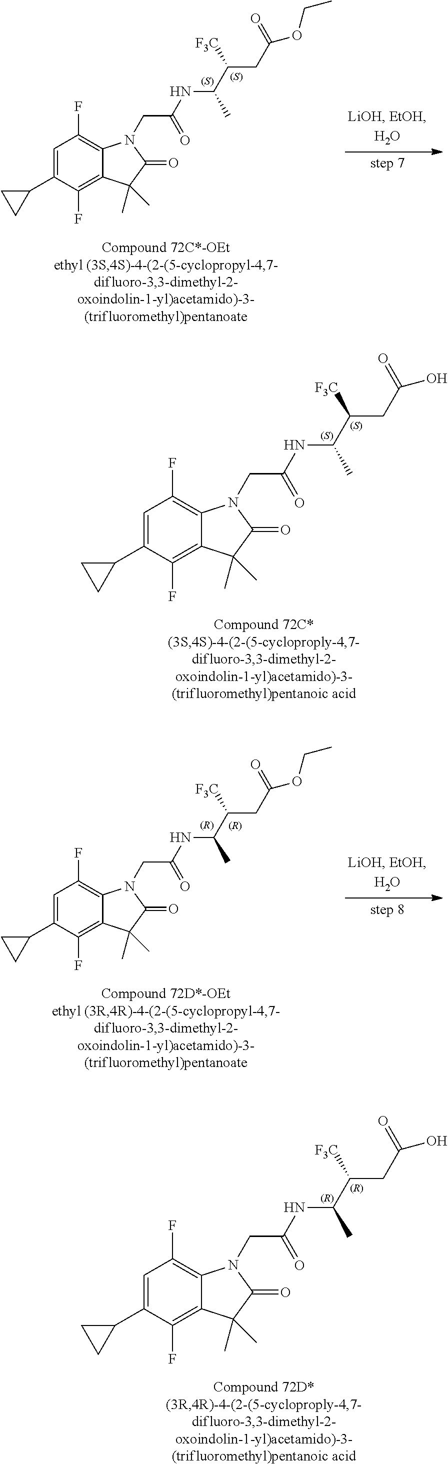

Step 1: Into a 40 mL vial was added ethyl (2E)-4,4,4-trifluorobut-2-enoate (5 g, 29.7 mmol, 1 equiv), nitroethane (6.70 g, 89.2 mmol, 3 equiv), methyltrioctylammonium chloride (1.2 g, 2.97 mmol, 0.1 equiv), and K₂CO₃ (4.11 g, 29.7 mmol, 1 equiv) at room temperature. The resulting mixture was stirred for 16 h at room temperature under nitrogen atmosphere. The reaction was monitored by LCMS. The reaction was quenched with water (60 mL) at room temperature, and the resulting mixture was adjusted pH=3 with HCl (2M), extracted with ethyl acetate (EtOAc) (3×100 mL), and the combined organic layers were washed with brine (2×100 mL) and dried over anhydrous Na₂SO₄. After filtration, the filtrate was concentrated under reduced pressure to provide a residue, which was purified by reversed-phase flash chromatography (C18 silica gel; mobile phase, MeCN in water, 0% to 100% gradient in 30 min; detector, UV 254/220 nm) to provide ethyl 4-nitro-3-(trifluoromethyl)pentanoate (mixture of 4 stereoisomers) (3 g, 41% yield) as a yellow oil. LCMS:(ES, m/z): RT=0.816 min, m/z=244.1 [M+H]⁺. ¹H NMR (400 MHz, DMSO-d₆) δ 5.19-5.06 (m, 1H), 4.20-4.07 (m, 2H), 3.86-3.65 (m, 1H), 2.94-2.65 (m, 2H), 1.60-1.50 (m, 3H), 1.28-1.17 (m, 3H).

Step 2: Into a 250 mL round-bottom flask was added ethyl 4-nitro-3-(trifluoromethyl)pentanoate (500 mg, 2.05 mmol, 1 equiv), EtOH (50 mL), and Raney Ni (881 mg, 10.3 mmol, 5 equiv) at room temperature. The resulting mixture was stirred for 1 h at room temperature under hydrogen atmosphere, and the reaction was monitored by LCMS. The resulting mixture was filtered, the filter cake was washed with ethanol (2×10 mL), HCl (2M, 2.0 mL), and the filtrate then was concentrated under reduced pressure to provide ethyl 4-amino-3-(trifluoromethyl)pentanoate hydrochloride salt (200 mg, 39% yield) as a brown oil. LCMS:(ES, m/z):RT=0.471 min, m/z=214.1 [M+H]⁺.

Step 3: Into a 20 mL vial was added 2-(5-cyclopropyl-4,7-difluoro-3,3-dimethyl-2-oxoindolin-1-yl)acetic acid (product of Step 6, Example 14) (200 mg, 0.67 mmol, 1 equiv), ethyl 4-amino-3-(trifluoromethyl)pentanoate hydrochloride salt (338 mg, 1.35 mmol, 2 equiv), hydroxybenzotriazle (HOBT) (183 mg, 1.35 mmol, 2 equiv), 1-ethyl-3-(3-dimethylaminopropyl)carbodiimide (EDCI) (258 mg, 1.35 mmol, 2 equiv), N,N-diisopropylethylamine (DIPEA) (438 mg, 3.38 mmol, 5 equiv), and N,N-dimethyl formamide (DMF) (3 mL) at room temperature. The resulting mixture was stirred for 1 h at room temperature under nitrogen atmosphere. The reaction was monitored by LCMS. The reaction was quenched with water (20 mL) at room temperature. The resulting mixture was extracted with ethyl acetate (EtOAc) (3×30 mL), and the combined organic layers were washed with brine (80 mL), and dried over anhydrous Na₂SO₄. After filtration, the filtrate was concentrated under reduced pressure to provide a residue, which was purified by reversed-phase flash chromatography (C18 silica gel; mobile phase, MeCN in water, 0% to 100% gradient in 40 min; detector, UV 220/254 nm) to provide ethyl 4-[2-(5-cyclopropyl-4,7-difluoro-3,3-dimethyl-2-oxoindol-1-yl)acetamido]-3-(trifluoromethyl)pentanoate (Compound 72-OEt) (120 mg, 36% yield) as a brown solid. LCMS:(ES, m/z):RT=0.972 min, m/z=491.2 [M+H]⁺. ¹H NMR (400 MHz, Methanol-d₄) δ 6.68 (dd, J=12.0 Hz, 1H), 4.58-4.28 (m, 3H), 4.24-4.10 (m, 2H), 3.18-3.05 (m, 1H), 2.69-2.49 (m, 2H), 1.51 (s, 6H), 1.42-1.38 (m, 1H), 1.31-1.18 (m, 6H), 1.04-0.93 (m, 2H), 0.74-0.63 (m, 2H).

Step 4: The four diastereomers of ethyl 4-(2-(5-cyclopropyl-4,7-difluoro-3,3-dimethyl-2-oxoindolin-1-yl)acetamido)-3-(trifluoromethyl)pentanoate (Compound 72-OEt) (120 mg) were separated by Prep-Chiral-HPLC with the following conditions (CHIRALPAK IF, 2*25 cm; Mobile Phase A: Hexanes (0.5% 2M NH₃-MeOH), Mobile Phase B: EtOH:DCM=1:1; Flow rate: 20 mL/min; Gradient: 10% B to 10% B in 9.5 min; Wave Length: 220/254 nm) to afford a mixture of Compound 72A*-OEt, Compound 72B*-OEt and Compound 72C*-OEt (50 mg, 42% yield; Chiral HPLC RT (min)=5.97; LCMS:(ES, m/z):RT=1.122 min, m/z=491.2 [M+H]⁺) as a white solid and Compound 72D*-OEt, (40 mg, yield=33% yield; Chiral HPLC RT (min)=7.04; LCMS:(ES, m/z):RT=1.109 min, m/z=491.2 [M+H]⁺) as a white solid. The mixture of Compound 72A*-OEt, Compound 72B*-OEt and Compound 72C*-OEt (50 mg) was further separated by Prep-Chiral-HPLC (Lux 5 μm Cellulose-4, 2.12*25 cm; Mobile Phase A: Hexanes (0.5% 2M NH₃-MeOH), Mobile Phase B: EtOH:Hexanes=1:1; Flow rate: 20 mL/min; Gradient: 10% B to 10% B in 17 min; Wave Length: 220/254 nm) to afford ethyl (3S,4R)-4-[2-(5-cyclopropyl-4,7-difluoro-3,3-dimethyl-2-oxoindol-1-yl)acetamido]-3-(trifluoromethyl)pentanoate (Compound 72A*-OEt) (7.0 mg, 14% yield; Chiral HPLC RT (min): 11.95; LCMS:(ES, m/z):RT=1.598 min, m/z=491.2 [M+H]⁺) as a white solid, ethyl (3R,4S)-4-(2-(5-cyclopropyl-4,7-difluoro-3,3-dimethyl-2-oxoindolin-1-yl)acetamido)-3-(trifluoromethyl)pentanoate (Compound 72B*-OEt) (20 mg, 40% yield; Chiral HPLC RT (min): 13.83; LCMS:(ES, m/z):RT=1.604 min, m/z=491.2 [M+H]⁺) as a white solid and ethyl (3S,4S)-4-[2-(5-cyclopropyl-4,7-difluoro-3,3-dimethyl-2-oxoindol-1-yl)acetamido]-3-(trifluoromethyl)pentanoate (Compound 72C*-OEt) (10 mg, 20% yield; Chiral HPLC RT (min): 15.46; LCMS:(ES, m/z):RT=1.603 min, m/z=491.2 [M+H]⁺) as a white solid.

Step 5: Into a 8 mL vial was added ethyl (3S,4R)-4-[2-(5-cyclopropyl-4,7-difluoro-3,3-dimethyl-2-oxoindol-1-yl)acetamido]-3-(trifluoromethyl)pentanoate (Compound 72A*-OEt) (10.0 mg, 0.02 mmol, 1 equiv), ethanol (EtOH) (0.80 mL), H₂O (0.20 mL), and LiOH (2.44 mg, 0.10 mmol, 5 equiv) at room temperature. The resulting mixture was stirred for 1 h at room temperature. The reaction was monitored by LCMS. The mixture was adjusted to pH=4 with HCl (2M, aq.). The resulting mixture was extracted with EtOAc (2×20 mL). The combined organic layers were washed with brine (40 mL), dried over anhydrous Na₂SO₄. After filtration, the filtrate was concentrated under reduced pressure. This resulted in (3S,4R)-4-[2-(5-cyclopropyl-4,7-difluoro-3,3-dimethyl-2-oxoindol-1-yl)acetamido]-3-(trifluoromethyl)pentanoic acid (Compound 72A*) (5 mg, 70% purity) as a brown oil. The product was purified by Prep-Chiral-HPLC (Xselect CSH C18 OBD Column 30*150 mm; Mobile Phase A: water (0.05% TFA), Mobile Phase B: acetonitrile (MeCN); Flow rate: 60 mL/min; Gradient: 5% B to 5% B in 2 min, 38% B to 48% B in 10 min; Wave Length: 254/220 nm) to afford (3S,4R)-4-[2-(5-cyclopropyl-4,7-difluoro-3,3-dimethyl-2-oxoindol-1-yl)acetamido]-3-(trifluoromethyl)pentanoic acid (Compound 72A*) (1.0 mg, 11% yield; Chiral HPLC RT (min): 10.2) as a white solid.

Step 6: Into a 8 mL vial was added ethyl (3R,4S)-4-[2-(5-cyclopropyl-4,7-difluoro-3,3-dimethyl-2-oxoindol-1-yl)acetamido]-3-(trifluoromethyl)pentanoate (Compound 72B*-OEt) (7.0 mg, 0.01 mmol, 1 equiv), ethanol (EtOH) (0.80 mL), H₂O (0.20 mL), and LiOH (1.71 mg, 0.05 mmol, 5 equiv) at room temperature. The resulting mixture was stirred for 1 h at room temperature under nitrogen atmosphere. The reaction was monitored by LCMS. The mixture/residue was adjusted to pH=4 with HCl (2M, aq.). The resulting mixture was extracted with EtOAc (2×20 mL). The combined organic layers were washed with brine (50 mL), dried over anhydrous Na₂SO₄. After filtration, the filtrate was concentrated under reduced pressure. This resulted in (3R,4S)-4-[2-(5-cyclopropyl-4,7-difluoro-3,3-dimethyl-2-oxoindol-1-yl)acetamido]-3-(trifluoromethyl)pentanoic acid (Compound 72B*) (6.0 mg, 80% purity) as a brown oil. The product was purified by Prep-Chiral-HPLC (Xselect CSH C18 OBD Column 30*150 mm; Mobile Phase A: water (0.05% TFA), Mobile Phase B: acetonitrile (MeCN); Flow rate: 60 mL/min; Gradient: 5% B to 5% B in 2 min, 38% B to 48% B in 10 min; Wave Length: 254/220 nm) to afford (3R,4S)-4-[2-(5-cyclopropyl-4,7-difluoro-3,3-dimethyl-2-oxoindol-1-yl)acetamido]-3-(trifluoromethyl)pentanoic acid (Compound 72B*) (1.9 mg, 29% yield; Chiral HPLC RT (min): 10.2) as a white solid.

Step 7: Into a 8 mL vial was added ethyl (3S,4S)-4-[2-(5-cyclopropyl-4,7-difluoro-3,3-dimethyl-2-oxoindol-1-yl)acetamido]-3-(trifluoromethyl)pentanoate (Compound 72C*-OEt) (20 mg, 0.04 mmol, 1 equiv), ethanol (EtOH) (0.80 mL), H₂O (0.20 mL), and LiOH (4.9 mg, 0.20 mmol, 5 equiv) at room temperature. The resulting mixture was stirred for 1 h at room temperature. The reaction was monitored by LCMS. The mixture was adjusted to pH=4 with HCl (2M, aq.). The resulting mixture was extracted with EtOAc (2×20 mL). The combined organic layers were washed with brine (40 mL), dried over anhydrous Na₂SO₄. After filtration, the filtrate was concentrated under reduced pressure. This resulted in (3S,4S)-4-[2-(5-cyclopropyl-4,7-difluoro-3,3-dimethyl-2-oxoindol-1-yl)acetamido]-3-(trifluoromethyl)pentanoic acid (Compound 72C*) (18 mg, 85% purity) as a brown oil. The product was purified by Prep-HPLC with the following conditions (Xselect CSH C18 OBD Column 30*150 mm 5 μm; Mobile Phase A: water (0.05% TFA), Mobile Phase B: acetonitrile (MeCN); Flow rate: 60 mL/min; Gradient: 5% B to 5% B in 2 min, 38% B to 48% B in 10 min; Wave Length: 254/220 nm) to afford (3S,4S)-4-[2-(5-cyclopropyl-4,7-difluoro-3,3-dimethyl-2-oxoindol-1-yl)acetamido]-3-(trifluoromethyl)pentanoic acid (Compound 72C*) (16 mg, 84% yield; Chiral HPLC RT (min): 10.2) as a white solid.

Step 8: Into a 8 mL vial was added ethyl (3R,4R)-4-[2-(5-cyclopropyl-4,7-difluoro-3,3-dimethyl-2-oxoindol-1-yl)acetamido]-3-(trifluoromethyl)pentanoate (Compound 72D*-OEt) (40 mg, 0.08 mmol, 1 equiv), ethanol (EtOH) (0.80 mL), H₂O (0.20 mL), and LiOH (9.8 mg, 0.41 mmol, 5 equiv) at room temperature. The resulting mixture was stirred for 1 h at room temperature. The reaction was monitored by LCMS. The resulting mixture was diluted with water (20 mL). The mixture was adjusted to pH=4 with HCl (2M, aq.). The resulting mixture was extracted with EtOAc (2×30 mL). The combined organic layers were washed with brine (50 mL), dried over anhydrous Na₂SO₄. After filtration, the filtrate was concentrated under reduced pressure. This resulted in (3R,4R)-4-[2-(5-cyclopropyl-4,7-difluoro-3,3-dimethyl-2-oxoindol-1-yl)acetamido]-3-(trifluoromethyl)pentanoic acid (Compound 72D*) (35 mg, 80% purity) as a brown oil. The product was purified by Prep-HPLC (Xselect CSH C18 OBD Column 30*150 mm 5 μm; Mobile Phase A: water (0.05% TFA), Mobile Phase B: acetonitrile (MeCN); Flow rate: 60 mL/min; Gradient: 5% B to 5% B in 2 min, 38% B to 48% B in 10 min; Wave Length: 254/220 nm) to afford (3R,4R)-4-[2-(5-cyclopropyl-4,7-difluoro-3,3-dimethyl-2-oxoindol-1-yl)acetamido]-3-(trifluoromethyl)pentanoic acid (Compound 72D*) (21 mg, 56% yield; Chiral HPLC RT (min): 9.2) as a white solid.

Compound 72A*: LCMS:(ES, m/z):RT=0.925 min, m/z=463.2 [M+H]⁺. ¹H NMR (400 MHz, Methanol-d₄) δ 6.68 (dd, J=5.6 Hz, 1H), 4.50 (d, J=1.6 Hz, 2H), 4.42-4.29 (m, 1H), 3.06-2.91 (m, 1H), 2.64-2.49 (m, 2H), 2.09-1.99 (m, 1H), 1.51 (s, 6H), 1.36-1.23 (m, 3H), 1.06-0.94 (m, 2H), 0.77-0.63 (m, 2H).

Compound 72B*: LCMS:(ES, m/z):RT=0.928 min, m/z=463.2 [M+H]. ¹H NMR (400 MHz, Methanol-d₄) δ 6.68 (dd, J=5.6 Hz, 1H), 4.50 (d, J=1.6 Hz, 2H), 4.41-4.31 (m, 1H), 3.03-2.90 (m, 1H), 2.63-2.49 (m, 2H), 2.09-2.00 (m, 1H), 1.51 (s, 6H), 1.28 (d, J=6.8 Hz, 3H), 1.03-0.95 (m, 2H), 0.76-0.63 (m, 2H).

Compound 72C*: LCMS:(ES, m/z):RT=0.936 min, m/z=463.2 [M+H]⁺. ¹H NMR (400 MHz, Methanol-d₄) δ 6.68 (dd, J=5.6 Hz, 1H), 4.57-4.44 (m, 2H), 4.43-4.33 (m, 1H), 3.19-3.06 (m, 1H), 2.64-2.48 (m, 2H), 2.10-1.99 (m, 11H), 1.51 (s, 6H), 1.32-1.21 (m, 3H), 1.04-0.94 (m, 2H), 0.73-0.64 (in, 2H).

Compound 72D*: LCMS (ES, m/z):RT=0.931 min, m/z=463.2 [M+H]⁺. ¹H NMR (400 MHz, Methanol-d₄) δ 6.68 (dd, J=6.0 Hz, 11H), 4.58-4.43 (m, 2H), 4.43-4.35 (m, 11H), 3.20-3.05 (m, 11H), 2.63-2.48 (m, 2H), 2.08-1.99 (m, 11H), 1.51 (s, 6H), 1.26 (d, J=7.2 Hz, 3H), 1.03-0.95 (m, 2H), 0.73-0.66 (in, 2H).

Additional compounds provided in the below Table A have been prepared, or may be prepared, following General Schemes 1-12, as provided in the Methods of Preparation, and the Examples, as described above. LCMS data is provided for each compound synthesized according to the described Preparative Method. The Asterix (*) next to the Compound Number (4) signifies that arbitrary stereochemistry has been assigned. For purposes of the Examples and data provided in the Assay Methods, “Rac” signifies a mixture of two or more stereoisomers (not necessarily of equal amounts).

TABLE A
Additional Compounds

| # | Compound Name | LCMS | Preparative Method |
|---|---|---|---|
| Rac-3 | 3-(3-(2-(5-bromo-3,3-dimethyl-2- | 441.37 | Compound 3 (Rac-3) was prepared |
| | oxoindolin-1- | | following General Scheme 2 using 2- |
| | yl)acetamido)tetrahydrofuran-3- | | (5-bromo-3,3-dimethyl-2- |
| | yl)propanoic acid | | oxoindolin-1-yl)acetic acid as the |
| | Mixture of: | | indolinone(acetic acid) (i) reagent and |
| | (S)-3-(3-(2-(5-bromo-3,3-dimethyl- | | methyl 3-(3-aminotetrahydrofuran- |
| | 2-oxoindolin-1- | | 3-yl)propanoate as the aminoester |
| | yl)acetamido)tetrahydrofuran-3- | | (iv) reagent |
| | yl)propanoic acid (Compound 3A*) | | Compound 3A* and Compound 3B* |
| | (R)-3-(3-(2-(5-bromo-3,3-dimethyl- | | may be separated by Chiral Prep- |
| | 2-oxoindolin-1- | | HPLC. |
| | yl)acetamido)tetrahydrofuran-3- | | |
| | yl)propanoic acid (Compound 3B*) | | |
| 4 | 4-(2-(5-chloro-3,3-dimethyl-2- | 339.38 | Compound 4 was prepared following |
| | oxoindolin-1-yl)acetamido)butanoic | | General Scheme 2 using 2-(5- |
| | acid | | chloro-3,3-dimethyl-2-oxoindolin- |
| | | | 1-yl)acetic acid as the |
| | | | indolinone(acetic acid) (i) reagent and |
| | | | methyl 4-aminobutanoate as the |
| | | | aminoester (iv) reagent |
| 6 | 4-(2-(5′-bromo-2′- | 381.35 | Compound 6 was prepared following |
| | oxospiro[cyclopropane-1,3′-indolin]-1′- | | General Scheme 3 using 5′- |
| | yl)acetamido)butanoic acid | | bromospiro[cyclopropane-1,3′- |
| | | | indolin]-2′-one as the indolinone (vi) |
| | | | reagent and methyl 4-aminobutanoate |
| | | | as the aminoester (iv) reagent |
| 7 | 4-(2-(3,3,5-trimethyl-2-oxoindolin-1- | 319.40 | Compound 7 was prepared following |
| | yl)acetamido)butanoic acid | | General Scheme 2 using 2-(5- |
| | | | methyl-3,3-dimethyl-2-oxoindolin- |
| | | | 1-yl)acetic acid as the |
| | | | indolinone(acetic acid) (i) reagent and |
| | | | methyl 4-aminobutanoate as the |
| | | | aminoester (iv) reagent |
| 10 | 4-(2-(5-isopropyl-3,3-dimethyl-2- | 375.20 | Compound 10 was prepared |
| | oxoindolin-1-yl)acetamido)-4- | | following General Scheme 2 using 2- |
| | methylpentanoic acid | | (5-isopropyl-3,3-dimethyl-2- |
| | | | oxoindolin-1-yl)acetic acid as the |
| | | | indolinone(acetic acid) (i) reagent and |
| | | | methyl 4-amino-4-methylpentanoate |
| | | | as the aminoester (iv) reagent |
| 11A | (R)-N-(1-(1H-tetrazol-5-yl)piperidin- | 450.40 | Compound 11A was prepared |
| | 3-yl)-2-(5-bromo-3,3-dimethyl-2- | | following General Scheme 1 using 2- |
| | oxoindolin-1-yl)acetamide (tautomer 1) | | (5-bromo-3,3-dimethyl-2- |
| | (R)-N-(1-(2H-tetrazol-5-yl)piperidin- | | oxoindolin-1-yl)acetic acid as the |
| | 3-yl)-2-(5-bromo-3,3-dimethyl-2- | | indolinone(acetic acid) (i) reagent and |
| | oxoindolin-1-yl)acetamide (tautomer 2) | | (R)-1-(1H-tetrazol-5-yl)piperidin- |
| | | | 3-amine as the aminotetrazole (ii) |
| | | | reagent |
| 11B | (S)-N-(1-(1H-tetrazol-5-yl)piperidin- | | Compound 11B may prepared |
| | 3-yl)-2-(5-bromo-3,3-dimethyl-2- | | following General Scheme 1 using 2- |
| | oxoindolin-1-yl)acetamide (tautomer 1) | | (5-bromo-3,3-dimethyl-2- |
| | (S)-N-(1-(2H-tetrazol-5-yl)piperidin- | | oxoindolin-1-yl)acetic acid as the |
| | 3-yl)-2-(5-bromo-3,3-dimethyl-2- | | indolinone(acetic acid) (i) reagent and |
| | oxoindolin-1-yl)acetamide (tautomer 2) | | (S)-1-(1H-tetrazol-5-yl)piperidin- |
| | | | 3-amine as the aminotetrazole (ii) |
| | | | reagent |
| 12 | N-(3-(1H-tetrazol-5-yl)propyl)-2-(5- | 362.95 | Compound 12 was prepared |
| | chloro-3,3-dimethyl-2-oxoindolin-1- | | following General Scheme 1 using 2- |
| | yl)acetamide (tautomer 1) | | (5-chloro-3,3-dimethyl-2- |
| | N-(3-(2H-tetrazol-5-yl)propyl)-2-(5- | | oxoindolin-1-yl)acetic acid as the |
| | chloro-3,3-dimethyl-2-oxoindolin-1- | | indolinone(acetic acid) (i) reagent 3- |
| | yl)acetamide (tautomer 2) | | (1H-tetrazol-5-yl)propan-1-amine |
| | | | as the aminotetrazole (ii) reagent |
| 13 | 4-(2-(5-bromo-3,3-dimethyl-2- | 413.05 | Compound 13 was prepared |
| | oxoindolin-1-yl)acetamido)-3,3- | | following General Scheme 2 using 2- |
| | dimethylbutanoic acid | | (5-bromo-3,3-dimethyl-2- |
| | | | oxoindolin-1-yl)acetic acid as the |
| | | | indolinone(acetic acid) (i) reagent and |
| | | | methyl 4-amino-3,3- |
| | | | dimethylbutanoate as the aminoester |
| | | | (iv) reagent |
| 14 | 4-(2-(5-bromo-3,3-dimethyl-2- | 413.05 | Compound 14 was prepared |
| | oxoindolin-1-yl)acetamido)-2,2- | | following General Scheme 2 using 2- |
| | dimethylbutanoic acid | | (5-bromo-3,3-dimethyl-2- |
| | | | oxoindolin-1-yl)acetic acid as the |
| | | | indolinone(acetic acid) (i) reagent and |
| | | | methyl 4-amino-2,2- |
| | | | dimethylbutanoate as the aminoester |
| | | | (iv) reagent |
| 17 | N-(2-(1H-tetrazol-5-yl)phenyl)-2-(5- | 443.00 | Compound 17 was prepared |
| | bromo-3,3-dimethyl-2-oxoindolin-1- | | following General Scheme 1 using 2- |
| | yl)acetamide (tautomer 1) | | (5-bromo-3,3-dimethyl-2- |
| | N-(2-(2H-tetrazol-5-yl)phenyl)-2-(5- | | oxoindolin-1-yl)acetic acid as the |
| | bromo-3,3-dimethyl-2-oxoindolin-1- | | indolinone(acetic acid) (i) reagent 2- |
| | yl)acetamide (tautomer 2) | | (1H-tetrazol-5-yl)aniline as the |
| | | | aminotetrazole (ii) reagent |
| 18 | N-(3-(1H-tetrazol-5-yl)phenyl)-2-(5- | 441.00 | Compound 18 was prepared |
| | bromo-3,3-dimethyl-2-oxoindolin-1- | | following General Scheme 1 using 2- |
| | yl)acetamide (tautomer 1) | | (5-bromo-3,3-dimethyl-2- |
| | N-(3-(2H-tetrazol-5-yl)phenyl)-2-(5- | | oxoindolin-1-yl)acetic acid as the |
| | bromo-3,3-dimethyl-2-oxoindolin-1- | | indolinone(acetic acid) (i) reagent and |
| | yl)acetamide (tautomer 2) | | 3-(1H-tetrazol-5-yl)aniline as the |
| | | | aminotetrazole (ii) reagent |
| 19 | N-(4-(1H-tetrazol-5-yl)phenyl)-2-(5- | 443.05 | Compound 19 was prepared |
| | bromo-3,3-dimethyl-2-oxoindolin-1- | | following General Scheme 1 using 2- |
| | yl)acetamide (tautomer 1) | | (5-bromo-3,3-dimethyl-2- |
| | N-(4-(2H-tetrazol-5-yl)phenyl)-2-(5- | | oxoindolin-1-yl)acetic acid as the |
| | bromo-3,3-dimethyl-2-oxoindolin-1- | | indolinone(acetic acid) (i) reagent and |
| | yl)acetamide (tautomer 2) | | 4-(1H-tetrazol-5-yl)aniline as the |
| | | | aminotetrazole (ii) reagent |
| 20 | 4-(2-(5-bromo-6-fluoro-3,3- | 401.00 | Compound 20 was prepared |
| | dimethyl-2-oxoindolin-1- | | following General Scheme 3 using 5- |
| | yl)acetamido)butanoic acid | | bromo-6-fluoro-3,3- |
| | | | dimethylindolin-2-one as the |
| | | | indolinone (vi) reagent and methyl 4- |
| | | | aminobutanoate as the aminoester (iv) |
| | | | reagent |
| 21 | 4-(2-(5-bromo-4-fluoro-3,3- | 403.00 | Compound 21 was prepared |
| | dimethyl-2-oxoindolin-1- | | following General Scheme 3 using 5- |
| | yl)acetamido)butanoic acid | | bromo-4-fluoro-3,3- |
| | | | dimethylindolin-2-one as the |
| | | | indolinone (vi) reagent and methyl 4- |
| | | | aminobutanoate as the aminoester (iv) |
| | | | reagent |
| 22 | 4-(2-(5-iodo-3,3-dimethyl-2- | 430.95 | Compound 22 was prepared |
| | oxoindolin-1-yl)acetamido)butanoic | | following General Scheme 2 using 2- |
| | acid | | (5-iodo-3,3-dimethyl-2-oxoindolin- |
| | | | 1-yl)acetic acid as the |
| | | | indolinone(acetic acid) (i) reagent and |
| | | | methyl 4-aminobutanoate as the |
| | | | aminoester (iv) reagent. |
| 23 | N-(3-(1H-tetrazol-5-yl)propyl)-2-(5- | 420.90 | Compound 23 was prepared |
| | bromo-3,3-dimethyl-2-oxoindolin-1- | | following General Scheme 1 using 2- |
| | yl)-N-methylacetamide (tautomer 1) | | (5-chloro-3,3-dimethyl-2- |
| | N-(3-(2H-tetrazol-5-yl)propyl)-2-(5- | | oxoindolin-1-yl)acetic acid as the |
| | bromo-3,3-dimethyl-2-oxoindolin-1- | | indolinone(acetic acid) (i) reagent and |
| | yl)-N-methylacetamide (tautomer 2) | | N-methyl-3-(1H-tetrazol-5- |
| | | | yl)propan-1-amine as the |
| | | | aminotetrazole (ii) reagent |
| 25 | 4-(2-(5-cyclopentyl-3,3-dimethyl-2- | 373.20 | Compound 25 was prepared |
| | oxoindolin-1-yl)acetamido)butanoic | | following General Scheme 5 using |
| | acid | | methyl 2-(5-bromo-3,3-dimethyl-2- |
| | | | oxoindolin-1-yl)acetate as the |
| | | | indolinone (vii) reagent, 2- |
| | | | (cyclopent-1-en-1-yl)-4,4,5,5- |
| | | | tetramethyl-1,3-dioxolane as the |
| | | | coupling partner (xii) and methyl 4- |
| | | | aminobutanoate as the aminoester (iv) |
| | | | reagent, using a hydrogenation step |
| 26 | 2-(5-bromo-7-hydroxy-3,3-dimethyl- | 480.10 | Compound 26 was prepared |
| | 2-oxoindolin-1-yl)-N-((3R,5S)-5- | | following General Scheme 5 using 5- |
| | methyl-1-(1H-tetrazol-5-yl)piperidin- | | bromo-3,3-dimethylindolin-2-one as |
| | 3-yl)acetamide (tautomer 1) | | the indolinone (xx) reagent and |
| | 2-(5-bromo-7-hydroxy-3,3-dimethyl- | | (3R,5S)-5-methyl-1-(1H-tetrazol- |
| | 2-oxoindolin-1-yl)-N-((3R,5S)-5- | | 5-yl)piperidin-3-amine as the |
| | methyl-1-(2H-tetrazol-5-yl)piperidin- | | aminotetrazole (ii) reagent |
| | 3-yl)acetamide (tautomer 2) | | |
| 30 | 4-(2-(5-bromo-7-hydroxy-3,3- | 414.95 | Compound 30 was prepared |
| | dimethyl-2-oxoindolin-1-yl)-N- | | following General Scheme 7 using 5- |
| | methylacetamido)butanoic acid | | bromo-3,3-dimethylindolin-2-one as |
| | | | the indolinone (xx) reagent and |
| | | | methyl 4-(methylamino)butanoate as |
| | | | the aminoester (iv) reagent |
| 31 | 4-(2-(5-bromo-7-hydroxy-3,3- | 429.05 | Compound 31 was prepared |
| | dimethyl-2-oxoindolin-1- | | following General Scheme 7 using 5- |
| | yl)acetamido)-4-methylpentanoic acid | | bromo-3,3-dimethylindolin-2-one as |
| | | | the indolinone (xx) reagent and |
| | | | methyl 4-amino-4-methylpentanoate |
| | | | as the aminoester (iv) reagent |
| 32 | 2-(5-bromo-7-hydroxy-3,3-dimethyl- | 451.10 | Compound 32 was prepared |
| | 2-oxoindolin-1-yl)-N-(2-methyl-4- | | following General Scheme 7 using 5- |
| | (1H-tetrazol-5-yl)butan-2- | | bromo-3,3-dimethylindolin-2-one as |
| | yl)acetamide (tautomer 1) | | the indolinone (xx) reagent and 2- |
| | 2-(5-bromo-7-hydroxy-3,3-dimethyl- | | methyl-4-(1H-tetrazol-5-yl)butan- |
| | 2-oxoindolin-1-yl)-N-(2-methyl-4- | | 2-amine as the aminotetrazole (ii) |
| | (2H-tetrazol-5-yl)butan-2- | | reagent |
| | yl)acetamide (tautomer 2) | | |
| 33 | N-(3-(1H-tetrazol-5-yl)propyl)-2-(5- | 425.00 | Compound 33 was prepared |
| | bromo-7-hydroxy-3,3-dimethyl-2- | | following General Scheme 7 using 5- |
| | oxoindolin-1-yl)acetamide (tautomer 1) | | bromo-3,3-dimethylindolin-2-one as |
| | N-(3-(2H-tetrazol-5-yl)propyl)-2-(5- | | the indolinone (xx) reagent and 3- |
| | bromo-7-hydroxy-3,3-dimethyl-2- | | (2H-tetrazol-5-yl)propan-1-amine |
| | oxoindolin-1-yl)acetamide (tautomer 2) | | as the aminotetrazole (ii) reagent |
| 35 | 2-(1-((2-(5-bromo-7-hydroxy-3,3- | 425.05 | Compound 35 was prepared |
| | dimethyl-2-oxoindolin-1- | | following General Scheme 7 using 5- |
| | yl)acetamido)methyl)cyclopropyl)acetic | | bromo-3,3-dimethylindolin-2-one as |
| | acid | | the indolinone (xx) reagent and 2-(1- |
| | | | (aminomethyl)cyclopropyl)acetic acid |
| | | | as the aminoester (iv) reagent |
| 37 | N-(2-(1H-tetrazol-5-yl)pyridin-3-yl)- | 460.05 | Compound 37 was prepared |
| | 2-(5-bromo-7-hydroxy-3,3-dimethyl- | | following General Scheme 7 using 5- |
| | 2-oxoindolin-1-yl)acetamide (tautomer | | bromo-3,3-dimethylindolin-2-one as |
| | 1) | | the indolinone (xx) reagent and 2- |
| | N-(2-(2H-tetrazol-5-yl)pyridin-3-yl)- | | (1H-tetrazol-5-yl)pyridin-3-amine |
| | 2-(5-bromo-7-hydroxy-3,3-dimethyl- | | as the aminotetrazole (ii) reagent |
| | 2-oxoindolin-1-yl)acetamide (tautomer | | |
| | 2) | | |
| 38 | N-(3-(1H-tetrazol-5-yl)pyridin-2-yl)- | 458.00 | Compound 38 was prepared |
| | 2-(5-bromo-7-hydroxy-3,3-dimethyl- | | following General Scheme 7 using 5- |
| | 2-oxoindolin-1-yl)acetamide (tautomer | | bromo-3,3-dimethylindolin-2-one as |
| | 1) | | the indolinone (xx) reagent and 3- |
| | N-(3-(2H-tetrazol-5-yl)pyridin-2-yl)- | | (1H-tetrazol-5-yl)218yridine-2- |
| | 2-(5-bromo-7-hydroxy-3,3-dimethyl- | | amine as the aminotetrazole (ii) |
| | 2-oxoindolin-1-yl)acetamide (tautomer | | reagent |
| | 2) | | |
| 39 | 4-(2-(7-hydroxy-3,3,5-trimethyl-2- | 335.15 | Compound 39 was prepared |
| | oxoindolin-1-yl)acetamido)butanoic | | following General Scheme 7 using |
| | acid | | 3,3,5-trimethylindolin-2-one as the |
| | | | indolinone (xx) reagent and methyl 4- |
| | | | aminobutanoate as the aminoester (iv) |
| | | | reagent |
| 40A* | (R)-3-(3-(2-(5-bromo-7-hydroxy- | 457.10 | Compounds 40A* and 40B* were |
| | 3,3-dimethyl-2-oxoindolin-1- | | prepared following General Scheme 7 |
| | yl)acetamido)tetrahydrofuran-3- | | using 5-bromo-3,3-dimethylindolin- |
| | yl)propanoic acid | | 2-one as the indolinone (xx) reagent |
| 40B* | (S)-3-(3-(2-(5-bromo-7-hydroxy-3,3- | 457.05 | and methyl 3-(3- |
| | dimethyl-2-oxoindolin-1- | | aminotetrahydrofuran-3- |
| | yl)acetamido)tetrahydrofuran-3- | | yl)propanoate as the aminoester (iv) |
| | yl)propanoic acid | | reagent. |
| | | | The mixture was purified by Prep- |
| | | | HPLC (CHIRALPAK IA-3, |
| | | | 4.6*50 mm, 3 um; Mobile Phase A: |
| | | | Hexanes (0.1% TFA): (MeOH: |
| | | | dichloromethane) 80: 20; Flow rate: 1 |
| | | | mL/min; Gradient: 0% B to 0% B) to |
| | | | afford 40A* as first eluting peak and |
| | | | 40B* as second eluting peak. |
| 41A | (R)-4-(2-(5-bromo-7-hydroxy-3,3- | 415.00 | Compound 41A was prepared |
| | dimethyl-2-oxoindolin-1- | | following General Scheme 7 using 5- |
| | yl)acetamido)pentanoic acid | | bromo-3,3-dimethylindolin-2-one as |
| | | | the indolinone (xx) reagent and |
| | | | methyl (R)-4-aminopentanoate as the |
| | | | aminoester (iv) reagent |
| 41B | (S)-4-(2-(5-bromo-7-hydroxy-3,3- | 415.10 | Compound 41B was prepared |
| | dimethyl-2-oxoindolin-1- | | following General Scheme 7 using 5- |
| | yl)acetamido)pentanoic acid | | bromo-3,3-dimethylindolin-2-one as |
| | | | the indolinone (xx) reagent and (S)- |
| | | | 4-aminopentanoic acid as the |
| | | | aminoester (iv) reagent |
| 42A* | (R)-4-(2-(5-bromo-7-hydroxy-3,3- | 415.00 | Compounds 42A* and 42B* were |
| | dimethyl-2-oxoindolin-1- | | prepared following General Scheme 7 |
| | yl)acetamido)-3-methylbutanoic acid | | using 5-bromo-3,3-dimethylindolin- |
| 42B* | (S)-4-(2-(5-bromo-7-hydroxy-3,3- | 415.00 | 2-one as the indolinone (xx) reagent |
| | dimethyl-2-oxoindolin-1- | | and methyl 4-amino-3- |
| | yl)acetamido)-3-methylbutanoic acid | | methylbutanoate as the aminoester |
| | | | (iv) reagent. |
| | | | The mixture was then purified by |
| | | | Prep-HPLC (Xselect CSH C18 OBD |
| | | | Column 30*150 mm 5 μm; Mobile |
| | | | Phase A: acetonitrile, Mobile Phase |
| | | | B: water (0.05% TFA); Flow rate: 60 |
| | | | mL/min; Gradient: 26% B to 36% B |
| | | | in 10 min, 36% B; Wave Length: |
| | | | 254/220 nm; to afford Compound |
| | | | 42A* as first eluting peak and |
| | | | Compound 42B* as second eluting |
| | | | peak. |
| 44 | 3-(1-(2-(5-bromo-7-hydroxy-3,3- | 425.05 | Compound 44 was prepared 7 |
| | dimethyl-2-oxoindolin-1- | | following General Scheme 7 using 5- |
| | yl)acetamido)cyclopropyl)propanoic | | bromo-3,3-dimethylindolin-2-one as |
| | acid | | the indolinone (xx) reagent and |
| | | | methyl 3-(1- |
| | | | aminocyclopropyl)propanoate (iv) |
| 45 | N-(3-(1H-tetrazol-5-yl)pyridin-4-yl)- | 460.00 | Compound 42 was prepared |
| | 2-(5-bromo-7-hydroxy-3,3-dimethyl- | | following General Scheme 7 using 5- |
| | 2-oxoindolin-1-yl)acetamide (tautomer | | bromo-3,3-dimethylindolin-2-one as |
| | 1) | | the indolinone (xx) reagent and 3- |
| | N-(3-(2H-tetrazol-5-yl)pyridin-4-yl)- | | (1H-tetrazol-5-yl)pyridin-4-amine |
| | 2-(5-bromo-7-hydroxy-3,3-dimethyl- | | as the aminotetrazole (ii) reagent |
| | 2-oxoindolin-1-yl)acetamide (tautomer | | |
| | 2) | | |
| 47 | 4-(2-(5-chloro-7-hydroxy-3,3- | 355.00 | Compound 47 was prepared |
| | dimethyl-2-oxoindolin-1- | | following General Scheme 7 using 5- |
| | yl)acetamido)butanoic acid | | chloro-3,3-dimethylindolin-2-one as |
| | | | the indolinone (xx) reagent and |
| | | | methyl 4-aminobutanoate as the |
| | | | aminoester (iv) reagent |
| 48 | 4-(2-(5-chloro-7-fluoro-3,3-dimethyl- | 356.95 | Compound 48 was prepared |
| | 2-oxoindolin-1-yl)acetamido)butanoic | | following General Scheme 8 using 7- |
| | acid | | fluoro-3,3-dimethylindolin-2-one as |
| | | | the indolinone (xxvi) reagent and |
| | | | methyl 4-aminobutanoate as the |
| | | | aminoester (iv) reagent |
| 49 | 2-((2-(5-bromo-3,3-dimethyl-2- | 433.00 | Compound 49 was prepared |
| | oxoindolin-1- | | following General Scheme 2 using 2- |
| | yl)acetamido)methyl)benzoic acid | | (5-bromo-3,3-dimethyl-2- |
| | | | oxoindolin-1-yl)acetic acid as the |
| | | | indolinone(acetic acid) (i) reagent and |
| | | | 2-(aminomethyl)benzoic acid as the |
| | | | aminoester (iv) reagent |
| 50 | 4-(2-(5-bromo-3,3-dimethyl-2-oxo- | 451.05 | Compound 50 was prepared |
| | 7-(trifluoromethyl)indolin-1- | | following General Scheme 7 using 2- |
| | yl)acetamido)butanoic acid | | (5-bromo-3,3-dimethyl-2-oxo-7- |
| | | | (trifluoromethyl)indolin-1-yl)acetic |
| | | | acid as the indolinone (xx) reagent |
| | | | and methyl 4-aminobutanoate as the |
| | | | aminoester (iv) reagent. |
| | | | 2-(5-bromo-3,3-dimethyl-2-oxo-7- |
| | | | (trifluoromethyl)indolin-1-yl)acetic |
| | | | acid was prepared using standard |
| | | | procedures for trifluoromethylation |
| | | | (using TMSCF₃, KF and CuI in DMF |
| | | | and heating at 80° C.) from tert-butyl |
| | | | 2-(5-bromo-7-iodo-3,3-dimethyl- |
| | | | 2-oxoindol-1-yl)acetate (product of |
| | | | Step 2, Example 7) |
| 51 | 4-(2-(5-bromo-3,3-difluoro-2- | 392.95 | Compound 51 was prepared |
| | oxoindolin-1-yl)acetamido)butanoic | | following General Scheme 3 using 5- |
| | acid | | bromo-3,3-difluoroindolin-2-one as |
| | | | the indolinone (vi) reagent and methyl |
| | | | 4-aminobutanoate as the aminoester |
| | | | (iv) reagent |
| 52A* | (1S,2R)-2-(2-(5-bromo-3,3-dimethyl- | 411.00 | Compound 52A * was prepared |
| | 2-oxoindolin-1- | | following General Scheme 2 using 2- |
| | yl)acetamido)cyclopentane-1- | | (5-bromo-3,3-dimethyl-2- |
| | carboxylic acid | | oxoindolin-1-yl)acetic acid as the |
| | | | indolinone(acetic acid) (i) reagent and |
| | | | methyl (1S,2R)-2- |
| | | | aminocyclopentane-1-carboxylate |
| | | | the aminoester (iv) reagent |
| 52B* | (1R,2S)-2-(2-(5-bromo-3,3-dimethyl- | 409.05 | Compound 52B* was prepared |
| | 2-oxoindolin-1- | | following General Scheme 2 using 2- |
| | yl)acetamido)cyclopentane-1- | | (5-bromo-3,3-dimethyl-2- |
| | carboxylic acid | | oxoindolin-1-yl)acetic acid as the |
| | | | indolinone(acetic acid) (i) reagent and |
| | | | methyl (1R,2S)-2- |
| | | | aminocyclopentane-1-carboxylate |
| | | | the aminoester (iv) reagent |
| 52C* | (1S,2S)-2-(2-(5-bromo-3,3-dimethyl- | 411.00 | Compound 52C* was prepared |
| | 2-oxoindolin-1- | | following General Scheme 2 using 2- |
| | yl)acetamido)cyclopentane-1- | | (5-bromo-3,3-dimethyl-2- |
| | carboxylic acid | | oxoindolin-1-yl)acetic acid as the |
| | | | indolinone(acetic acid) (i) reagent and |
| | | | methyl (1S,2S)-2- |
| | | | aminocyclopentane-1-carboxylate |
| | | | the aminoester (iv) reagent |
| 52D* | (1R,2R)-2-(2-(5-bromo-3,3-dimethyl- | 410.95 | Compound 52D* was prepared |
| | 2-oxoindolin-1- | | following General Scheme 2 using 2- |
| | yl)acetamido)cyclopentane-1- | | (5-bromo-3,3-dimethyl-2- |
| | carboxylic acid | | oxoindolin-1-yl)acetic acid as the |
| | | | indolinone(acetic acid) (i) reagent and |
| | | | methyl (1R,2R)-2- |
| | | | aminocyclopentane-1-carboxylate |
| | | | the aminoester (iv) reagent |
| 65 | N-(3-(1H-tetrazol-5-yl)propyl)-2-(5- | 405.10 | Compound 65 was prepared |
| | cyclopropyl-4,7-difluoro-3,3- | | following General Scheme 1 using 2- |
| | dimethyl-2-oxoindolin-1-yl)acetamide | | (5-cyclopropyl-4,7-difluoro-3,3- |
| | (tautomer 1) | | dimethyl-2-oxoindolin-1-yl)acetic |
| | N-(3-(2H-tetrazol-5-yl)propyl)-2-(5- | | acid as the indolinone(acetic acid) (i) |
| | cyclopropyl-4,7-difluoro-3,3- | | reagent and 3-(1H-tetrazol-5- |
| | dimethyl-2-oxoindolin-1-yl)acetamide | | yl)propan-1-amine as the |
| | (tautomer 2) | | aminotetrazole (ii) reagent |
| 68A* | (3S,4S)-4-cyclopropyl-4-(2-(5- | — | Compounds 68A*, 68B*, 68C*, |
| | cyclopropyl-4,7-difluoro-3,3- | | 68D* may be prepared following |
| | dimethyl-2-oxoindolin-1- | | General Scheme 2 using 2-(5- |
| | yl)acetamido)-3-methylbutanoic acid | | cyclopropyl-4,7-difluoro-3,3- |
| 68B* | (3R,4R)-4-cyclopropyl-4-(2-(5- | — | dimethyl-2-oxoindolin-1- |
| | cyclopropyl-4,7-difluoro-3,3- | | yl)acetamide as the indolinone(acetic |
| | dimethyl-2-oxoindolin-1- | | acid) (i) reagent and tert-butyl 4- |
| | yl)acetamido)-3-methylbutanoic acid | | amino-4-cyclopropyl-3- |
| 68C* | (3S,4R)-4-cyclopropyl-4-(2-(5- | — | methylbutanoate (product of Step 6, |
| | cyclopropyl-4,7-difluoro-3,3- | | Example 17) as the aminoester (iv) |
| | dimethyl-2-oxoindolin-1- | | reagent |
| | yl)acetamido)-3-methylbutanoic acid | | |
| 68D* | (3S,4R)-4-cyclopropyl-4-(2-(5- | — | |
| | cyclopropyl-4,7-difluoro-3,3- | | |
| | dimethyl-2-oxoindolin-1- | | |
| | yl)acetamido)-3-methylbutanoic acid | | |

### Biological Assay Methods

### (i) Human PBMC NLRP3 Assay

The objective of this assay to is demonstrate if a test compound is able to interfere with human NLRP3 function in a cellular system.

Reagents: Human PBMCs (Normal): iXCells Cat #10HU-003; RPMI 1640 medium (ATCC modification): ThermoFisher Cat #A1049101 (Complete Media: 10% FBS, 1% Pen/Strep; Assay Media: 1% Pen/Strep; 75 cm² cell culture flask: Corning Cat #430641U; 96-well cell culture plates: VWR Cat #10062-900; LPS (E. coli 026:B6): Sigma Cat #L₂₆₅₄, stock 5 mg/mL in PBS; Nigericin sodium salt: Sigma Cat #N7143, stock 5 mg/mL (6.7 mM) in ethanol.

Cryopreserved PBMCs (10×10⁶ cells/vial) were rapidly thawed in a 37° C. water bath for 2 min and transferred in 10 mL Complete Media. Cells were then centrifuged at 1200 RPM for 5 min, resuspended in 40 mL of Complete Media, transferred in a 75 cm² cell culture flask and incubated overnight at 37° C. with 5% CO₂. The next day, PBMCs were centrifuged, resuspended in 30 mL Assay Media (˜3.3×10⁵ cells/mL) and distributed at 100 μL/well in a 96-well cell culture plate. 50 μl of Assay Media containing 300 ng/ml of LPS was then added to each well except for the untreated control wells (50 μl Assay Media without LPS). Cells were then primed with LPS for 4 h at 37° C. with 5% CO₂. A concentration response curve (CRC) was prepared of 500× test compound in 100% dimethyl sulfoxide (DMSO). The CRC was then diluted 1:25 in Assay Media and then further diluted 1:5 in Assay Media resulting in a final 4× CRC in 0.8% DMSO/Assay Media. 50 μL/well of 4× test compound CRC or vehicle (0.8% DMSO/Assay Media) was then transferred into each well and subsequently incubated for 30 min at 37° C. with 5% CO₂. Cells were stimulated by adding 4 μL of 500 μM Nigericin, for 1 h at 37° C. with 5% CO₂ Plates were then centrifuged at 1200 RPM for 5 min and 100 μl of the media was transferred to a clean 96-well storage plate and stored at −80° C. until analyzed. Cytokine measurements were performed using the mesoscale platform. Compounds were added 30 minutes before priming the cells with LPS when TNFα was required to be quantified in the cytokine panel to confirm selectivity.

Human PBMC NLRP3 Assay: A represents an IC₅₀ value <0.3 μM; B represents an IC₅₀ value ≥0.3 μM and <0.5 μM; C represents an IC₅₀ value ≥0.5 μM and <1.0 μM; D represents an IC₅₀ value >1.0 μM and <10 μM; E represents an IC₅₀ value >10 μM.

### (ii) Mouse Whole Blood (mWB) NLRP3 Assay

The objective of this assay to is demonstrate if a test compound is able to interfere with mouse NLRP3 function in a whole blood system.

Reagents: The following reagents were used: blood collection tubes (Heparin); U-bottom 96-well tissue culture (Falcon 353077); HBSS for LPS, ATP and compound dilutions (Gibco 24020-117); and LPS, E. coli serotype 026:B6 (Sigma L-2654).

Mouse IL-1b MSD assay: Blood was drawn from female CD1 mice (9 to 10 weeks) by cardiac puncture. Blood was plated (135 μL) per well in 96-well U-bottom plates. 7.5 μL of 20× LPS (20 μg/mL, final concentration of 1 μg/mL) was added and mixed by gentle pipetting, and the mixture was incubated in a TC incubator for five hours. 7.5 μL of 20× compound or vehicle per well was added and mixed by gentle pipetting. Compounds were diluted 1/50 in HBSS to prepare a 20× dilution curve in 2% DMSO. The mixture was incubated for 30 minutes in a TC incubator with shaking (450 rpm). 5 μl of 31× ATP (155 mM, final concentration of 5 mM) was added and mixed by gentle pipetting. The mixture was incubated in a TC incubator for 30 minutes with shaking (450 rpm). The plate was centrifuged for 10 minutes at 800×g and ˜70 μL of plasma was removed. Plasma was frozen if required. Mouse IL-1b was analyzed with IL-1b MSD assay (K152TUK).

Compounds were added 30 minutes before priming the cells with LPS when TNFα was required to be quantified in the cytokine panel to confirm selectivity.

mWB IL-1β Assay: A represents an IC₅₀ value <0.3 μM; B represents an IC₅₀ value ≥0.3 μM and <0.5 μM; C represents an IC₅₀ value ≥0.5 μM and <1.0 μM; D represents an IC₅₀ value ≥1.0 μM.

### (iii) Human Whole Blood (hWB) NLRP3 Assay

The objective of this assay to is demonstrate if a test compound is able to interfere with human NLRP3 function in a whole blood system.

Human whole blood was drawn from healthy volunteers after obtaining written informed consent. Heparin lithium coated tubes were used to collect blood from volunteers. Blood samples were distributed on 96 well plates using 90 μl per well. Priming was performed by adding 5 μl of LPS (O26:B6; Sigma L-2654) at a final concentration of 1 μg/ml for 4.5 hours in a humidified incubator with 37° C., 5% CO₂. Thirty minutes prior to NLRP3 activation, 5 μl of a 20× compound solution or vehicle (2% DMSO) was added to each well and plates were incubated on a shaker (450 rpm) in a humidified incubator with 37° C., 5% CO₂. Activation was then performed by adding 3.3 μl of a 31× ATP solution per well. At the end of the 30 minutes stimulation, the plates were centrifuged (800 g, 10 min, room temperature) and the plasma from each well was frozen at −80° C. IL-1β levels in the supernatant were analyzed using a mesoscale discovery assay (MSD K151TUK) according to the manufacturers' instructions.

hWB IL-1β Assay: A represents an IC₅₀ value <0.3 μM; B represents an IC₅₀ value ≥0.3 μM and <0.5 μM; C represents an IC₅₀ value ≥0.5 μM and <1.0 μM; D represents an IC₅₀ value >1.0 μM.

### (iv) Assay Data

Various compounds described herein were tested according to the above-described assays.

TABLE B
Activity Data

| | PBMC IL- | mWB IL- | hWB IL- |
|---|---|---|---|
| | 1β IC₅₀ | 1β IC₅₀ | 1β IC₅₀ |
| Compound # | (μM) | (μM) | (μM) |
| 1 | A | A | D |
| 2 | A | A | D |
| Rac-3 | A | — | C |
| 4 | — | — | D |
| 5 | E | — | — |
| 6 | C | — | — |
| 7 | D | — | — |
| 8 | C | — | — |
| 9 | C | — | — |
| 10 | B | — | — |
| 11A | — | A | D |
| 11B | — | — | — |
| 12 | — | — | — |
| 13 | A | — | — |
| 14 | D | — | — |
| 15 | E | — | — |
| 16 | A | C | C |
| 17 | — | — | — |
| 18 | — | — | — |
| 19 | — | — | — |
| 20 | D | — | — |
| 21 | A | — | A |
| 22 | A | — | A |
| 23 | — | — | — |
| 24 | A | A | — |
| 25 | E | — | — |
| 26 | | C | — |
| 27 | E | — | — |
| 28 | A | — | D |
| 29 | — | — | D |
| 30 | A | — | D |
| 31 | A | — | C |
| 32 | — | — | D |
| 33 | A | — | D |
| 35 | A | — | D |
| 36 | E | — | — |
| 37 | — | — | — |
| 38 | — | — | — |
| 39 | — | — | — |
| 40A* | — | — | — |
| 40B* | — | — | — |
| 41A | A | — | — |
| 41B | A | — | D |
| 42A* | A | — | — |
| 42B* | A | — | — |
| 44 | A | — | — |
| 45 | — | — | — |
| 47 | — | — | B |
| 48 | — | — | — |
| 49 | E | | — |
| 50 | E | | — |
| 51 | B | — | — |
| 52A* | E | — | — |
| 52B* | — | — | — |
| 52C* | — | — | — |
| 52D* | — | — | — |
| 56 | A | A | C |
| 57 | A | A | B |
| 58A* | A | — | C |
| 58B* | — | — | — |
| 58C* | — | — | — |
| 58D* | — | — | — |
| 59A | A | A | B |
| 59B | — | — | — |
| 60A* | A | A | C |
| 60B* | — | — | — |
| 60C* | — | — | — |
| 60D* | — | — | — |
| 61A | — | A | B |
| 61B | — | — | C |
| 62A | — | A | B |
| 62B | — | — | — |
| 63A | A | A | C |
| 63B | — | — | — |
| 64A | A | A | C |
| 64B | — | — | — |
| 65 | A | A | C |
| 66 | A | A | A |
| 67A* | — | — | — |
| 67B* | — | — | — |
| 67C* | — | — | D |
| 67D* | — | — | B |
| 68A* | — | — | — |
| 68B* | — | — | — |
| 68C* | — | — | — |
| 68D* | — | — | — |
| 69A* | — | — | — |
| 69B* | — | — | — |
| 70A* | — | — | — |
| 70B* | — | — | — |
| 70C* | — | — | — |
| 70D* | — | — | — |
| 71A* | — | — | — |
| 71B* | — | — | — |
| 72A* | — | — | — |
| 72B* | — | — | — |
| 72C* | — | — | — |
| 72D* | — | — | — |

### (v) Comparative Examples

Certain features of compounds of Formula (I) have been found important for improved inhibitory activity against NLRP3.

For example, compounds of Formula (I) comprise, at least, an R¹ substitution and geminal substitution at the R⁵/R⁵ position. As can be seen from Table C₁, compounds with hydrogen at the corresponding R¹ position (Comparative Example 1) have been found to be less potent than compounds with substitution at the corresponding R¹ position (e.g., bromo substitution; See, e.g., Comparative Examples 2 and 3). However, it is the combination of R¹ substitution and substitution at the R⁵/R⁵ positions which lead to a boost in NLRP3 inhibition. Compare, for example, the lower activity of compounds with bromo substitution at the R¹ position but with hydrogen at the R⁵/R⁵ position (Comparative Examples 2 and 3) or with an oxo group at the R/R position (Comparative Example 4) with the higher activity of compounds of Formula (I) comprising both bromo substitution at the R¹ position and an R⁵/R⁵ dimethyl group (Compound 2), an R⁵/R⁵ difluoro group (Compound 51), or an R⁵/R⁵ group cyclized to form a cyclopropyl (Compound 6). Furthermore, in addition to bromo, various R¹ groups have been found to be well tolerated, such as trifluoromethyl (Compound 8) and cyclopropyl (Compound 9).

TABLE C1
The R¹ and R⁵/R⁵ positions

| Compound | PBMC IL-1β IC₅₀ (uM) |
|---|---|
| 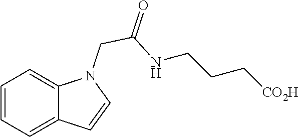 [SMILES: O=C(O)CCCNC(=O)Cn1ccc2ccccc21] | >100 (“E“ Activity) |
| 4-(2-(1H-indol-1-yl)acetamido)butanoic acid | |
| COMPARATIVE EXAMPLE 1 | |
| 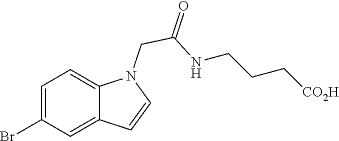 [SMILES: O=C(O)CCCNC(=O)Cn1ccc2cc(Br)ccc21] | 7.2 (“D” Activity) |
| 4-(2-(5-bromo-1H-indol-1-yl)acetamido)butanoic acid | |
| COMPARATIVE EXAMPLE 2 | |
| 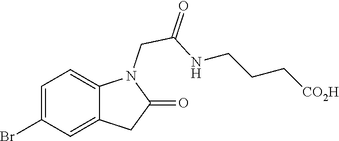 [SMILES: O=C(O)CCCNC(=O)CN1C(=O)Cc2cc(Br)ccc21] | 47 (“E“ Activity) |
| 4-(2-(5-bromo-2-oxoindolin-1-yl)acetamido)butanoic acid | |
| COMPARATIVE EXAMPLE 3 | |
| 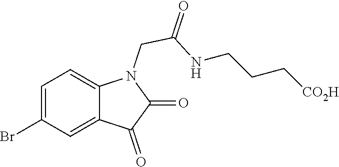 [SMILES: O=C(O)CCCNC(=O)CN1C(=O)C(=O)c2cc(Br)ccc21] | >100 (“E“ Activity) |
| 4-(2-(5-bromo-2,3-dioxoindolin-1-yl)acetamido)butanoic acid | |
| COMPARATIVE EXAMPLE 4 | |
| 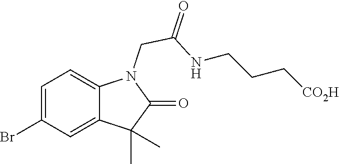 [SMILES: CC1(C)C(=O)N(CC(=O)NCCCC(=O)O)c2ccc(Br)cc21] | 0.15 (“A” Activity) |
| 4-(2-(5-bromo-3,3-dimethyl-2-oxoindolin-1- | |
| yl)acetamido)butanoic acid | |
| Compound 2 | |
| 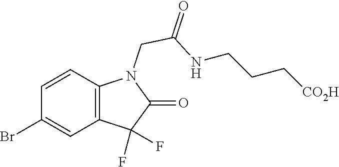 [SMILES: O=C(O)CCCNC(=O)CN1C(=O)C(F)(F)c2cc(Br)ccc21] | 0.41 (“B” Activity) |
| 4-(2-(5-bromo-3,3-difluoro-2-oxoindolin-1- | |
| yl)acetamido)butanoic acid | |
| Compound 51 | |
| 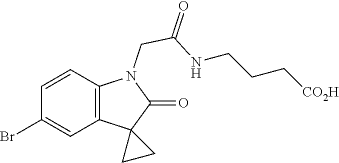 [SMILES: O=C(O)CCCNC(=O)CN1C(=O)C2(CC2)c2cc(Br)ccc21] | 0.57 (“C” Activity) |
| 4-(2-(5′-bromo-2′-oxospiro[cyclopropane-1,3′-indolin]-1′- | |
| yl)acetamido)butanoic acid | |
| Compound 6 | |
| 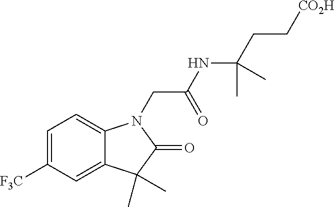 [SMILES: CC(C)(CCC(=O)O)NC(=O)CN1C(=O)C(C)(C)c2cc(C(F)(F)F)ccc21] | 0.69 (“C” Activity) |
| 4-(2-(3,3-dimethyl-2-oxo-5-(trifluoromethyl)indolin-1- | |
| yl)acetamido)-4-methylpentanoic acid | |
| Compound 8 | |
| 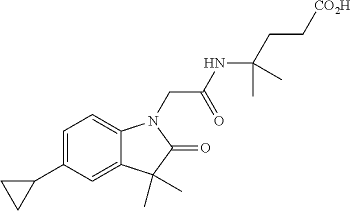 [SMILES: CC(C)(CCC(=O)O)NC(=O)CN1C(=O)C(C)(C)c2cc(C3CC3)ccc21] | 0.67 (“C” Activity) |
| 4-(2-(5-cyclopropyl-3,3-dimethyl-2-oxoindolin-1- | |
| yl)acetamido)-4-methylpentanoic acid | |
| Compound 9 | |

In addition, the linker between the amide N(R⁷) group and the terminal tetrazole or carboxylic acid group (e.g., a linker with at least three consecutively atoms in length) shows improvement in NLRP3 activity over shorter linker lengths. See, for example, Table C2, demonstrating improved NLRP3 activity of Comparative Example 2 and dramatically improved activity of Compound 2, each having linkers which are three consecutively carbon atoms in length, to Comparative Example 5, having a 2-atom carbon linker. Di-methyl substitution on the linker, as exemplified by Compound 13 (and as compared to Compound 2), is also well tolerated.

TABLE C2
The R4/Z Linker Lengths

| Compound | PBMC IL-1β IC₅₀ (uM) |
|---|---|
| 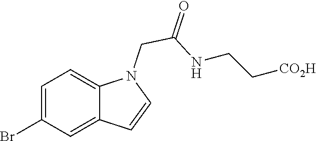 [SMILES: O=C(O)CCNC(=O)Cn1ccc2cc(Br)ccc21] | >100 (“E” Activity) |
| 3-(2-(5-bromo-1H-indol-1-yl)acetamido)propanoic acid | |
| COMPARATIVE EXAMPLE 5 | |
| 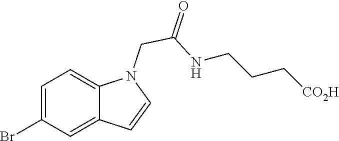 [SMILES: O=C(O)CCCNC(=O)Cn1ccc2cc(Br)ccc21] | 7.2 (“D” Activity) |
| 4-(2-(5-bromo-1H-indol-1-yl)acetamido)butanoic acid | |
| COMPARATIVE EXAMPLE 2 | |
| 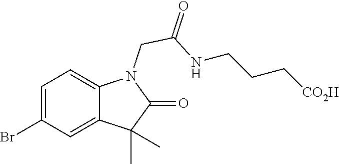 [SMILES: CC1(C)C(=O)N(CC(=O)NCCCC(=O)O)c2ccc(Br)cc21] | 0.15 (“A” Activity) |
| 4-(2-(5-bromo-3,3-dimethyl-2-oxoindolin-1-yl)acetamido)butanoic | |
| acid | |
| Compound 2 | |
| 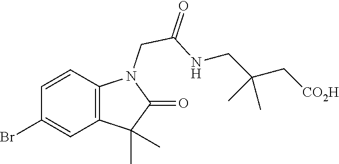 [SMILES: CC(C)(CNC(=O)CN1C(=O)C(C)(C)c2cc(Br)ccc21)CC(=O)O] | 0.11 (“A” Activity) |
| 4-(2-(5-bromo-3,3-dimethyl-2-oxoindolin-1-yl)acetamido)-3,3- | |
| dimethylbutanoic acid | |
| Compound 13 | |

Particular substitution patterns on the bicyclic core have also been found to improve NLRP3 activity. For example, as can been seen in Table C3, monosubstitution at the R¹ position provides good NLRP3 activity, but gains in activity are achieved with di- and tri-substitution of the core oxoindolinyl ring. Compare, for example, the activity of Compound 2 to the activity of Compounds 24, 56, 57, and 66.

TABLE C3
Bicyclic Core Substitution

| Compound | PBMC IL-1β IC₅₀ (uM) |
|---|---|
| 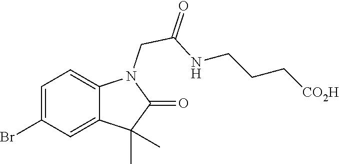 [SMILES: CC1(C)C(=O)N(CC(=O)NCCCC(=O)O)c2ccc(Br)cc21] | 0.15 (“A” Activity) |
| 4-(2-(5-bromo-3,3-dimethyl-2-oxoindolin-1-yl)acetamido)butanoic | |
| acid | |
| Compound 2 | |
| 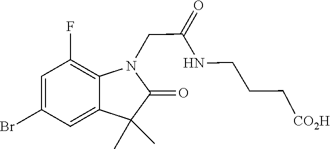 [SMILES: CC1(C)C(=O)N(CC(=O)NCCCC(=O)O)c2c(F)cc(Br)cc21] | 0.020 (“A” Activity) |
| 4-(2-(5-bromo-7-fluoro-3,3-dimethyl-2-oxoindolin-1- | |
| yl)acetamido)butanoic acid | |
| Compound 24 | |
| 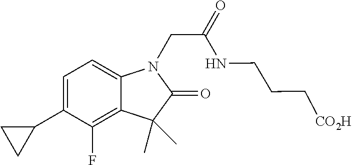 [SMILES: CC1(C)C(=O)N(CC(=O)NCCCC(=O)O)c2ccc(C3CC3)c(F)c21] | 0.046 (“A” Activity) |
| 4-(2-(5-cyclopropyl-4-fluoro-3,3-dimethyl-2-oxoindolin-1- | |
| yl)acetamido)butanoic acid | |
| Compound 56 | |
| 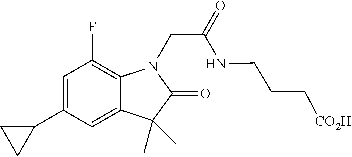 [SMILES: CC1(C)C(=O)N(CC(=O)NCCCC(=O)O)c2c(F)cc(C3CC3)cc21] | 0.024 (“A” Activity) |
| 4-(2-(5-cyclopropyl-7-fluoro-3,3-dimethyl-2-oxoindolin-1- | |
| yl)acetamido)butanoic acid | |
| Compound 66 | |
| 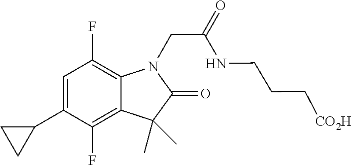 [SMILES: CC1(C)C(=O)N(CC(=O)NCCCC(=O)O)c2c(F)cc(C3CC3)c(F)c21] | 0.059 (“A” Activity) |
| 4-(2-(5-cyclopropyl-4,7-difluoro-3,3-dimethyl-2-oxoindolin-1- | |
| yl)acetamido)butanoic acid | |
| Compound 57 | |

Terminal tetrazole groups have also been found to provide improved activity over CH-homologues. See, e.g., the data provided in Table C4, demonstrating lower NLRP3 activity of Comparative Example 6 (a CH homologue) and Comparative Example 7 (a CH homologue) compared to the tetrazole containing Compound 65.

TABLE C4
Tetrazolyl groups

| Compound | PBMC IL-1β IC₅₀ (uM) |
|---|---|
| 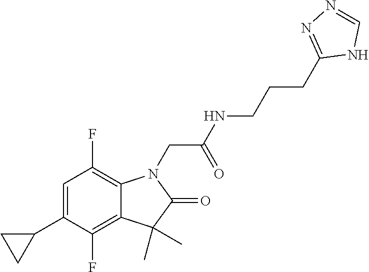 [SMILES: CC1(C)C(=O)N(CC(=O)NCCCc2nnc[nH]2)c2c(F)cc(C3CC3)c(F)c21] | 0.941 (“C” Activity) |
| N-(3-(4H-1,2,4-triazol-3-yl)propyl)-2-(5-cyclopropyl-4,7-difluoro- | |
| 3,3-dimethyl-2-oxoindolin-1-yl)acetamide | |
| COMPARATIVE EXAMPLE 6 | |
| 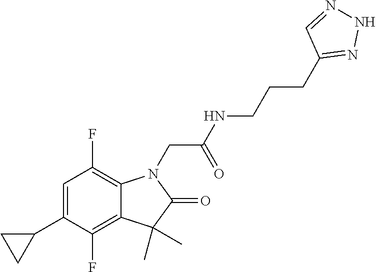 [SMILES: CC1(C)C(=O)N(CC(=O)NCCCc2cn[nH]n2)c2c(F)cc(C3CC3)c(F)c21] | 0.676 (“C” Activity) |
| N-(3-(2H-1,2,3-triazol-4-yl)propyl)-2-(5-cyclopropyl-4,7-difluoro- | |
| 3,3-dimethyl-2-oxoindolin-1-yl)acetamide | |
| COMPARATIVE EXAMPLE 7 | |
| 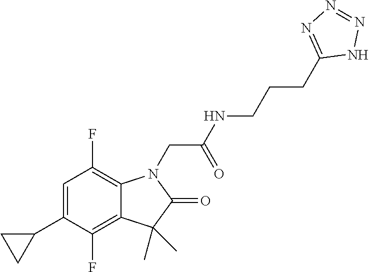 [SMILES: CC1(C)C(=O)N(CC(=O)NCCCc2nnn[nH]2)c2c(F)cc(C3CC3)c(F)c21] | 0.015 (“A” Activity) |
| N-(3-(1H-tetrazol-5-yl)propyl)-2-(5-cyclopropyl-4,7-difluoro-3,3- | |
| dimethyl-2-oxoindolin-1-yl)acetamide | |
| Compound 65 (tautomer 1) | |
| 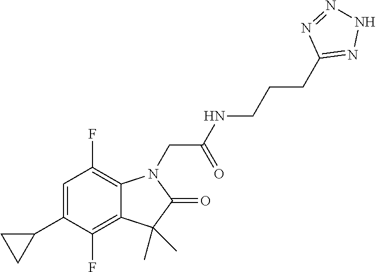 [SMILES: CC1(C)C(=O)N(CC(=O)NCCCc2nn[nH]n2)c2c(F)cc(C3CC3)c(F)c21] | |
| N-(3-(2H-tetrazol-5-yl)propyl)-2-(5-cyclopropyl-4,7-difluoro-3,3- | |
| dimethyl-2-oxoindolin-1-yl)acetamide | |
| Compound 65 (tautomer 2) | |

### OTHER EMBODIMENTS

Unless otherwise defined, all technical and scientific terms used herein have the same meaning as commonly understood by one of ordinary skill in the art to which this disclosure belongs. In the specification, the singular forms also include the plural unless the context clearly dictates otherwise.

All publications, patent applications, patents and other references mentioned herein are incorporated by reference. The references cited herein are not admitted to be prior art to the claimed invention. In the case of conflict, the present specification, including definitions, will control.

In the case of conflict between the chemical structures and names of the compounds disclosed herein, the chemical structures will control.

The foregoing description has been presented only for the purposes of illustration and is not intended to limit the disclosure to the precise form disclosed, but by the claims appended hereto. Other features and advantages of the disclosure will be apparent from the detailed description and the claims which follow.

1. A compound of Formula (I): 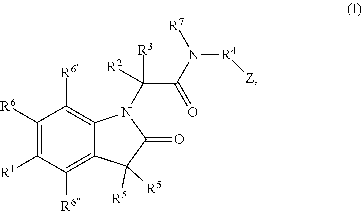
or a pharmaceutically acceptable salt, stereoisomer, or isotopic derivative thereof, wherein:
R¹ is halo, —CN, C₁-C₆ alkyl, C₂-C₆ alkenyl, C₂-C₆ alkynyl, C₁-C₆ haloalkyl, C₁-C₆ alkoxy, or C₃-C₁₂ cycloalkyl;
each R² and R³ independently is H, C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or R² and R³ cyclize, together with the atom to which they are attached, to form a C₃-C₁₂ cycloalkyl or 3- to 12-membered heterocyclyl, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁴ is —C₁-C₆ alkyl-, -(L₁)ₚ-(C₃-C₁₂ cycloalkyl)-, -(L₁)ₚ-(3- to 12-membered heterocyclyl)-, -(L₁)ₚ-(C₆-C₁₀ aryl)-, or -(L₁)ₚ-(5- to 10-membered heteroaryl)-, wherein each instance of L₁ is independently —(C(R^{L})₂)—, further wherein each instance of R^{L1} is independently H, halo or C₁-C₃ alkyl, or two R^{L1} groups, together with the atom to which they are attached, form a C₃₋₄ cycloalkyl; and wherein the alkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
Z is a -(L₂)ₙ-(carboxylic acid) or -(L₂)ₙ-tetrazole, wherein each instance of L₂ is independently —(C(R^{L2})₂)—, further wherein each instance of R^{L2} is independently H, halo or C₁-C₃ alkyl, or two R^{L2} groups, together with the atom to which they are attached, to form a C₃₋₄ cycloalkyl; and wherein the alkyl or cycloalkyl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
each R⁵ is independently halo or C₁-C₆ alkyl, wherein the alkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or both R⁵ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁶ is H, halo, —OH, C₁-C₆ alkyl, C₂-C₆ alkenyl, C₂-C₆ alkynyl, C₁-C₆ haloalkyl, or C₁-C₆ alkoxy;
R^{6′} is H, halo, —OH, C₁-C₆ alkyl, C₂-C₆ alkenyl, C₂-C₆ alkynyl, C₁-C₆ haloalkyl, or C₁-C₆ alkoxy;
R^{6″} is H, halo, —OH, C₁-C₆ alkyl, C₂-C₆ alkenyl, C₂-C₆ alkynyl, C₁-C₆ haloalkyl, or C₁-C₆ alkoxy;
R⁷ is H or C₁-C₄ alkyl;
n is an integer from 0, 1, 2, or 3; and
p is an integer from 0, 1, or 2.

2. The compound of claim 1, or a pharmaceutically acceptable salt, stereoisomer, or isotopic derivative thereof, wherein each instance of R² and R³ is H.

3. The compound of claim 1, or a pharmaceutically acceptable salt, stereoisomer, or isotopic derivative thereof, wherein —R⁴—Z is a group of formula (i): 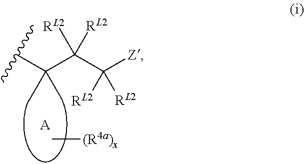
wherein Z′ is tetrazole or carboxylic acid; Ring A is C₃-C₅ cycloalkyl or 4- to 5-membered heterocyclyl; each R^{4a} is independently halo, C₁-C₃ alkyl, C₁-C₃ haloalkyl, or C₃ cycloalkyl; and x is 0, 1, 2, or 3.

4. The compound of claim 1, or a pharmaceutically acceptable salt, stereoisomer, or isotopic derivative thereof, wherein —R⁴—Z is a group of formula (ii): 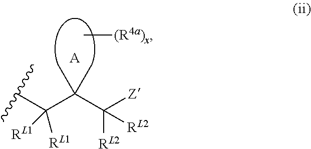
wherein Z′ is tetrazole or carboxylic acid; Ring A is C₃-C₅ cycloalkyl or 4- to 5-membered heterocyclyl; each R^{4a} is independently halo, C₁-C₃ alkyl, C₁-C₃ haloalkyl, or C₃ cycloalkyl; and x is 0, 1, 2, or 3.

5. The compound of claim 1, or a pharmaceutically acceptable salt, stereoisomer, or isotopic derivative thereof, wherein —R⁴—Z is a group of formula (iii): 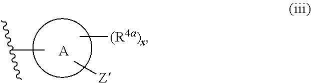
wherein Z′ is tetrazole or carboxylic acid; Ring A is C₃-C₅ cycloalkyl or 4- to 5-membered heterocyclyl; each R^{4a} is independently halo, C₁-C₃ alkyl, C₁-C₃ haloalkyl, or C₃ cycloalkyl; and x is 0, 1, 2, or 3.

6. The compound of claim 1, or a pharmaceutically acceptable salt, stereoisomer, or isotopic derivative thereof, wherein —R⁴—Z is a group of formula (iv): 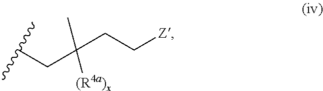
wherein Z′ is tetrazole or carboxylic acid; each R^{4a} is independently halo, C₁-C₃ alkyl, C₁-C₃ haloalkyl, or C₃ cycloalkyl; and x is 0, 1, 2, or 3.

7. The compound of claim 6, or a pharmaceutically acceptable salt, stereoisomer, or isotopic derivative thereof, wherein the group of formula (iv) is:

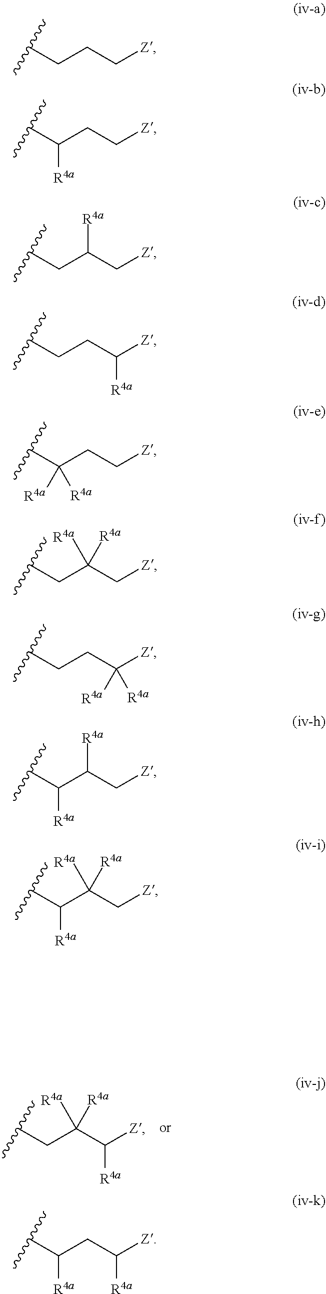

8. The compound of claim 7, or a pharmaceutically acceptable salt, stereoisomer, or isotopic derivative thereof, wherein the group of formula (iv) is:

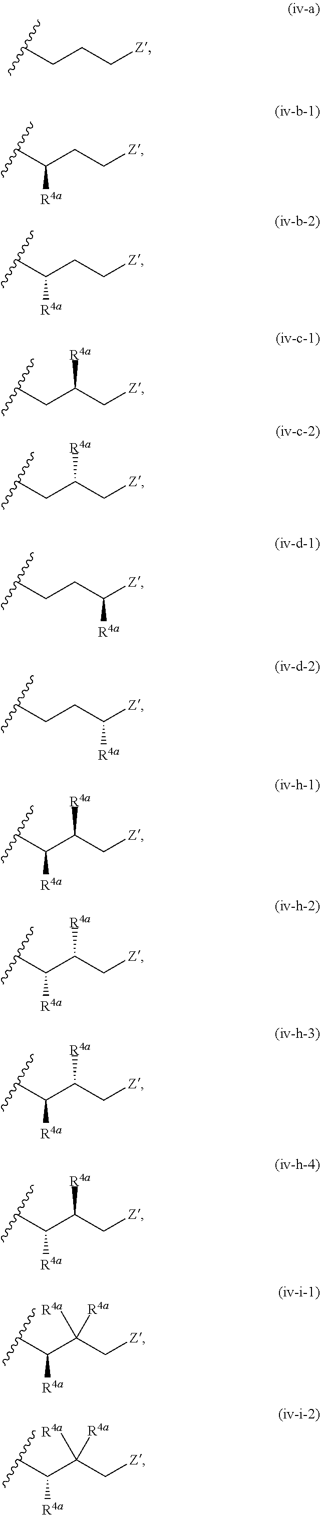

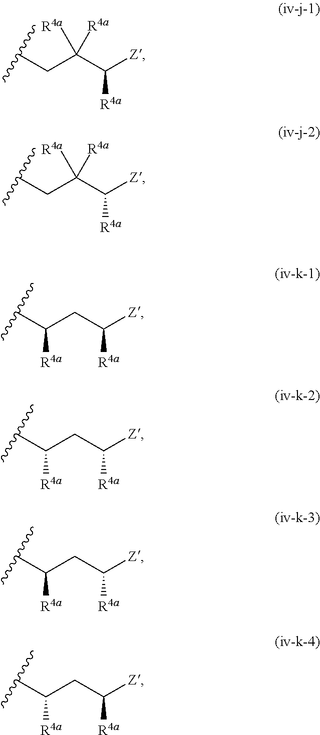

9. The compound of claim 8, or a pharmaceutically acceptable salt, stereoisomer, or isotopic derivative thereof, wherein the group of formula (iv) is:

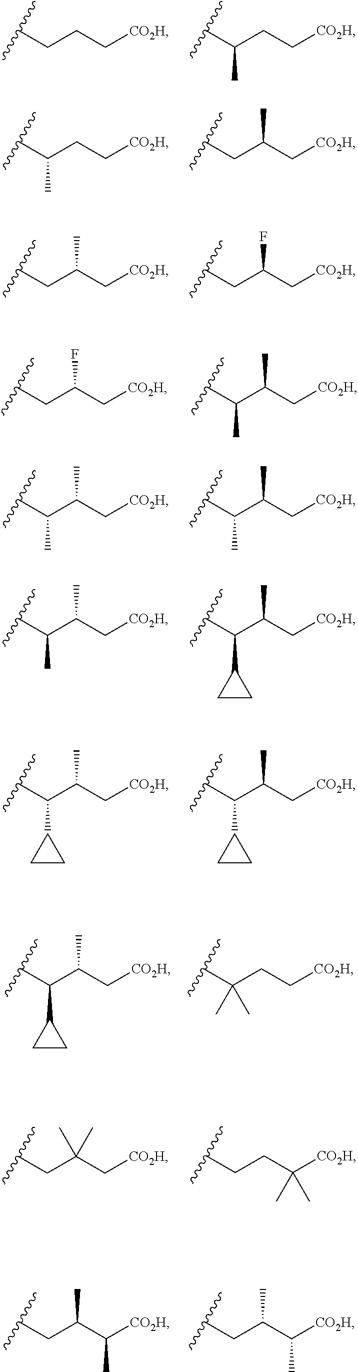

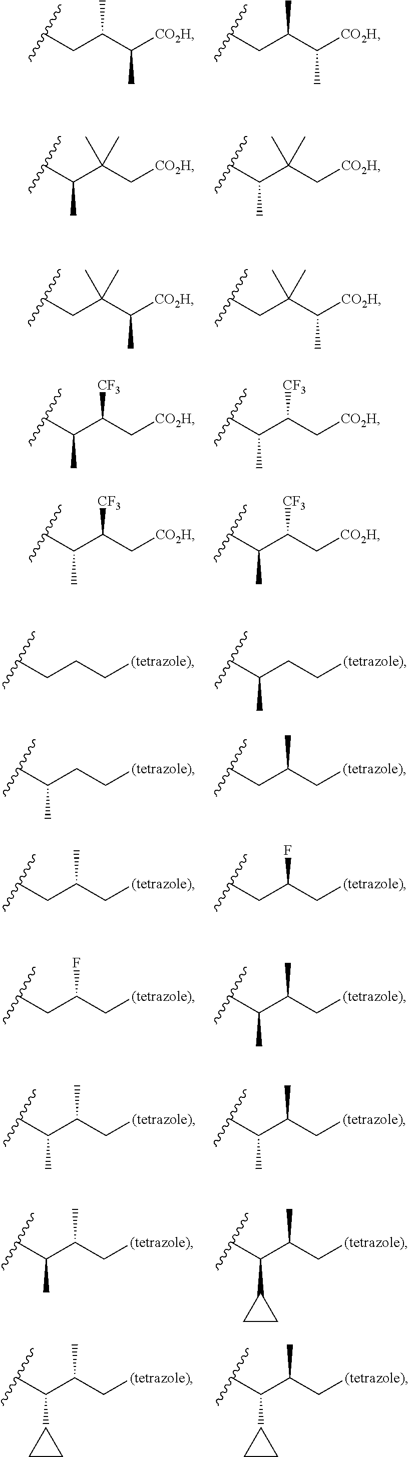

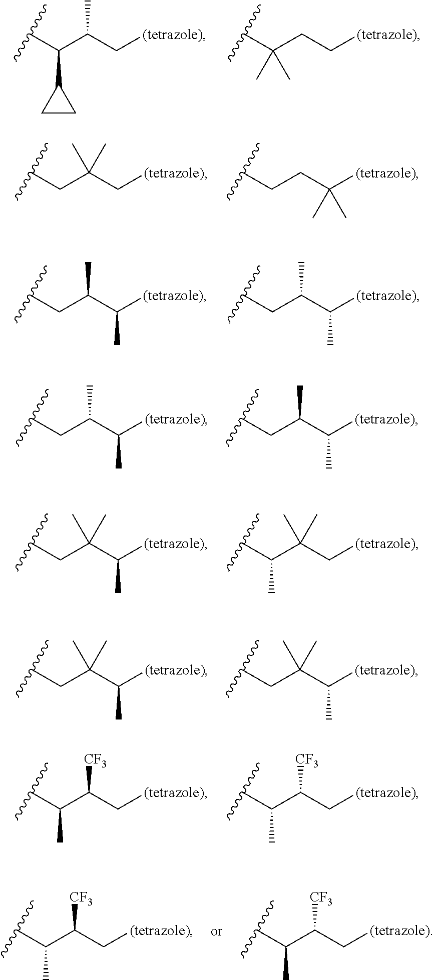

10. The compound of claim 1, or a pharmaceutically acceptable salt, stereoisomer, or isotopic derivative thereof, wherein —R⁴—Z group of formula: 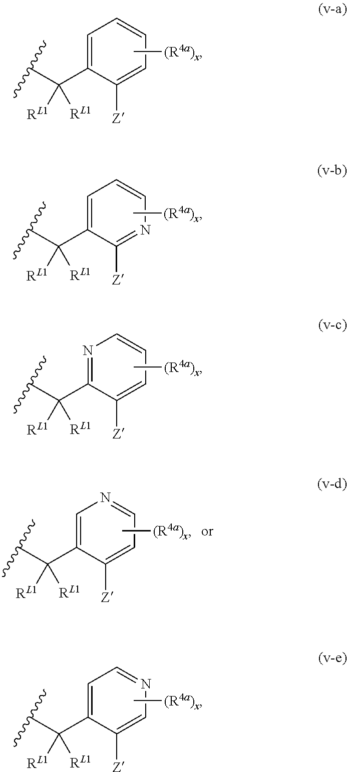
wherein Z′ is tetrazole or carboxylic acid; each R^{4a} is independently halo, C₁-C₃ alkyl, C₁-C₃ haloalkyl; and x is 0, 1, 2, or 3.

11. The compound of claim 1, or a pharmaceutically acceptable salt, stereoisomer, or isotopic derivative thereof, wherein —R⁴—Z is a group of formula: 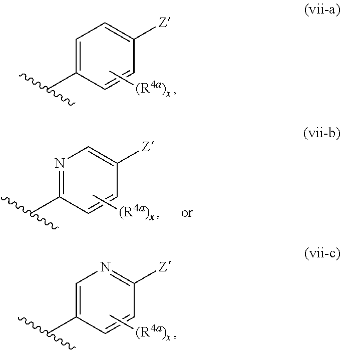
wherein Z′ is tetrazole or carboxylic acid; and each R^{4a} is independently halo, C₁-C₃ alkyl, C₁-C₃ haloalkyl, and x is 0, 1, 2, or 3.

12. The compound of claim 1, or a pharmaceutically acceptable salt, stereoisomer, or isotopic derivative thereof, wherein each instance of R⁵ is selected from the group consisting of fluoro and C₁-C₆ alkyl, or both R⁵ cyclize, together with the atom to which they are attached, to form a C₃ cycloalkyl.

13. The compound of claim 12, or a pharmaceutically acceptable salt, stereoisomer, or isotopic derivative thereof, wherein each instance of R⁵ is fluoro or —CH₃.

14. The compound of claim 12, or a pharmaceutically acceptable salt, stereoisomer, or isotopic derivative thereof, wherein the 6,5-bicyclic core of formula (viii): 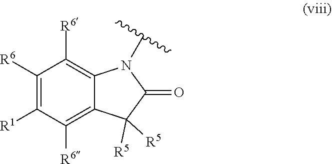
is of formula: 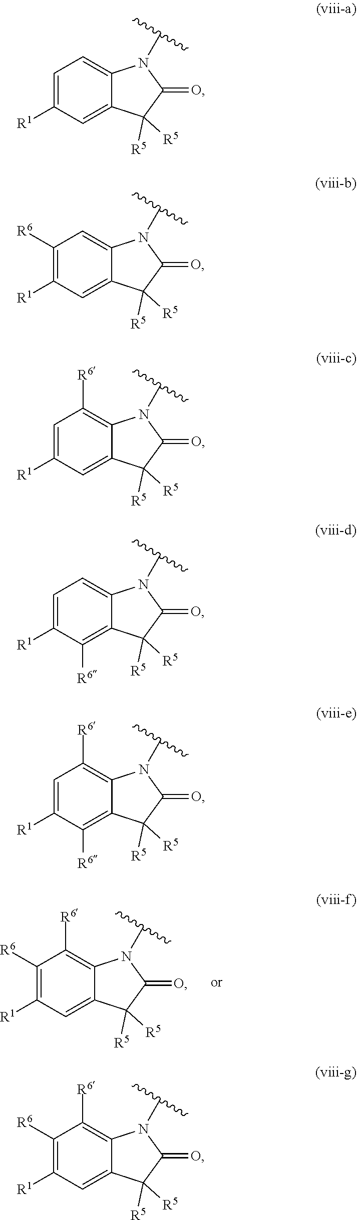
wherein at least one of R⁶, R^{6′} and R^{6″} is not H.

15. The compound of claim 14, or a pharmaceutically acceptable salt, stereoisomer, or isotopic derivative thereof, wherein the 6,5-bicyclic core is of formula:

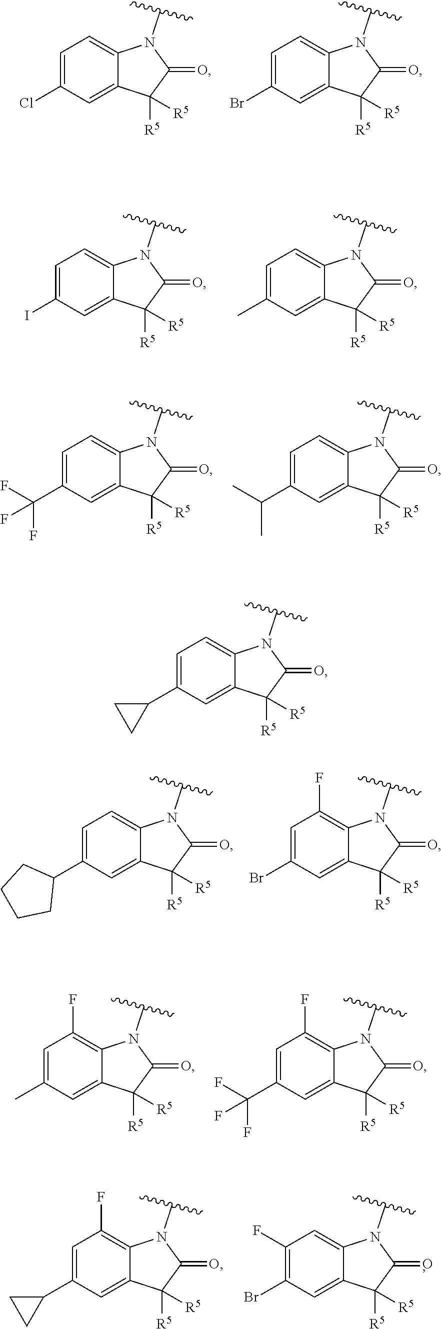

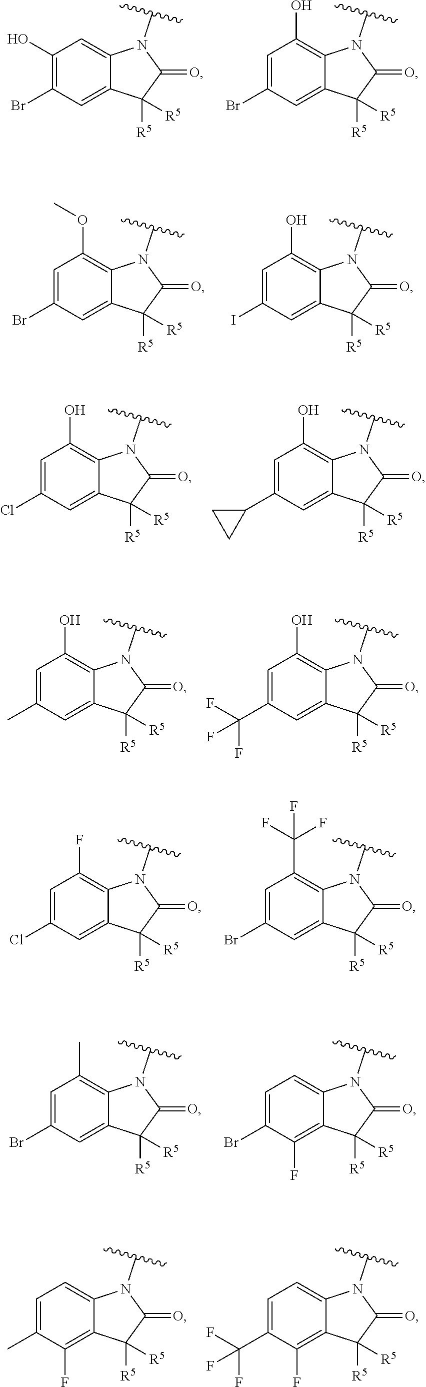

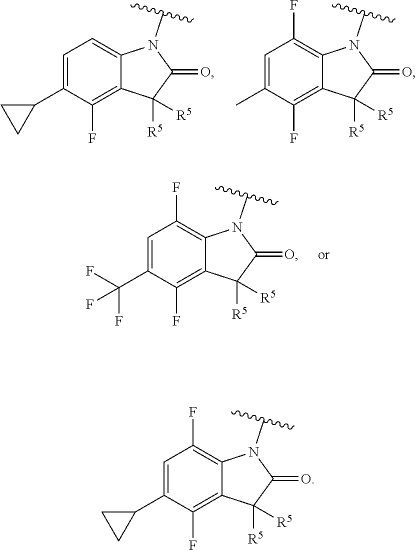

16. The compound of claim 1, wherein the compound of Formula (I) is of Formula: 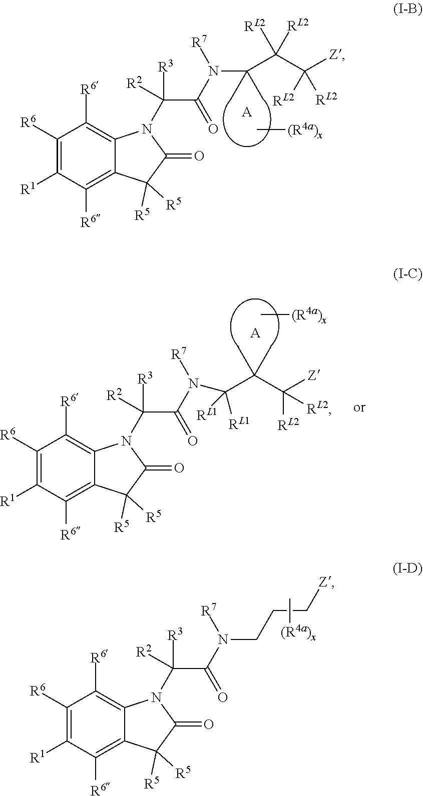
or a pharmaceutically acceptable salt, stereoisomer, or isotopic derivative thereof, wherein:
Z′ is tetrazole or carboxylic acid;
Ring A is C₃-C₅ cycloalkyl or 4- to 5-membered heterocyclyl;
each R^{4a} is independently halo, C₁-C₃ alkyl, C₁-C₃ haloalkyl, or C₃ cycloalkyl; and
x is 0, 1, 2, or 3.

17. The compound of claim 16, wherein the compound of Formula (I) is of Formula: 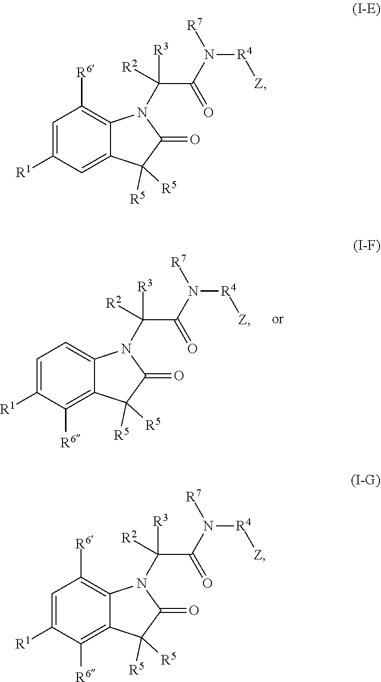
or a pharmaceutically acceptable salt, stereoisomer, or isotopic derivative thereof, wherein at least one of R⁶, R^{6′} and R^{6″} is not H.

18. The compound of claim 1, selected from the group consisting of: 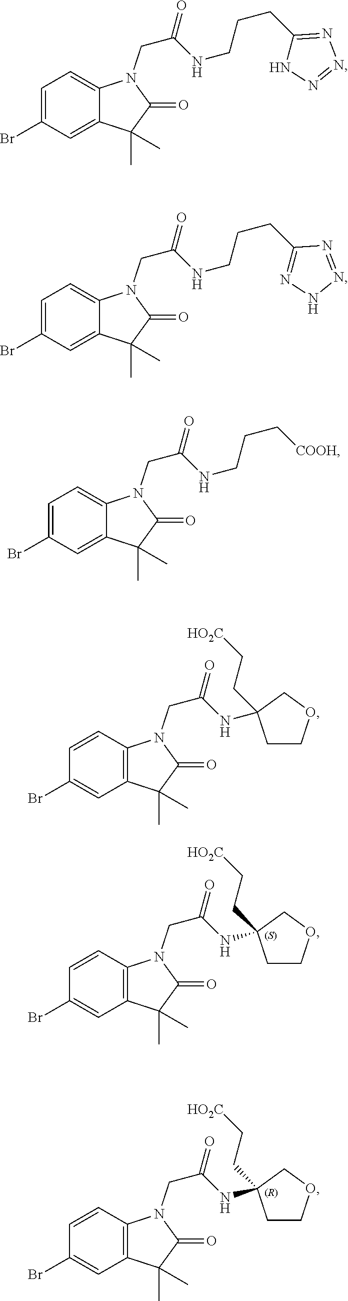 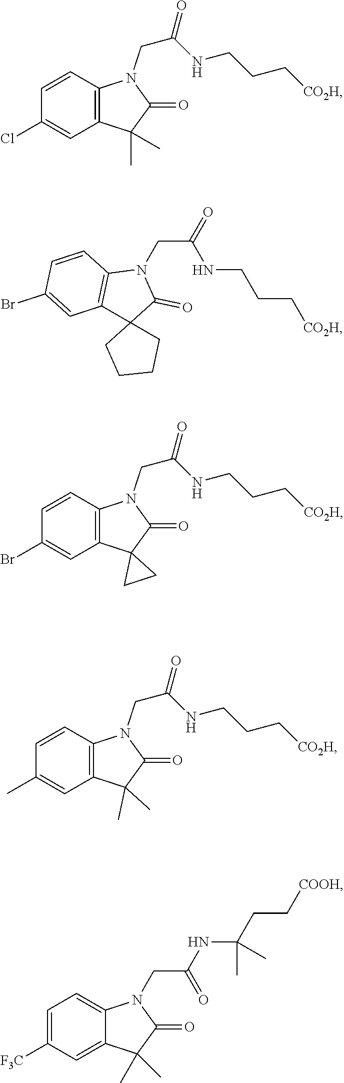 [SMILES: CC1(C)C(=O)N(CC(=O)NCCCC(=O)O)c2ccc(Cl)cc21 ; O=C(O)CCCNC(=O)CN1C(=O)C2(CCCC2)c2cc(Br)ccc21 ; O=C(O)CCCNC(=O)CN1C(=O)C2(CC2)c2cc(Br)ccc21 ; Cc1ccc2c(c1)C(C)(C)C(=O)N2CC(=O)NCCCC(=O)O ; CC(C)(CCC(=O)O)NC(=O)CN1C(=O)C(C)(C)c2cc(C(F)(F)F)ccc21] 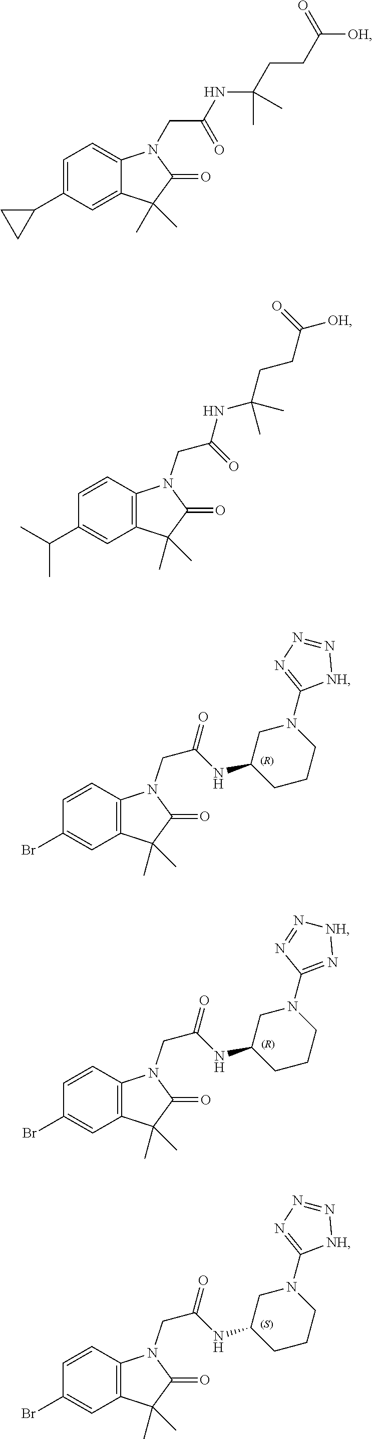 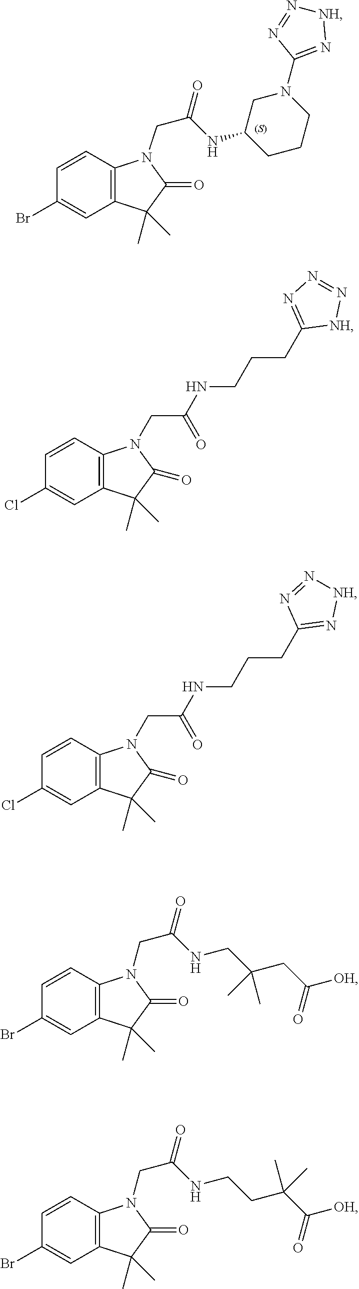 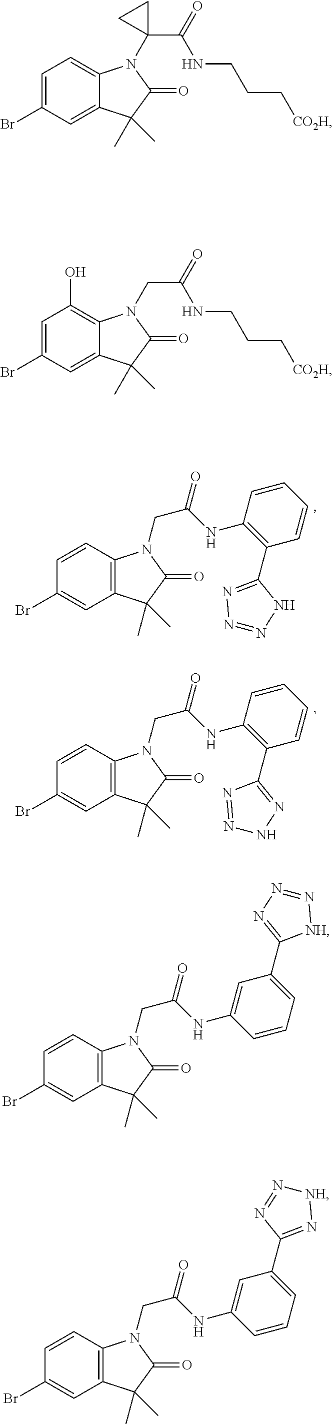 [SMILES: CC1(C)C(=O)N(C2(C(=O)NCCCC(=O)O)CC2)c2ccc(Br)cc21 ; CC1(C)C(=O)N(CC(=O)NCCCC(=O)O)c2c(O)cc(Br)cc21 ; CC1(C)C(=O)N(CC(=O)Nc2ccccc2-c2nnn[nH]2)c2ccc(Br)cc21 ; CC1(C)C(=O)N(CC(=O)Nc2ccccc2-c2nn[nH]n2)c2ccc(Br)cc21 ; CC1(C)C(=O)N(CC(=O)Nc2cccc(-c3nnn[nH]3)c2)c2ccc(Br)cc21 ; CC1(C)C(=O)N(CC(=O)Nc2cccc(-c3nn[nH]n3)c2)c2ccc(Br)cc21] 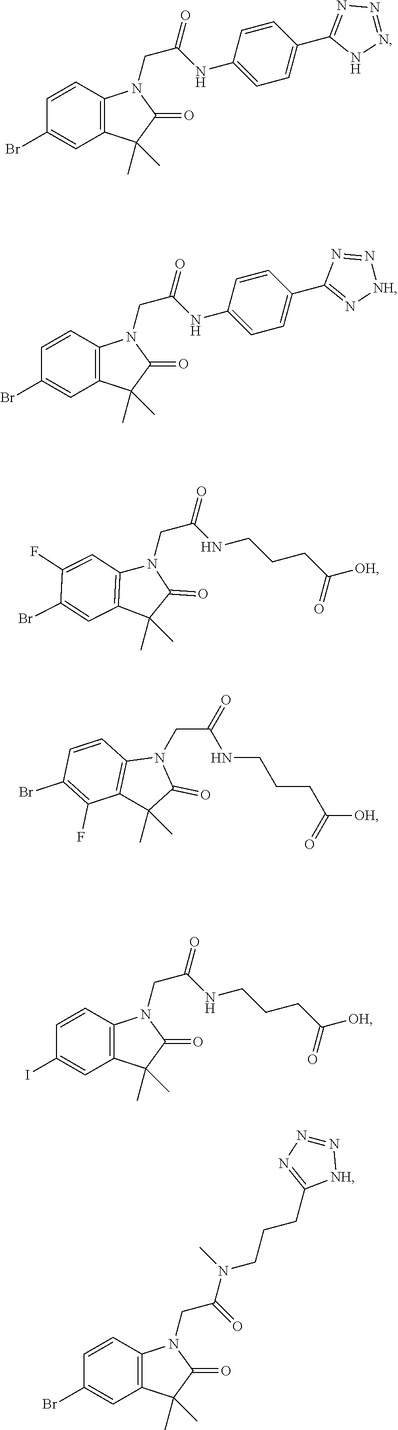 [SMILES: CC1(C)C(=O)N(CC(=O)Nc2ccc(-c3nnn[nH]3)cc2)c2ccc(Br)cc21 ; CC1(C)C(=O)N(CC(=O)Nc2ccc(-c3nn[nH]n3)cc2)c2ccc(Br)cc21 ; CC1(C)C(=O)N(CC(=O)NCCCC(=O)O)c2cc(F)c(Br)cc21 ; CC1(C)C(=O)N(CC(=O)NCCCC(=O)O)c2ccc(Br)c(F)c21 ; CC1(C)C(=O)N(CC(=O)NCCCC(=O)O)c2ccc(I)cc21 ; CN(CCCc1nnn[nH]1)C(=O)CN1C(=O)C(C)(C)c2cc(Br)ccc21] 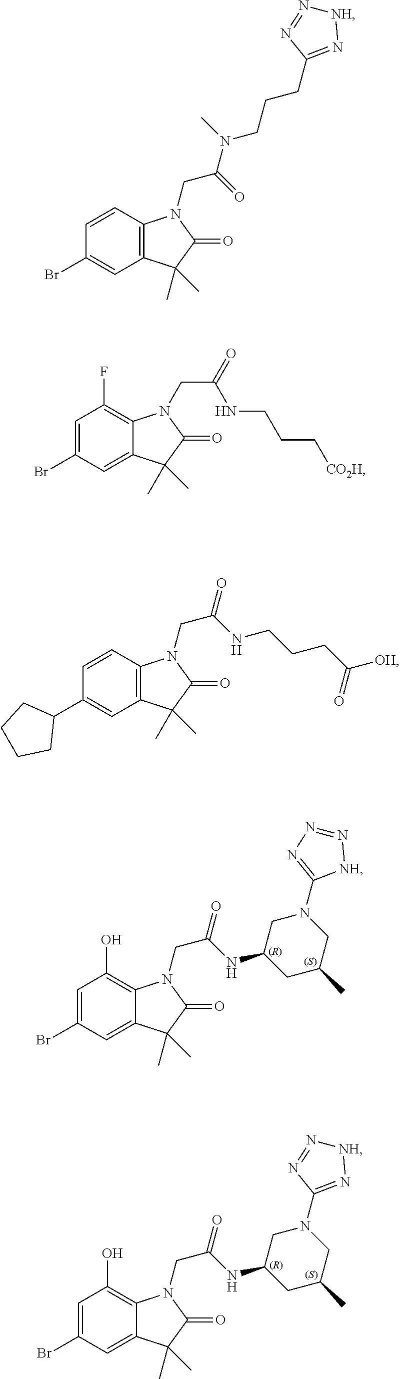 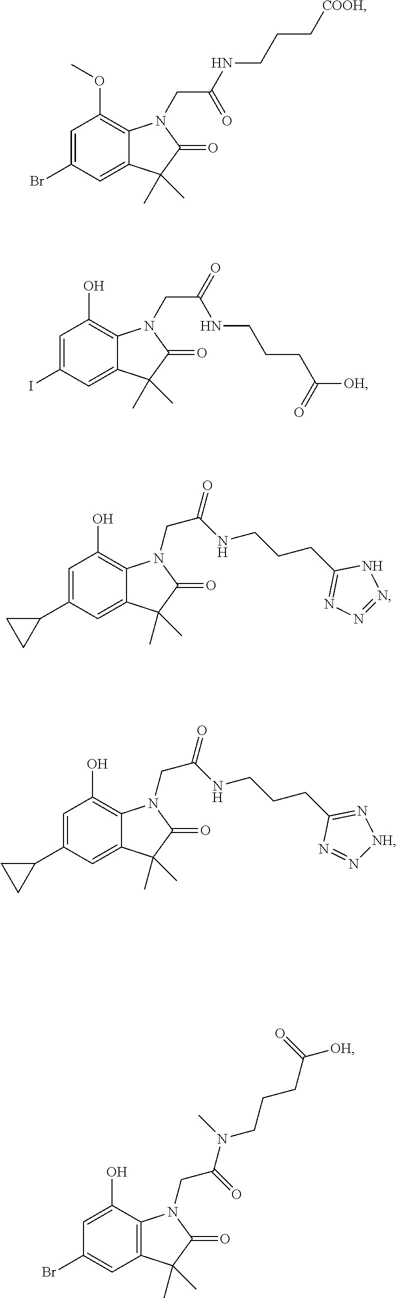 [SMILES: COc1cc(Br)cc2c1N(CC(=O)NCCCC(=O)O)C(=O)C2(C)C ; CC1(C)C(=O)N(CC(=O)NCCCC(=O)O)c2c(O)cc(I)cc21 ; CC1(C)C(=O)N(CC(=O)NCCCc2nnn[nH]2)c2c(O)cc(C3CC3)cc21 ; CC1(C)C(=O)N(CC(=O)NCCCc2nn[nH]n2)c2c(O)cc(C3CC3)cc21 ; CN(CCCC(=O)O)C(=O)CN1C(=O)C(C)(C)c2cc(Br)cc(O)c21] 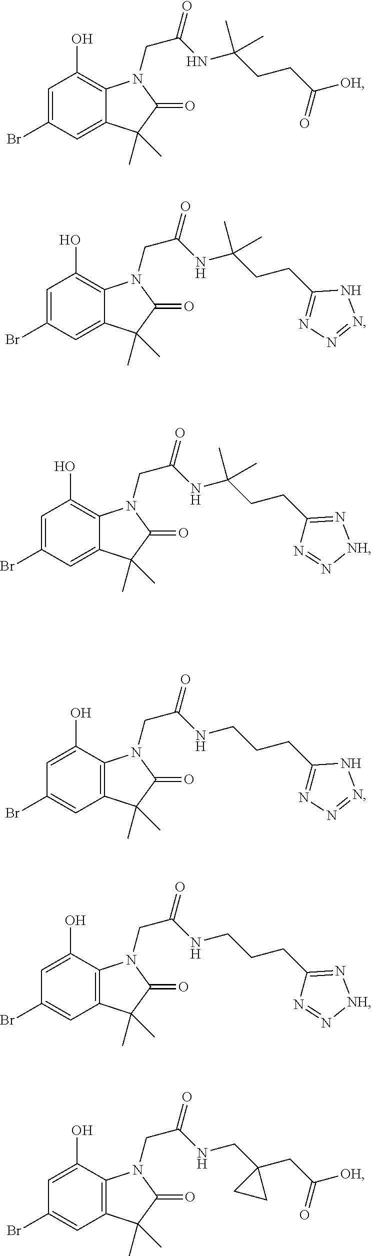 [SMILES: CC(C)(CCC(=O)O)NC(=O)CN1C(=O)C(C)(C)c2cc(Br)cc(O)c21 ; CC(C)(CCc1nnn[nH]1)NC(=O)CN1C(=O)C(C)(C)c2cc(Br)cc(O)c21 ; CC(C)(CCc1nn[nH]n1)NC(=O)CN1C(=O)C(C)(C)c2cc(Br)cc(O)c21 ; CC1(C)C(=O)N(CC(=O)NCCCc2nnn[nH]2)c2c(O)cc(Br)cc21 ; CC1(C)C(=O)N(CC(=O)NCCCc2nn[nH]n2)c2c(O)cc(Br)cc21 ; CC1(C)C(=O)N(CC(=O)NCC2(CC(=O)O)CC2)c2c(O)cc(Br)cc21] 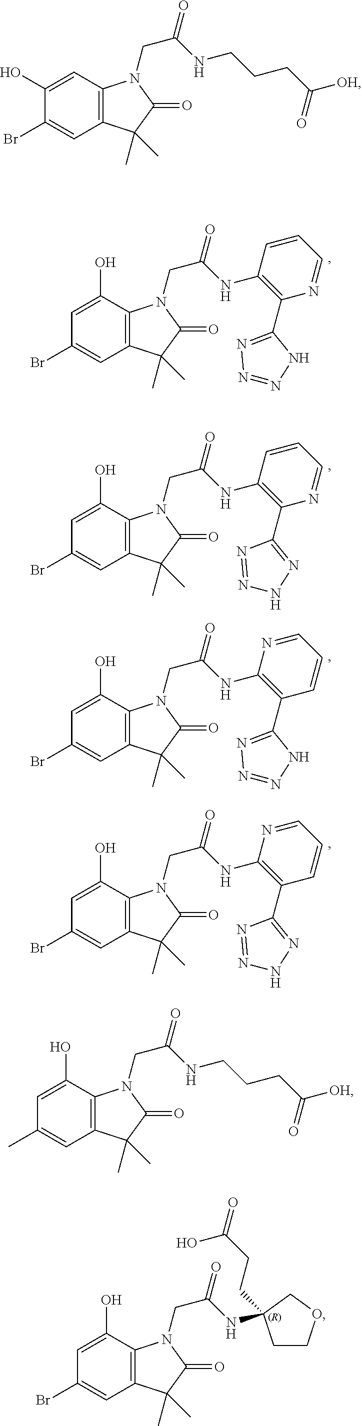 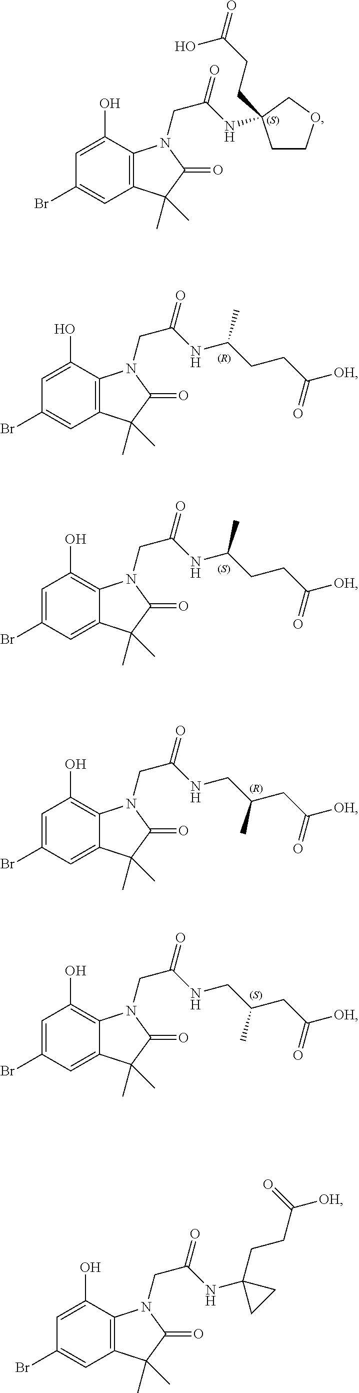 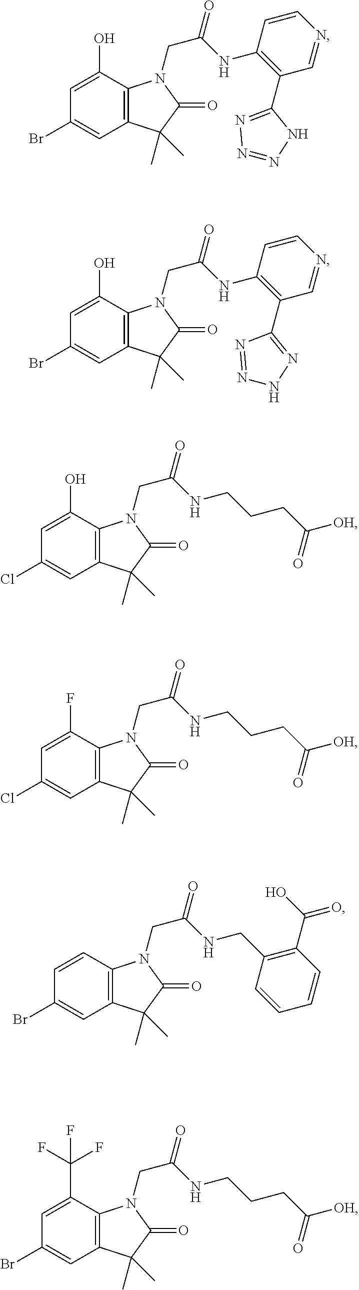 [SMILES: CC1(C)C(=O)N(CC(=O)Nc2ccncc2-c2nnn[nH]2)c2c(O)cc(Br)cc21 ; CC1(C)C(=O)N(CC(=O)Nc2ccncc2-c2nn[nH]n2)c2c(O)cc(Br)cc21 ; CC1(C)C(=O)N(CC(=O)NCCCC(=O)O)c2c(O)cc(Cl)cc21 ; CC1(C)C(=O)N(CC(=O)NCCCC(=O)O)c2c(F)cc(Cl)cc21 ; CC1(C)C(=O)N(CC(=O)NCc2ccccc2C(=O)O)c2ccc(Br)cc21 ; CC1(C)C(=O)N(CC(=O)NCCCC(=O)O)c2c(C(F)(F)F)cc(Br)cc21] 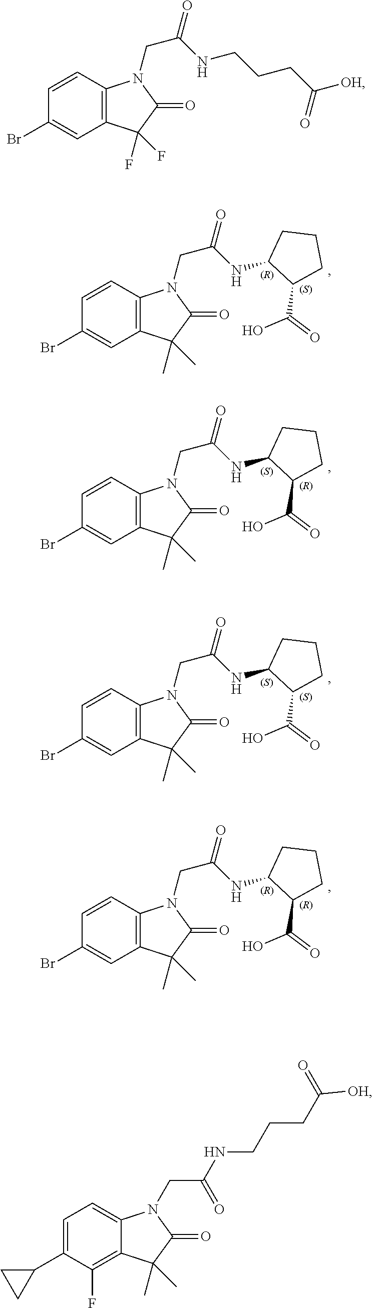 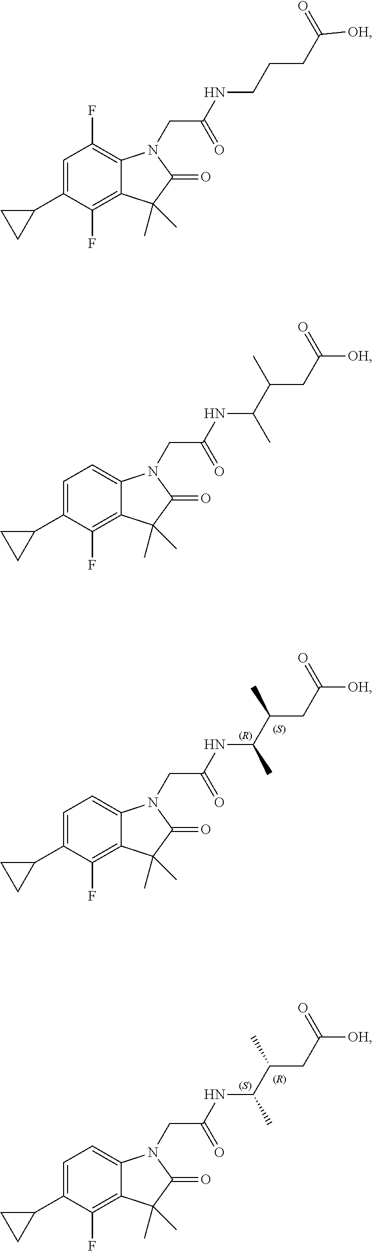 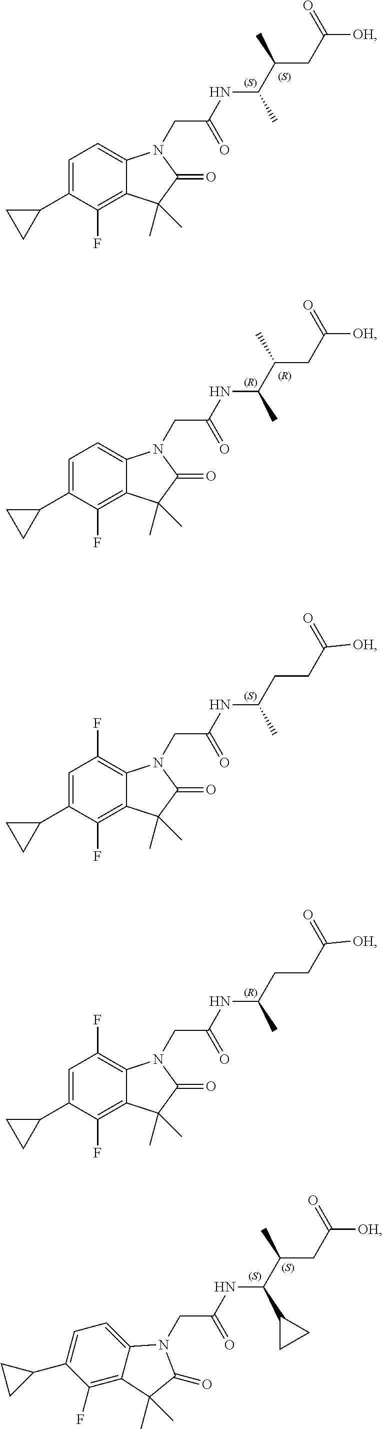 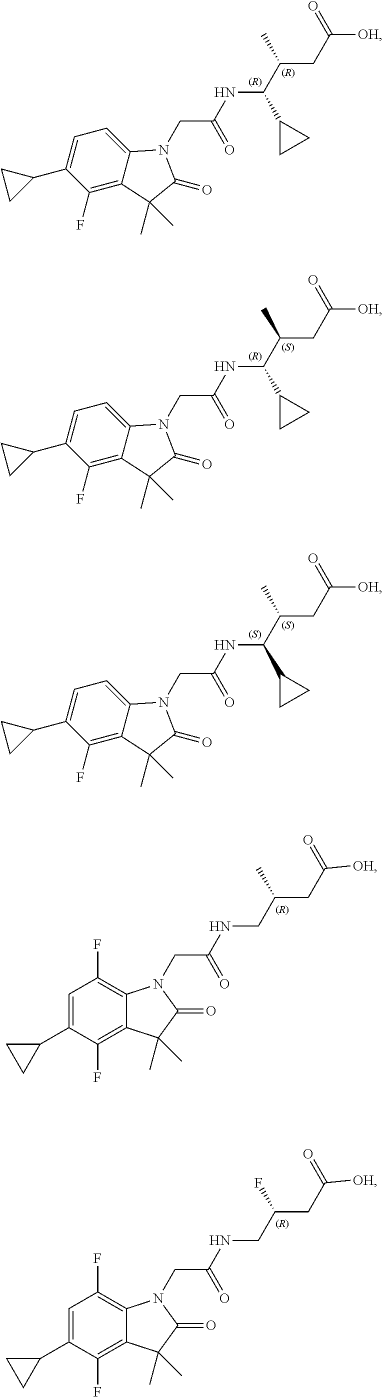 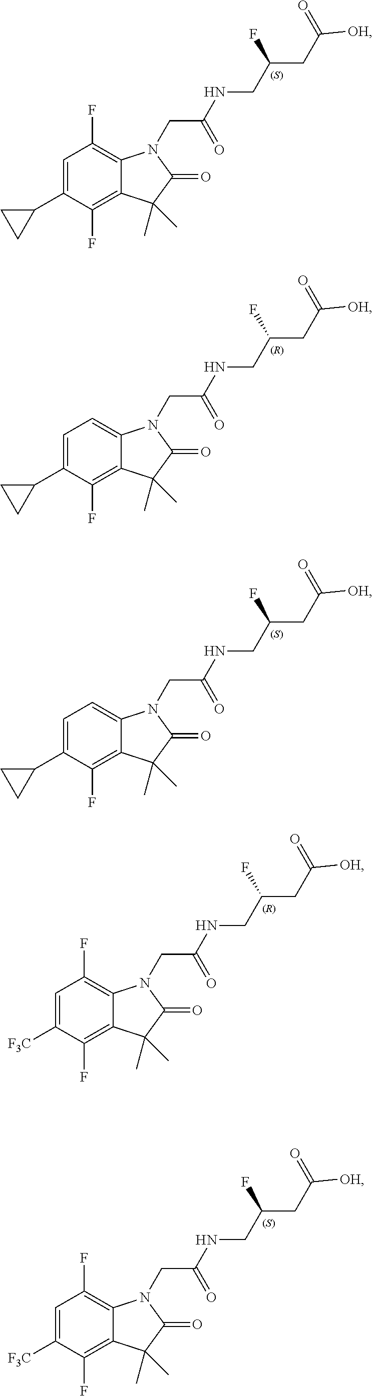 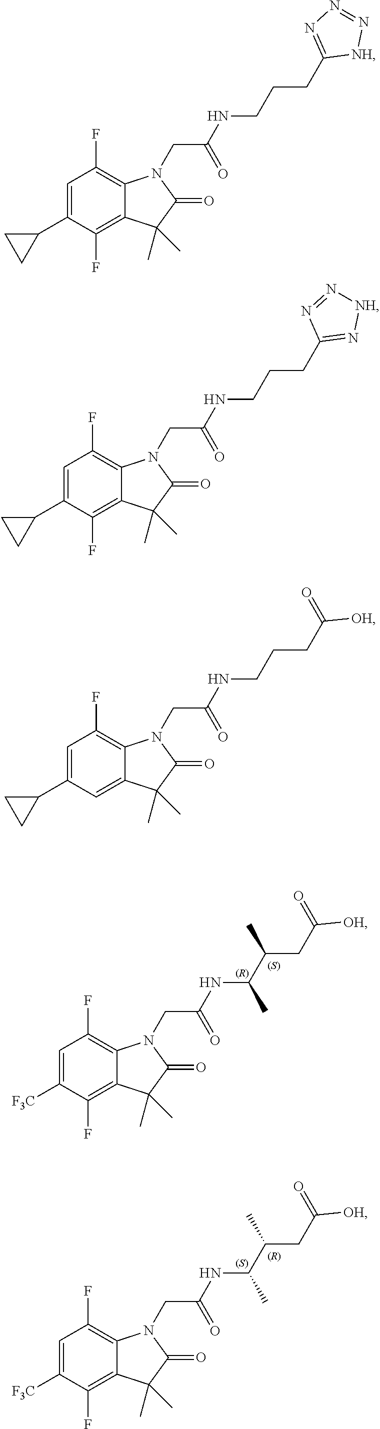 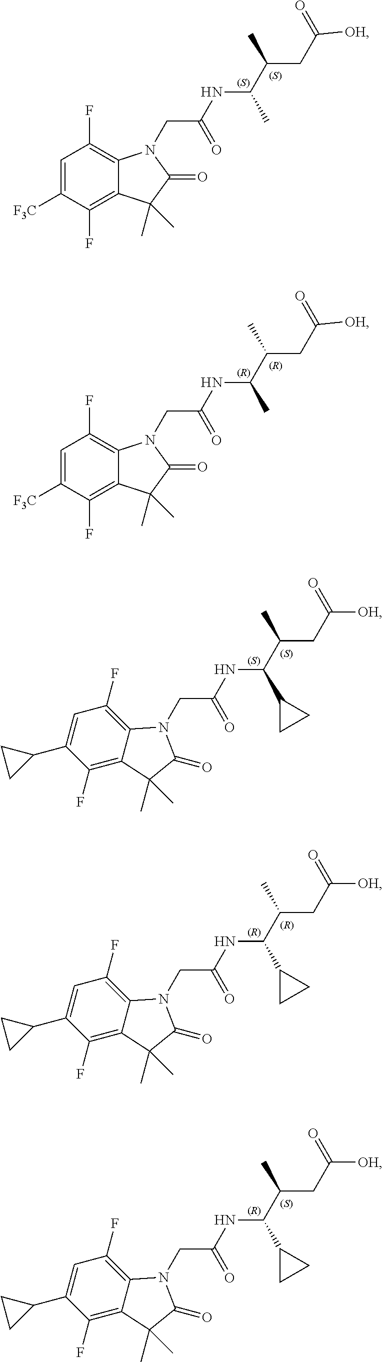 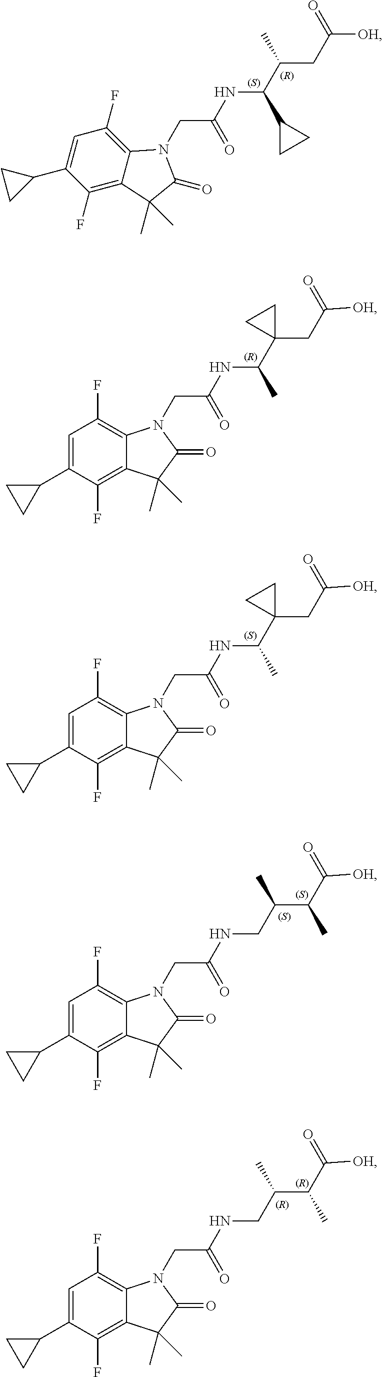 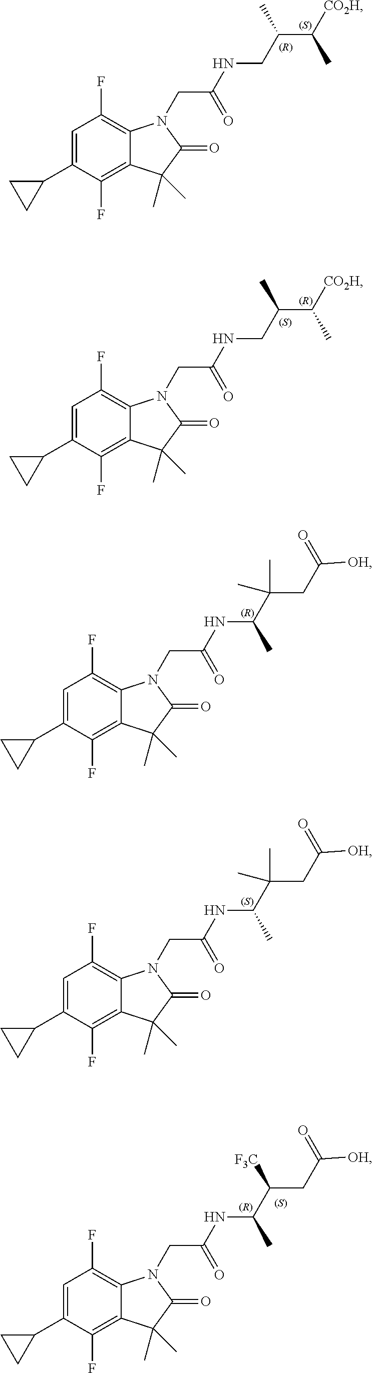 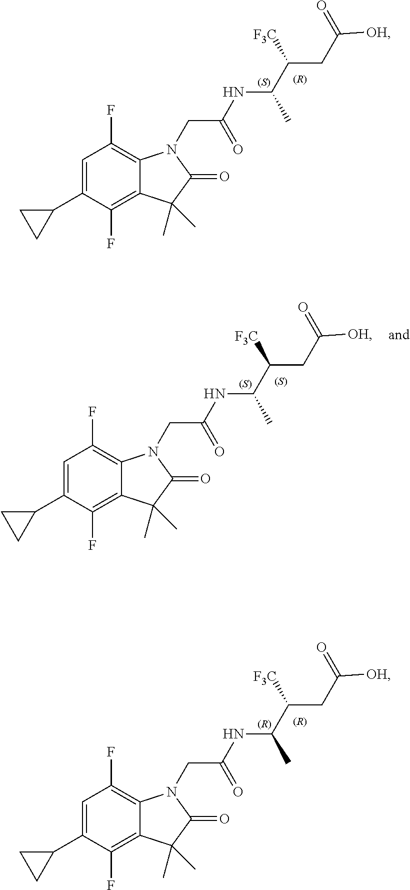
or a pharmaceutically acceptable salt, stereoisomer, or isotopic derivative of any of the foregoing.

19. A pharmaceutical composition comprising a compound of claim 1, or a pharmaceutically acceptable salt, stereoisomer, or isotopic derivative thereof, and one or more pharmaceutically acceptable excipients.

20. A method of treating a disease or disorder in a subject in need thereof, comprising administering to the subject a compound of claim 1, or a pharmaceutically acceptable salt, stereoisomer, or isotopic derivative thereof.

21. A method of preparing a compound of Formula (I) of claim 1, or a pharmaceutically acceptable salt, stereoisomer, or isotopic derivative thereof, wherein Z is -(L₂)ₙ-tetrazole, the method comprising coupling of a substituted indolinone (acetic acid) (i): 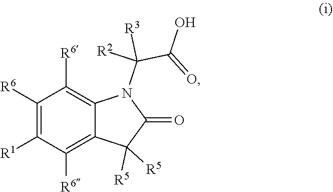
or salt thereof;
with an aminotetrazole (ii): 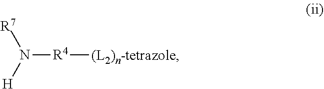
or salt thereof,
to provide the tetrazole analog: 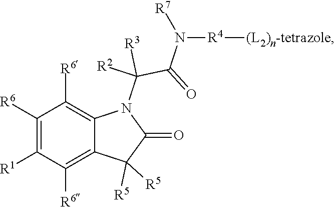
or salt thereof.

22. A method of preparing a compound of Formula (I) of claim 1, or a pharmaceutically acceptable salt, stereoisomer, or isotopic derivative thereof, wherein Z is -(L₂)ₙ-carboxylic acid, the method comprising deprotecting a compound of Formula (v-o): 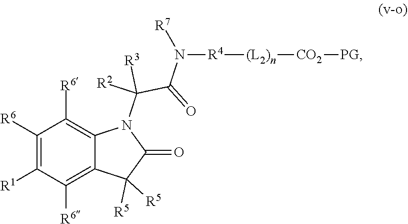
or salt thereof,
wherein PG is C₁₋₆ alkyl, C₆ aryl, or aryl-C₁₋₆ alkyl, wherein the alkyl and aryl are optionally substituted with one or more halo, C₁₋₆ alkyl, or C₁₋₆ alkoxy.

23. A compound of Formula (v-o): 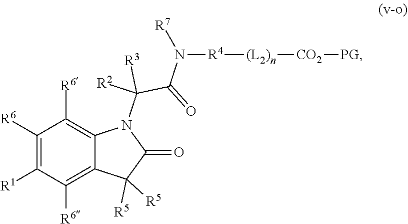
or salt thereof,
wherein
R¹ is halo, —CN, C₁-C₆ alkyl, C₂-C₆ alkenyl, C₂-C₆ alkynyl, C₁-C₆ haloalkyl, C₁-C₆ alkoxy, or C₃-C₁₂ cycloalkyl;
each R² and R³ independently is H, C₁-C₆ alkyl, C₂-C₆ alkenyl, C₂-C₆ alkynyl, C₁-C₆ haloalkyl, C₁-C₆ alkoxy, C₃-C₁₂ cycloalkyl, 3- to 12-membered heterocyclyl, C₆-C₁₀ aryl, or 5- to 10-membered heteroaryl, where the alkyl, alkenyl, alkynyl, haloalkyl, alkoxy, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or R² and R³ cyclize, together with the atom to which they are attached, to form a C₃-C₁₂ cycloalkyl or 3- to 12-membered heterocyclyl, wherein the cycloalkyl or heterocyclyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁴ is —C₁-C₆ alkyl-, -(L₁)ₚ-(C₃-C₁₂ cycloalkyl)-, -(L₁)ₚ-(3- to 12-membered heterocyclyl)-, -(L₁)ₚ-(C₆-C₁₀ aryl)-, or -(L₁)ₚ-(5- to 10-membered heteroaryl)-, wherein each instance of L₁ is independently —(C(R^{L})₂)—, further wherein each instance of R^{L1} is independently H, halo or C₁-C₃ alkyl, or two R^{L1} groups, together with the atom to which they are attached, form a C₃₋₄ cycloalkyl; and wherein the alkyl, cycloalkyl, heterocyclyl, aryl, or heteroaryl is optionally substituted with one or more halo, —CN, —OH, amino, C₁-C₆ alkyl, C₂-C₆ alkenyl, C₂-C₆ alkynyl, C₁-C₆ haloalkyl, C₁-C₆ alkoxy, or C₃ cycloalkyl;
each R⁵ is independently halo or C₁-C₆ alkyl, wherein the alkyl is optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy, or both R⁵ cyclize, together with the atom to which they are attached, to form a C₃-C₁₂ cycloalkyl optionally substituted with one or more halo, —CN, —OH, amino, C₁-C₆ alkyl, C₂-C₆ alkenyl, C₂-C₆ alkynyl, C₁-C₆ haloalkyl, or C₁-C₆ alkoxy;
R⁶ is H, halo, —OH, C₁-C₆ alkyl, C₂-C₆ alkenyl, C₂-C₆ alkynyl, C₁-C₆ haloalkyl, or C₁-C₆ alkoxy;
R^{6′} is H, halo, —OH, C₁-C₆ alkyl, C₂-C₆ alkenyl, C₂-C₆ alkynyl, C₁-C₆ haloalkyl, or C₁-C₆ alkoxy;
R^{6″} is H, halo, —OH, C₁-C₆ alkyl, C₂-C₆ alkenyl, C₂-C₆ alkynyl, C₁-C₆ haloalkyl, or C₁-C₆ alkoxy;
R⁷ is H or C₁-C₄ alkyl;
n is an integer from 0, 1, 2, or 3; and
PG is C₁₋₆ alkyl, C₆ aryl, or aryl-C₁₋₆ alkyl, wherein the alkyl and aryl are optionally substituted with one or more halo, C₁₋₆ alkyl, or C₁₋₆ alkoxy.

24. The compound of claim 17, wherein the compound is: 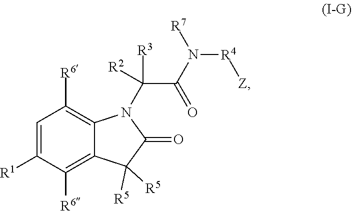
or a pharmaceutically acceptable salt, stereoisomer, or isotopic derivative thereof; wherein:
R¹ is halo, C₁-C₃ alkyl, C₁-C₃haloalkyl, or C₃-C₅ cycloalkyl;
R^{6′} is halo;
R^{6″} is halo;
each instance of R⁵ is independently the same or different selected from fluoro or C₁₋₃ alkyl;
each instance of R² and R³ is H;
R⁷ is H or C₁-C₄ alkyl;
R⁴—Z group is of formula: 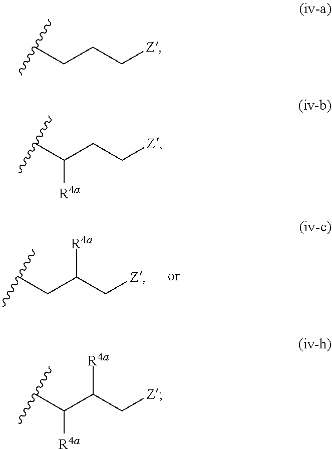
each R^{4a} is independently halo, C₁-C₃ alkyl, C₁-C₃ haloalkyl, or C₃ cycloalkyl; and
Z′ is tetrazole or carboxylic acid.

25. The compound of claim 24, selected from the group consisting of 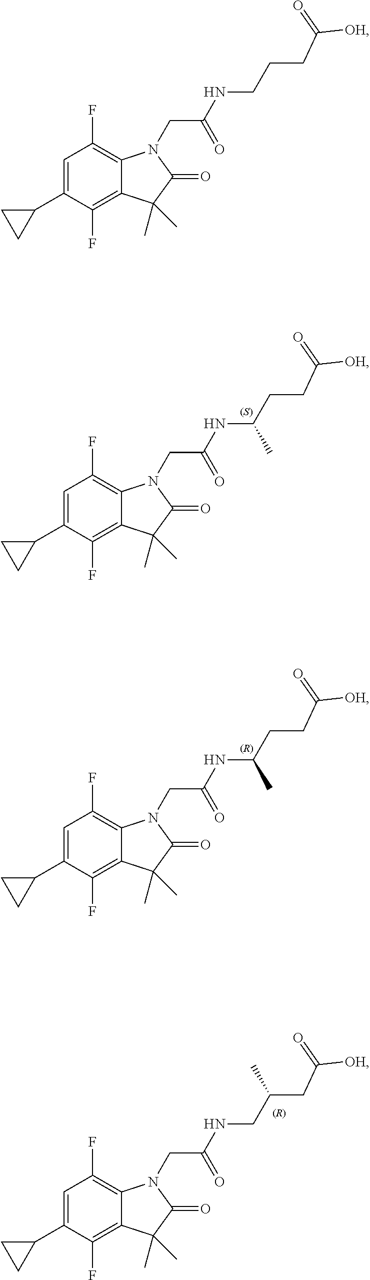 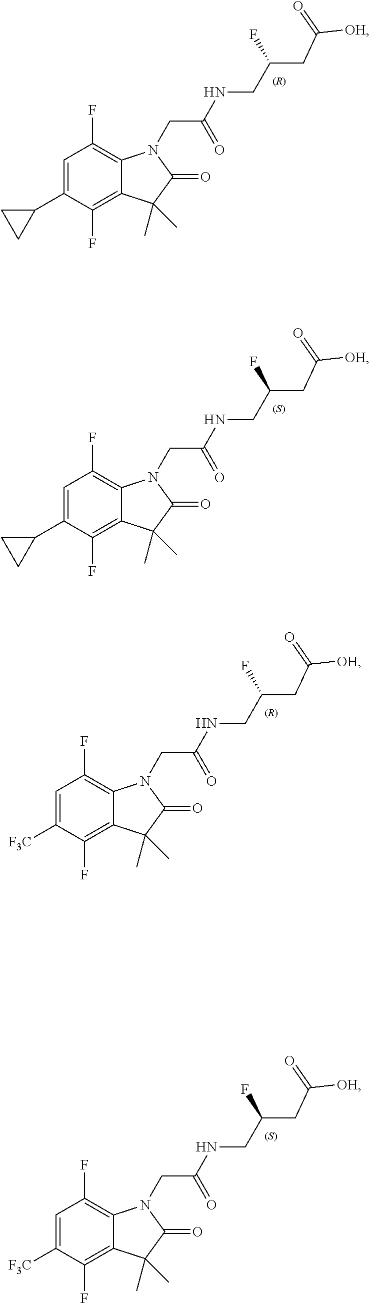 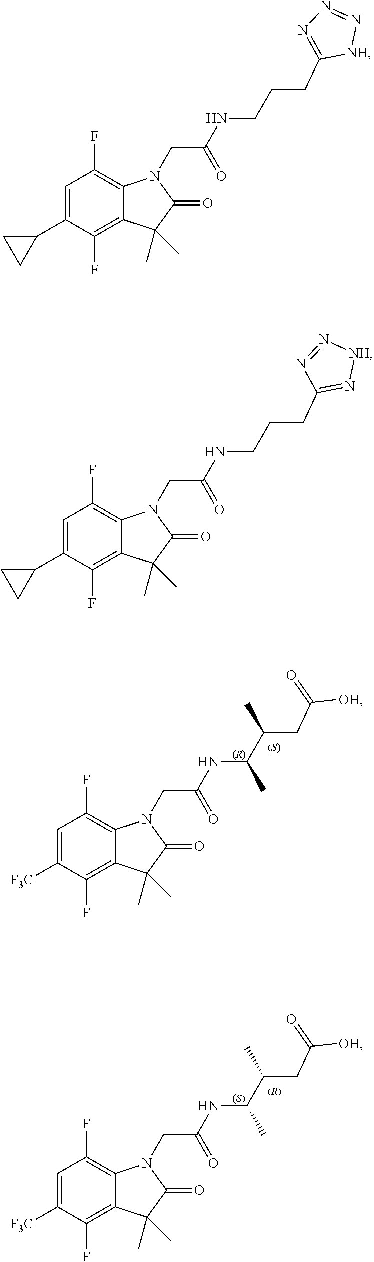 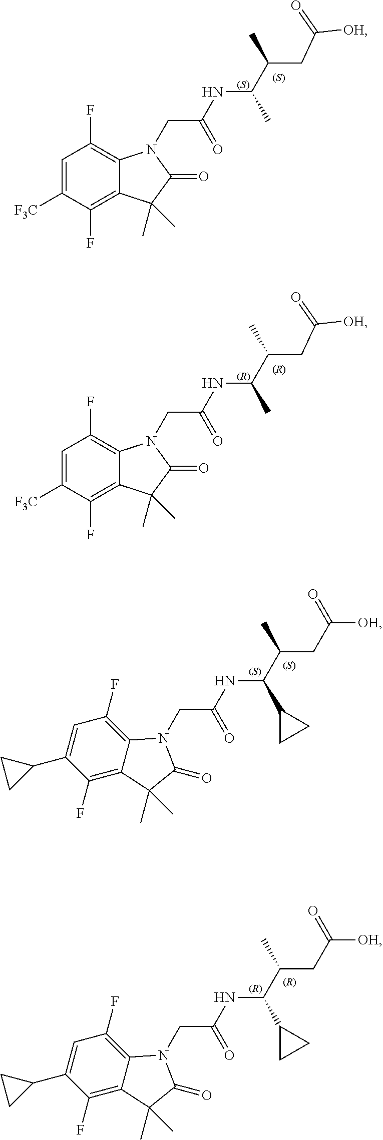 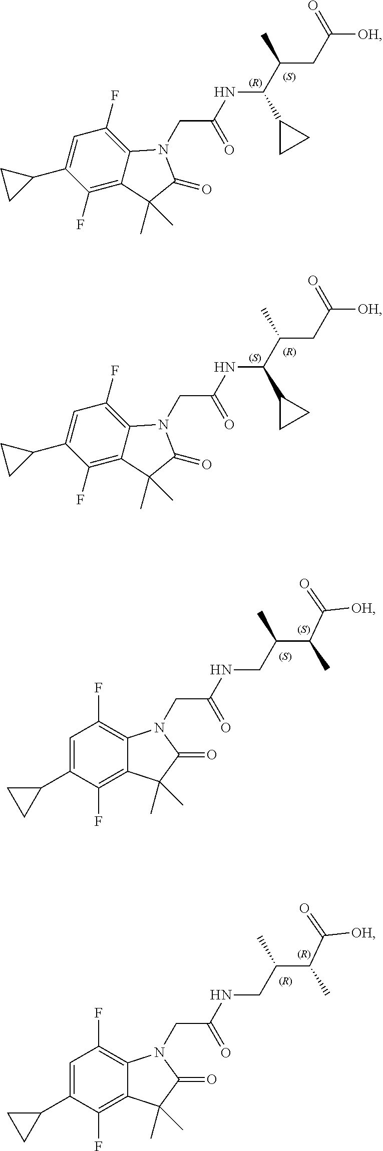 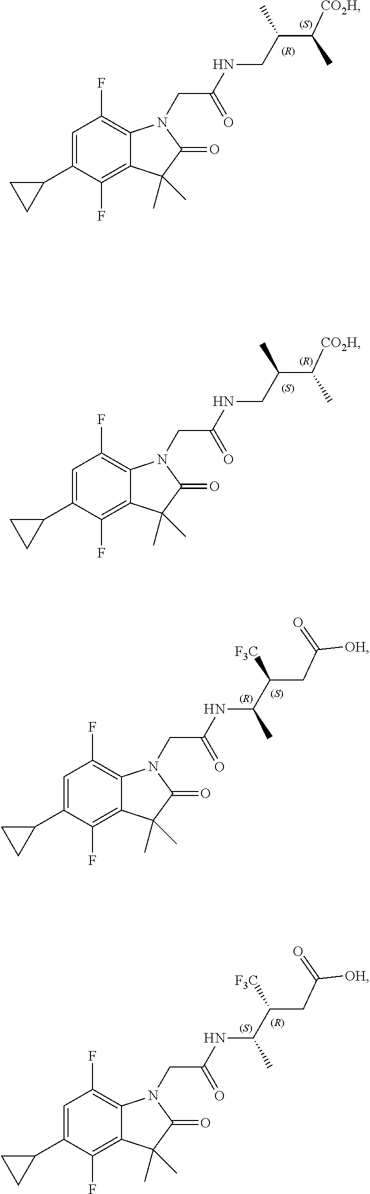 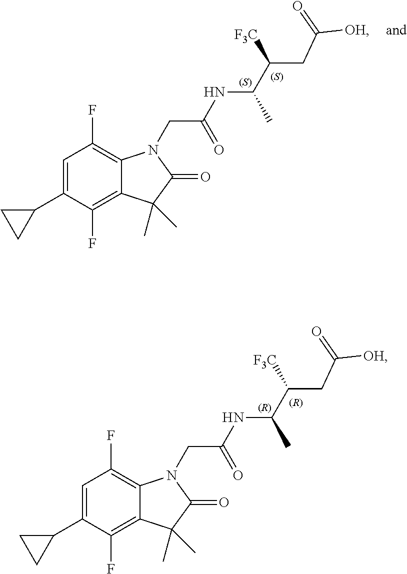
or a pharmaceutically acceptable salt of any of the foregoing.

26. The compound of claim 25, wherein the compound is: 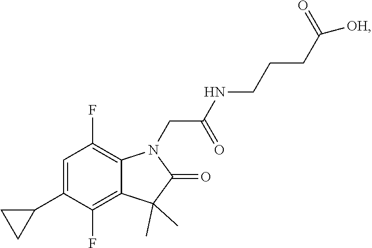 [SMILES: CC1(C)C(=O)N(CC(=O)NCCCC(=O)O)c2c(F)cc(C3CC3)c(F)c21]
or a pharmaceutically acceptable salt thereof.

27. The compound of claim 25, wherein the compound is: 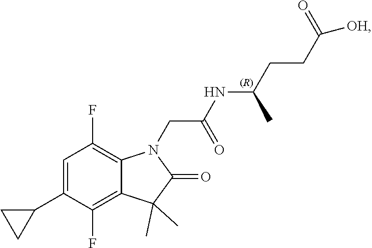
or a pharmaceutically acceptable salt thereof.

28. The compound of claim 25, wherein the compound is: 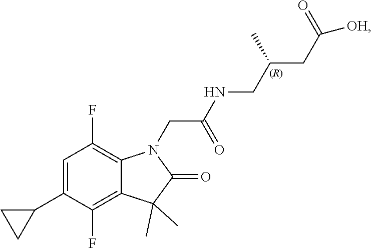
or a pharmaceutically acceptable salt thereof.
